(19) United States
(12) Patent Application Publication
(10) Pub. No.: US 2016/0215306 A1
(43) Pub. Date: Jul. 28, 2016

(54) METHOD FOR PRODUCING MODIFIED RESVERATROL

(71) Applicant: EVOLVA SA, Reinach (CH)

(72) Inventors: Richard Jan Steven Baerends, Koege (DK); Ernesto Simon, Glostrup (DK); Jean Phillippe Meyer, Mulhouse (FR); Carlos Casado Vazquez, Copenhagen (DK)

(73) Assignee: Evolva SA, Reinach (CH)

(21) Appl. No.: 14/915,208

(22) PCT Filed: Aug. 15, 2014
(86) PCT No.: PCT/EP2014/067520

§ 371 Date: Feb. 26, 2016

Related U.S. Application Data
(60) Provisional application No. 61/872,452, filed on Aug. 30, 2013.
(60) Provisional application No. 61/873,348, filed on Sep. 3, 2013.
(60) Provisional application No. 61/917,928, filed on Dec. 18, 2013.
(60) Provisional application No. 61/923,486, filed on Jan. 3, 2014.

(51) Int. Cl. C12P 7/22; C12P 19/44; C12N 9/10

(52) U.S. Cl. CPC C12P 7/22; C12N 9/1007; C12N 9/1051; C12P 19/44

(57) ABSTRACT

Methods for producing glycosylated and methylated resveratrol in a genetically engineered cell, by bioconversion, and in vitro are disclosed herein.

### BACKGROUND OF THE INVENTION

1. Field of the Invention

The invention disclosed herein relates generally to the fields of genetic engineering. Particularly, the invention disclosed herein provides purified preparations of glycosylated or methylated resveratrol and methods for producing and recovering glycosylated or methylated resveratrol from a genetically modified cell. More particularly, the invention disclosed herein provides glycosylated resveratrol preparations having improved solubility for use in foodstuffs and other commercial products and methods for using glycosylated resveratrol of the invention in producing said products.

2. Description of Related Art

Resveratrol (3,5,4′-trihydroxy-stilbene) is a phytophenol belonging to the group of stilbene phytoalexins, which are low-molecular-mass secondary metabolites that constitute the active defense mechanism in plants in response to fungal and other infections or other stress-related events (see, e.g., U.S. 2008/0286844). In addition to its antifungal properties, resveratrol has been recognized for its cardioprotective and cancer chemopreventive activities; it acts as a phytoestrogen, an inhibitor of platelet aggregation (Kopp et al., 1998, European J Endocrinol. 138: 619-620; Gehm et al., 1997, Proc Natl Acad Sci USA 94: 14138-14143; Lobo et al., 1995, Am. J. Obstet. Gynecol. 173: 982-989; Gao & Ming, 2010, Mini Rev Med Chem 10(6):550-67), and an antioxidant (Tang et al., 1997, Science 275: 218-220; Huang, 1997, Food Sci. 24: 713-727).

Plants, the skin of red grapes, and other fruits produce resveratrol naturally. Certain glycosylated resveratrol species or resveratrol glycosides are also found in nature, in plants (mostly in grapevine plants, such as Vitis vinifera and Vitis pseudoreticulata, and mulberry plants). Methylated resveratrol species are also found in nature. For example, pterostilbene, a stilbenoid found in blueberries and grapes, is a double-methylated version of resveratrol that exhibits a higher bioavailability and is more resistant to degradation and elimination (Kapetanovic et al., 2011, Cancer Chemother Pharmacol 68(3):593-601).

Known naturally occurring resveratrol glycosides include: cis/trans-resveratrol-3-O-β-glucoside; resveratrol 3-O-β-D-glucopyranoside; piceid (Kirino et al., 2012, J Nutr Sci Vitaminol 58: 278-286; Larronde et al., 2005, Planta Med. 71: 888-890; Zhou et al., 2001, Planta Med. 67: 158-61; Orsini et al., 1997, J. Nat. Prod. 60: 1082-1087; Waffo-Teguo et al., 1996, Phytochemistry 42: 1591-1593), cis/trans-resveratrol-4′-O-β-glucoside; resveratroloside (Kirino et al., 2012, J Nutr Sci Vitaminol 58: 278-286; Larronde et al., 2005, Planta Med. 71: 888-890; Waffo-Teguo et al., 1998, J. Nat. Prod. 61: 655-657), cis/trans-resveratrol-3,4′-di-O-β-glucoside; Mulberroside E (Larronde et al., 2005, Planta Med. 71: 888-890; Decendit et al., 2002, Phytochemistry 60: 795-798; Zhou et al., 2001, Planta Med. 67: 158-61; Hano et al., 1997, Cell. Mol. Life Sci. 53: 237-241), cis/trans-resveratrol-3,5-di-O-β-glucoside (Larronde et al., 2005, Planta Med. 71: 888-890;); and cis/trans-resveratrol-3,5,4′-tri-O-β-glucoside (Larronde et al., 2005, Planta Med. 71: 888-890).

Resveratrol glycosides that have been produced in vitro or in vivo include: trans-resveratrol-3-O-β-glucoside; piceid (Zhou et al., 2013, J. Nat. Prod. 76: 279-286; Hansen et al. 2009, Phytochemistry 70: 473-482; Weis et al., 2006, Angew. Chem. Int. Ed. 45: 3534-3538; Regev-Shoshani et al., 2003, Biochem J. 374: 157-163; Becker et al., 2003, FEMS Yeast Res. 4: 79-85), trans-resveratrol-4′-O-β-glucoside; resveratroloside (Zhou et al., 2013, J. Nat. Prod. 76: 279-286; Hansen et al., 2009, Phytochemistry 70: 473-482; Weis et al., 2006, Angew. Chem. Int. Ed. 45: 3534-3538; Regev-Shoshani et al., 2003, Biochem J. 374: 157-163), trans-resveratrol-3,4′-di-O-β-glucoside; Mulberroside E (Zhou et al., 2013, J. Nat. Prod. 76: 279-286), trans-glucosyl-α-(1-4)-piceid (Hyunsu et al., 2012, J. Microbiol. Biotechnol. 22: 1698-1704), trans-α-D-maltosyl-(1-4)-piceid (Park et al., 2012, J. Agric. Food Chem. 60: 8183-8189).

Generally, resveratrol is produced in plants and yeast through the phenylpropanoid pathway as illustrated by the reactions shown in FIGS. 1 and 2 and as described in U.S. 2008/0286844, which is incorporated by reference in its entirety herein.

Present production processes rely mostly upon extraction of resveratrol, either from the skin of grape berries or from the plant, Fallopia japonica, known as “Japanese knotweed.” Current extraction and purification methods use organic solvents to extract resveratrol and separate it from the biomass and/or cell debris. Examples of these solvents include, among others, ethanol, methanol, ethyl acetate, and petroleum ether. This is a labor-intensive process and generates low yields. Moreover, since resveratrol or its mono-glucosides (e.g., piceid and resveratroloside) have low water-solubility (see, e.g., Gao & Ming, 2010, Mini Rev Med Chem 10(6):550-67), it forms aggregates/crystals upon addition to water and/or formation by a recombinant resveratrol producing- and secreting-microorganism. Separation of resveratrol aggregates/crystals from recombinant or other cells (such as microorganisms or plant cells) by centrifugation is inefficient.

In yeast, the starting metabolites are malonyl-CoA and phenylalanine or tyrosine (aromatic amino acids). The amino acid L-phenylalanine is converted into trans-cinnamic acid through non-oxidative deamination by L-phenylalanine ammonia lyase (PAL). Next, trans-cinnamic acid is hydroxylated at the para-position to 4-coumaric acid (4-hydroxycinnamic acid) by cinnamate-4-hydroxylase (C4H), a cytochrome P450 monooxygenase enzyme, in conjunction with NADPH:cytochrome P450 reductase (CPR). Alternatively, the amino acid L-tyrosine is converted into 4-coumaric acid by tyrosine ammonia lyase (TAL). The 4-coumaric acid from either alternative pathway is subsequently activated to 4-coumaroyl-CoA by the action of 4-coumarate-CoA ligase (4CL). Finally, stilbene synthase (STS), also known as resveratrol synthase (RS), catalyzes condensation of a phenylpropane unit of 4-coumaroyl-CoA with malonyl-CoA resulting in formation of resveratrol.

Previously, a yeast strain was disclosed that could produce resveratrol from 4-coumaric acid that is found in small quantities in grape must (Becker et al., 2003, FEMS Yeast Res. 4: 79-85). Production of 4-coumaroyl-CoA, and concomitantly resveratrol, in laboratory strains of Saccharomyces cerevisiae, has been achieved by co-expressing a heterologous coenzyme-A ligase gene from hybrid poplar, together with the grapevine resveratrol synthase gene (vst1) (Becker et al., 2003, FEMS Yeast Res. 4: 79-85). Another substrate for resveratrol synthase, malonyl-CoA, is endogenously produced in yeast. Becker et al., 2003, Id., indicated that S. cerevisiae cells produced minute amounts of resveratrol in the piceid form when cultured in synthetic media supplemented with 4-coumaric acid. However, said yeast strain would not be suitable for commercial application because it suffers from low resveratrol yield and requires the addition of 4-coumaric acid, which is expensive and not often present in industrial media. Therefore, there remains a need for an in vivo expression system that produces high yields of resveratrol.

### SUMMARY OF THE INVENTION

It is against the above background that the present invention provides certain advantages and advancements over the prior art.

Although this invention disclosed herein is not limited to specific advantages or functionality, the invention provides a method for producing a glycosylated stilbene, comprising:
(a) growing a recombinant host in a culture medium, under conditions in which the host produces a stilbene,
   wherein the host comprises a gene encoding a glycosyltransferase (UGT) polypeptide capable of in vivo glycosylation of the stilbene comprising a recombinant expression construct;
   wherein the gene encoding the UGT polypeptide is expressed in the host, wherein the stilbene is glycosylated in the host thereby; and
(b) recovering the glycosylated stilbene from the culture media.

In some aspects of the method for producing the glycosylated stilbene disclosed herein, the recombinant host does not express an exo-1,3-beta-glucanase.

In some aspects of the method for producing the glycosylated stilbene disclosed herein, the UGT polypeptide comprises:
(a) a UGT72B2 polypeptide having at least 55% identity to the amino acid sequence set forth in SEQ ID NO: 18;
(b) a UGT71E1 polypeptide having at least 60% identity to the amino acid sequence set forth in SEQ ID NO: 4;
(c) a UGT73B5 polypeptide having at least 55% identity to the amino acid sequence set forth in SEQ ID NO: 20;
(d) a UGT84B1 polypeptide having at least 40% identity to the amino acid sequence set forth in SEQ ID NO: 32;
(e) a UGT75B2 polypeptide having at least 50% identity to the amino acid sequence set forth in SEQ ID NO: 22;
(f) a UGT73C5 polypeptide having at least 60% identity to the amino acid sequence set forth in SEQ ID NO: 40; or
(g) a UGT73C3 polypeptide having at least 60% identity to the amino acid sequence set forth in SEQ ID NO: 38.

In some aspects of the method for producing a glycosylated stilbene disclosed herein, the stilbene comprises 3, 4′, and 5 hydroxyl groups, wherein the glycosylated stilbene comprises one or a plurality of sugar moieties covalently linked to the one or more of the 3, 4′, or 5 hydroxyl groups of the stilbene.

In some aspects of the method for producing the glycosylated stilbene disclosed herein, the glycosylated stilbene is monoglycosylated at one of the 3, 4′, or 5 hydroxyl groups, diglycosylated at the 3,4′, 3,5, or 4′,5 hydroxyl groups, or triglyosylated at the 3, 4′, 5 hydroxyl groups.

In some embodiments, the method for producing the glycosylated stilbene disclosed herein further comprises the step of cleavage of sugar moieties of the glycosylated stilbene, wherein the stilbene can be recovered from the culture media.

In some aspects of the method for producing the glycosylated stilbene disclosed herein, cleavage of the sugar moieties of the glycosylated stilbene comprises enzymatic cleavage.

In some aspects of the method for producing the glycosylated stilbene disclosed herein, enzymatic cleavage comprises treating the culture medium with an enzyme capable of cleaving sugar moieties.

In some aspects of the method for producing the glycosylated stilbene disclosed herein, the enzyme used in enzymatic cleavage of the sugar moieties of the glycosylated stilbene comprises β-glucosidase, cellulase, glusulase, cellobiase, β-galactosidase, β-glucuronidase, or EXG1.

In some aspects of the method for producing the glycosylated stilbene disclosed herein, cleavage of the sugar moieties of the glycosylated stilbene comprises chemical cleavage.

In some aspects of the method for producing the glycosylated stilbene disclosed herein, chemical cleavage comprises treating the culture medium with a weak acid or under other conditions capable of cleaving sugar moieties.

In some aspects of the method for producing the glycosylated stilbene disclosed herein, the weak acid used in chemical cleavage of the sugar moieties of the glycosylated stilbene comprises an organic acid or an inorganic acid.

In some embodiments, the method for producing the glycosylated stilbene disclosed herein further comprises the step of detecting the recovered stilbene by thin layer chromatography (TLC), high-performance liquid chromatography (HPLC), ultraviolet-visible spectroscopy/spectrophotometry (UV-Vis), liquid chromatography-mass spectrometry (LC-MS), or nuclear magnetic resonance (NMR).

In some aspects of the method for producing the glycosylated stilbene disclosed herein, the stilbene comprises resveratrol.

In some aspects of the method for producing the glycosylated stilbene disclosed herein, the glycosylated stilbene comprises piceid (3-resveratrol monoglucoside or 5-resverarol monoglucoside), resveratroloside (4′-resveratrol monoglucoside), Mulberroside E (3,4′-resveratrol diglucoside), 3,5-resveratrol diglucoside, and 3,5,4′-resveratrol triglucoside.

The invention further provides a method for producing a glycosylated stilbene from a bioconversion reaction, comprising
(a) growing a recombinant host in a culture medium, under conditions in which glycosyltransferase enzymes (UGTs) are produced in said host,
   wherein the host comprises a gene encoding a glycosyltransferase (UGT) polypeptide capable of in vivo glycosylation of a stilbene comprising a recombinant expression construct;
   wherein the gene encoding the UGT polypeptide is expressed in the host;
(b) contacting the host with a stilbene in a reaction buffer to produce a glycosylated stilbene; and
(c) purifying the glycosylated stilbene.

In some aspects of the method for producing the glycosylated stilbene from a bioconversion reaction, the host takes up and glycosylates the stilbene in the cell, and the glycosylated stilbene is released into the culture medium.

In some aspects of the method for producing the glycosylated stilbene from a bioconversion reaction, the UGT polypeptide comprises:
(a) a UGT71E1 polypeptide having at least 60% identity to the amino acid sequence set forth in SEQ ID NO: 4;
(b) a UGT88A1 polypeptide having at least 50% identity to the amino acid sequence set forth in SEQ ID NO: 8;
(c) a CaUGT2 polypeptide having at least 60% identity to the amino acid sequence set forth in SEQ ID NO: 10;
(d) a UGT73B2 polypeptide having at least 55% identity to the amino acid sequence set forth in SEQ ID NO: 14; or
(e) a PaGT3 polypeptide having at least 60% identity to the amino acid sequence set forth in SEQ ID NO: 120.

In some aspects of the method for producing the glycosylated stilbene from a bioconversion reaction, the stilbene comprises a plant-derived or synthetic stilbene.

In some aspects of the method for producing the glycosylated stilbene from a bioconversion reaction, the glycosylated stilbene produced comprises mono-, di-, tri- or poly-glycosylated stilbene molecules.

In some aspects of the method for producing the glycosylated stilbene from a bioconversion reaction, the glycosylated stilbene produced is separated from the culture media through filtration or centrifugation.

In some embodiments of the method for producing the glycosylated stilbene from a bioconversion reaction further comprises the step of cleaving sugar moieties of the glycosylated stilbene, wherein cleavage comprises treating the glycosylated stilbene with an enzyme capable of cleaving sugar moieties.

In some aspects of the method for producing the glycosylated stilbene from a bioconversion reaction the enzyme used to cleave sugar moieties of the glycosylated stilbene comprises β-glucosidase, cellulase, glusulase, cellobiase, β-galactosidase, β-glucuronidase, or EXG1.

In some aspects of the method for producing the glycosylated stilbene from a bioconversion reaction, the stilbene comprises resveratrol.

In some aspects of the method for producing the glycosylated stilbene from a bioconversion reaction, the glycosylated stilbene comprises piceid (3-resveratrol monoglucoside or 5-resverarol monoglucoside), resveratroloside (4′-resveratrol monoglucoside), Mulberroside E (3,4′-resveratrol diglucoside), 3,5-resveratrol diglucoside, and 3,5,4′-resveratrol triglucoside.

The invention further provides a method for producing a methylated stilbene, comprising
(a) growing a recombinant host in a culture medium, under conditions in which the host produces a stilbene,
   wherein the host comprises a gene encoding a methyltransferase polypeptide capable of in vivo methylation of the stilbene comprising a recombinant expression construct;
   wherein the gene encoding the methyltransferase polypeptide is expressed in the host, wherein the stilbene is methylated in the host; and
(b) recovering the methylated stilbene from the culture media.

In some aspects of the method for producing a methylated stilbene, the gene encoding the methyltransferase polypeptide comprises a gene encoding a resveratrol O-methyltransferase (ROMT) polypeptide.

In some aspects of the method for producing a methylated stilbene, the ROMT polypeptide comprises Vitis vinifera ROMT polypeptide having at least 70% identity to the amino acid sequence set forth in SEQ ID NO: 6.

In some aspects of the method for producing a methylated stilbene, the methylated stilbene is methylated at hydroxyl groups of the stilbene, wherein methylation comprises covalently attaching one or a plurality of methyl groups at one or more of the hydroxyl groups of the stilbene.

In some aspects of the method for producing a methylated stilbene, the stilbene comprises 3, 4′, and 5 hydroxyl groups, wherein the methylated stilbene is monomethylated at 3, 4′, or 5 hydroxyl groups; dimethylated at 3,4′, 3,5, or 4′,5 hydroxyl groups; or is trimethylated at 3, 4′, 5 hydroxyl groups.

In some embodiments, the method for producing a methylated stilbene further comprises the step of detecting recovered the methylated stilbene by thin layer chromatography (TLC), high-performance liquid chromatography (HPLC), ultraviolet-visible spectroscopy/spectrophotometry (UV-Vis), liquid chromatography-mass spectrometry (LC-MS), or nuclear magnetic resonance (NMR).

In some aspects of the method for producing a methylated stilbene, the stilbene is resveratrol.

In some aspects of the method for producing a methylated stilbene, the methylated stilbene comprises pterostilbene (3,5-dimethoxy-4′-hydroxy-trans-stilbene), 3,5,4′-trimethoxystilbene, pinostilbene, tetramethoxystilbene, pentamethoxystilbene, and N-Hydroxy-N-(trimethoxphenyl)-trimethoxy-benzamidine.

The invention further provides a method for producing a methylated stilbene from a bioconversion reaction, comprising
(a) growing a recombinant host in a culture medium, under conditions in which methyltransferase enzymes are produced in said host,
   wherein the host comprises a gene encoding a methyltransferase polypeptide capable of in vivo methylation of a stilbene comprising a recombinant expression construct;
   wherein the gene encoding the methyltransferase polypeptide is expressed in the host;
(c) contacting the host with a stilbene in a reaction buffer to produce a methylated stilbene; and
(d) purifying the methylated stilbene.

In some aspects of the method for producing the methylated stilbene from a bioconversion reaction, the host takes up and methylates the stilbene in the cell, and the methylated stilbene is released into the culture medium.

In some aspects of the method for producing the methylated stilbene from the bioconversion reaction, the methyltransferase polypeptide comprises a resveratrol O-methyltransferase (ROMT) polypeptide having at least 70% identity to the amino acid sequence set forth in SEQ ID NO: 6.

In some aspects of the method for producing the methylated stilbene from the bioconversion reaction, the stilbene comprises a plant-derived or synthetic stilbene.

In some aspects of the method for producing the methylated stilbene from the bioconversion reaction, the methylated stilbene comprises mono-, di-, tri- or poly-methylated stilbene molecules.

In some aspects of the method for producing the methylated stilbene from the bioconversion reaction, the stilbene comprises resveratrol.

In some aspects of the method for producing the methylated stilbene from the bioconversion reaction, the methylated stilbene comprises pterostilbene (3,5-dimethoxy-4′-hydroxy-trans-stilbene), 3,5,4′-trimethoxystilbene, pinostilbene, tetramethoxystilbene, pentamethoxystilbene, and N-Hydroxy-N-(trimethoxphenyl)-trimethoxy-benzamidine.

The recombinant host used in the methods disclosed herein can be a microorganism that is a yeast cell, a plant cell, a mammalian cell, an insect cell, a fungal cell, or a bacterial cell.

In some aspects, the bacterial cell used in the methods disclosed herein comprises Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacterial cells.

In some aspects, the yeast cell used in the methods disclosed herein is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

In some aspects, the yeast cell used in the methods disclosed herein is a Saccharomycete.

In some aspects, the yeast cell used in the methods disclosed herein is a cell from the Saccharomyces cerevisiae species.

In some aspects, the yeast cell used in the methods disclosed herein the yeast cell comprises a S. cerevisiae yeast cell that does not express EXG1.

The invention further provides a recombinant host comprising:
(a) a gene encoding a glycosyltransferase (UGT) polypeptide, wherein the UGT polypeptide is capable of in vivo glycosylation of a stilbene; and/or
(b) a gene encoding a methyltransferase polypeptide, wherein the methyltransferase polypeptide is capable of in vivo methylation of a stilbene;

wherein at least one of said genes is a recombinant gene, wherein the host is capable of producing a stilbene.

In some aspect, the recombinant host disclosed herein comprises the UGT polypeptide comprising
(a) a UGT72B2 polypeptide having at least 55% identity to the amino acid sequence set forth in SEQ ID NO: 18;
(b) a UGT71E1 polypeptide having at least 60% identity to the amino acid sequence set forth in SEQ ID NO: 4;
(c) a UGT73B5 polypeptide having at least 55% identity to the amino acid sequence set forth in SEQ ID NO: 20;
(d) a UGT84B1 polypeptide having at least 40% identity to the amino acid sequence set forth in SEQ ID NO: 32;
(e) a UGT75B2 polypeptide having at least 50% identity to the amino acid sequence set forth in SEQ ID NO: 22;
(f) a UGT73C5 polypeptide having at least 60% identity to the amino acid sequence set forth in SEQ ID NO: 40; or
(g) a UGT73C3 polypeptide having at least 60% identity to the amino acid sequence set forth in SEQ ID NO: 38.

In some aspect, the recombinant host disclosed herein comprises the gene encoding the methyltransferase polypeptide comprising a ROMT polypeptide having at least 70% identity to the amino acid sequence set forth in SEQ ID NO: 6.

In some aspects, the recombinant host disclosed herein comprises recombinant genes encoding the UGT polypeptide or the methyltransferase polypeptide capable of in vivo glycosylation and/or methylation of a stilbene, wherein the stilbene is resveratrol.

The invention further provides a recombinant host comprising one or more of:
(a) a gene encoding a L-phenylalanine ammonia lyase (PAL) polypeptide;
(b) a gene encoding a cinnamate-4-hydroxylase (C4H) polypeptide;
(c) a gene encoding a NADPH:cytochrome P450 reductase polypeptide;
(d) a gene encoding a tyrosine ammonia lyase (TAL);
(e) a gene encoding a 4-coumarate-CoA ligase (4CL); or
(f) a gene encoding stilbene synthase (STS);

wherein at least one of said genes is a recombinant gene, wherein the host is capable of producing a stilbene.

In some aspects, the host disclosed herein produces the stilbene from a carbon source when fed a precursor, wherein the precursor comprises coumaric acid.

In some aspects, the host disclosed herein is a microorganism that is a yeast cell, a plant cell, a mammalian cell, an insect cell, a fungal cell, or a bacterial cell.

In some embodiments, the bacterial cell comprises Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacterial cells.

In some embodiments, the the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

In some embodiments, the yeast cell is a Saccharomycete.

In some embodiments, the yeast cell is a cell from the Saccharomyces cerevisiae species.

In some embodiments, the yeast cell comprises an S. cerevisiae yeast cell that does not express EXG1.

The invention further provides a method for producing a glycosylated stilbene from an in vitro reaction comprising contacting a stilbene with one or more UGT polypeptides in the presence of one or more UDP-sugars.

In some aspects of the method for producing the glycosylated stilbene from the in vitro reaction, the one or more UGT polypeptides comprises:
(a) a UGT72B2 polypeptide having at least 55% identity to the amino acid sequence set forth in SEQ ID NO: 18;
(b) a UGT71E1 polypeptide having at least 60% identity to the amino acid sequence set forth in SEQ ID NO: 4;
(c) a UGT73B5 polypeptide having at least 55% identity to the amino acid sequence set forth in SEQ ID NO: 20;
(d) a UGT84B1 polypeptide having at least 40% identity to the amino acid sequence set forth in SEQ ID NO: 32;
(e) a UGT88A1 polypeptide having at least 50% identity to the amino acid sequence set forth in SEQ ID NO: 8;
(f) a UGT75B2 polypeptide having at least 50% identity to the amino acid sequence set forth in SEQ ID NO: 22;
(g) a UGT78D2 polypeptide having at least 55% identity to the amino acid sequence set forth in SEQ ID NO: 30;
(h) a UGT73C5 polypeptide having at least 60% identity to the amino acid sequence set forth in SEQ ID NO: 40;
(i) a UGT73C3 polypeptide having at least 60% identity to the amino acid sequence set forth in SEQ ID NO: 38; or
(j) a UGT73B2 polypeptide having at least 55% identity to the amino acid sequence set forth in SEQ ID NO: 14;

wherein at least one of the UGT polypeptides is a recombinant UGT polypeptide.

In some aspects of the method for producing the glycosylated stilbene from the in vitro reaction disclosed herein, the stilbene comprises a plant-derived or synthetic stilbene.

In some aspects of the method for producing the glycosylated stilbene from the in vitro reaction disclosed herein, the glycosylated stilbene produced comprises mono-, di-, tri- or poly-glycosylated stilbene molecules.

In some aspects, the one or more UDP-sugars used in the method for producing the glycosylated stilbene from the in vitro reaction disclosed herein comprise UDP-glucose, UDP-rhamnose, or UDP-xylose.

In some embodiments of the method for producing the glycosylated stilbene from the in vitro reaction disclosed herein, the stilbene comprises resveratrol.

In some embodiments of the method for producing the glycosylated stilbene from the in vitro reaction disclosed herein, the glycosylated stilbene comprises piceid (3-resveratrol monoglucoside or 5-resverarol monoglucoside), resveratroloside (4′-resveratrol monoglucoside), Mulberroside E (3,4′-resveratrol diglucoside), 3,5-resveratrol diglucoside, and 3,5,4′-resveratrol triglucoside.

The invention further provides a method for producing a methylated stilbene from an in vitro reaction comprising contacting a stilbene with one or more methyltransferase polypeptides.

In some aspects of the method for producing the methylated stilbene from the in vitro reaction disclosed herein, the one or more methyltransferase polypeptides comprises an ROMT polypeptide having at least 70% identity to the amino acid sequence set forth in SEQ ID NO: 6.

In some aspects of the method for producing the methylated stilbene from the in vitro reaction disclosed herein, the stilbene comprises a plant-derived or synthetic stilbene.

In some aspects of the method for producing the methylated stilbene from the in vitro reaction disclosed herein, the methylated stilbene produced comprises mono-, di-, tri- or poly-methylated stilbene molecules.

In some aspects of the method for producing the methylated stilbene from the in vitro reaction disclosed herein, the stilbene comprises resveratrol.

In some aspects of the method for producing the methylated stilbene from the in vitro reaction disclosed herein, the methylated stilbene comprises pterostilbene (3,5-dimethoxy-4′-hydroxy-trans-stilbene), 3,5,4′-trimethoxystilbene, pinostilbene, tetramethoxystilbene, pentamethoxystilbene, and N-Hydroxy-N-(trimethoxphenyl)-trimethoxy-benzamidine.

The invention further provides a method for producing resveratrol glycosides comprising bioconversion of resveratrol or a plant extract using one or more UGT polypeptides and one or more UDP-sugars, wherein the bioconversion comprises contacting the resveratrol or the plant extract with the one or more UGT polypeptides in the presence of the one or more UDP-sugars, wherein the bioconversion comprises in vitro enzymatic or whole cell bioconversion.

In some aspects, the one or more UGT polypeptides used in the method for producing resveratrol glycosides through bioconversion disclosed herein comprises:
(a) a UGT71E1 polypeptide having at least 60% identity to the amino acid sequence set forth in SEQ ID NO: 4;
(b) a UGT88A1 polypeptide having at least 50% identity to the amino acid sequence set forth in SEQ ID NO: 8;
(c) a CaUGT2 polypeptide having at least 60% identity to the amino acid sequence set forth in SEQ ID NO: 10;
(d) a UGT73B2 polypeptide having at least 55% identity to the amino acid sequence set forth in SEQ ID NO: 14; or
(e) a PaGT3 polypeptide having at least 60% identity to the amino acid sequence set forth in SEQ ID NO: 120;

wherein at least one of the UGT polypeptides is a recombinant UGT polypeptide.

In some aspects, the one or more UDP-sugars used in the method for producing resveratrol glycosides through bioconversion disclosed herein comprise UDP-glucose, UDP-rhamnose, or UDP-xylose.

The invention further provides a method for producing methylated resveratrol comprising bioconversion of a resveratrol or a plant extract using one or more methyltransferase polypeptides, wherein the bioconversion comprises contacting the resveratrol or the plant extract with the one or more methyltransferase polypeptides, wherein the bioconversion comprises in vitro enzymatic or whole cell bioconversion.

In some aspects, the one or more methyltransferase polypeptides used in the method for producing methylated resveratrol through bioconversion disclosed herein comprises an ROMT polypeptide having at least 70% identity to the amino acid sequence set forth in SEQ ID NO: 6.

The invention further provides a method for producing glycosylated pterostilbene comprising bioconversion of a pterostilbene using one or more UGT polypeptides and one or more UDP-sugars, wherein the bioconversion comprises contacting the pterostilbene with the one or more UGT polypeptides in the presence of the one or more UDP-sugars, wherein the bioconversion comprises in vitro enzymatic or whole cell bioconversion.

In some aspects, the one or more UGT polypeptides used in the method for producing glycosylated pterostilbene through bioconversion disclosed herein comprises:
(a) a UGT71E1 polypeptide having at least 60% identity to the amino acid sequence set forth in SEQ ID NO: 4;
(b) a UGT88A1 polypeptide having at least 50% identity to the amino acid sequence set forth in SEQ ID NO: 8;
(c) a CaUGT2 polypeptide having at least 60% identity to the amino acid sequence set forth in SEQ ID NO: 10;
(d) a UGT73B2 polypeptide having at least 55% identity to the amino acid sequence set forth in SEQ ID NO: 14; or
(e) a PaGT3 polypeptide having at least 60% identity to the amino acid sequence set forth in SEQ ID NO: 120;

wherein at least one of the UGT polypeptides is a recombinant UGT polypeptide.

In some aspects, the one or more UDP-sugars used in the method for producing glycosylated pterostilbene through bioconversion disclosed herein comprise UDP-glucose, UDP-rhamnose, or UDP-xylose.

The invention further provides a composition comprising glycosylated or methylated resveratrol, wherein the resveratrol composition does not contain plant-derived contaminant compounds.

In some aspects, the resveratrol composition disclosed herein is mono, di, tri or poly-glycosylated and/or mono, di, or tri-methylated.

In some aspects, the resveratrol composition disclosed herein is covalently attached to sugar moieties, wherein the sugar moieties are monosaccharides, disaccharides, or polysaccharides.

In some aspects, the monosaccharide is glucose, fructose, xylose, rhamnose, arabinose, glucuronic acid, erythrose, ribose, or galactose.

In some aspects, the disaccharide is sucrose, maltose, or lactose.

In the methods disclosed herein, a gene encoding a UDP-glycosyltransferase UGT polypeptide or a methyltransferase polypeptide comprises a sequence of amino acid-encoding codons that have been optimized for expression in the cell.

In the methods disclosed herein, a gene encoding resveratrol O-methyltransferase (ROMT) polypeptide comprises a sequence of amino acid-encoding codons that have been optimized for expression in the cell.

The invention further provides methods for purifying resveratrol from a cell, comprising
(a) growing a recombinant host in a culture medium, under conditions in which the host produces resveratrol,
   wherein the host comprises a gene encoding a glycosyltransferase (UGT) polypeptide capable of in vivo glycosylation of resveratrol comprising a recombinant expression construct;
   wherein the gene encoding the UGT polypeptide is expressed in the host, wherein resveratrol is glycosylated in the host thereby,
(b) recovering glycosylated resveratrol from the culture media;
(c) treating the glycosylated resveratrol with an enzyme capable of cleaving sugar moieties; and
(d) recovering insoluble resveratrol aglycon.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic diagram of the resveratrol pathway from L-phenylalanine or L-tyrosine in plants and yeast.

FIG. 2 shows a schematic diagram of a pathway for producing resveratrol from glucose in yeast.

FIG. 3A indicates three hydroxyl (—OH) groups (3, 5 and 4′) of resveratrol that can be glycosylated and shows reaction catalyzed by a UGT to produce piceid from resveratrol. FIG. 3B shows the chemical structures for Glc(α1,4)-piceid and maltosyl(a 1,4)-piceid.

FIG. 4 is a chromatogram showing formation of 3,5-resveratrol diglucoside, 3,4′-resveratrol diglucoside, and 3,5,4′-resveratrol triglucoside from piceid.

FIG. 5 shows the names, CAS Registry numbers, molecular weights, and aqueous solubilities of various resveratrol glycoside molecules.

FIG. 6A shows the addition of a glucose molecule on resveratroloside (substrate) by BpUGT94B1 R25S (SEQ ID NOs: 15, 16). FIG. 6B shows addition of a glucose molecule on 3,4′-resveratrol diglucoside (substrate) by BpUGT94B1 R25S (SEQ ID NOs: 15, 16). FIG. 6C shows that a glucuronic acid molecule is not added by BpUGT94B1 (SEQ ID NOs: 1, 2). FIG. 6D shows the addition of a glucuronic acid molecule on 3,4′-resveratrol diglucoside (substrate) by BpUGT94B1 (SEQ ID NOs: 1, 2).

FIG. 7 shows that addition of multiple glucose moieties to resveratrol improves solubility by a factor on the order of several thousand.

FIG. 8 depicts a method for separating resveratrol glycosides from cells and subsequent purification and recovery of resveratrol from resveratrol glycosides.

FIG. 9A shows HPLC chromatograms of a Mulberroside E (3,4′-resveratrol diglucoside) sample before and after incubation with a cellulase. FIG. 9B quantifies soluble and insoluble resveratrol following centrifugation of a cellulase-treated Mulberroside E sample.

FIG. 10 quantifies resveratrol, resveratroloside, piceid, and 3,5-resveratrol diglucoside levels from a resveratrol-producing yeast strain expressing the indicated UGT polypeptides, as described in Example 5.

FIG. 11 shows a chromatogram analyzing broth of a resveratrol-producing strain not expressing a UGT polypeptide, as described in Example 5.

FIG. 12 shows a chromatogram analyzing broth of a resveratrol-producing strain expressing UGT71E1 (SEQ ID NOs: 3, 4), as described in Example 5.

FIG. 13 shows a chromatogram analyzing broth of a resveratrol-producing strain expressing UGT84B1 (SEQ ID NOs: 31, 32), as described in Example 5.

FIG. 14 shows a chromatogram of analyzing broth resveratrol-producing strain expressing UGT73B5 (SEQ ID NOs: 19, 20), as described in Example 5.

FIG. 15 shows a chromatogram analyzing broth of a resveratrol-producing strain not expressing a UGT polypeptide, as described in Example 6.

FIG. 16 shows formation of resveratroloside and resveratrol by a resveratrol-producing strain expressing UGT72B2_GA (SEQ ID NOs: 63, 18), as described in Example 6.

FIG. 17 shows formation of 3,5-resveratrol diglucoside, piceid, and resveratrol by a resveratrol-producing strain expressing UGT71E1 (SEQ ID NOs: 3, 4).

FIG. 18 shows a schematic overview of in vivo resveratrol production and recovery of resveratrol as described in Example 7.

FIGS. 19A and 19B show piceid, resveratroloside, and 3,5-resveratrol diglucoside formation following bioconversion of resveratrol with yeast expressing UGT71E1 (SEQ ID NOs: 3, 4). FIG. 19C shows piceid and resveratroloside formation following bioconversion of resveratrol from knotweed root extracts. FIG. 19D shows formation of resveratrol from resveratrol glucosides in knotweed root extract samples treated with β-glucosidase.

FIG. 20A is an HPCL chromatogram showing piceid and resveratroloside production by E. coli cells expressing UGT PaGT3 (SEQ ID NOs: 119, 120) and supplemented with resveratrol. FIG. 20B shows a chromatogram analyzing the broth of E. coli cells that do not express a UGT polypeptide yet are supplemented with resveratrol.

FIG. 21 shows plasma levels of resveratrol, resveratrol glucoside, and metabolites following oral or intravenous administration of resveratrol (A, B), resveratroloside (C, D), piceid (E, F), 3,5-resveratrol diglucoside (G, H), or 3,4′-resveratrol diglucoside (I, J). FIGS. 21K and 21L compare resveratrol, resveratroloside, piceid, 3,5-resveratrol diglucoside, and 3,4′-resveratrol diglucoside levels in plasma following oral or intravenous administration.

FIG. 22 quantifies resveratrol, resveratroloside, piceid, 3,5-resveratrol diglucoside, and 3,4′-resveratrol diglucoside levels in plasma 0.5, 1, 2, 4 h post-administration.

FIG. 23 compares the molecular structures of pterostilbene and resveratrol.

FIG. 24A shows a chromatogram of a pterostilbene standard at 306 nm. FIG. 24B shows a UV-Vis spectrum of the pterostilbene standard at 306 nm.

FIG. 25 shows a chromatogram of resveratrol-producing strain expressing an ROMT polypeptide (SEQ ID NOs: 5, 6), as described in Example 11.

FIG. 26A shows an HPLC chromatogram analyzing broth of an ROMT-expressing yeast strain supplemented with resveratrol, and FIG. 26B shows an HPLC chromatogram of a pterostilbene standard. FIG. 26C shows a UV-Vis spectrum of broth of an ROMT-expressing yeast strain supplemented with resveratrol, and FIG. 26D shows a UV-Vis spectrum of a pterostilbene standard.

FIGS. 27A and 27B show an HPLC chromatogram and a UV-Vis spectrum, respectively, of a glycosylated pterostilbene produced by bioconversion.

FIGS. 28A and 28B show an HPLC chromatogram and a UV-Vis spectrum, respectively, of a glycosylated pterostilbene sample treated with a β-glucosidase.

FIG. 29A shows a mass spectrometry total ion current plot for a glycosylated pterostilbene (see Example 13). FIG. 29B shows the molecular weight of the glycosylated pterostilbene peak identified in FIG. 29A.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Methods well known to those skilled in the art can be used to construct genetic expression constructs and recombinant cells according to this invention. These methods include in vitro recombinant DNA techniques, synthetic techniques, in vivo recombination techniques, and PCR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

### Resveratrol Modifications

In some embodiments, a stilbene or a modified stilbene is produced in vitro, by bioconversion, or in a cell. In some embodiments, the modified stilbene is glycosylated and/or methylated. In some embodiments, the stilbene is resveratrol or a resveratrol derivative. As used herein, the terms “modified resveratrol,” “resveratrol derivative,” and “resveratrol analog” can be used interchangeably to refer to a compound that can be derived from resveratrol or a compound with a similar structure to resveratrol. As used herein, the terms “resveratrol derivative” or “resveratrol analog” can be used interchangeably to refer to resveratrol-like molecules such as to glycosylated resveratrol molecules, methylated resveratrol molecules, or resveratrol molecules that are glycosylated and methylated.

As used herein, the terms “glycosylation,” “glycosylate,” “glycosylated,” and “protection group(s)” can be used interchangeably to refer to the chemical reaction in which a carbohydrate molecule is covalently attached to a hydroxyl group or attached to another functional group in a molecule capable of being covalently attached to a carbohydrate molecule. The term “mono” used in reference to glycosylation refers to the attachment of one carbohydrate molecule. The term “di” used in reference to glycosylation refers to the attachment of two carbohydrate molecules. The term “tri” used in reference to glycosylation refers to the attachment of three carbohydrate molecules. Additionally, the terms “oligo” and “poly” used in reference to a glycosylated molecule refers to the attachment of two or more carbohydrate molecules and can encompass embodiments comprising a mixture of resveratrol molecules having a variety of attached carbohydrate molecules.

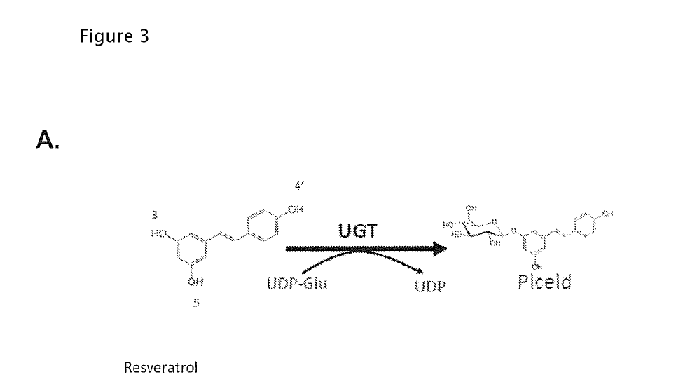

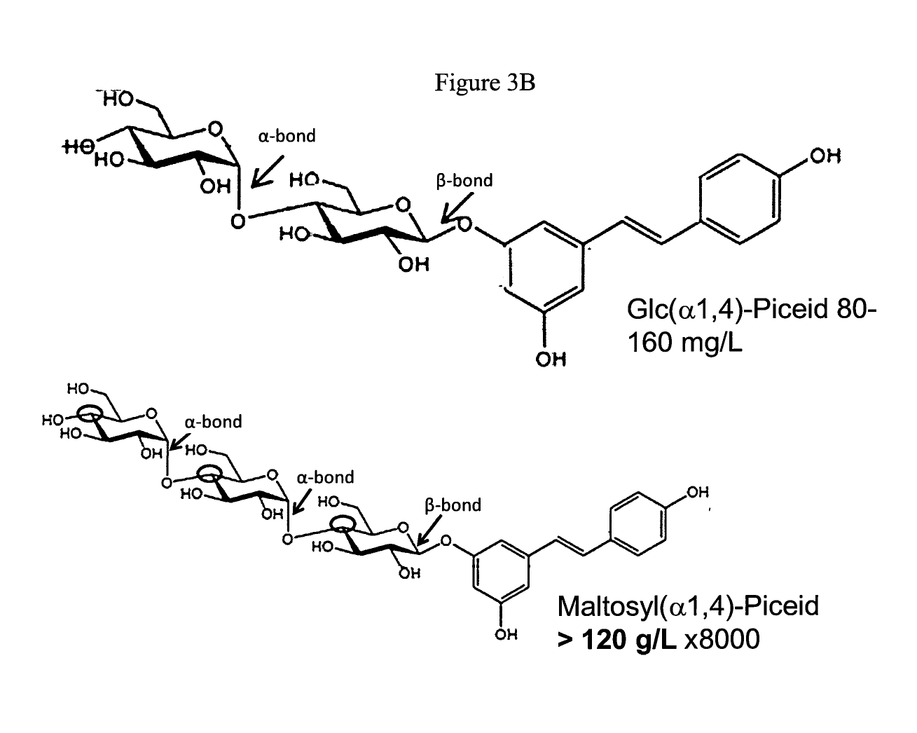

As disclosed herein, the term “glycosylated resveratrol” refers to resveratrol glycosylated at the 3 hydroxyl group, or the 4′ hydroxyl group, or the 5 hydroxyl group of resveratrol, wherein glycosylation comprises covalently attaching one or a plurality of sugar or saccharide residues at one or more of the 3, 4′, or 5 hydroxyl groups of resveratrol (FIG. 3). The saccharide moiety in each position can be independently zero, one, two, three, or multiple sugar residues, wherein all the sugar residues can be the same sugar residues or different sugar residues.

As disclosed herein, the terms “sugar” and “carbohydrate” encompass monosaccharides, disaccharides, and polysaccharides. One of skill in the art would appreciate that resveratrol can be modified with glucose, xylose, galactose, N-acetylglucosamine, rhamnose, glucuronic acid, or other sugar moieties. In addition, one or more additional sugar moieties can be linked to the glucose, xylose, galactose, N-acetylglucosamine, rhamnose, or other sugar moiety via various glycosidic linkages (such as 1,2 linkages, 1,4-linkages, 1,3-linkages, or 1,6-linkages between the sugar moieties). Furthermore, one of skill in the art will appreciate that resveratrol analogs or derivatives (e.g., pterostilbene, 3,5-dihydroxypterostilbene, or other resveratrol derivatives such as piceatannol) also can be glycosylated as described herein for resveratrol.

As used herein, the term “resveratrol glycoside” can be used to refer to a molecule of resveratrol to which a sugar is bound to another functional group through a glycosidic bond. Examples of resveratrol derivatives include, but are not limited to, cis/trans-resveratrol-3-O-β-glucoside, resveratrol 3-O-β-D-glucopyranoside (piceid), cis/trans-resveratrol-4′-O-β-glucoside (resveratroloside), cis/trans-resveratrol-3,4′-di-O-β-glucoside (Mulberroside E), cis/trans-resveratrol-3,5-di-O-β-glucoside, cis/trans-resveratrol-3,5,4′-tri-O-β-glucoside, trans-glucosyl-α-(1-4)-piceid, trans-resveratrol-4′,5-di-O-β-glucoside, and 3-glucuronide-resveratrol. In certain embodiments of the invention, the resveratrol derivative is polydatin, piceid (also known as 2-[3-Hydroxy-5-[(E)-2-(4-hydroxyphenyl)ethenyl]phenoxy]-6-(hydroxymethyl)oxane-3,4,5-triol), resveratrol 3-β-mono-D-glucoside, or cis-piceid, trans-piceid, 3,5,4′-trihydroxystilbene-3-O-β-D-glucopyranoside.

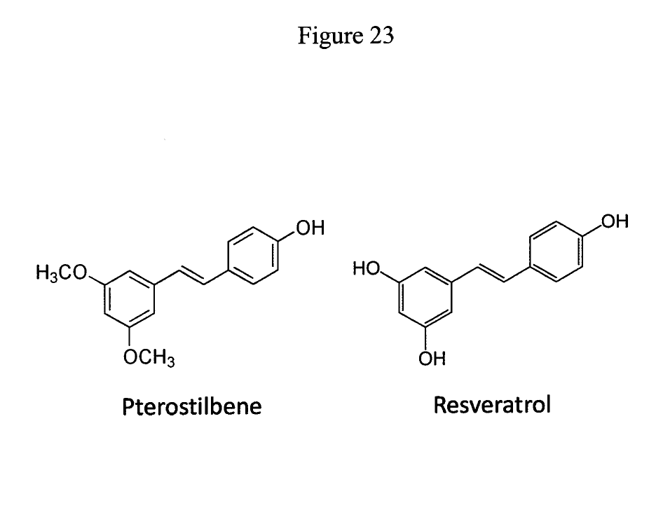

As disclosed herein, the term “methylation,” “methylated,” “methoxylation,” or “methoxylated” can be used interchangeably to refer to a form of alkylation with a methyl group rather than a larger carbon chain. Methylation can encompass adding methyl groups (—CH₃) to the 3, 4′, or 5 hydroxyl groups of resveratrol, or any combination thereof. As used herein, “methylated resveratrol” refers to the substitution of a hydrogen of a 3, 4′, or 5 hydroxyl group (—OH) of resveratrol with a methyl group (—CH₃). The term “mono” used in reference to methylation refers to the attachment of one methyl group. The term “di” used in reference to methylation refers to the attachment of two methyl groups. The term “tri” used in reference to methylation refers to the attachment of three methyl groups. In some embodiments, a stilbene able to be methylated is resveratrol, piceatannol, pinosylvin, dihydroresveratrol, or a stilbene oligomer. Examples methylated resveratrol include, but are not limited to, pterostilbene (3,5-dimethoxy-4′-hydroxy-trans-stilbene, FIG. 23), pinostilbene, 3,5,4′-trimethoxystilbene, tetramethoxystilbene, pentamethoxystilbene, or N-Hydroxy-N-(trimethoxphenyl)-trimethoxy-benzamidine.

Additional non-limiting examples of resveratrol analogs or derivatives thereof include hydroxylated resveratrol analogs or derivatives such as hydroxystilbene, dihydroxystilbene, 3,5-dihydroxypterostilbene, tetrahydroxystilbene, pentahydroxystilbene, or hexahydroxystilbene, fluorinated stilbenes, bridged stilbenes, digalloylresveratrol (ester of gallic acid and resveratrol), or resveratrol triacetate.

In other aspects, resveratrol derivatives can be salts and esters of resveratrol or analogs or derivatives thereof (e.g., salts or esters of a glycosylated resveratrol).

### Production of Resveratrol and Modified Resveratrol

Resveratrol, resveratrol glycosides, methylated resveratrol, or other resveratrol derivatives can be synthesized in vitro, produced biosynthetically, or in some instances, purified from their natural origin. For example, resveratrol or glycosylated resveratrol can be biosynthetically produced in a recombinant host using an exogenous nucleic acid encoding a resveratrol synthase (also known as stilbene synthase). Glycosylated derivatives of resveratrol can be biosynthetically produced in a recombinant host using, for example, one or more uridine diphosphate (UDP)-sugar glycosyltransferases (UGTs). See, for example, Hansen et al., 2009, Phytochemistry 70: 473-482. Glycosylated derivatives of resveratrol can be biosynthetically produced using a resveratrol synthase and one or more UGTs, as described herein. See also, e.g., WO 2008/009728, WO 2009/124879, WO 2009/124967, WO 2009/016108, WO 2006/089898, which are incorporated by reference in their entirety.

As used herein, the term “recombinant host” is intended to refer to a host cell, the genome of which has been augmented by at least one incorporated DNA sequence. Such DNA sequences include, but are not limited to, genes that are not naturally present, DNA sequences that are not normally transcribed into RNA or translated into a protein (“expressed”), and other genes or DNA sequences that are desired to be introduced into the cell to produce the recombinant host. It will be appreciated that the genome of a recombinant host described herein is typically augmented through stable introduction of one or more recombinant genes. Generally, the introduced DNA is not originally resident in the host that is the recipient of the DNA, but it is within the scope of the invention to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms, plant cells, and plants.

The term “recombinant gene” refers to a gene or DNA sequence that is introduced into a recipient host, regardless of whether the same or a similar gene or DNA sequence may already be present in such a host. “Introduced” or “augmented” in this context is known in the art to mean introduced or augmented by the hand of man. Thus, a recombinant gene may be a DNA sequence from another species, or may be a DNA sequence that originated from or is present in the same species, but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. In a preferred embodiment, the DNA is a cDNA copy of an mRNA transcript of a gene produced in a cell.

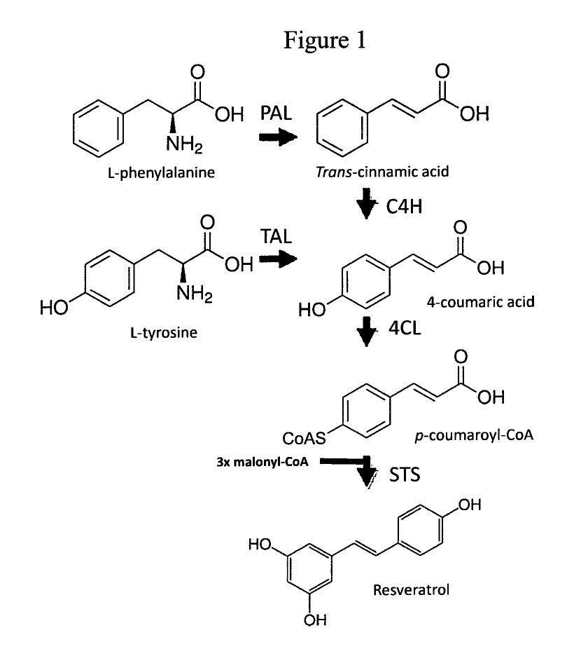

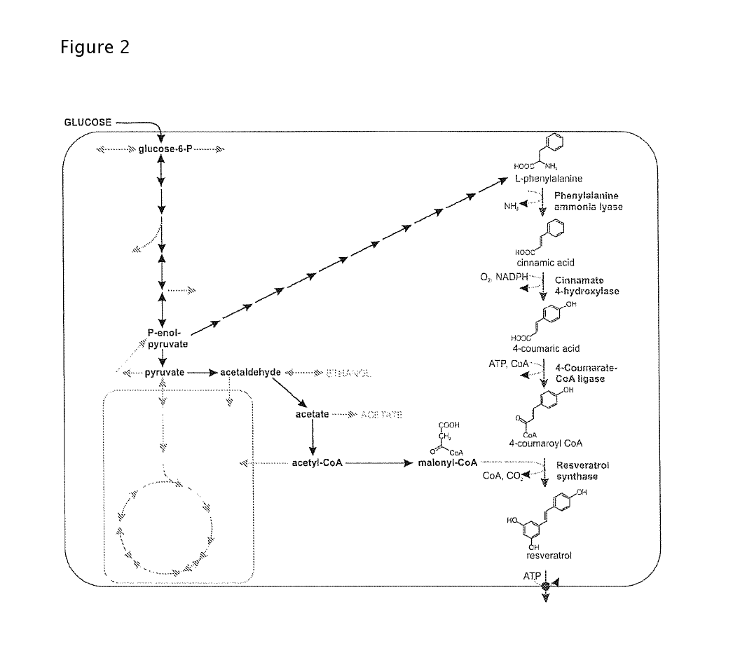

As used herein, the terms “resveratrol producing strain,” “resveratrol producing cells,” “resveratrol producing host,” and “resveratrol producing microorganism” can be used interchangeably to refer to cells that express genes encoding proteins involved in resveratrol production (see, e.g., FIGS. 1, 2). For example, a resveratrol producing strain can express genes encoding one or more of an L-phenylalanine ammonia lyase (PAL) polypeptide, a cinnamate-4-hydroxylase (C4H) polypeptide, a cytochrome P450 monooxygenase polypeptide, an NADPH:cytochrome P450 reductase polypeptide, a 4-coumarate-CoA ligase (4CL) polypeptide, and a stilbene synthase (STS) polypeptide. In another example, a resveratrol producing strain can express genes encoding one or more of a tyrosine ammonia lyase (TAL), a 4-coumarate-CoA ligase (4CL) polypeptide, and a stilbene synthase (STS) polypeptide. One or more of the genes encoding proteins involved in resveratrol production can be recombinant. See, e.g., WO 2008/009728, WO 2009/124879, WO 2009/124967, WO 2009/016108, WO 2006/089898, which are incorporated by reference in their entirety.

In some embodiments, a stilbene producing host comprises a gene encoding a 4-coumarate-CoA ligase (4CL) and a gene encoding stilbene synthase (STS), wherein the host is capable of producing the stilbene from a carbon source when the host is fed, for example, but not limited to, coumaric acid. See, e.g., Wang et al., Annals of Microbiology, 2014, ISSN 1590-4261.

In some embodiments, an L-phenylalanine ammonia lyase (PAL) can be expressed, overexpressed, or recombinantly expressed in said microorganism. Alternatively, said PAL is a PAL (EC 4.3.1.5) from a plant belonging to the genus of Arabidopsis, Brassica, Citrus, Phaseolus, Pinus, Populus, Solanum, Prunus, Vitis, Zea, Agastache, Ananas, Asparagus, Bromheadia, Bambusa, Beta, Betula, Cucumis, Camellia, Capsicum, Cassia, Catharanthus, Cicer, Citrullus, Coffea, Cucurbita, Cynodon, Daucus, Dendrobium, Dianthus, Digitalis, Dioscorea, Eucalyptus, Gallus, Ginkgo, Glycine, Hordeum, Helianthus, Ipomoea, Lactuca, Lithospermum, Lotus, Lycopersicon, Medicago, Malus, Manihot, Medicago, Mesembryanthemum, Nicotiana, Olea, Oryza, Pisum, Persea, Petroselinum, Phalaenopsis, Phyllostachys, Physcomitrella, Picea, Pyrus, Quercus, Raphanus, Rehmannia, Rubus, Sorghum, Sphenostylis, Stellaria, Stylosanthes, Triticum, Trifolium, Triticum, Vaccinium, Vigna, or Zinnia or a microorganism belonging to the genus Agaricus, Aspergillus, Ustilago, Rhodobacter, or Rhodotorula. See, e.g., WO 2006/089898, which has been incorporated by reference in its entirety.

In some embodiments, a tyrosine ammonia lyase (TAL) can be expressed, overexpressed, or recombinantly expressed in said microorganism. Alternatively, said TAL is a TAL (EC 4.3.1.5) from a yeast belonging to the genus Rhodotorula or a bacterium belonging to the genus Rhodobacter. See, e.g., WO 2006/089898, which has been incorporated by reference in its entirety.

In some embodiments, a cinnamate 4-hydroxylase (C4H) can be expressed, overexpressed, or recombinantly expressed in said microorganism. Alternatively, said C4H is a C4H (EC 1.14.13.11) from a plant belonging to the genus of Arabidopsis, Citrus, Phaseolus, Pinus, Populus, Solanum, Vitis, Zea, Ammi, Avicennia, Camellia, Camptotheca, Catharanthus, Glycine, Helianthus, Lotus, Mesembryanthemum, Physcomitrella, Ruta, Saccharum, or Vigna or from a microorganism belonging to the genus Aspergillus. See, e.g., WO 2006/089898, which has been incorporated by reference in its entirety.

In some embodiments, a 4-coumarate-CoA ligase (4CL) polypeptide can be expressed, overexpressed, or recombinantly expressed in said microorganism. Alternatively, said 4CL can be a 4CL (EC 6.2.1.12) from a plant belonging to the genus of Abies, Arabidopsis, Brassica, Citrus, Larix, Phaseolus, Pinus, Populus, Solanum, Vitis, Zea, e.g., Z. mays, Agastache, Amorpha, Cathaya, Cedrus, Crocus, Festuca, Glycine, Juglans, Keteleeria, Lithospermum, Lolium, Lotus, Lycopersicon, Malus, Medicago, Mesembryanthemum, Nicotiana, Nothotsuga, Oryza, Pelargonium, Petroselinum, Physcomitrella, Picea, Prunus, Pseudolarix, Pseudotsuga, Rosa, Rubus, Ryza, Saccharum, Suaeda, Thellungiefia, Triticum, or Tsuga, a microorganism belonging to the genus Aspergillus, Neurospora, Yarrowia, Mycosphaerella, Mycobacterium, Neisseria, Streptomyces, or Rhodobacter, or a nematode belonging to the genus Ancylostoma, Caenorhabditis, Haemonchus, Lumbricus, Meloidogyne, Strongyloidus, or Pristionchus. See, e.g., WO 2006/089898, which has been incorporated by reference in its entirety.

In some embodiments, a stilbene synthase (STS) can be expressed, overexpressed, or recombinantly expressed in said microorganism. Alternatively, said STS is an STS (EC 2.3.1.95) from a plant belonging to the genus of Arachis, Rheum, Vitis, Pinus, Piceea, Lilium, Eucalyptus, Parthenocissus, Cissus, Calochortus, Polygonum, Gnetum, Artocarpus, Nothofagus, Phoenix, Festuca, Carex, Veratrum, Bauhinia, or Pterolobium. See, e.g., WO 2006/089898, which has been incorporated by reference in its entirety.

In some embodiments, an NADPH:cytochrome P450 reductase (CPR) can be expressed, overexpressed, or recombinantly expressed in said microorganism. Alternatively, said CPR is a CPR (EC 1.6.2.4) from a plant belonging to genus Arabidopsis, e.g., A. thaliana, a plant belonging to genus Citrus, e.g., Citrus x sinensis, or Citrus x paradisi, a plant belonging to genus Phaseolus, e.g., P. vulgaris, a plant belonging to genus Pinus, e.g., P. taeda, a plant belonging to genus Populus, e.g., P. deltoides, R. tremuloides, or R. trichocarpa, a plant belonging to genus Solanum, e.g., S. tuberosum, a plant belonging to genus Vitis, e.g., Vitis vinifera, a plant belonging to genus Zea, e.g., Z. mays, or other plant genera, e.g., Ammi, Avicennia, Camellia, Camptotheca, Catharanthus, Glycine, Helianthus, Lotus, Mesembryanthemum, Physcomitrella, Ruta, Saccharum, or Vigna. See, e.g., WO 2006/089898, which has been incorporated by reference in its entirety.

In some embodiments, a recombinant host can express a gene encoding a glycosyltransferase polypeptide. As used herein, the terms “glycosyltransferase enzymes” or “UGTs” are used interchangeably to refer to any enzyme capable of transferring sugar residues and derivatives thereof (including but not limited to galactose, xylose, rhamnose, glucose, arabinose, glucuronic acid, and others as understood in the art, e.g., N-acetyl glucosamine) to acceptor molecules. Acceptor molecules, such as, but not limited to, phenylpropanoids and terpenes include, but are not limited to, other sugars, proteins, lipids, and other organic substrates, such as an alcohol and particularly resveratrol as disclosed herein. The acceptor molecule can be termed an aglycon (aglucone if the sugar is glucose). An aglycon, includes, but is not limited to, the non-carbohydrate part of a glycoside. A “glycoside” as used herein refers an organic molecule with a glycosyl group (organic chemical group derived from a sugar or polysaccharide molecule) connected thereto by way of, for example, an intervening oxygen, nitrogen or sulphur atom. The product of glycosyl transfer can be an O-, N-, S-, or C-glycoside, and the glycoside can be a part of a monosaccharide, disaccharide, oligosaccharide, or polysaccharide.

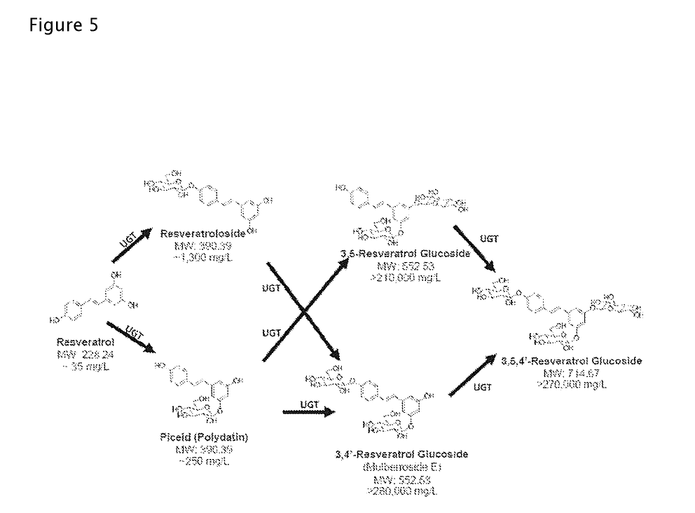

In some embodiments, resveratrol, resveratrol glycosides, methylated resveratrol, methylated resveratrol glycosides, or other resveratrol derivatives are produced in vivo (i.e., in a recombinant host) or in vitro (i.e., enzymatically). In some embodiments, resveratroloside, piceid, 3,5-resveratrol diglucoside, 3,4′-resveratrol diglucoside, 3,5,4′-resveratrol triglucoside, pterostilbene, and/or glycosylated pterostilbene are produced from resveratrol in vivo or in vitro. In some embodiments, 3,4′-resveratrol diglucoside, 3,5,4′-resveratrol triglucoside, and/or glycosylated pterostilbene are produced from resveratroloside in vivo or in vitro. In some embodiments, 3,5-resveratrol diglucoside, 3,4′-resveratrol diglucoside, and/or 3,5,4′-resveratrol triglucoside are produced from piceid in vivo or in vitro. In some embodiments, 3,5,4′-resveratrol triglucoside is produced from 3,5-resveratrol diglucoside or from 3,4′-resveratrol diglucoside in vivo or in vitro (see, e.g., FIG. 5). In some embodiments, the abovementioned compounds are produced in vivo or in vitro through expression of a UGT polypeptide or through contact with a UGT polypeptide.

In particular embodiments, the glycosyltransferase enzyme is Bellis perennis UDP-glucuronic acid:anthocyanin glucuronosyltransferase (BpUGAT or BpUGT94B1) (SEQ ID NOs: 1, 2), Stevia rebaudiana UDP-glycosyltransferase 71E1 (SEQ ID NOs: 3, 4), Arabidopsis thaliana UDP-glucosyl transferase 88A1 (SEQ ID NOs: 7, 8), Catharanthus roseus (Madagascar periwinkle) UDP-glucose glucosyltransferase CaUGT2 (SEQ ID NOs: 9, 10), Arabidopsis thaliana UDP glucose:flavonoid 7-O-glucosyltransferase UGT73B2 (SEQ ID NOs: 13, 14), UGT94B1_R25S (SEQ ID NOs: 15, 16), Arabidopsis thaliana UDP-glycosyltransferase 72B2_Long (SEQ ID NOs: 17, 18), Arabidopsis thaliana UDP-glucosyl transferase 73B5 (SEQ ID NOs: 19, 20), Arabidopsis thaliana UDP-glucosyl transferase 75B2 (SEQ ID NOs: 21, 22), Arabidopsis thaliana UDP-glucosyl transferase 76E1 (SEQ ID NOs: 23, 24), Stevia rebaudiana UDP-glycosyltransferase 76G1 (SEQ ID NOs: 25, 26), Stevia rebaudiana UDP-glycosyltransferase 76H1 (SEQ ID NOs: 27, 28), Arabidopsis thaliana anthocyanidin 3-O-glucosyltransferase 78D2 (SEQ ID NOs: 29, 30), Arabidopsis thaliana UDP-glucosyl transferase 84B1 (SEQ ID NOs: 31, 32), Arabidopsis thaliana UDP-glycosyltransferase 84A1 (SEQ ID NOs: 33, 34), 72EV6 (SEQ ID NOs: 35, 36), 73C3 (SEQ ID NOs: 37, 38), 73C5 (SEQ ID NOs: 39, 40), 89B1 (SEQ ID NOs: 41, 42), SA-GTase (SEQ ID NOs: 43, 44), 72B1 (SEQ ID NOs: 45, 46), 73B3 (SEQ ID NOs: 47, 48), 76E12 (SEQ ID NOs: 49, 50), 71C1 (SEQ ID NOs: 51, 52), 84B2 (SEQ ID NOs: 53, 54), 85A5 (SEQ ID NOs: 55, 56), Gtsatom (SEQ ID NOs: 57, 58), 74F1 (SEQ ID NOs: 59, 60), 84A3 (SEQ ID NOs: 61, 62), UGT72B2_GA (SEQ ID NOs: 63, 18), UGT71E1_GS (SEQ ID NOs: 64, 4), 72E2 (SEQ ID NOs: 65, 66), 71C1-255-71C2 (SEQ ID NOs: 67, 68), 71C1-255-71E1 (SEQ ID NOs: 69, 70), 71C2-255-71E1 (SEQ ID NOs: 71, 72), 71B5 (SEQ ID NOs: 73, 74), 71C1 (SEQ ID NOs: 75, 76), 73B1 (SEQ ID NOs: 77, 78), 73B4 (SEQ ID NOS: 79, 80), 73C1 (SEQ ID NOs: 81, 82), 75B1 (SEQ ID NOS: 83, 84), 75D1 (SEQ ID NOS: 85, 86), 76E5 (SEQ ID NOs: 87, 88), 76F2 (SEQ ID NOS: 89, 90), 78D3 (SEQ ID NOs: 91, 92), 84A2 (SEQ ID NOS: 93, 94), 85A1 (SEQ ID NOs: 95, 96), 87A2 (SEQ ID NOS: 97, 98), 90A2 (SEQ ID NOs: 99, 100), 91B1 (SEQ ID NOs: 101, 102), 71C1-188-71C2 (SEQ ID NOs: 103, 104), 74C1 (SEQ ID NOs: 105, 106), 74F2 (SEQ ID NOS: 107, 108), 74G1 (SEQ ID NOs: 109, 110), 75C1 (SEQ ID NOs: 111, 112), 76B1 (SEQ ID NOs: 113, 114), 76E4 (SEQ ID NOs: 115, 116), or UGT91D2e_b (SEQ ID NOs: 117, 118).

In some embodiments, the UGT polypeptides 72B1 (SEQ ID NOs: 45, 46), 73B3 (SEQ ID NOs: 47, 48), 73C3 (SEQ ID NOs: 37, 38), 74F1 (SEQ ID NOs: 59, 60), 75B2 (SEQ ID NOs: 21, 22), 76E1 (SEQ ID NOs: 23, 24), 71C1 (SEQ ID NOs: 51, 52), 76H1 (SEQ ID NOs: 27, 28), 84A3 (SEQ ID NOs: 61, 62), 85A5 (SEQ ID NOs: 55, 56), 88A1 (SEQ ID NOs: 7, 8), Gtsatom (SEQ ID NOs: 57, 58), 71C1-188-71C2 (SEQ ID NOs: 103, 104), 71C1-255-71C2 (SEQ ID NOs: 67, 68), 71C2-255-71E1 (SEQ ID NOs: 71, 72), 71C1-255-71E1 (SEQ ID NOs: 69, 70), 89B1 (SEQ ID NOs: 41, 42), 72EV6 (SEQ ID NOs: 35, 36), 76EV8 (SEQ ID NOs: 121, 122), 90A2 (SEQ ID NOs: 99, 100), 71B5 (SEQ ID NOs: 73, 74), 73B2 (SEQ ID NOs: 13, 14), 73C1 (SEQ ID NOs: 81, 82), 74G1 (SEQ ID NOs: 109, 110), 76B1 (SEQ ID NOs: 113, 114), 76E5 (SEQ ID NOs: 87, 88), 84A1 (SEQ ID NOs: 33, 34), 87A2 (SEQ ID NOs: 97, 98) used in the methods disclosed herein produced piceid from resveratrol in vitro. See Example 1, Table 1, Table 2.

In some embodiments, the UGT polypeptides 72B1 (SEQ ID NOs: 45, 46), 72B2_Long (SEQ ID NOs: 17, 18), 73B3 (SEQ ID NOs: 47, 48), 73C3 (SEQ ID NOs: 37, 38), 73C5 (SEQ ID NOs: 39, 40), 74F1 (SEQ ID NOs: 59, 60), 84A3 (SEQ ID NOs: 61, 62), 84B1 (SEQ ID NOs: 31, 32), 84B2 (SEQ ID NOs: 53, 54), Gtsatom (SEQ ID NOs: 57, 58), 71C1-255-71C2 (SEQ ID NOs: 67, 68), SA-GTase (SEQ ID NOs: 43, 44), 89B1 (SEQ ID NOs: 41, 42), 72EV6 (SEQ ID NOs: 35, 36), 76EV8 (SEQ ID NOs: 121, 122), 90A2 (SEQ ID NOs: 99, 100), 91B1 (SEQ ID NOs: 101, 102), 71C1 (SEQ ID NOs: 75, 76), 72E2 (SEQ ID NOs: 65, 66), 73B4 (SEQ ID NOs: 79, 80), 74C1 (SEQ ID NOs: 105, 106), 74G1 (SEQ ID NOs: 109, 110), 75C1 (SEQ ID NOs: 111, 112), 76B1 (SEQ ID NOs: 113, 114), 76E4 (SEQ ID NOs: 115, 116), 78D3 (SEQ ID NOs: 91, 92), 84A1 (SEQ ID NOs: 33, 34) used in the methods disclosed herein produced resveratroloside from resveratrol in vitro. See Example 1, Table 1, Table 2.

In some embodiments, the UGT polypeptides 71E1 (SEQ ID NOs: 3, 4), 73B5 (SEQ ID NOs: 19, 20), 84B1 (SEQ ID NOs: 31, 32), 71C2-255-71E1 (SEQ ID NOs: 71, 72) used in the methods disclosed herein produced 3,5-resveratrol diglucoside from resveratrol in vitro. See Example 1, Table 1, Table 2.

In some embodiments, the UGT polypeptides 71E1 (SEQ ID NOs: 3, 4), 73B3 (SEQ ID NOs: 47, 48), 73B5 (SEQ ID NOs: 19, 20), 76G1 (SEQ ID NOs: 25, 26), 88A1 (SEQ ID NOs: 7, 8), 71C2-255-71E1 (SEQ ID NOs: 71, 72), 76EV8 (SEQ ID NOs: 121, 122), 90A2 (SEQ ID NOs: 99, 100), 73B2 (SEQ ID NOs: 13, 14), 74G1 (SEQ ID NOs: 109, 110) used in the methods disclosed herein produced 3,5-resveratrol diglucoside from piceid in vitro. See Example 1, Table 1, Table 2.

In some embodiments, the UGT polypeptides 72B1 (SEQ ID NOs: 45, 46), 72B2_Long (SEQ ID NOs: 17, 18), 73B3 (SEQ ID NOs: 47, 48), 73B5 (SEQ ID NOs: 19, 20), 73C3 (SEQ ID NOs: 37, 38), 73C5 (SEQ ID NOs: 39, 40), 74F1 (SEQ ID NOs: 59, 60), 76E1 (SEQ ID NOs: 23, 24), 84B1 (SEQ ID NOs: 31, 32), 71C1-255-71E1 (SEQ ID NOs: 69, 70), 89B1 (SEQ ID NOs: 41, 42), 72EV6 (SEQ ID NOs: 35, 36) used in the methods disclosed herein produced 3,4′-resveratrol diglucoside from resveratrol in vitro. See Example 1, Table 1, Table 2.

In some embodiments, the UGT polypeptides 72B1 (SEQ ID NOs: 45, 46), 72B2_Long (SEQ ID NOs: 17, 18), 73B3 (SEQ ID NOs: 47, 48), 73B5 (SEQ ID NOs: 19, 20), 73C3 (SEQ ID NOs: 37, 38), 73C5 (SEQ ID NOs: 39, 40), 74F1 (SEQ ID NOs: 59, 60), 76E1 (SEQ ID NOs: 23, 24), 76E12 (SEQ ID NOs: 49, 50), 71C1 (SEQ ID NOs: 51, 52), 76H1 (SEQ ID NOs: 27, 28), 78D2 (SEQ ID NOs: 29, 30), 84A3 (SEQ ID NOs: 61, 62), 84B1 (SEQ ID NOs: 31, 32), 84B2 (SEQ ID NOs: 53, 54), 84A5 (SEQ ID NOs: 55, 56), Gtsatom (SEQ ID NOs: 57, 58), SA-Gtase (SEQ ID NOs: 43, 44), 72EV6 (SEQ ID NOs: 35, 36), 90A2 (SEQ ID NOs: 99, 100), 91B1 (SEQ ID NOs: 101, 102), 73B2 (SEQ ID NOs: 13, 14), 74G1 (SEQ ID NOs: 109, 110) used in the methods disclosed herein produced 3,4′-resveratrol diglucoside from piceid in vitro. See Example 1, Table 1, Table 2.

In some embodiments, the UGT polypeptides 71E1 (SEQ ID NOs: 3, 4), 72B2_Long (SEQ ID NOs: 17, 18), 73B3 (SEQ ID NOs: 47, 48), 73B5 (SEQ ID NOs: 19, 20), 73C3 (SEQ ID NOs: 37, 38), 74F1 (SEQ ID NOs: 59, 60), 75B2 (SEQ ID NOs: 21, 22), 76E1 (SEQ ID NOs: 23, 24), 71C1 (SEQ ID NOs: 51, 52), 76H1 (SEQ ID NOs: 27, 28), 78D2 (SEQ ID NOs: 29, 30), 84A3 (SEQ ID NOs: 61, 62), 84B1 (SEQ ID NOs: 31, 32), 88A1 (SEQ ID NOs: 7, 8), Gtsatom (SEQ ID NOs: 57, 58), 71C1-188-71C2 (SEQ ID NOs: 103, 104), 71C1-255-71C2 (SEQ ID NOs: 67, 68), 71C2-255-71E1 (SEQ ID NOs: 71, 72), 71C1-255-71E1 (SEQ ID NOs: 69, 70), SA-Gtase (SEQ ID NOs: 43, 44), 89B1 (SEQ ID NOs: 41, 42), 72EV6 (SEQ ID NOs: 35, 36), 90A2 (SEQ ID NOs: 99, 100), 73C1 (SEQ ID NOs: 81, 82), 85A1 (SEQ ID NOs: 95, 96) used in the methods disclosed herein produced 3,4′-resveratrol diglucoside from resveratroloside in vitro. See Example 1, Table 1, Table 2.

In some embodiments, the UGT polypeptide 84B1 (SEQ ID NOs: 31, 32) used in the methods disclosed herein produced 3,5,4′-resveratrol triglucoside from resveratrol or piceid in vitro. In some embodiments, the UGT polypeptide 73B5 (SEQ ID NOs: 19, 20) used in the methods disclosed herein produced 3,5,4′-resveratrol triglucoside from piceid or resveratroloside in vitro. In some embodiments, the UGT polypeptide 78D2 (SEQ ID NOs: 29, 30) used in the methods disclosed herein produced 3,5,4′-resveratrol triglucoside from resveratrol or resveratroloside in vitro. See Example 1, Table 1, Table 2.

In some embodiments, the UGT polypeptides BpUGAT 94B1 R25S (SEQ ID NOs: 15, 16) and 91D2e_b (SEQ ID NOs: 117, 118) produce 4′-bis-glucoside (glucose on glucose) from resveratroloside in vitro. In some embodiments, BpUGT94B1 (SEQ ID NOs: 1, 2) used in the methods disclosed herein is used to add a glucuronic acid molecule to the glucose at the 4′ position of 3,4′-resveratrol diglucoside in vitro. See Example 1, Table 1.

In some embodiments, the UGT polypeptides 71E1 (SEQ ID NOS: 3, 4), 73B5 (SEQ ID NOS: 19, 20), 74F1 (SEQ ID NOS: 59, 60), 75B2 (SEQ ID NOS: 21, 22), 71C1 (SEQ ID NOS: 51, 52), 78D2 (SEQ ID NOS: 29, 30), 84A3 (SEQ ID NOS: 61, 62), 84B1 (SEQ ID NOS: 31, 32), 84B2 (SEQ ID NOS: 53, 54), Gtsatom (SEQ ID NOS: 57, 58), SA-Gtase (SEQ ID NOS: 43, 44), 73B4 (SEQ ID NOS: 79, 80), 74F2 (SEQ ID NOS: 107, 108), 75B1 (SEQ ID NOS: 83, 84), 75C1 (SEQ ID NOS: 111, 112), 75D1 (SEQ ID NOS: 85, 86), 76F2 (SEQ ID NOS: 89, 90), 84A1 (SEQ ID NOS: 33, 34), 84A2 (SEQ ID NOS: 93, 94), 87A2 (SEQ ID NOS: 97, 98) used in the methods disclosed herein use cinnamic acid as a substrate in vitro. See Example 1, Table 1.

In some embodiments, the UGT polypeptides 71E1 (SEQ ID NOS: 3, 4), 73B5 (SEQ ID NOS: 19, 20), 74F1 (SEQ ID NOS: 59, 60), 75B2 (SEQ ID NOS: 21, 22), 71C1 (SEQ ID NOS: 51, 52), 78D2 (SEQ ID NOS: 29, 30), 84A3 (SEQ ID NOS: 61, 62), 84B1 (SEQ ID NOS: 31, 32), 84B2 (SEQ ID NOS: 53, 54), Gtsatom (SEQ ID NOS: 57, 58), 71C1-255-71C2 (SEQ ID NOs: 67, 68), SA-Gtase (SEQ ID NOS: 43, 44), 89B1 (SEQ ID NOs: 41, 42), 73B1 (SEQ ID NOs: 77, 78), 73B4 (SEQ ID NOS: 79, 80), 74F2 (SEQ ID NOS: 107, 108), 75B1 (SEQ ID NOS: 83, 84), 75C1 (SEQ ID NOS: 111, 112), 76F2 (SEQ ID NOS: 89, 90), 84A1 (SEQ ID NOS: 33, 34), 84A2 (SEQ ID NOS: 93, 94), 87A2 (SEQ ID NOS: 97, 98) used in the methods disclosed herein use p-coumaric acid as a substrate in vitro. See Example 1, Table 1.

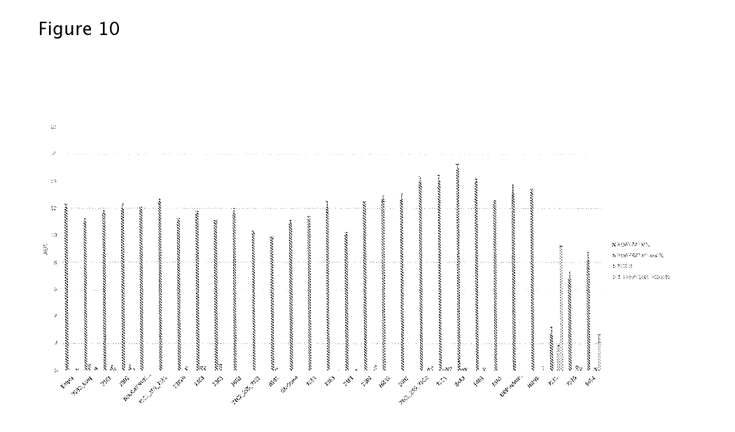

In some embodiments, expression of UGT72B2_Long (SEQ ID NOs: 17, 18), UGT72B2_GA (SEQ ID NOs: 63, 18), UGT73C3 (SEQ ID NOs: 37, 38), UGT73C5 (SEQ ID NOs: 39, 40), UGT89B1 (SEQ ID NOs: 41, 42), or UGT84A3 (SEQ ID NOs: 61, 62) in a resveratrol-producing yeast strain results in production of resveratroloside in vivo. See Examples 5-6, FIG. 10, Table 5.

In some embodiments, expression of UGT71E1 (SEQ ID NOs: 3, 4), UGT71E1_GS (SEQ ID NOs: 64, 4), UGT76E1 (SEQ ID NOs: 23, 24), UGT78D2 (SEQ ID NOs: 29, 30), UGT72EV6 (SEQ ID NOs: 35, 36), UGT73C3 (SEQ ID NOs: 37, 38), UGT71C1-255-71C2 (SEQ ID NOs: 67, 68), UGT71C1 (SEQ ID NOs: 51, 52), UGT84A3 (SEQ ID NOs: 61, 62), UGT84B2 (SEQ ID NOs: 53, 54), UGT73B5 (SEQ ID NOs: 19, 20), or UGT84B1 (SEQ ID NOs: 31, 32) in a resveratrol-producing yeast strain results in production of piceid in vivo. See Examples 5-6, FIG. 10, Table 5.

In some embodiments, expression of UGT71E1 (SEQ ID NOs: 3, 4), UGT71E1_GS (SEQ ID NOs: 64, 4), UGT84B1 (SEQ ID NOs: 31, 32), UGT72B2_Long (SEQ ID NOs: 17, 18), UGT76E1 (SEQ ID NOs: 23 24), UGT78D2 (SEQ ID NOs: 29, 30), UGT75B2 (SEQ ID NOs: 21, 22), UGT71C1-255-71C2 (SEQ ID NOs: 67, 68), UGT71C1 (SEQ ID NOs: 51, 52), or UGT73B5 (SEQ ID NOs: 19, 20) in a resveratrol-producing yeast strain results in production of 3,5-resveratrol diglucoside in vivo. See Examples 5-6, FIG. 10, Table 5.

In some embodiments, expression of UGT72B2_Long (SEQ ID NOs: 17, 18), UGT72B2_GA (SEQ ID NOs: 63, 18), (SEQ ID NOs: 3, 4), UGT71E1_GS (SEQ ID NOs: 64, 4), UGT73B5 (SEQ ID NOs: 19, 20), or UGT84B1 (SEQ ID NOs: 31, 32) in a resveratrol-producing yeast strain results in production one or more resveratrol glycosides with a retention time of approximately 3.78 min, 4.52 min, 5.42 min, or 5.75 min. See Example 6, Table 5.

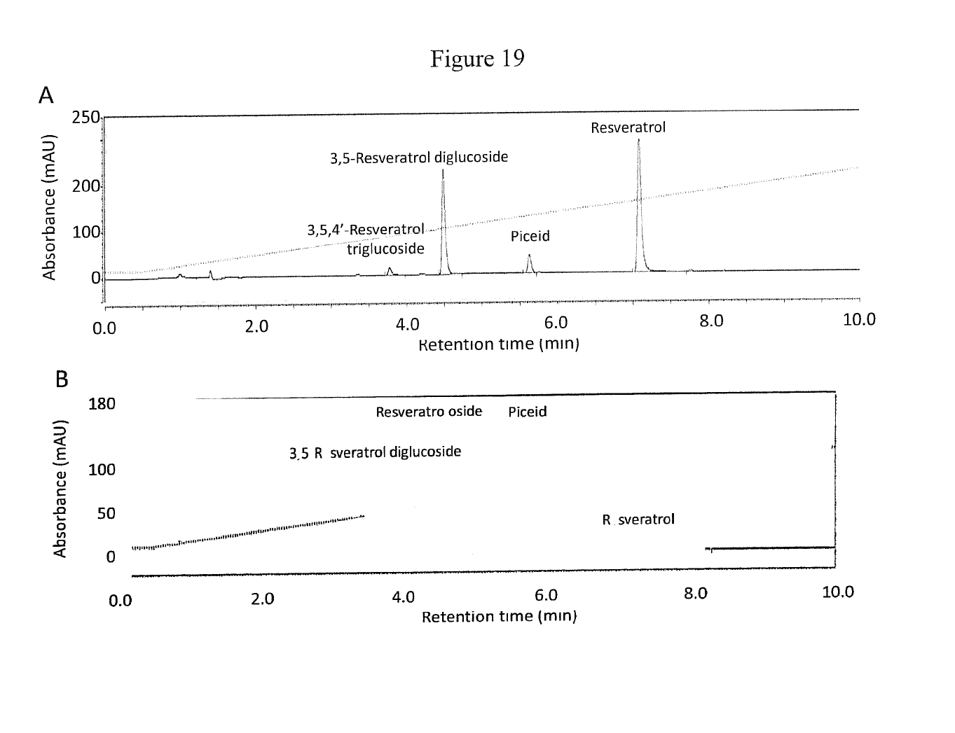

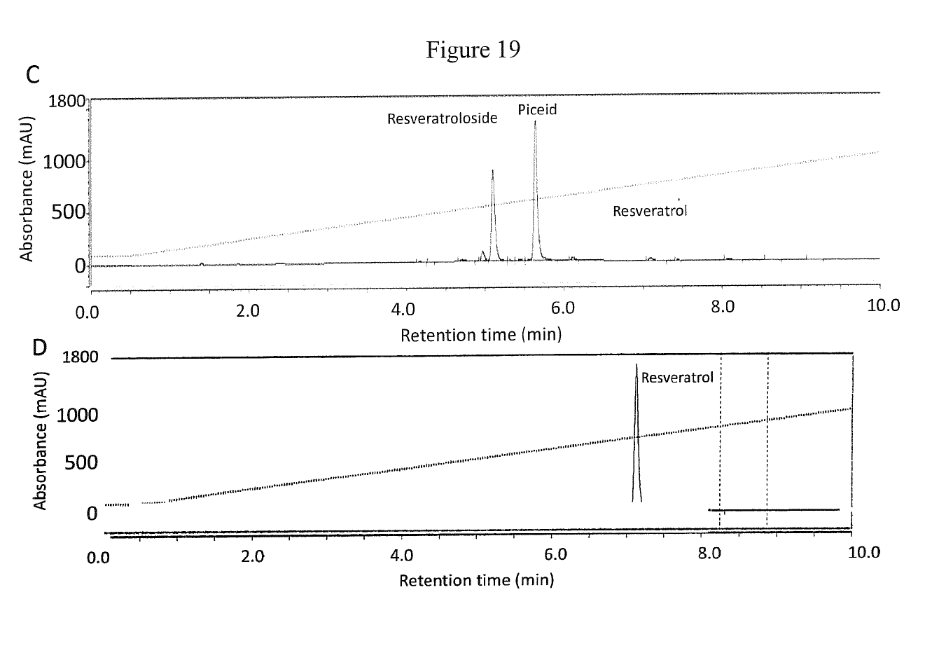

In some embodiments, a glycosylated stilbene, such as a resveratrol glucoside, is produced by bioconversion. In some aspects, a host cell expressing a UGT polypeptide takes up and glycosylates a stilbene in the cell, and following glycosylation in vivo, the glycosylated stilbene is released into the culture medium. In some embodiments, expression of UGT71E1 (SEQ ID NOs: 3, 4) in S. cerevisiae cells results in the bioconversion of resveratrol into piceid, resveratroloside, 3,5-resveratrol diglucoside, and/or 3,5,4′-resveratrol triglucoside. In some embodiments, expression of UGT88A1 (SEQ ID NOs: 7, 8), CaUGT2 (SEQ ID NOs: 9, 10), or UGT73B2 (SEQ ID NOs: 13, 14) in S. cerevisiae cells results in the bioconversion of resveratrol to piceid in vitro. In some embodiments, expression of UGT71E1 (SEQ ID NOs: 3, 4) in S. cerevisiae cells results in the bioconversion of resveratrol from knotweed root extracts to piceid and resveratroloside in vitro. In some embodiments, subsequent treatment with a β-glucosidase enzyme results in production of resveratrol from resveratrol glycosides produced by bioconversion of resveratrol. See Example 8, FIG. 19.

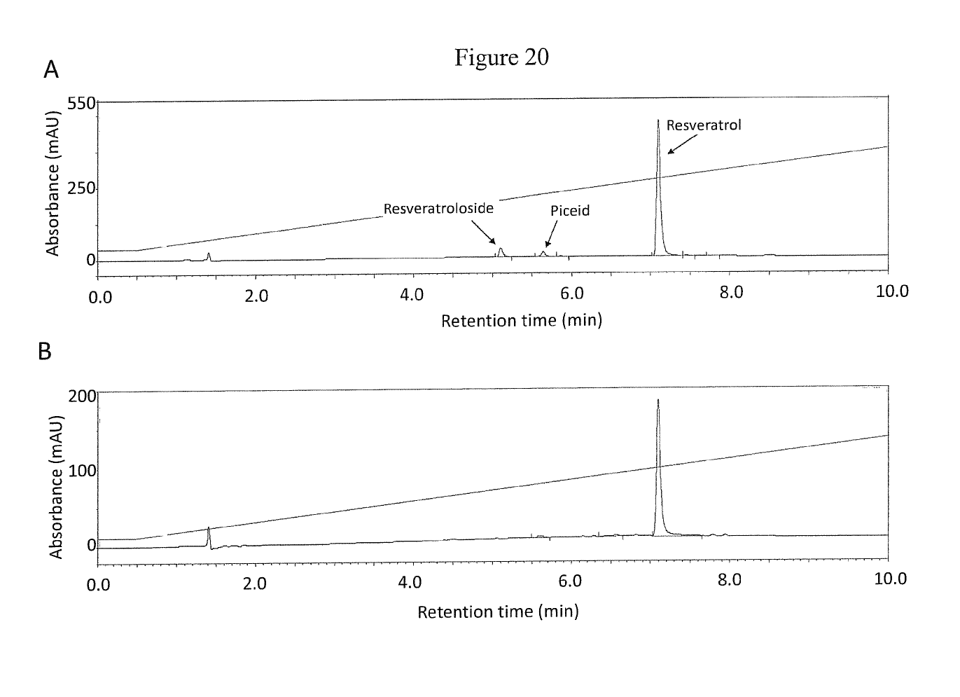

In some embodiments, supplementation of E. coli cells expressing Phytolacca americana glycosyltransferase PaGT3 (SEQ ID NOs: 119, 120) with resveratrol results in formation of piceid and resveratroloside. See Example 9, FIG. 20.

In particular embodiments of all aspects provided by the invention, the glycosyltransferase enzyme is a eukaryotic enzyme, i.e., an enzyme produced in a eukaryotic species including without limitation species from yeast, fungi, plants, and animals. In some embodiments, the glycosyltransferase enzyme is a bacterial enzyme.

As used herein, the terms “codon optimization” and “codon optimized” refers to a technique to maximize protein expression in fast-growing microorganisms such as Escherichia coli or Saccharomyces cerevisiae by increasing the translation efficiency of a particular gene. Codon optimization can be achieved, for example, by transforming nucleotide sequences of one species into the genetic sequence of a different species. Optimal codons help to achieve faster translation rates and high accuracy. As a result of these factors, translational selection is expected to be stronger in highly expressed genes. Examples of codon-optimized UGTs are UGT72B2_GA (SEQ ID NO: 63) and UGT71E1_GS (SEQ ID NO: 64).

In some embodiments, a microorganism endogenously facilitates glycosylation of resveratrol or resveratrol derivatives. For example, S. cerevisiae yeast (budding yeast) is capable of small molecule glycosylation. Previous studies have reached up to 30 g/L final titer of glycosylated compounds.

In some embodiments, the amino acid sequences for glycosyltransferase enzymes disclosed herein are variants that have at least 40% identity to the amino acid sequences set forth herein, wherein the variants retain the activity of the glycosyltransferase enzymes disclosed in herein.

In some embodiments, a gene encoding a UGT polypeptide is expressed, overexpressed, or recombinantly expressed in a cell that does not express an exo-1,3-beta-glucanase. In some embodiments, the cell is an S. cerevisiae cell and the exo-1,3-beta-glucanase is EXG1 (SEQ ID NOs: 123, 124), which codes for the major exo-1,3-beta-glucanase of the yeast cell wall. EXG1 has been shown to efficiently cleave glucose moieties from resveratrol glycosides.

In some embodiments, the glucose moieties of resveratrol glycosides are cleaved. Enzymes capable of cleaving a glucose molecule from resveratrol include, but are not limited to, β-glucosidase, Depol™ (cellulase), cellulase T. reesei, glusulase, cellobiase A. niger, β-galactosidase A. oryzae, β-glucuronidase, and EXG1 (SEQ ID NO: 124) broth.

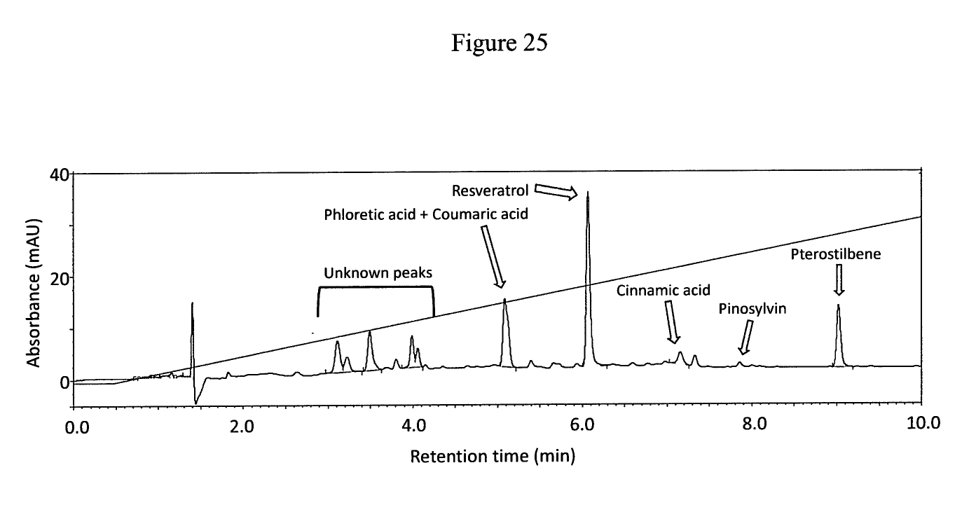

As used herein, the terms “resveratrol O-methyltransferase” and “ROMT” are used interchangeably to refer to any enzyme capable of transferring methyl groups to acceptor molecules. Acceptor molecules include, but are not limited to, phenylpropanoids, terpenes, sugars, proteins, lipids, and other organic substrates, such as alcohols and particularly resveratrol. An example of an ROMT enzyme that produces pterostilbene is Vitis vinifera ROMT (SEQ ID NOs: 5, 6). In some embodiments, an ROMT polypeptide catalyzes the methylation of compounds other than resveratrol (see, e.g., Example 11, FIG. 25).

In other particular embodiments of all aspects provided by the invention, the methyltransferase enzyme is a eukaryotic enzyme, i.e., an enzyme produced in a eukaryotic species including without limitation species from yeast, fungi, plants, and animals. In some embodiments, the methyltransferase enzyme is a bacterial enzyme or an enzyme encoded by a synthetic gene.

In some embodiments, supplementation of S. cerevisiae expressing Vitis vinifera ROMT (SEQ ID NOs: 5, 6) with resveratrol results in the production of pterostilbene. See Example 12.

In some embodiments, a methylated stilbene, such as methylated resveratrol, is produced by bioconversion. In some aspects, a host cell expressing a methyltransferase polypeptide takes up and methylates a stilbene in the cell, and following methylation in vivo, the methylated stilbene is released into the culture medium. In some embodiments, expression of ROMT (SEQ ID NOs: 5, 46) in S. cerevisiae cells results in the bioconversion of resveratrol into methylated resveratrol.

In some embodiments, purified UGT72B2_Long (SEQ ID NOs: 17, 18) incubated with pterostilbene in vitro results in the production of glycosylated pterostilbene. In some embodiments, treatment of the glycosylated pterostilbene produced in vitro with a β-glucosidase results in recovery of pterostilbene. See Example 13.

Thus, examples of in vitro and in vivo enzymatic resveratrol modifications include, but are not limited to, the addition of glucose, galactose, or xylose (sugar) to resveratrol by the enzymatic glycosylation of resveratrol using the sugar donors UDP-galactose or UDP-xylose, and the addition of second glucose or for example glucuronosyl unit to glucosyl moiety of piceid, resveratroloside, 3,5-resveratrol diglucoside, and 3,4′-resveratrol diglucoside by the aid of Bellis perennis UGT94B1 (SEQ ID NOs: 1, 2) (Sawada et al., 2005, J Biol Chem. 280: 899-906; Osmani et al. 2008, Plant Physiol. 148: 1295-1308). By the aid of a methyltransferase (e.g., Vitis vinifera resveratrol O-methyltransferase (Schmidlin et al., 2008, Plant Physiol 148: 1630-1639)), resveratrol hydroxyl-groups can be methylated to yield, for example, pterostilbene (3,5-dimethoxy-4′-hydroxy-trans-stilbene).

### Functional Homologs

Functional homologs of the polypeptides described above are also suitable for use in producing glycosylated resveratrol or methylated resveratrol. A functional homolog is a polypeptide that has sequence similarity to a reference polypeptide, and that carries out one or more of the biochemical or physiological function(s) of the reference polypeptide. A functional homolog and the reference polypeptide can be natural occurring polypeptides, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional UGT polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide:polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of polypeptides described herein. Sequence analysis can involve BLAST, Reciprocal BLAST, or PSI-BLAST analysis of nonredundant databases using the amino acid sequence of interest as the reference sequence. Amino acid sequence is, in some instances, deduced from the nucleotide sequence. Those polypeptides in the database that have greater than 40% sequence identity are candidates for further evaluation for suitability as polypeptide useful in the synthesis of resveratrol and resveratrol derivatives. Amino acid sequence similarity allows for conservative amino acid substitutions, such as substitution of one hydrophobic residue for another or substitution of one polar residue for another. When desired, manual inspection of such candidates can be carried out in order to narrow the number of candidates to be further evaluated. Manual inspection can be performed by selecting those candidates that appear to have conserved functional domains.

Conserved regions can be identified by locating a region within the primary amino acid sequence of a polypeptide described herein that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. The information included at the Pfam database is described in Sonnhammer et al., 1998, Nucl. Acids Res., 26:320-322; Sonnhammer et al., 1997, Proteins, 28:405-420; and Bateman et al., 1999, Nucl. Acids Res., 27:260-262. Conserved regions also can be determined by aligning sequences of the same or related polypeptides from closely related species. Closely related species preferably are from the same family. In some embodiments, alignment of sequences from two different species can be adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). See Chenna et al., 2003, Nucleic Acids Res., 31(13):3497-500. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100.

It will be appreciated that polypeptides described herein can include additional amino acids that are not involved in glycosylation, methylation or other enzymatic activities carried out by the enzyme, and thus such a polypeptide can be longer than would otherwise be the case. For example, a polypeptide can include a purification tag (e.g., HIS tag or GST tag), a chloroplast transit peptide, a mitochondrial transit peptide, an amyloplast peptide, signal peptide, or a secretion tag added to the amino or carboxy terminus. In some embodiments, a polypeptide includes an amino acid sequence that functions as a reporter, e.g., a green fluorescent protein or yellow fluorescent protein.

### Recombinant Microorganisms

A number of prokaryotes and eukaryotes are suitable for use in constructing the recombinant microorganisms described herein, e.g., bacteria, yeast and fungi. A species and strain selected for use as a strain for production of glycosylated resveratrol or methylated resveratrol compounds is first analyzed to determine which production genes are endogenous to the strain and which genes are not present (e.g., resveratrol production genes). Genes for which an endogenous counterpart is not present in the strain are assembled in one or more recombinant constructs, which are then transformed into the strain in order to supply the missing function(s).

In the present context the terms “microorganism” and “microorganism host” and “recombinant host” can be used interchangeably to refer to microscopic organisms, including bacteria or microscopic fungi, including yeast. Specifically, the microorganism can be a eukaryotic cell or immortalized cell.

Exemplary prokaryotic and eukaryotic species are described in more detail below. However, it will be appreciated that other species can be suitable. For example, suitable species can be in a genus including Agaricus, Aspergillus, Bacillus, Candida, Corynebacterium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces and Yarrowia. Exemplary species from such genera include Lentinus tigrinus, Laetiporus sulphureus, Phanerochaete chrysosporium, Pichia pastoris, Physcomitrella patens, Rhodoturula glutinis 32, Rhodoturula mucilaginosa, Phaffia rhodozyma UBV-AX, Xanthophyllomyces dendrorhous, Fusarium fujikuroi/Gibberella fujikuroi, Candida utilis and Yarrowia lipolytica. In some embodiments, a microorganism can be an Ascomycete such as Gibberella fujikuroi, Kluyveromyces lactis, Schizosaccharomyces pombe, Aspergillus niger, or Saccharomyces cerevisiae. In some embodiments, a microorganism can be a prokaryote such as Escherichia coli, Rhodobacter sphaeroides, or Rhodobacter capsulatus. It will be appreciated that certain microorganisms can be used to screen and test genes of interest in a high throughput manner, while other microorganisms with desired productivity or growth characteristics can be used for large-scale production of resveratrol or resveratrol derivatives or analogs.

In certain embodiments of this invention, microorganisms include, but are not limited to, S. cerevisiae, A. niger, A. oryzae, E. coli, L. lactis and B. subtilis. The constructed and genetically engineered microorganisms provided by the invention can be cultivated using conventional fermentation processes, including, inter alia, chemostat, batch, fed-batch cultivations, continuous perfusion fermentation, and continuous perfusion cell culture.

Exemplary embodiments comprising bacterial cells include, but are not limited to, cells of species, belonging to the genus Bacillus, the genus Escherichia, the genus Lactobacillus, the genus Lactobacillus, the genus Corynebaclerium, the genus Acetobacler, the genus Acinetobacler, or the genus Pseudomonas.

The microorganism can be a fungus, and more specifically, a filamentous fungus belonging to the genus of Aspergillus, e.g., A. niger, A. awamori, A. oryzae, or A. nidulans, a yeast belonging to the genus of Saccharomyces, e.g., S. cerevisiae, S. kluyveri, S. bayanus, S. exiguus, S. sevazzi, or S. uvarum, a yeast belonging to the genus Kluyveromyces, e.g., K. laclis, K. marxianus var. marxianus, or K. thermololerans, a yeast belonging to the genus Candida, e.g., C. ulilis, C. tropicalis, C. albicans, C. lipolylica, or C. versalilis, a yeast belonging to the genus Pichia, e.g., R. slipidis, R. pastoris, or P. sorbilophila, or other yeast genera, e.g., Cryptococcus, Debaromyces, Hansenula, Pichia, Yarrowia, Zygosaccharomyces, or Schizosaccharomyces. Concerning other microorganisms a non-exhaustive list of suitable filamentous fungi is supplied: a species belonging to the genus Penicillium, Rhizopus, Fusarium, Fusidium, Gibberella, Mucor, Morlierella, and Trichoderma.

Saccharomyces cerevisiae

Saccharomyces cerevisiae is a widely used chassis organism in synthetic biology, and can be used as the recombinant microorganism platform. There are libraries of mutants, plasmids, detailed computer models of metabolism and other information available for S. cerevisiae, allowing for rational design of various modules to enhance product yield. Methods are known for making recombinant microorganisms.

The genes described herein can be expressed in yeast using any of a number of known promoters. Strains that overproduce phenylpropanoids are known and can be used as acceptor molecules in the production of glycosylated resveratrol and/or methylated resveratrol.

Aspergillus spp.

Aspergillus species such as A. oryzae, A. niger and A. sojae are widely used microorganisms in food production, and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for the production of resveratrol and resveratrol derivatives.

Escherichia coli

Escherichia coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield.

Methods similar to those described above for Saccharomyces can be used to make recombinant E. coli microorganisms.

Agaricus, Gibberella, and Phanerochaete spp.

Agaricus, Gibberella, and Phanerochaete spp. can be useful because they are known to produce large amounts of gibberellin in culture. Thus, the precursors of terpenes used as acceptor molecules in the production of glycosylated resveratrol and/or methylated resveratrol are already produced by endogenous genes. Thus, modules containing recombinant genes for biosynthesis of terpenes can be introduced into species from such genera without the necessity of introducing other compounds or pathway genes.

Rhodobacter spp.

Rhodobacter can be used as the recombinant microorganism platform. Similar to E. coli, there are libraries of mutants available as well as suitable plasmid vectors, allowing for rational design of various modules to enhance product yield. Isoprenoid pathways have been engineered in membraneous bacterial species of Rhodobacter for increased production of carotenoid and CoQ10. See, U.S. Patent Publication Nos. 20050003474 and 20040078846. Methods similar to those described above for E. coli can be used to make recombinant Rhodobacter microorganisms.

Physcomitrella spp.

Physcomitrella mosses, when grown in suspension culture, have characteristics similar to yeast or other fungal cultures. This genera is becoming an important type of cell for production of plant secondary metabolites, which can be difficult to produce in other types of cells.

As will be apparent to one skilled in the art, the particulars of the selection process for specific UGTs capable of glycosylating resveratrol or for specific ROMTs depend on the identities of the selectable markers. Selection in all cases promotes or permits proliferation of cells comprising the marker while inhibiting or preventing proliferation of cells lacking the marker. If a selectable marker is an antibiotic resistance gene, the transfected host cell population can be cultured in the presence of an antibiotic to which resistance is conferred by the selectable marker. If a selectable marker is a gene that complements an auxotrophy of the host cells, the transfected host cell population can be cultivated in the absence of the compound for which the host cells are auxotrophic.

After selection, recombinant host cells can be cloned according to any appropriate method known in the art. For example, recombinant microbial host cells can be plated on solid media under selection conditions, after which single clones can be selected for further selection, characterization, or use. This process can be repeated one or more times to enhance stability of the expression construct within the host cell. To produce UGTs or ROMTs, recombinant host cells comprising one or more expression vectors can be cultured to expand cell numbers in any appropriate culturing apparatus known in the art, such as a shaken culture flask or a fermenter.

Culture media used for various recombinant host cells are well known in the art. Culture media used to culture recombinant bacterial cells will depend on the identity of the bacteria. Culture media used to culture recombinant yeast cells will depend on the identity of the yeast. Culture media generally comprise inorganic salts and compounds, amino acids, carbohydrates, vitamins and other compounds that are either necessary for the growth of the host cells or improve health or growth or both of the host cells. In particular, culture media typically comprise manganese (Mn²⁺) and magnesium (Mg²⁺) ions, which are co-factors for many, but not all, glycosyltransferases.

As used herein, the term “fed-batch culture” or “semi-batch culture” are used interchangeably to refer to as an operational technique in biotechnological processes where one or more nutrients (substrates) are fed (supplied) to the bioreactor during cultivation and in which the product(s) remain in the bioreactor until the end of the run. In some embodiments, all the nutrients are fed into the bioreactor.

### Recovery and Purification of Resveratrol and Modified Resveratrol

Resveratrol produced according to the methods disclosed herein can be cis-resveratrol or trans-resveratrol, wherein the trans-resveratrol is a predominant species. Resveratrol, resveratrol glycosides, methylated resveratrol, and other resveratrol derivatives formed and/or recovered according to the invention can be analyzed by techniques generally available to one skilled in the art, for example, but not limited to, thin layer chromatography (TLC), high-performance liquid chromatography (HPLC), ultraviolet-visible spectroscopy/spectrophotometry (UV-Vis), liquid chromatography-mass spectrometry (LC-MS), and nuclear magnetic resonance (NMR).

As set forth herein, the methods of this invention utilize low solubility (in aqueous environments) of resveratrol and the very high aqueous solubility of glycosylated resveratrol, to provide improved and advantageous resveratrol isolation and purification.

In attempts to increase the solubility of resveratrol, monoglucoside variants have previously been isolated from natural plant sources, but these variants demonstrated only minor improvements in solubility (Hyunsu et al., 2012, J. Microbiol. Biotechnol. 22: 1698-1704; Weis et al., 2006, Angew. Chem. Int. Ed. 45: 3534-3538). As set forth herein, higher order glycosylated resverstrol glycosides, inter alia, piceid (3 Glu) or (5 Glu), resveratroloside (4′ Glu), 3,4′-resveratrol glucoside, 3,5-resveratrol diglucoside, 4′,5-resveratrol diglucoside, and 3,5,4′-resveratrol triglucoside, can be produced using a heterologously expressed uridyl diphosphate (UDP)-glycosyltransferase in vitro. These diglycosides and triglycoside of resveratrol have an unexpectedly increased solubility to a level that enables separation from producing microorganisms or insoluble plant material, and subsequent recovery of resveratrol from the soluble fraction by application of glycosidases that cleave the attached glucose groups.

The methods provided herein can also improve the capacity for glycosylated resveratrol to be separated from cells producing resveratrol, particular recombinant cells (microorganisms), or from insoluble material in extracts such as plant extracts, inter alia, by centrifugation or filtration. Once so separated, resveratrol can be recovered from the soluble fraction by application of a β-glucosidase that cleaves sugar moieties from the recovered resveratrol glycoside, said recovered deglycosylated resveratrol having decreased solubility that can cause it to precipitate from the aqueous environment. Recovery of said precipitated aglycone resveratrol is then effected by conventional means such as centrifugation or filtration. See, for instance, Example 4.

Methods for recovering soluble resveratrol glycosides from culture media supporting growth of recombinant cells of the invention expressing UGTs and producing glycosylated resveratrol are dependent upon host cell type and expression construct. As used herein, the terms “recover,” “recovery,” or “recovering” are used interchangeably to refer to obtaining glycosylated resveratrol from the culture media or insoluble resveratrol after enzymatically cleaving the glucoside(s) and/or glycoside(s). In particular embodiments, cell walls can be removed, weakened, or otherwise disrupted to release soluble resveratrol glycoside precursors located in the cytoplasm or periplasm. Said disruption can be accomplished by any means known in the art, including for example, but not limited to, enzymatic treatment, sonication, microfluidization, lysis in a French press or similar apparatus, or disruption by vigorous agitation/milling with glass beads. Lysis or disruption of recombinant host cells is preferably carried out in a buffer of sufficient ionic strength to allow the resveratrol glycosides to remain in soluble form (e.g., more than 0.1 M NaCl, and less than 4.0 M total salts including the buffer).

In some embodiments, addition of two or more glucose residues to resveratrol increases solubility several thousand fold (Table 3), corresponding to approximately 100 g/L resveratrol aglycon. Likewise, the addition of one glucuronic acid residue increases solubility several hundred fold. In some embodiments, the solubilities of Mulberroside E, 3,5-resveratrol diglucoside, and 3,5,4′-resveratrol triglucoside are higher than the values reported in Table 3.

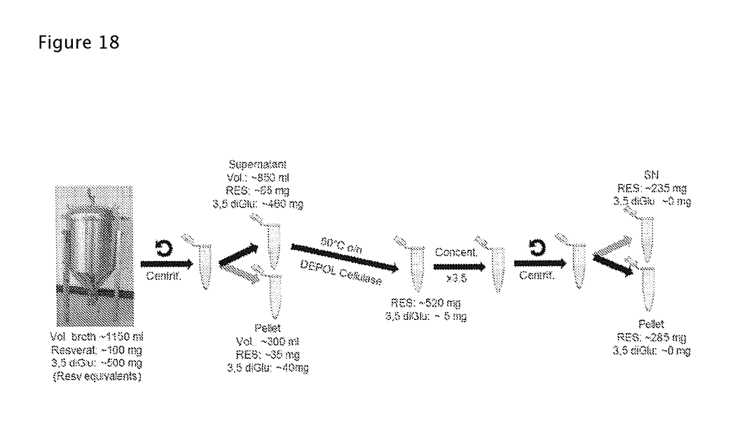

In some embodiments, cleavage of glucose moieties of glycosylated resveratrol (including piceid, resveratroloside, Mulberroside E, and 3,5-resveratrol diglucoside) is achieved upon incubation with recombinant β-glucosidase, Depol™ cellulase (Biocatalysts), Cellulase T. reesei (C2730, Sigma-Aldrich), Glusulase (NEE154001EA, Perkin Elmer), Cellobiase from A. niger (C6105, Sigma-Aldrich), β-galactosidase from A. oryzae (G5160 Sigma-Aldrich), β-glucuronidase, or broth enriched with EXG1 (SEQ ID NO: 123, 124). Incubation of these β-glucosidase enzymes yields significant quantities of insoluble resveratrol. In some embodiments, β-glucosidase-treatment at 50° C. overnight results in near complete release of resveratrol (see, e.g., Example 7, Table 6, FIG. 18).

Advantageously, the resveratrol preparations of the invention have a purity defined herein as a lack or absence of chemical, biochemical or biologic contaminants present in resveratrol preparations prepared from natural sources. In exemplary embodiments, resveratrol preparations provided by the invention do not contain emodin, a plant contaminant present in resveratrol extracted from knotweed having laxative properties not desired for many applications of resveratrol.

Glycosylation of an aglycon of resveratrol and derivatives thereof can lead to improved bioavailability. That is, an increased amount of a glycosylated resveratrol aglycon or glycosylated resveratrol or a derivative thereof can reach the systemic circulation after administration, e.g., oral administration. A glycosylated resveratrol aglycon or glycosylate resveratrol that is ingested by a subject would have the sugars fully or partially removed by the enzymes within the gastrointestinal tract of the subject and subsequently absorbed by the gastrointestinal tract of the subject.

Methods exist for predicting bioavailability of a biomolecule in humans. For example, the Caco-2 cell permeability screen is widely used to assess intestinal transport and predict absorption rates (see, e.g., Hai-Zhi et al., 2000, Rapid Communications in Mass Spectrometry 14:523-28). The fraction of a compound absorbed in a human could be predicted by in vitro Caco-2 cell permeability; if compound permeability in Caco-2 cells reaches 13.3-18.1×10⁻⁶ cm/s, it is predicted that in vivo, permeability in humans would reach 2×10⁻⁴ cm/s, and the predicted fraction of drug absorbed would be >90%, which is defined as highly permeable (Sun et al., 2004, Curr. Opin. Drug Discov. Devel. 7: 75-85). Therefore, in vitro absorption testing is a valuable tool for comparison of structural analogues for improved bioavailability, and to identify biomolecules for clinical studies at early-stage compound discovery and development.

### Resveratrol Compositions and Uses

The invention set forth herein provides methods for producing glycosylated resveratrol and resveratrol derivatives having increased solubility in water and aqueous environments by heterologously expressed uridyl diphosphate (UDP)-glycosyltransferases in vitro. The skilled worker will recognize that low aqueous solubility can complicate commercial use of resveratrol and other like molecules (Gao et al., 2010, Mini Rev Med Chem. 10:550-567) and that an increase of solubility often correlates with a significant improvement in bioavailability (Park et al., 2012, J. Microbiol. Biotechnol. 22: 1698-1704; Yeo et al., 2013, J Chromatogrh B 931: 68-74; Yeo et al., 2013, Mol Nutr Food Res 57: 1015-1025). Therefore, glycosylation of resveratrol, as provided herein, can advantageously increase said bioavailability and provide resveratrol productions that can better be used commercially in foods, beverages, and cosmetics.

A composition containing resveratrol or an analog or derivative thereof can be formulated into a composition and administered to a subject by any suitable route of administration, including oral or parenteral routes of administration. Specific administration modalities include subcutaneous, intravenous, intramuscular, intraperitoneal, transdermal, intrathecal, oral, rectal, buccal, topical, nasal, ophthalmic, intra articular, intra-arterial, sub arachnoid, bronchial, lymphatic, vaginal, and intra uterine administration. In some embodiments, the composition can be in the form of a capsule, liquid (e.g., a beverage), tablet, pill, gel, pellet, foodstuff, dry or wet animal feed, or formulated for prolonged release. In some embodiments, a resveratrol composition can be a solution.

Any of the compositions described herein can be included in a container, pack, or dispenser together with instructions for administration. In some embodiments, the composition is packaged as a single use vial.

In some embodiments, resveratrol, resveratroloside, and piceid are administered once, either orally or intravenously, to CD1 male mice (10 mg/kg, 250 μL/25 g). Blood samples collected by cardiac puncture using heparin treated syringes under terminal inhaled anaesthesia 15 min, 30 min, 1 h, 2 h, 4 h, 8 h, and 24 h post-treatment reveal i) undetectable resveratrol levels in plasma after oral administration, ii) low resveratrol levels in plasma after intravenous administration, iii) detectable piceid levels in plasma after oral and intravenous administration, and iv) systemic conversion of piceid to trans-resveratrol after oral and intravenous administration. (See, e.g., Example 10, Table 7).

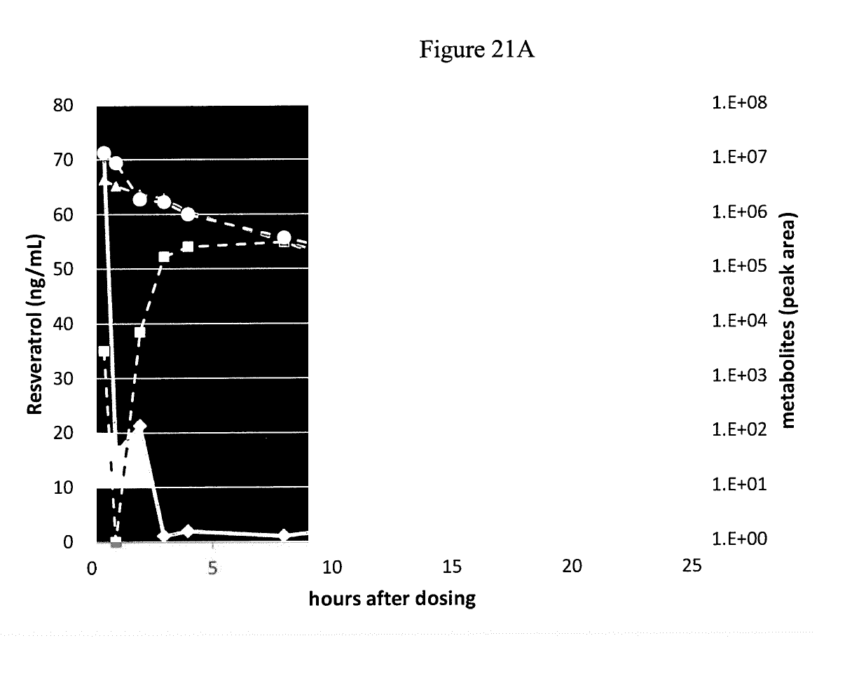

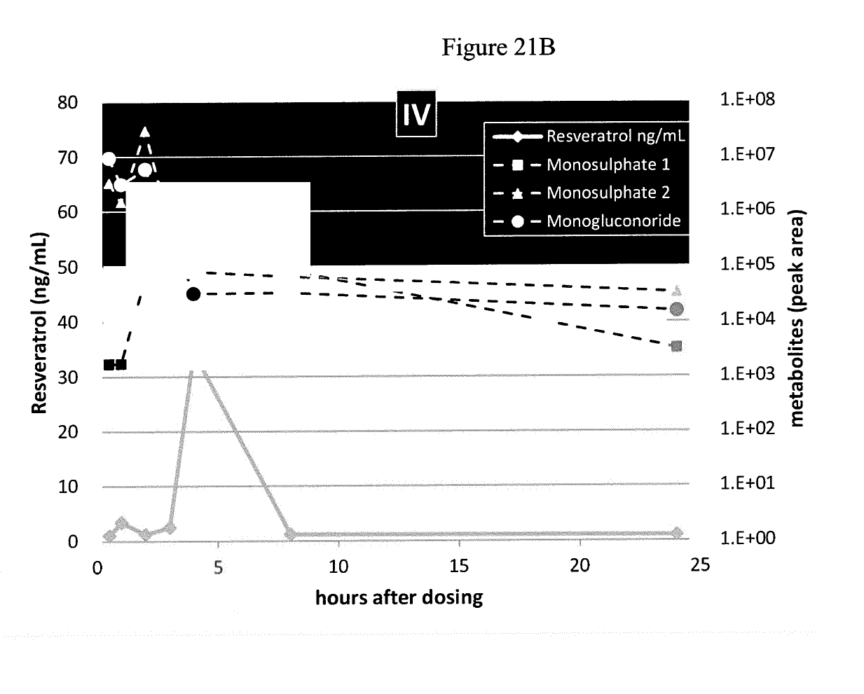

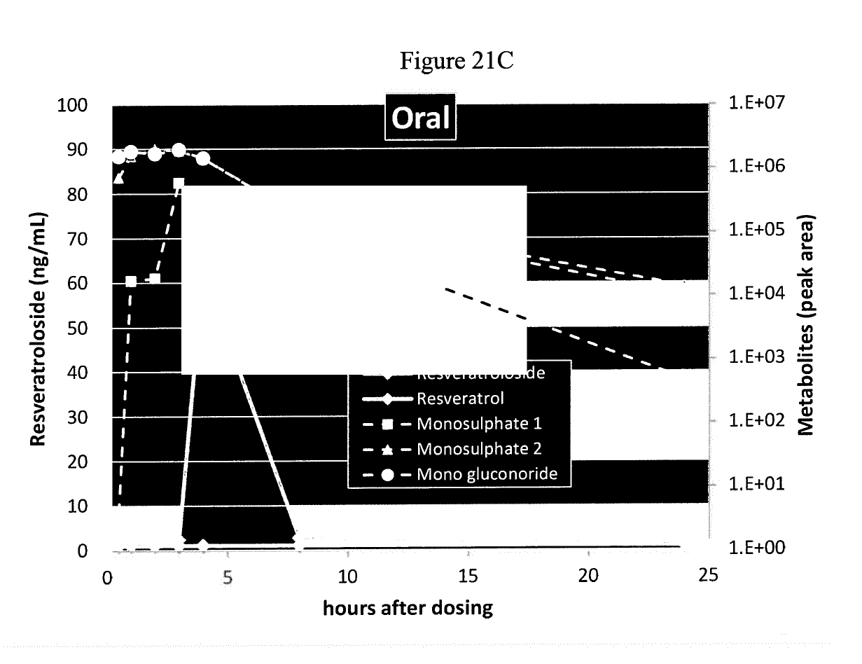

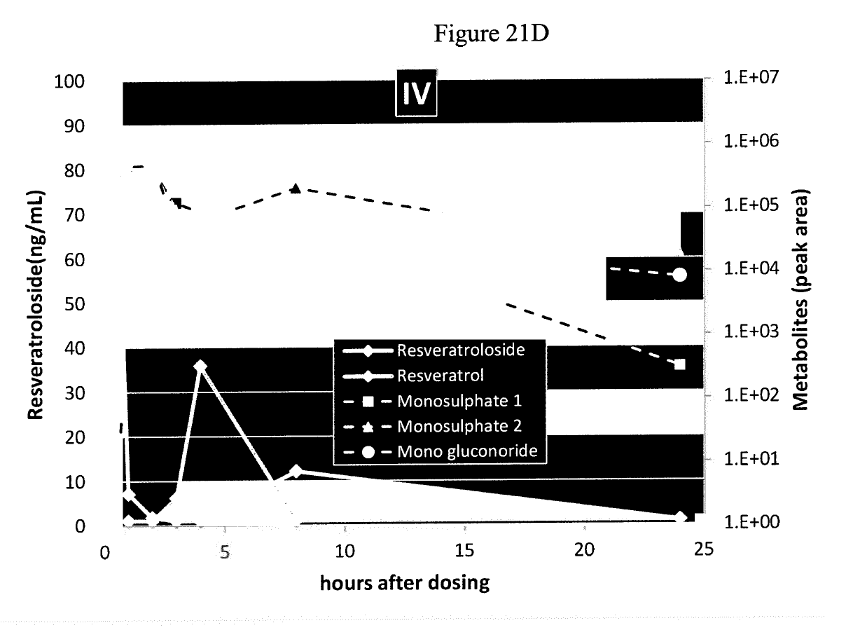

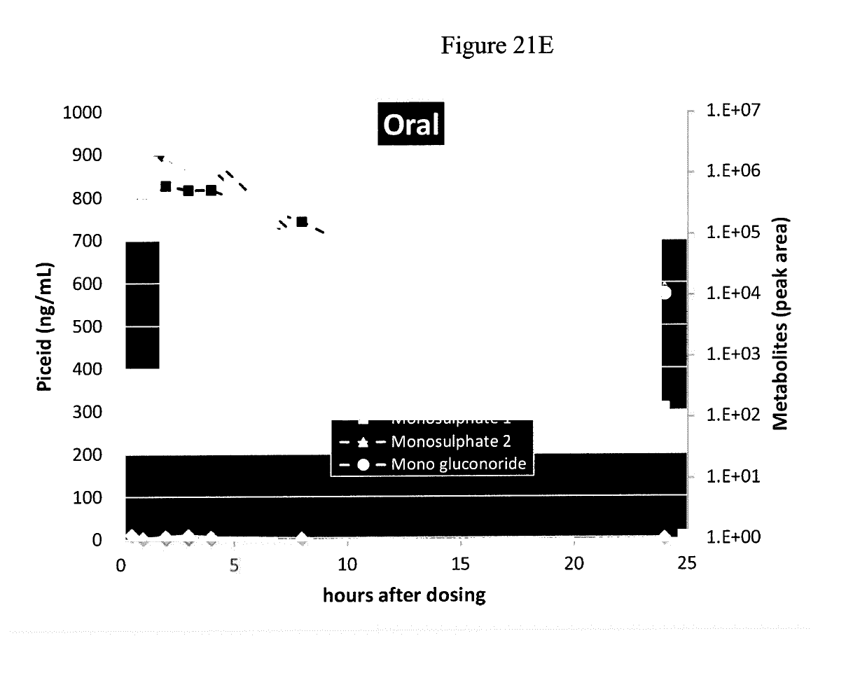

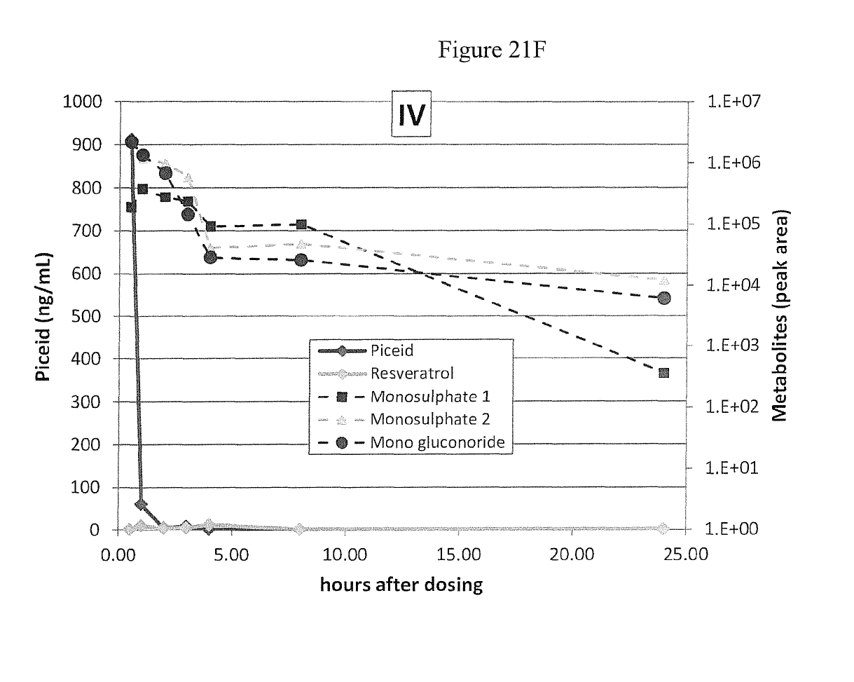

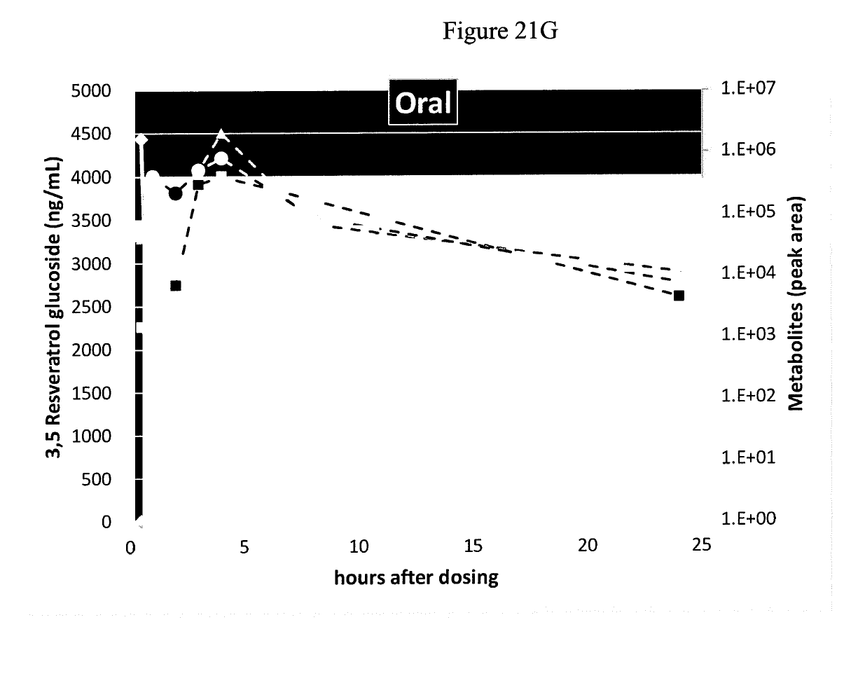

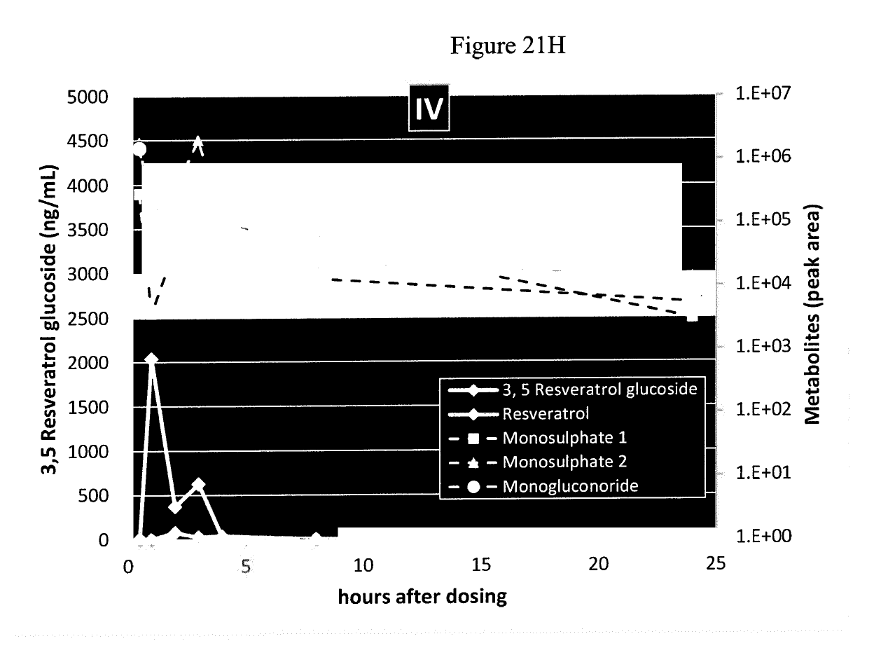

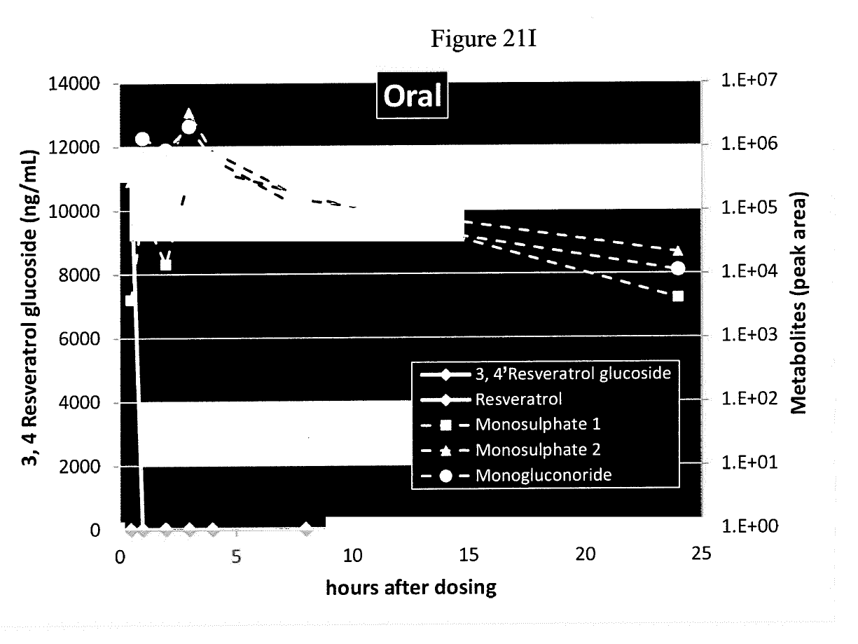

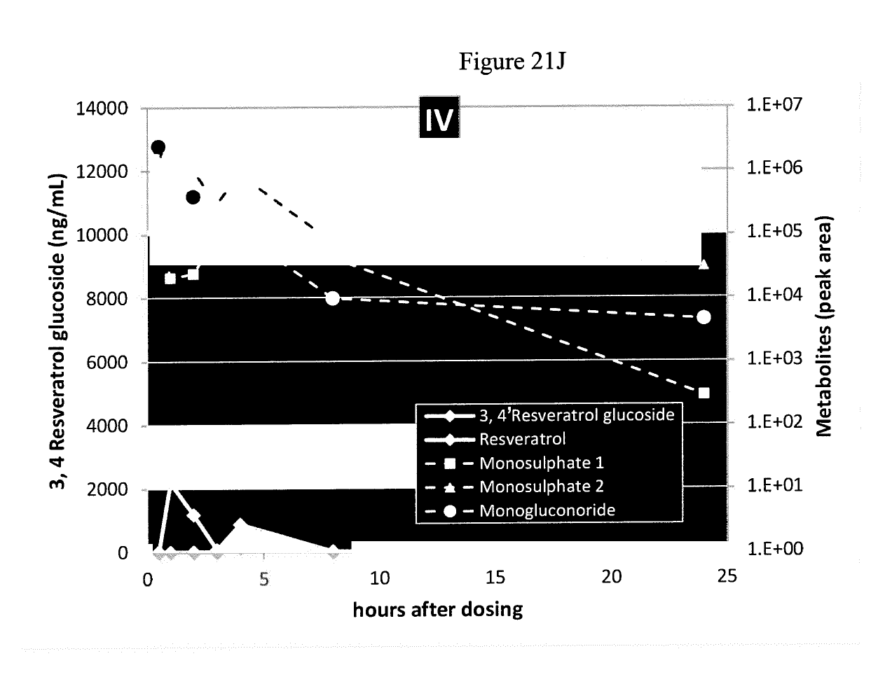

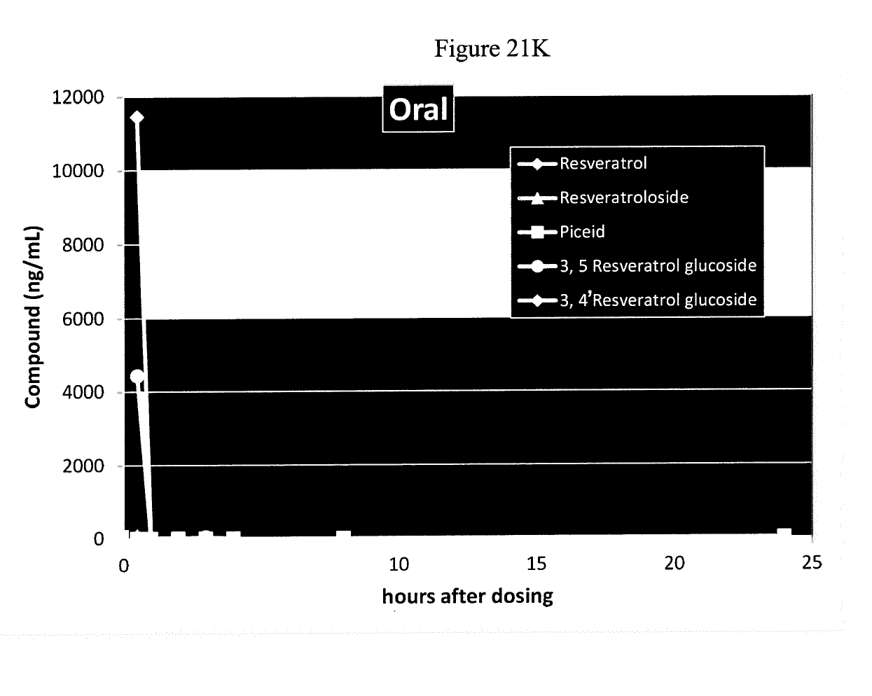

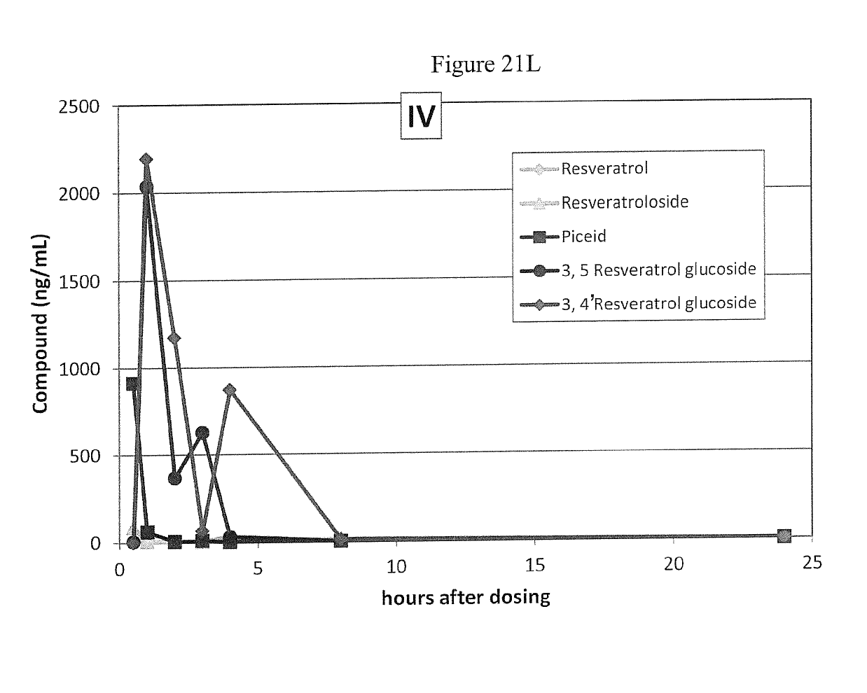

In some embodiments, plasma levels of resveratrol, resveratroloside, piceid, 3,5-resveratrol diglucoside, 3,4′-resveratrol diglucoside, and the metabolites resveratrol 3-sulfate, resveratrol 4′-sulfate, resveratrol 3-glucuronide, monosulphate 1, monosulphate 2, and monogluconoride are measured 0.5, 1, 2, 3, 4, 8, and 24 h post-oral or post-IV administration (see, e.g., Example 10, FIG. 21). In these embodiments, i) resveratrol administered orally clears quickly, ii) intravenous administration of resveratrol results in an increase in resveratrol plasma levels 4 h-post administration, iii) resveratroloside administration orally or intravenously results in detectable levels of resveratrol in plasma, iv) piceid administered orally results in low levels of piceid in plasma, v) piceid administered intravenously results in detectable levels of piceid in plasma, vi) oral and intravenous administration of 3,5-resveratrol diglucoside result in high initial levels of 3,5-resveratrol diglucoside in plasma, vii) oral and intravenous administration of 3,4′-resveratrol diglucoside result in high plasma levels of 3,4′-resveratrol diglucoside. In this embodiment, plasma levels of 3,5-resveratrol diglucoside and 3,4′-resveratrol diglucoside are significantly higher than those of resveratrol, resveratroloside, and piceid following oral and intravenous administration.

The invention will be further described in the following examples, which do not limit the scope of the invention described in the claims.

### EXAMPLES

The Examples that follow are illustrative of specific embodiments of the invention and various uses thereof. They are set forth for explanatory purposes only, and are not to be taken as limiting the invention.

### Example 1

### UDP-Glucose Glycosyltransferase Protein Expression, Purification, and Analysis in Vitro

Recombinant genes encoding UGT enzymes (Table 1) were expressed in E. coli XJb(DE3) Autolysis™ cells harboring the pET30a+ expression vector (Novagen, Nottingham, UK), which carries an N-terminal 6×His tag sequence for affinity purification. The cultures were grown in 1500 mL NZCYM broth (pH 7.0) comprising 15 g Tryptone, 7.5 g NaCl, 7.5 g yeast extract, 1.5 g casamino acids, 3 g MgSO₄ and fortified with 30 mg/L kanamycin, 0.1 mM isopropyl b-D-1-thiogalactopyranoside (IPTG), and 3 mM L-arabinose. After incubation (20 h, 20° C.), cells were pelleted and lysed in 25 mL lysis buffer (10 mM Tris-HCl (pH 7.5)), 5 mM MgCl₂, 1 mM CaCl₂, 3 tablets/100 mL COMPLETE® mini protease inhibitor cocktail (Roche Diagnostics), 14 mg/L deoxyribonuclease (Calbiochem, Nottingham, UK) by a single freeze-thaw cycle to release lysozyme from cell cytoplasm. Purification was performed by adding ⅓ volume of 4× binding buffer (2 M NaCl, 80 mM Tris-HCl (pH 7.5)) to the lysate supernatant, followed by incubation (2 h) with HIS-SELECT® Nickel affinity gel (Sigma-Aldrich, Brøndby, Denmark).

TABLE 1
UGT polypeptides capable of in vitro glycosylation of resveratrol and
resveratrol glycosides

| | | Glucoside Products | | | | |
|---|---|---|---|---|---|---|
| | | A = piceid | | | | |
| | | B = resveratroloside | | | | |
| | | C = 3,5-diglucoside | | | | |
| | | D = 3,4′-diglucoside | | | | |
| | | E = 3,5,4′-triglucoside | | | Activity on | |
| | SEQ ID | F = 4′-bisglucoside | | | Cinnamic | p-Coumaric |
| UGT | NOs | Resv. | Piceid | Resveratroloside | acid | acid |
| 71E1 | 3, 4 | C | C | D | YES | YES |
| 72B1 | 45, 46 | A, B, D | D | — | — | — |
| 72B2_Long | 17, 18 | B, D | D | D | — | — |
| 73B3 | 47, 48 | A, B, D | C, D | D | — | — |
| 73B5 | 19, 20 | C, D | C, D, E | D, E | YES | YES |
| 73C3 | 37, 38 | A, B, D | D | D | — | — |
| 73C5 | 39, 40 | B, D | D | — | — | — |
| 74F1 | 59, 60 | A, B, D | D | D | YES | MINIMAL |
| 75B2 | 21, 22 | A | — | D | YES | YES |
| 76E1 | 23, 24 | A, D | D | D | — | — |
| 76E12 | 49, 50 | | D | — | — | — |
| 71C1 | 51, 52 | A | D | D | YES | YES |
| 76G1 | 25, 26 | — | C | — | — | — |
| 76H1 | 27, 28 | A | D | D | — | — |
| 78D2 | 29, 30 | E | D | D, E | YES | YES |
| 84A3 | 61, 62 | A, B | D | D | YES | YES |
| 84B1 | 31, 32 | B, C, D, E | D, E | D | YES | YES |
| 84B2 | 53, 54 | B | D | — | YES | YES |
| 85A5 | 55, 56 | A | D | — | — | — |
| 88A1 | 7, 8 | A | C | D | — | — |
| BpUGAT 94B1 R25S | 15, 16 | — | — | F | — | — |
| Gtsatom | 57, 58 | A, B | D | D | YES | YES |
| 71C1-188-71C2 | 103, 104 | A | — | D | — | — |
| 71C1-255-71C2 | 67, 68 | A, B | — | D | — | MINIMAL |
| 71C2-255-71E1 | 71, 72 | A, C | C | D | — | — |
| 71C1-255-71E1 | 69, 70 | A, D | — | D | — | — |
| SA-Gtase | 43, 44 | B | D | D | YES | YES |
| 89B1 | 41, 42 | A, B, D | — | D | — | YES |
| 72EV6 | 35, 36 | A, B, D | D | D | — | — |
| 76EV8 | 121, 122 | A, B | C | — | — | — |
| 90A2 | 99, 100 | A, B | C, D | D | — | — |
| 91D2e_b | 117, 118 | — | — | F | — | — |
| 91B1 | 101, 102 | B | D | — | — | — |
| 71B5 | 73, 74 | A | — | — | — | — |
| 72C1 | 75, 76 | B | — | — | — | — |
| 72E2 | 65, 66 | B | — | — | — | — |
| 73B1 | 77, 78 | — | — | — | — | MINIMAL |
| 73B2 | 13, 14 | A | C, D | — | — | — |
| 73B4 | 79, 80 | B | — | — | YES | MINIMAL |
| 73C1 | 81, 82 | A | — | D | — | — |
| 74C1 | 105, 106 | B | — | — | — | — |
| 74F2 | 107, 108 | — | — | — | YES | YES |
| 74G1 | 109, 110 | A, B | C, D | — | — | — |
| 75B1 | 83, 84 | — | — | — | YES | YES |
| 75C1 | 111, 112 | B | — | — | YES | MINIMAL |
| 75D1 | 85, 86 | — | — | — | YES | — |
| 76B1 | 113, 114 | A, B | — | — | — | — |
| 76E4 | 115, 116 | B | — | — | — | — |
| 76E5 | 87, 88 | A | — | — | — | — |
| 76F2 | 89, 90 | — | — | — | YES | MINIMAL |
| 78D3 | 91, 92 | B | — | — | — | — |
| 84A1 | 33, 34 | A, B | — | — | YES | YES |
| 84A2 | 93, 94 | — | — | — | YES | YES |
| 85A1 | 95, 96 | — | — | D | — | — |
| 87A2 | 97, 98 | A | — | — | YES | YES |
| CaUGT2 | 9, 10 | — | — | — | — | — |
| BpUGAT 94B1 | 1, 2 | — | — | — | — | — |

The affinity gel was recovered by centrifugation, and UGT polypeptides were eluted by addition of elution buffer (7.5 ml 20 mM Tris-HCl (pH 7.5), 500 mM NaCl and 250 mM imidazole). Eluted polypeptides were stabilized by addition of glycerol to a final concentration of 50%. SDS-PAGE was performed using NuPAGE® 4-12% Bis-Tris 1.0 mm precast gels (Invitrogen), NuPAGE MOPS (Invitrogen) running buffer, and Simplyblue Safestain (Invitrogen) for Coomassie based gel staining. UGT concentration was semi-quantitatively measured from the staining intensity of the observed UGT band using bovine serum albumin (Sigma-Aldrich, Brøndby, Denmark) as a reference.

In Vitro Glycosylation Assay: Glycosylation reactions were performed in 96 well microtiter plates. Enzyme assays (total volume: 50 μL) comprised 5 μL enzyme solution (approximately 1.25 μg enzyme per reaction), 100 mM Tris-HCl (pH 8), 5 mM MgCl₂, 1 mM KCl, 0.5 U (1 U/μL) calf intestine phosphatase (Fermentas, Helsingborg, Sweden), 1.5 mM UDP-glucose (Roche, Hvidovre, Denmark), and 0.5 mM acceptor substrate (dissolved in DMSO, final concentration 10%). The following acceptor substrates were tested: trans-resveratrol (Fluxome, Stenløse, Denmark), piceid/polydatin (Sigma-Aldrich, Brøndby, Denmark), resveratroloside (purified from a 25 mL enzymatic glycosylation reaction employing Arabidopsis thaliana UGT72B2_Long (SEQ ID NOs: 17, 18) as described by Hansen et al. Phytochemistry 70 (2009) 473-482), cinnamic acid (Sigma-Aldrich, Brøndby, Denmark), and p-coumaric acid (Sigma-Aldrich, Brøndby, Denmark). After incubation of the reaction mixtures (30° C., 20 h), 50 μL 96% ethanol was pipetted to wells, the contents of the well were mixed, and the plates were centrifuged for 5 min at 4000 rpm to separate precipitated enzymes and/or insoluble compounds from the supernatant. 50 μL of each supernatant were transferred to a fresh microtiter plate for LC-MS analysis.

Liquid-Chromatography Mass-Spectrometry (LC-MS) Analysis of Glycosidic Products:

Enzyme-catalyzed resveratrol glycoside formation was analyzed by LC-MS using an Agilent 1100 Series HPLC system (Agilent Technologies) fitted with a Hypersil gold C18 column (100×2.1 mm, 3 μm particles, 80 Å pore size) (ThermoFisher Scientific, Waltham Mass., USA) and hyphenated to a TSQ Quantum (ThermoFisher Scientific) triple quadropole mass spectrometer with electrospray ionization. Elution was carried out using a mobile phase (flow rate: 0.5 mL/min, 30° C.) containing MeCN and H₂O adjusted to pH 2.3 with H₂SO₄ by applying a gradient composed of 10% MeCN for 0.5 min, linear gradient of MeCN from 10% to 100% for 6 min, and 100% MeCN for 1 min. A mass spectrometer and a diode array detector were used to monitor elution of compounds. Glycosides formed were quantified using the absorption measured at the same wavelength at which their respective aglycons had absorption maxima. The assumption that the glycoside and aglycon absorbed equally was validated by comparing the amount of glycoside formed with the amount of aglycon that had decreased. The absorption wavelengths used for quantification were: resveratrol (307 nm); piceid (307 nm); resveratroloside (307 nm); cinnamic acid (277 nm); coumaric acid (307 nm).

Scaled-Up In Vitro Glycosylation Assay:

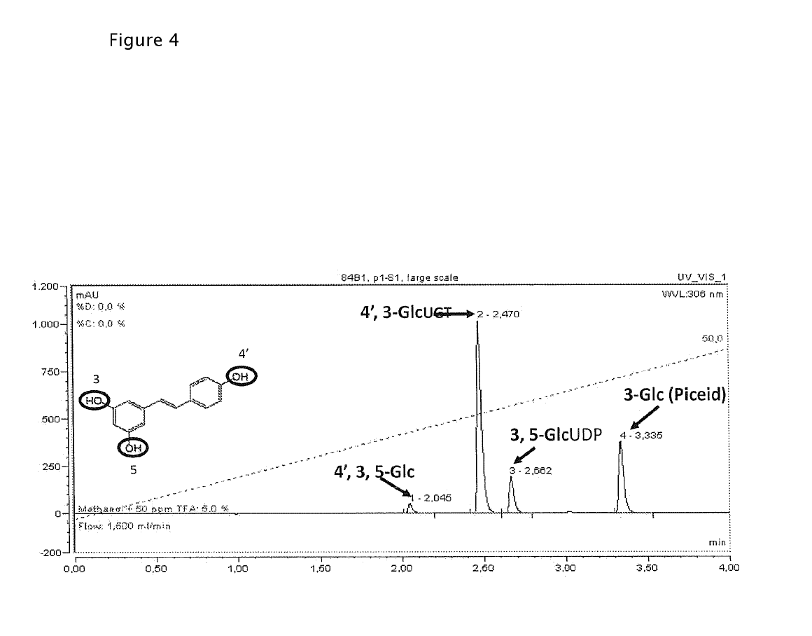

UGT polypeptides that demonstrated glycosylation of resveratrol, piceid, and/or resveratroloside (4′-Glu) were re-analyzed on a larger scale (50 mL assay). Table 2 shows levels of resveratrol and resveratrol glucosides following incubation with the indicated enzymes. Results were not quantitative, as enzyme concentration was not standardized. FIG. 4 shows the activity of UGT84B1 analyzed on a 50 mL scale using piceid as the substrate. In vitro experiments demonstrated that UGTs can glycosylate the resveratrol backbone at all three hydroxyl groups. (FIG. 5).

TABLE 2
In vitro screen UGT enzymes revealing mono-, di-, and tri-glycosides.

| | 3,5,4′- | 3,4′- | 3,5- | | | | 3,5,4′- | 3,4′- | 3,5- | | 3,5,4′- | | |
|---|---|---|---|---|---|---|---|---|---|---|---|---|---|
| | Tri | Di | Di | 4′- | | | Tri | Di | Di | | Tri | 3,4′-Di | |
| UGT | Glu | Glu | Glu | Glu | Piceid | Resveratrol | Glu | Glu | Glu | Piceid | Glu | Glu | 4′-Glu |
| 71E1 | | | 120 | | | | | | 120 | | | 120 | |
| (SEQ | | | | | | | | | | | | | |
| ID NOs: | | | | | | | | | | | | | |
| 3, 4) | | | | | | | | | | | | | |
| 72B2_Long | | 20 | | 80 | | | | 120 | | 20 | | 60 | 60 |
| (SEQ | | | | | | | | | | | | | |
| ID NOs: | | | | | | | | | | | | | |
| 17, 18) | | | | | | | | | | | | | |
| 73B5 | | 20 | 80 | | | | 4 | 20 | 100 | | 8 | 90 | |
| (SEQ | | | | | | | | | | | | | |
| ID NOs: | | | | | | | | | | | | | |
| 19, 20) | | | | | | | | | | | | | |
| 75B2 | | | | | 100 | | | | | 120 | | 100 | 20 |
| (SEQ | | | | | | | | | | | | | |
| ID NOs: | | | | | | | | | | | | | |
| 21, 22) | | | | | | | | | | | | | |
| 76E1 | | 80 | | | 14 | | | 120 | | 10 | | 120 | |
| (SEQ | | | | | | | | | | | | | |
| ID NOs: | | | | | | | | | | | | | |
| 23, 24) | | | | | | | | | | | | | |
| 76E12 | | | | 100 | | 20 | | 60 | | 60 | | | 120 |
| (SEQ | | | | | | | | | | | | | |
| ID NOs: | | | | | | | | | | | | | |
| 49, 50) | | | | | | | | | | | | | |
| 76G1 | | | | | | 110 | | | 100 | 25 | | | 120 |
| (SEQ | | | | | | | | | | | | | |
| ID NOs: | | | | | | | | | | | | | |
| 25, 26) | | | | | | | | | | | | | |
| 76H1 | | | | | 16 | 94 | | | | 120 | | 100 | 20 |
| (SEQ | | | | | | | | | | | | | |
| ID NOs: | | | | | | | | | | | | | |
| 27, 28) | | | | | | | | | | | | | |
| 78D2 | 10 | 80 | | | | | | 120 | | 10 | 8 | 100 | |
| (SEQ | | | | | | | | | | | | | |
| ID NOs: | | | | | | | | | | | | | |
| 29, 30) | | | | | | | | | | | | | |
| 84B1 | 30 | 50 | 10 | 20 | | | 10 | 100 | | | | 60 | 70 |
| (SEQ | | | | | | | | | | | | | |
| ID NOs: | | | | | | | | | | | | | |
| 31, 32) | | | | | | | | | | | | | |
| 88A1 | | | | | 120 | | | | 10 | 110 | | 80 | 50 |
| (SEQ | | | | | | | | | | | | | |
| ID NOs: | | | | | | | | | | | | | |
| 7, 8) | | | | | | | | | | | | | |
| 71C1- | | | | | 120 | | | | | 120 | | 120 | |
| 188- | | | | | | | | | | | | | |
| 71C2 | | | | | | | | | | | | | |
| (SEQ | | | | | | | | | | | | | |
| ID NOs: | | | | | | | | | | | | | |
| 103, | | | | | | | | | | | | | |
| 104) | | | | | | | | | | | | | |
| 71C1- | | | | 10 | 110 | | | | | 120 | | 90 | 30 |
| 255- | | | | | | | | | | | | | |
| 71C2 | | | | | | | | | | | | | |
| (SEQ | | | | | | | | | | | | | |
| ID NOs: | | | | | | | | | | | | | |
| 67, 68) | | | | | | | | | | | | | |
| 71C2- | | | 10 | | 40 | 50 | | | 120 | | | 10 | 110 |
| 255- | | | | | | | | | | | | | |
| 71E1 | | | | | | | | | | | | | |
| (SEQ | | | | | | | | | | | | | |
| ID NO: | | | | | | | | | | | | | |
| 71, 72) | | | | | | | | | | | | | |
| 71C1- | | 40 | | | 60 | | | | | 120 | | 120 | |
| 255- | | | | | | | | | | | | | |
| 71E1 | | | | | | | | | | | | | |
| (SEQ | | | | | | | | | | | | | |
| ID NOs: | | | | | | | | | | | | | |
| 69, 70) | | | | | | | | | | | | | |
| 72EV6 | | 40 | | 20 | 50 | | | 90 | | 40 | | | 80 |
| (SEQ | | | | | | | | | | | | | |
| ID NOs: | | | | | | | | | | | | | |
| 35, 36) | | | | | | | | | | | | | |
| Control | | | | | | 100 | | | | | | | |

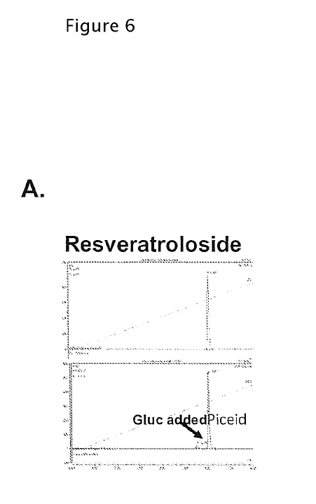

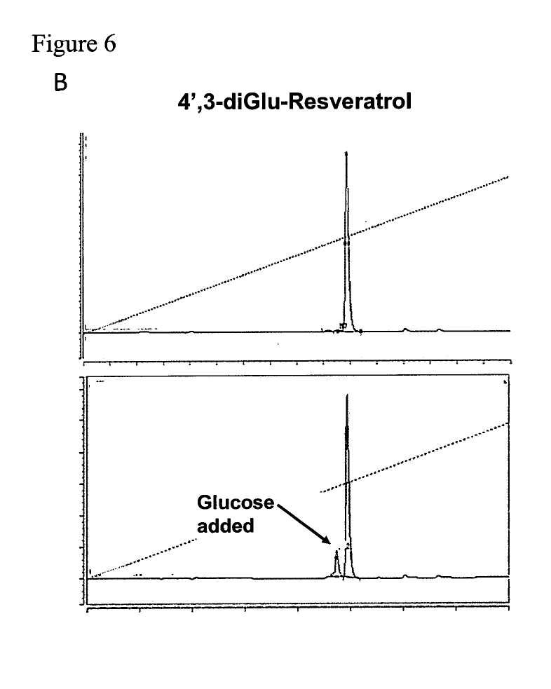

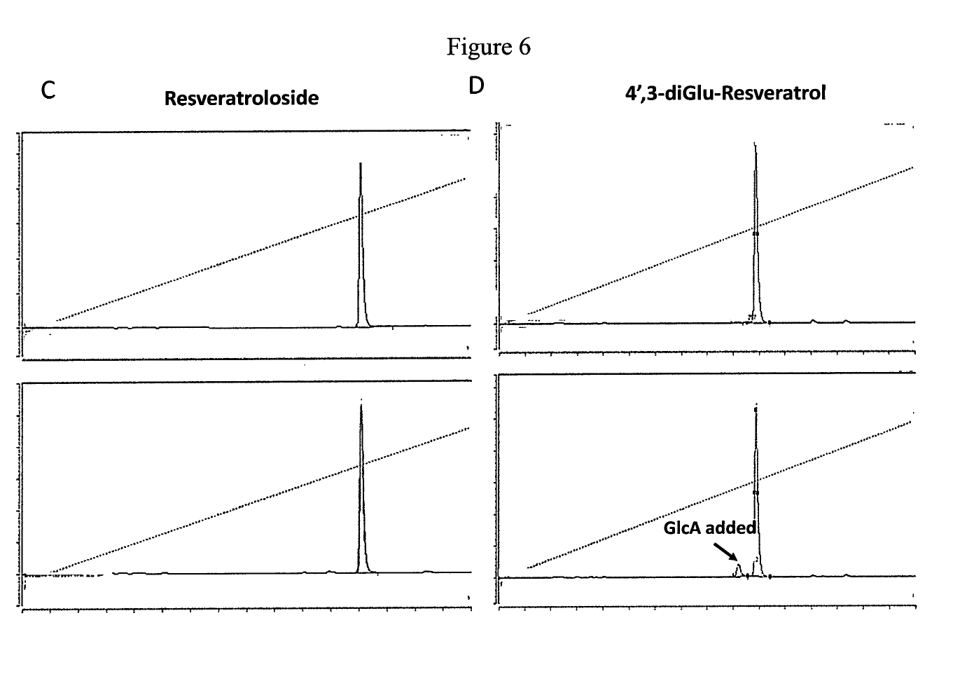

Results of the in vitro screening with BpUGT94B1 R25S (SEQ ID NOs: 15, 16) and UGT91D2e_b (SEQ ID NOs: 117, 118) revealed unidentified minor peaks. These peaks were observed with resveratroloside as the substrate and UDP-glucose as the donor. Peaks indicated a bis-glucoside (glucose on glucose in the 4′ position). When BpUGT94B1 R25S was re-purified and tested, results revealed reproducible minor peaks indicating resveratrol 4′-bis-glucoside being made from resveratroloside and piceid, 4′-bis-glucoside being made from 3,4′-resveratrol diglucoside (FIGS. 6A, B).

BpUGT94B1 WT enzyme (SEQ ID NOs: 1, 2) was also purified and tested. UDP-glucuronic acid (UDP-GlcA) was used as sugar donor. This experiment was conducted in vitro, and a glucuronic acid molecule (rather than glucose) was added to the glucose at the 4′ position. A very minor peak was observed for 3,4′-resveratrol diglucoside but not for resveratroloside (FIGS. 6C, D).

Resveratrol glycosides produced in 50 mL volumes were subsequently purified (200-300 mg). Identity and structure of purified resveratrol glycosides was confirmed by mass spectrometry (MS) and nuclear magnetic resonance (NMR).

Purification of Enzymatically-Produced Resveratrol Glycosides:

Following incubation (20 h, 30° C.) of reaction mixtures on the 50 mL scale, the reaction mixtures were filtered (Amicon Ultra-15 centrifugal filter, 30 kDa cutoff; Millipore, Cork, Ireland) to remove protein. The filters were washed with DMSO to recover any precipitated glycosylated product. The resveratrol glycosides produced were purified by preparative HPLC with an Agilent 1200 series preparative HPLC system (Agilent Technologies, Nrum, Denmark) fitted with a Thermo Biobasic 018-silica column (150×30 mm, 10 μm particles, 150 Å pore size) (ThermoFisher Scientific, Waltham Mass., USA). Elution was carried out using a mobile phase (flow rate: 20 ml/min) containing MeCN and H₂O (0.01% TFA) by applying a gradient composed of 5% MeCN for 5 min and linear gradient from 5% to 100% for 45 min. A diode array detector was used to monitor elution of compounds by UV-absorption. Fractions containing glycosides were collected and evaporated to dryness using a vacuum centrifuge (Heto-vac, Heto-Holten, Denmark).

NMR Analysis of Resveratrol Glycosides:

All NMR experiments were performed in DMSO-d6 at 25° C. using a Bruker Avance III 800 MHz NMR spectrometer equipped with a 5 mm cryogenic TCI probe. The structures of 3,5-resveratrol diglucoside, 3,4′-resveratrol diglucoside, resveratrol-3-O-β-glucoside, and resveratrol-4′-O-3-glucoside were solved by means of standard homo- and heteronuclear multipulse NMR experiments, namely 1H,1H-COSY, 1H, 13C-HSQC and 1H, 13C-HMBC experiments. The ¹³C-NMR spectrum (201.21 MHz) of 3,5-resveratrol diglucoside showed signals at 158.9 (2C) 157.7 (1C) 139.7 (1C) 129.6 (1C) 129.5 (1C) 128.4 (2C) 125.2 (1C) 115.9 (2C) 108.0 (2C) 103.3 (1C) 100.8 (2C) 77.8 (2C) 77.4 (2C) 73.8 (2C) 70.2 (2C) 61.1 (2C); Double bond: trans (3J(H,H)=16.4 Hz). The ¹H-NMR spectrum (800 MHz) showed multiple peaks at 3.15 (t, J=9.29 Hz, 2H) 3.23-3.26 (m, 2H) 3.27-3.30 (m, 2H) 3.38-3.41 (m, 2H) 3.47 (dd, J=11.86, 6.24 Hz, 2H) 3.73 (dd, J=11.62, 1.59 Hz, 2H) 4.88 (d, J=7.58 Hz, 2H) 6.58 (t, J=2.08 Hz, 1H) 6.77 (d, J=8.56 Hz, 2H) 6.87 (d, J=1.96 Hz, 2H) 6.92 (d, J=16.38 Hz, 1H) 7.13 (d, J=16.38 Hz, 1H) 7.41 (d, J=8.56 Hz, 2H). The ¹³C-NMR spectrum (201.21 MHz) of 3,4′-resveratrol diglucoside showed signals at 159.3 (1C) 158.6 (1C) 157.4 (1C) 139.4 (1C) 131.2 (1C) 128.5 (1C) 128.1 (2C) 127.2 (1C) 116.8 (2C) 107.8 (1C) 105.3 (1C) 103.3 (1C) 101.0 (10) 100.6 (1C) 77.6 (1C) 77.5 (1C) 77.4 (1C) 77.1 (1C) 73.7 (1C) 73.7 (1C) 70.1 (1C) 70.1 (10) 61.2 (1C) 61.1 (1C); Double bond: trans (3J(H,H)=16.4 Hz. The ¹H-NMR spectrum (800 MHz) showed multiple peaks at 3.15-3.19 (m, 2H) 3.23 (ddd, J=17.06, 9.11, 7.83 Hz, 2H) 3.26-3.31 (m, 2H) 3.32-3.36 (m, 2H) 3.48 (m, J=11.90, 11.90, 5.90 Hz, 2H) 3.68-3.76 (m, 2H) 4.81 (d, J=7.83 Hz, 1H) 4.89 (d, J=7.58 Hz, 1H) 6.36 (t, J=2.20 Hz, 1H) 6.60 (t, J=1.50 Hz, 1H) 6.77 (t, J=1.50 Hz, 1H) 6.98 (d, J=16.14 Hz, 1H) 7.03 (d, J=8.80 Hz, 2H) 7.10 (d, J=16.38 Hz, 1H) 7.52 (d, J=8.80 Hz, 2H). The ¹³C-NMR spectrum (151 MHz) of resveratrol-3-O-glucoside showed signals at 160.5 159.6 158.5 141.5 130.4 130.0 128.9 126.7 116.5 108.4 107.1 104.1 102.4 78.3 78.1 75.0 71.5 and 62.6 ppm (12 aglycon signals and 6 glucose signals). The ¹H-NMR spectrum (600 MHz) showed multiple peaks (9H) in the range 6.4-7.4 ppm corresponding to the resveratrol aglycon moiety and multiple peaks (6H) in the range 3.3-4 ppm corresponding to the glucose residue. The ¹³C-NMR spectrum (151 MHz) of resveratrol-4′-O-glucoside showed signals at 159.7 158.7 141.0 133.2 128.9 128.6 128.5 118.0 106.0 103.0 102.3 78.2 78.0 75.0 71.4 and 62.6 ppm (10 resveratrol aglycon signals and 6 glucose signals). The ¹H-NMR spectrum (600 MHz) showed multiple peaks in the range 6.2-7.5 ppm corresponding to the resveratrol aglycon moiety and multiple peaks (6H) in the range 3.4-3.9 ppm corresponding to the glucose residue. The signal of the anomeric proton was positioned at 4.91 ppm.

### Example 2

### Solubility of Purified Enzymatically-Produced Resveratrol Glycosides

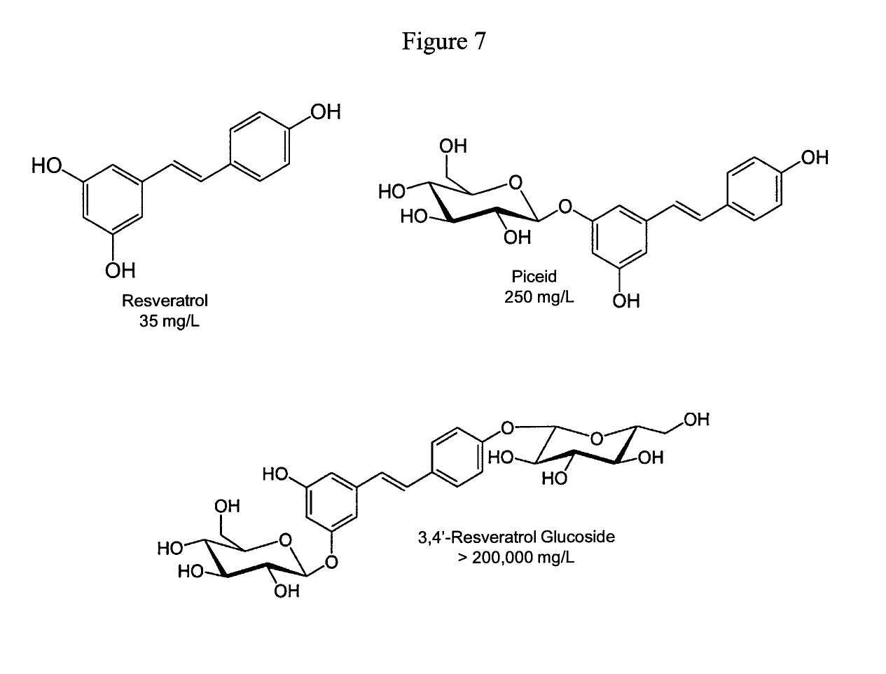

Purified resveratrol glycosides were further tested for solubility. The solubility resveratrol, piceid, resveratroloside, Mulberroside E (3,4′-resveratrol diglucoside), 3,5-resveratrol diglucoside, and 3,5,4′-resveratrol triglucoside was tested as follows. All compounds were lyophilized from nanopure H₂O, acetonitrile, and Trifluoroacetic acid (TFA). Purity of all compounds was tested by HPLC, being at least 95% in every case, and identities of all purified compounds were verified by NMR. A minimal amount of nanopure H₂O (1 mL) was added to the purified compounds. Samples were vortexed for 1 min to facilitate solubilization and centrifuged to remove non-dissolved material. The concentration of the compounds was measured by HPLC. The solubility values are shown in Table 3, FIG. 7.

TABLE 3
Solubility in Water of All Purified Compounds.

| | Solubility | Solubility | | |
|---|---|---|---|---|
| Resveratrol | mg/L | mg/L | MW | |
| derivative | (Literatrure) | (in house) | g/mol | Origin |
| Resveratrol | not found | >270,000 | 715 | in vitro |
| 3,5,4′ tri- | | | | (UGT) + |
| glucoside | | | | purified |
| Resveratrol | not found | >210,000 | 553 | in vitro |
| 3,5 di- | | | | (UGT) + |
| glucoside | | | | purified |
| Resveratrol | not found | >280,000 | 553 | in vitro |
| 5,4′ di- | | | | (UGT) + |
| glucoside | | | | purified |
| Resvera- | not found | 1,300 | 390 | in vitro |
| troloside | | | | (UGT) + |
| (4′-Glu) | | | | purified |
| Piceid (3-Glu) | 69 | 200-300 | 390 | Sigma |
| Resveratrol | 15-30 | 30-40 | 228 | Evolva/Fluxome |
| Maltosyl-α- | 128,100 | not yet | ~714.67 | not yet |
| 1-4-Piceid | | tested | | obtained |
| 3-glucuronide- | not found | >10,000 | 404.4 | Commercial |
| Resveratrol | | | | |

The results in Table 3 demonstrated a dramatic increase in H₂O solubility upon addition of two or more glucose moieties to the resveratrol backbone. Solubility of purified resveratrol glycosides and other derivatives was analyzed by H₂O saturation and high-performance liquid chromatography (HPLC) quantification on both lyophilized and non-lyophilized material. Results revealed that the addition of two or more glucose residues increased solubility several thousand fold (Table 3), corresponding to approximately 100 g/L resveratrol aglycon. Likewise, the addition of one glucuronic acid residue increased solubility several hundred fold. It was reasoned that increased solubility of resveratrol when glycosylated correlates with increased bioavailability. Moreover, the wide difference in solubility in aqueous environments between resveratrol and its di- or tri-glucosides were exploited in a purification method as described in Example 4 below.

This experiment represents the first time that resveratroloside, Mulberroside E, 3,5-resveratrol diglucoside, and 3,5,4′-resveratrol triglucoside were purified to levels that allow for their solubility in H₂O to be analyzed. For Mulberroside E, 3,5-resveratrol diglucoside, and 3,5,4′-resveratrol triglucoside, no insoluble pellet was observed. This indicates that the purified material was completely dissolved in the volume of nanopure H₂O used and that the saturation point was not reached for the compounds; thus, the solubility values for provided in Table 3 for Mulberroside E, 3,5-resveratrol diglucoside, and 3,5,4′-resveratrol triglucoside are likely underestimated.

### Example 3

### Cleavage of Resveratrol Glycosides by β-Glucosidases

To determine whether resveratrol could be recovered from resveratrol glycosides, cleavage of the glucose moieties was studied, an essential step in purification of insoluble resveratrol. It is known that β-bound glucoses, as the ones added by the UGT enzymes, are cleaved by β-glucosidases. S. cerevisiae has several β-glucosidases including the ones encoded by the genes EXG1 (SEQ ID NOs: 123, 124), BGL2 (SEQ ID NOs: 125, 126), EXG2 (SEQ ID NOs: 127, 128), SPR1 (SEQ ID NOs: 129, 130), ACF2 (SEQ ID NOs: 131, 132), DSE4 (SEQ ID NOs: 133, 134), and SCW11 (SEQ ID NOs: 135, 136).

Cells from two S. cerevisiae strains, one with a functional EXG1 (SEQ ID NOs: 123, 124) and one lacking the EXG1 gene, were incubated in the presence of 125 mg/L Piceid (Polydatin, 15721 Sigma-Aldrich) overnight, and piceid and resveratrol were subsequently measured in the supernatant. After incubation of piceid with the EXG1 cells, approximately 25% of the compound had been cleaved, yielding resveratrol. However, when piceid was incubated with the EXG1 cells, less than 0.5% of the compound was cleaved, demonstrating that EXG1 is a main β-glucosidase for cleaving piceid (and other resveratrol glucosides) in S. cerevisiae. As well, upon deletion of EXG1 in yeast, no β-glucosidase activity was observed. Therefore, absence of EXG1 activity is required to prevent intracellular cleavage of resveratrol glucosides produced in yeast.

To screen for β-glucosidase enzymes capable of cleaving glucose moieties from piceid, resveratroloside, Mulberroside E, and 3,5-resveratrol diglucoside, these resveratrol glucosides were incubated with the following enzymes: recombinant β-glucosidase (GO16L, IFF); Depol™ cellulase (Biocatalysts); Cellulase T. reesei (C2730, Sigma-Aldrich); Glusulase (NEE154001EA, Perkin Elmer); Cellobiase from A. niger (C6105, Sigma-Aldrich); β-galactosidase from A. oryzae (G5160 Sigma-Aldrich); β-glucuronidase (G0751 Sigma-Aldrich); and broth enriched with EXG1 (SEQ ID NOs: 123, 124). Following incubation, resveratrol and resveratrol glucoside concentrations were measured. Each of the enzymes tested cleaved glucose moieties from the resveratrol glucosides, yielding insoluble resveratrol (Table 4).

TABLE 4
Cleavage of Glucose from Resveratrol
Glucosides by β-Glucosidases in vitro.
% cleavage in conditions tested

| | | Resvera- | 3,5-di- | 3,4′-di- |
|---|---|---|---|---|
| β-glucosidase | Piceid | troloside | Glc-Resv | Glc-Resv |
| 1 - β-glucosidase | ✓ | ✓ | ✓ | ✓ |
| (IFF) | | | | |
| 2 - Depol IFF | ✓ | ✓ | ✓ | ✓ |
| (cellulase) | | | | |
| 3 - Cellulase | ✓ | ✓ | ✓ | ✓ |
| T. reesei | | | | |
| 3b - Cellulase | ✓ | ✓ | ✓ | ✓ |
| T. resei | | | | |
| 4 - Glusulase | ✓ | ✓ | ✓ | ✓ |
| 5 - Cellobiase | ✓ | ✓ | ✓ | ✓ |
| A. niger | | | | |
| 6 - β-galactosidase | ✓ | ✓ | ✓ | ✓ |
| A. oryzae | | | | |
| 7 - β-glucuronidase | ✓ | ✓ | ✓ | ✓ |
| 8 - EXG1 broth | ♦ | • | • | ♦ |

✓ = >95%
♦ = 50-95%
• = 5-49%
X = 0%

This experiment demonstrated that significant quantities of resveratrol aglycon were produced and purified upon incubation of resveratrol glucosides with β-glucosidase enzymes and was the first known experiment demonstrating that glucose moieties can be cleaved enzymatically from resveratrol diglucosides.

### Example 4

### Resveratrol Purification

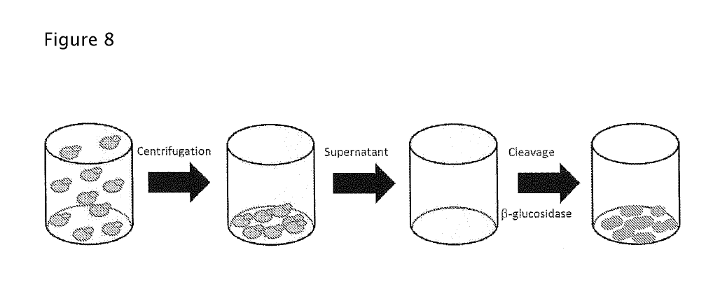

The solubility of resveratrol glycosides is exploited in the purification of resveratrol. Resveratrol glycosylated in vivo (i.e., production of resveratrol glycosides by an engineered plant or microorganism or bioconversion of resveratrol-containing plant extract) or in vitro (i.e., glycosylation of added resveratrol or a plant extract) is more soluble in H₂O than resveratrol, as confirmed by Example 2. Soluble resveratrol glycosides were separated from cells or plant debris by centrifugation or filtration. Upon separation of resveratrol glycosides from cells or plant debris, glucose moieties were cleaved enzymatically, releasing insoluble resveratrol that was subsequently separated from the soluble fraction (FIG. 8).

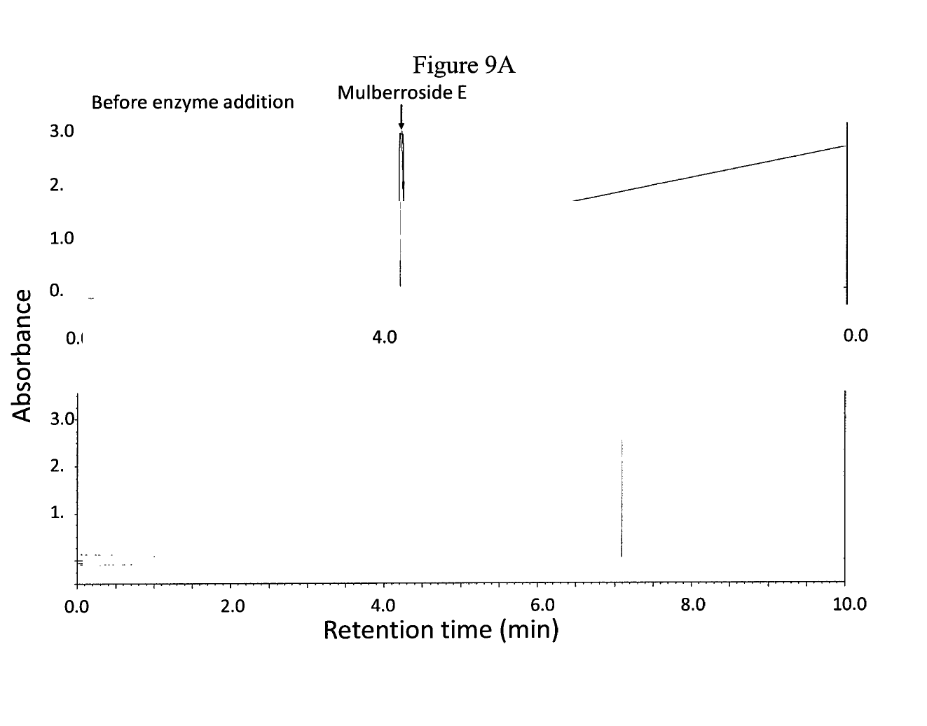

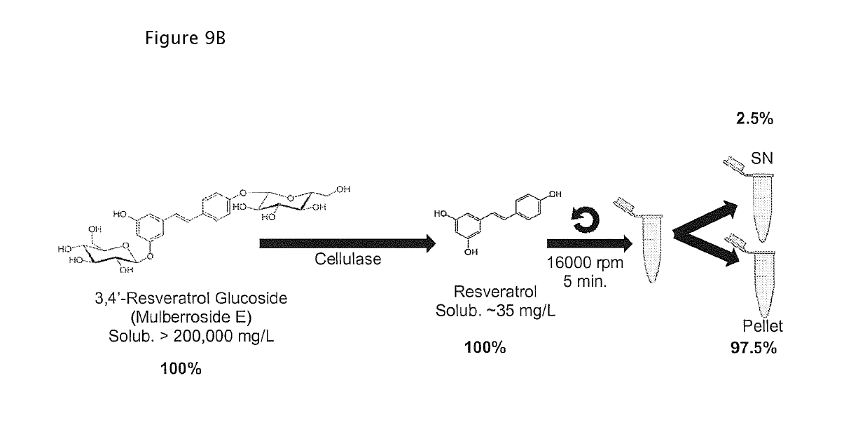

To recover resveratrol from Mulberroside E in vivo, 0.5 mL of a buffer containing 25,000 mg/L Mulberroside E were incubated overnight with 10 μL of Depol™ cellulase (Biocatalysts). HPLC analysis indicated that virtually all Mulberroside E was cleaved, releasing resveratrol (FIG. 9A). The tube was then centrifuged for 5 min at 16000 rpm; insoluble resveratrol in the resulting pellet and soluble resveratrol in the supernatant were quantified by HPLC. Approximately 97.5% of resveratrol was found in the insoluble pellet, and approximately 2.5% of resveratrol was found in the soluble fraction (FIG. 9B). This indicates that it is possible to achieve a high recovery of resveratrol in the absence of solvents or other chemicals.

### Example 5

### In Vivo Glycosylation of Resveratrol

Genes encoding UGT polypeptides (FIG. 10) were amplified by PCR, cloned individually into a p415 GPD vector, and transformed into a resveratrol-producing EXG1 knockout S. cerevisiae strain. EXG1 (YLR300W, SEQ ID NOs: 123, 124), which codes for the major exo-1,3-beta-glucanase of the yeast cell wall, was shown to be highly efficient in releasing glucose moieties from glycosylated resveratrol molecules. All cultures were grown in Delft media for 72 h at 30° C. and 300 rpm in 24 deep-well plates. Samples were prepared for HPLC by mixing 500 μL of each culture with 500 μL 96% ethanol and centrifuging for 5 min at 13000 rpm. The supernatant of each sample was analyzed by HPLC using a mobile phase (flow rate of 1 mL/min) comprising acetonitrile and H₂O and applying a gradient composed of acetonitrile from 5 to 70% for 10 min. Presence of resveratrol and resveratrol glycosides was analyzed by absorbance at 306 nm. Results in FIG. 10 are the mean of three independent cultures.

Formation of resveratrol monoglucosides was detected. Resveratroloside (4′-resveratrol monoglucoside) was produced by UGT72B2_Long (SEQ ID NOs: 17, 18), UGT73C3 (SEQ ID NOs: 37, 38), UGT73C5 (SEQ ID NOs: 39, 40), UGT89B1 (SEQ ID NOs: 41, 42), and UGT84A3 (SEQ ID NOs: 61, 62) in minute amounts (FIG. 10). Piceid (3-Glc) was produced by several UGTs in minute amounts and in a larger amount with UGT71E1 (SEQ ID NOs: 3, 4) (FIG. 10).

3,5-resveratrol diglucoside was detected upon expression of several UGT polypeptides. In most cases, only minute amounts were observed in contrast to the more substantial amount of 3,5-resveratrol diglucoside produced by UGT71E1 (SEQ ID NOs: 3, 4) and UGT84B1 (SEQ ID NOs: 31, 32) (FIG. 10).

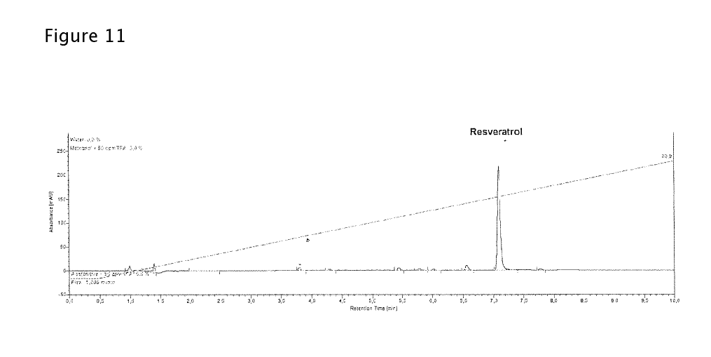

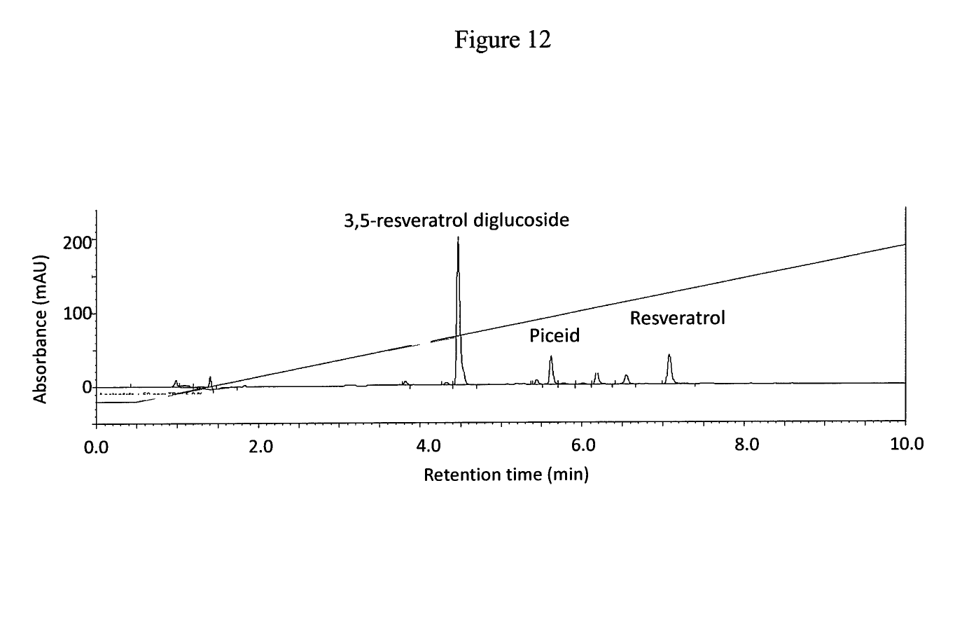

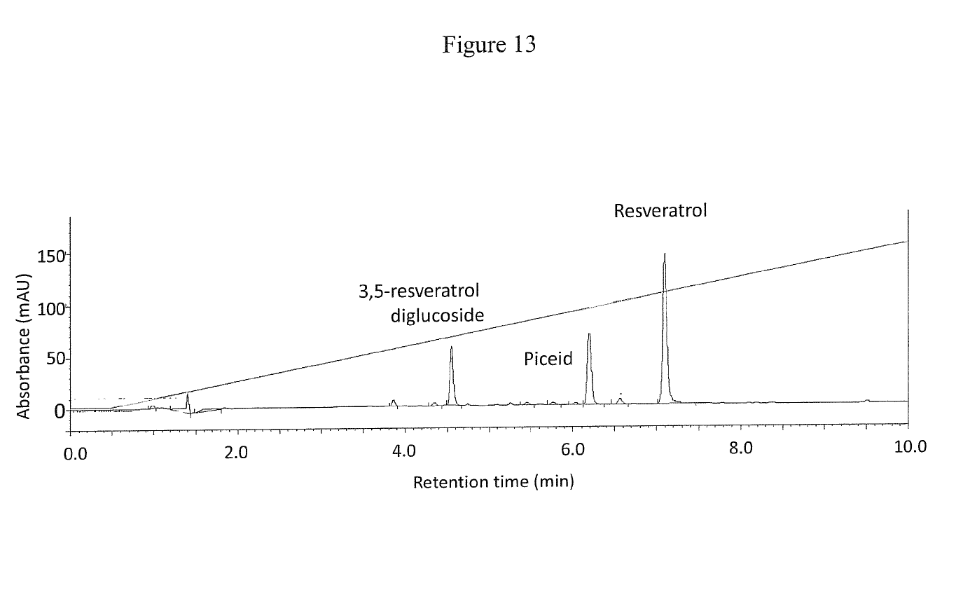

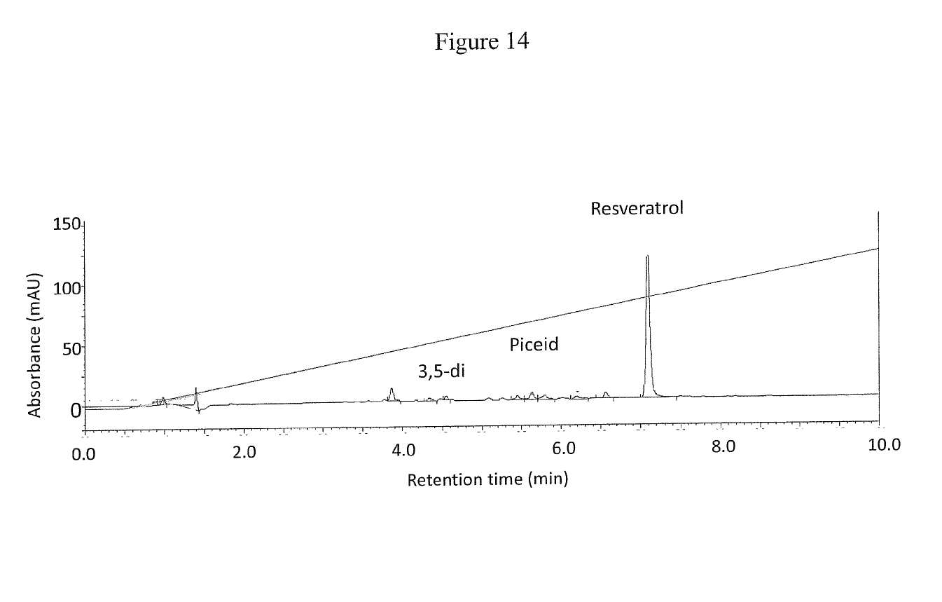

FIGS. 11-14 show characteristic HPLC chromatograms analyzing broth from the resveratrol-producing strain not expressing a UGT polypeptide (empty p415 GPD vector, FIG. 11), expressing UGT71E1 (SEQ ID NOs: 3, 4; FIG. 12), expressing UGT84B1 (SEQ ID NOs: 31, 32, FIG. 13), or expressing UGT73B5 (SEQ ID NOs: 19, 20, FIG. 14). UGT71E1 (SEQ ID NOs: 3, 4) consumed more resveratrol than other UGTs tested and produced piceid (3-Glc) and 3,5-resveratrol diglucoside (FIGS. 10, 12). UGT84B1 (SEQ ID NOs: 31, 32) also produced a substantial amount of the piceid and 3,5-resveratrol diglucoside (FIGS. 10, 13), whereas UGT73B5 (SEQ ID NOs: 19, 20) produced lesser amounts of glycosylated resveratrol (FIGS. 10, 14). Other UGTs, including the codon-optimized UGT72B2_Long (UGT72B2_GA, SEQ ID NOs: 63, 18), were shown to produce resveratroloside in minute amounts, but production of 3,4′-resveratrol diglucoside was undetected.

### Example 6

### In Vivo Glycosylation of Resveratrol in a Production Strain

UGTs shown to be functional upon expression in yeast (i.e., UGT72B2_Long (SEQ ID NOs: 17, 18)) were expressed to achieve glycosylation of resveratrol. In parallel, genes encoding codon-optimized UGT72B2_Long (UGT72B2_GA, SEQ ID NOs: 63, 18), UGT71E1 (SEQ ID NOs: 3, 4), codon-optimized UGT71E1 (UGT71E1_GS, SEQ ID NOs: 64, 4), UGT73B5 (SEQ ID NOs: 19, 20), and UGT84B1 (SEQ ID NOs: 31, 32) polypeptides were amplified, cloned, and individually integrated in the genome while simultaneously knocking-out the EXG1 gene (SEQ ID NOs: 123, 124).

Plasmids comprising genes encoding UGTs were linearized by restriction enzyme digestion used to transform a resveratrol-producing strain. Transformed cells were grown on plates with selective media. Obtained transformants (6 of each) were re-streaked on fresh masterplates, which were used to inoculate 24-deep well plates supplemented with 3 mL Delft medium comprising 4% glucose and grown for 3 days at 30° C. and shaking at 320 rpm. The cultures were subsequently harvested and prepared for HPLC analysis. 700 μL of broth was combined with 700 μL 96% ethanol, and the samples were mixed by vortexing and centrifugated for 5 min at 13,000 rpm. The supernatants were analyzed by HPLC with a mobile phase (flow rate of 1 mL/min) comprising acetonitrile and H₂O and applying a gradient composed of acetonitrile from 5 to 95% for 10 min. Resveratrol, piceid (3-resveratrol monoglucoside), resveratroloside (4′-resveratrol monoglucoside), 3,5-resveratrol diglucoside, 3,4′-resveratrol diglucoside, and 3,5,4′-resveratrol triglucoside content was measured as “area under the curve” at 306 nm (Table 5). Table 5 displays production of 3,5-resveratrol diglucoside and piceid by UGT71E1 (SEQ ID NOs: 3, 4) and UGT71E1_GS (SEQ ID NOs: 64, 4), piceid produced by expression of UGT73B5 (SEQ ID NOs: 19, 20), and resveratroloside production upon expression of UGT72B2_GA (SEQ ID NOs: 63, 18) at the indicated retention times.

TABLE 5
HPLC analysis of broth of transformants expressing the indicated UGT
polypeptides.

| | | | | 3,5- | | | | | | |
|---|---|---|---|---|---|---|---|---|---|---|
| | | | Unkn. | Glu | Unkn. | Resveratroloside | Unkn. | Piceid | Unkn. | Resv. |
| Strain | # | OD₆₀₀ | (3.78) | (4.46) | (4.52) | (5.06) | (5.42) | (5.61) | (5.75) | (7.06) |
| 72B2_Long | 1 | 44.6 | | | | | 0.37 | | 0.11 | 10.8 |
| (SEQ ID | 2 | 43.8 | | | | | 0.25 | | 0.00 | 7.17 |
| NOs: 17, | 3 | 45.3 | | | | | 0.55 | | 0.30 | 15.3 |
| 18) | 4 | 49.1 | | | | | 0.30 | | 0.00 | 8.67 |
| | 5 | 47.1 | | | | | 0.27 | | 0.00 | 7.99 |
| | 6 | 45.7 | | | | | 0.35 | | 0.11 | 9.72 |
| 72B2_GA | 1 | 46.7 | 0.26 | | | 0.62 | 0.36 | | 0.12 | 10.6 |
| (SEQ ID | 2 | 49.4 | 0.21 | | | 0.43 | 0.26 | | 0.12 | 7.67 |
| NOs: 63, | 3 | 45.7 | 0.25 | | | 0.61 | 0.37 | | 0.12 | 10.7 |
| 18) | 4 | 49.2 | 0.42 | | | 1.00 | 0.62 | | 0.23 | 17.3 |
| | 5 | 49.2 | 0.19 | | | 0.38 | 0.23 | | 0.11 | 7.08 |
| | 6 | 44.4 | 0.15 | | | 0.43 | 0.24 | | 0.00 | 6.91 |
| 71E1 | 1 | 45.8 | | 3.00 | | | 0.37 | 0.83 | 0.68 | 7.45 |
| (SEQ ID | 2 | 50.5 | | 5.17 | | | 0.53 | 1.27 | 1.04 | 11.1 |
| NOs: 3, | 3 | 45.1 | | 2.75 | | | 0.27 | 0.70 | 1.00 | 5.16 |
| 4) | 4 | 47.0 | | 5.12 | | | 0.53 | 1.30 | 1.60 | 10.1 |
| | 5 | 49.4 | | 4.68 | | | 0.47 | 1.20 | 0.64 | 9.59 |
| | 6 | 48.4 | | 5.94 | | | 0.60 | 1.50 | 0.83 | 12.4 |
| 71E1_GS | 1 | 45.5 | | 6.30 | | | 0.43 | 1.25 | 0.63 | 5.71 |
| (SEQ ID | 2 | 50.4 | | 1.71 | | | 0.11 | 0.32 | 0.32 | 1.70 |
| NOs: 64, | 3 | 49.1 | | 6.45 | | | 0.40 | 1.21 | 0.34 | 5.69 |
| 4) | 4 | 52.4 | | 4.78 | | | 0.33 | 0.96 | 0.19 | 4.83 |
| | 5 | 48.1 | | 10.3 | | | 0.61 | 1.89 | 1.20 | 8.07 |
| | 6 | 49.2 | | 5.69 | | | 0.34 | 1.04 | 0.57 | 4.50 |
| 73B5 | 1 | 46.7 | 0.49 | | 0.40 | | 0.46 | 0.09 | 0.74 | 13.5 |
| (SEQ ID | 2 | 43.8 | 0.45 | | 0.35 | | 0.54 | 0.09 | 0.94 | 13.7 |
| NOs: 19, | 3 | 46.1 | 0.51 | | 0.42 | | 0.45 | 0.09 | 1.52 | 12.1 |
| 20) | 4 | 47.7 | 0.47 | | 0.40 | | 0.43 | 0.08 | 1.38 | 11.3 |
| | 5 | 49.1 | 0.52 | | 0.41 | | 0.40 | 0.11 | 1.36 | 11.4 |
| | 6 | 46.9 | 0.27 | | 0.21 | | 0.21 | | 0.59 | 6.30 |
| 84B1 | 1 | 43.4 | | | 4.04 | | 0.37 | | 0.00 | 12.1 |
| (SEQ ID | 2 | 39.0 | | | 4.08 | | 0.40 | | 0.13 | 12.9 |
| NOs: 31, | 3 | 43.8 | | | 3.50 | | 0.31 | | 0.00 | 10.2 |
| 32) | 4 | 42.9 | | | 3.93 | | 0.35 | | 0.10 | 11.6 |
| | 5 | 44.6 | | | 4.14 | | 0.35 | | 0.00 | 11.5 |
| | 6 | 45.1 | | | 4.77 | | 0.39 | | 0.00 | 13.0 |
| — | 1 | 41.8 | | | | | 0.42 | | 1.07 | 10.4 |
| | 2 | 43.7 | | | | | 0.54 | | 1.04 | 13.5 |
| | 3 | 45.9 | | | | | 0.27 | | 0.48 | 6.69 |
| | 4 | 46.4 | | | | | 0.50 | | 1.41 | 12.6 |
| | 5 | 42.6 | | | | | 0.26 | | 0.57 | 6.15 |
| | 6 | 42.8 | | | | | 0.57 | | 1.62 | 14.2 |
| | 7 | 40.6 | | | | | 0.49 | | 0.85 | 12.1 |
| | 8 | 43.4 | | | | | 0.35 | | 0.76 | 9.15 |
| | 9 | 44.9 | | | | | 0.39 | | 1.11 | 9.60 |
| | 10 | 39.3 | | | | | 0.46 | | 1.21 | 11.3 |
| | 11 | 38.6 | | | | | 0.25 | | 0.61 | 6.10 |
| | 12 | 40.0 | | | | | 0.33 | | 0.85 | 8.22 |

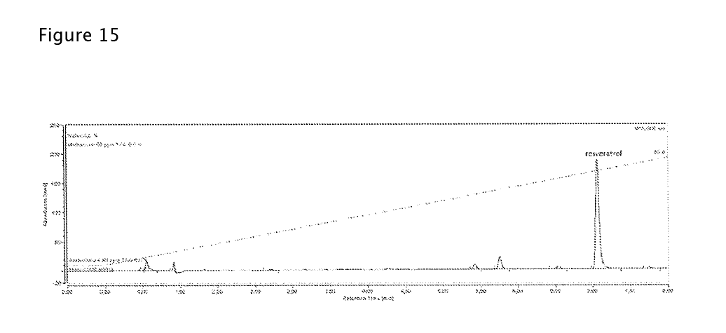

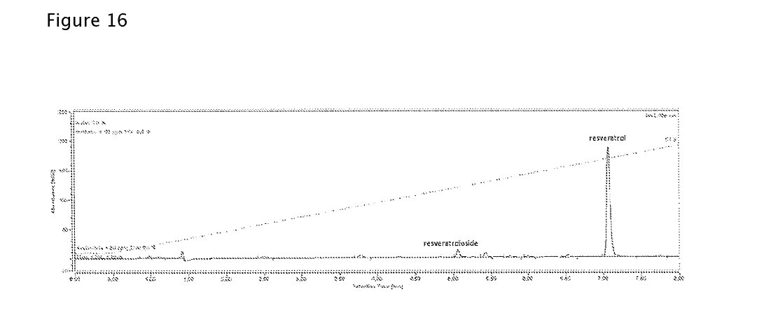

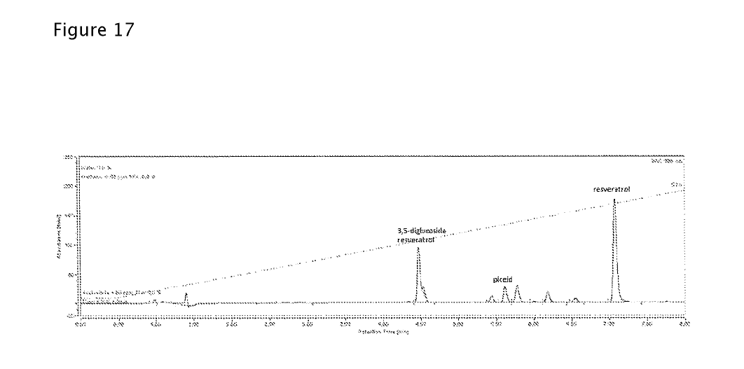

FIGS. 15-17 show characteristic chromatograms analyzing broth from the resveratrol-producing parental strain (FIG. 15), broth from the strain expressing UGT72B2_GA (SEQ ID NOs: 63, 18), and broth from the strain expressing UGT71E1 (SEQ ID NOs: 3, 4). FIG. 16 shows production of resveratroloside by UGT72B2_GA expression (SEQ ID NOs: 63, 18), and FIG. 17 shows production of piceid and 3,5-resveratrol diglucoside by UGT71E1 (SEQ ID NOs: 3, 4). These results show that UGT71E1 (SEQ ID NOs: 3, 4) was able to glycosylate resveratrol to piceid (3-resveratrol monoglucoside) and 3,5-resveratrol diglucoside in vivo. The codon-optimized UGT71E1 (UGT71E1_GS, SEQ ID NOs: 64, 4) was more active. This trend is also seen for UGT72B2_Long and the codon-optimized UGT72B2_Long (UGT72B2_GA, SEQ ID NOs: 63, 18) in the production of resveratroloside.

### Example 7

### Purification of Resveratrol from In Vivo Glycosylated Product

An initial trial to isolate resveratrol by first pelleting resveratrol-producing cells expressing UGT71E1_GS (SEQ ID NOs: 64, 4) and subsequently cleaving the glucose-moieties of 3,5-resveratrol diglucoside revealed that a higher titer was needed. Thus, a 1.5 L fed-batch culture was utilized.

UGT71E1_GS (SEQ ID NOs: 64, 4) was integrated into a resveratrol-producing strain, and EXG1 (SEQ ID NOs: 123, 124) was simultaneously knocked out as in Example 8. The strain was cultivated in fed-batch (1.5 L) and after 5 days of fermentation, the broth was harvested and analyzed by HPLC. The broth was shown to comprise resveratrol, piceid (3-resveratrol monoglucoside), and 3,5-resveratrol diglucoside. Purification of resveratrol was evaluated as described in Examples 4 (i.e., centrifugation, β-glucosidase-treatment, and a second centrifugation to pellet precipitated resveratrol). All obtained fractions were analyzed using HPLC with a mobile phase (flow rate of 1 mL/min) comprising acetonitrile and H₂O and applying a gradient composed of acetonitrile from 5 to 70% for 10 min. Pellet fractions were dissolved 1:1 in 50% ethanol.

The recovery (and loss) of 3,5-resveratrol diglucoside and resveratrol are represented in Table 8. Prior to purification, the fermentation broth comprised 1106 mg/L 3,5-resveratrol diglucoside, 94 mg resveratrol, and 124.5 mg/L piceid. After the first centrifugation, the supernatant comprised 1166 mg/L 3,5-resveratrol diglucoside and 43 mg resveratrol. Results are summarized and shown schematically in FIG. 18. Almost 50% of the resveratrol resulted from the deglycosylation of the 3,5-resveratrol diglucoside was recovered was recovered in a final pellet (FIG. 18).

Low titer of 3,5-resveratrol diglucoside (and subsequent resveratrol production after β-glucosidase-treatment at 30° C. overnight) resulted to an equal distribution behavior between the pellet and the soluble fractions. In order to improve distribution behavior between the pellet and the soluble fractions, several centrifugation conditions were evaluated (11,000, 13,000 and 20,000 rpm) as well as acidification of the sample to a pH of 4.1. Near complete release of resveratrol was achieved when β-glucosidase-treatment was performed at 50° C. overnight.

TABLE 6
Resveratrol and 3,5-resveratrol diglucoside amounts during purification.

| | | Content | | | | |
|---|---|---|---|---|---|---|
| | | 3,5-di-glucoside | | | Distribution in fractions | |
| | | resveratrol (mg/L) | | | (%) | |
| | | 3,5-di- | In | | 3,5-di- | |
| | | glucoside | resveratrol | Resveratrol | glucoside | |
| Sample | Step | resveratrol | equiv. | (mg) | resveratrol | Resveratrol |
| Culture broth | | 1106 | 457 | 94 | 100 | 100 |
| | Cell | | | | | |
| | separation by | | | | | |
| | centrifugation | | | | | |
| | (20 min, | | | | | |
| | 10,000 rpm) | | | | | |
| Cells | | 272 | 112 | 59 | 19 | 58 |
| Supematant | | 1166 | 482 | 43 | 81 | 42 |
| | β- | | | | | |
| | glucosidase- | | | | | |
| | treatment | | | | | |
| Supematant | | 1202 | 497 | 45 | 100 | 100 |
| Supematant | | 0 | | 436 | 0 | 100 + 79 |
| after | | | | | | |
| treatment | | | | | | |
| | Concentration | | | | | |
| | of treated | | | | | |
| | supernatant | | | | | |
| | (3.5x) | | | | | |
| Supematant | | 0 | | 1552 | | |
| concentrated | | | | | | |
| | Pelleting | | | | | |
| | resveratrol by | | | | | |
| | centrifugation | | | | | |
| | (20 min, | | | | | |
| | 13,000 rpm) | | | | | |
| Pellet | | 0 | | 817 | | 55 |
| Supematant | | 0 | | 672 | | 45 |

### Example 8

### Bioconversion of Resveratrol to Resveratrol Glycosides Using Yeast Cells and Knotweed Root Extracts

UGT71E1 (SEQ ID NOs: 3, 4) was expressed in an EXG1 knockout S. cerevisiae strain. Delft media (20 mL) comprising 4% glucose was inoculated with S. cerevisiae cells (that do not produce resveratrol) expressing UGT71E1, and the culture was grown overnight at 30° C. and 140 rpm. The culture was then supplemented with either resveratrol (2.5 g) in 50% ethanol or knotweed root extract (250 or 500 μL) and incubated with agitation at 30° C. for 48 h. The cultures were diluted 1:1 with 96% ethanol, and the samples were vortexed and centrifuged. HPLC chromatograms analyzing the broth of resveratrol and knotweed root extract supplemented with resveratrol or knotweed root extract are shown in FIG. 19.

FIGS. 19A and 19B show resveratrol glucoside formation following bioconversion of resveratrol by yeast expressing UGT71E1 (SEQ ID NOs: 3, 4). FIG. 19C shows piceid and resveratroloside formation following bioconversion of resveratrol of knotweed root extracts. It is also possible that 3,5-resveratrol diglucoside was formed in minute amounts. To verify that peaks observed in FIG. 19C represent resveratrol glucosides, samples were treated with β-glucosidase (Depol cellulase, IFF) overnight at 60° C. As shown in FIG. 19D, resveratrol glucosides were substantially converted to resveratrol. Thus, resveratrol glucosides are capable of being produced by bioconversion of resveratrol and resveratrol-comprising plant extracts.

Bioconversion of resveratrol was also demonstrated with UGT88A1 (SEQ ID NOs: 7, 8), CaUGT2 (SEQ ID NOs: 9, 10), and UGT73B2 (SEQ ID NOs: 13, 14). UGT88A1 (SEQ ID NOs: 7, 8), CaUGT2 (SEQ ID NOs: 9, 10), and UGT73B2 (SEQ ID NOs: 13, 14) were individually cloned into a pJH526 vector and overexpressed in an S. cerevisiae strain that does not express EXG1 (SEQ ID NOs: 123, 124). Transformants were selected on agar plates and picked for growth in 24-deep well plates containing 3 mL SC-ura media supplemented with ascorbic acid (2 mM final concentration) and resveratrol (3 mM final concentration). Resveratrol was supplied using a 60 mM solution in 96% ethanol (5% final ethanol concentration). The plates were covered with breathable seals (Starlab, Saveen & Werner ApS, Denmark) and incubated for 48 h at 30° C. and shaking at 320 rpm. Samples for HPLC analysis were prepared by diluting the cell broth 1:1 with 96% ethanol. Piceid was produced by bioconversion of resveratrol using S. cerevisiae cells expressing UGT88A1 (SEQ ID NOs: 7, 8), UGT2 (SEQ ID NOs: 9, 10), and UGT73B2 (SEQ ID NOs: 13, 14).

### Example 9

### Bioconversion of Resveratrol to Resveratrol Glycosides Using Bacterial Cells

Phytolacca americana glycosyltransferase PaGT3 (SEQ ID NOs: 119, 120) was cloned into a pET30a vector, and E. coli BL21 (DE3, New England Biolabs) cells were transformed with PaGT3 plasmid DNA. NZCYM media (6 mL) comprising kanamycin (50 μg/mL) was inoculated with PaGT3-carrying colonies and incubated overnight at 30° C. and 140 rpm. 14 mL NZCYM media comprising kanamycin (50 μg/mL), arabinose (3 mM final concentration), IPTG (0.1M final concentration), and resveratrol (2.5 g) were then added to each culture, and the culture was incubated for 24 h at 30° C. and 140 rpm. Culture broth was then diluted 1:1 with ethanol, and samples mixed by vortexing and centrifuged for 5 min at 13,000 rpm. The supernatant was analyzed by HPLC using a mobile phase (flow rate of 1 mL/min) comprising acetonitrile and H₂O and applying a gradient composed of acetonitrile from 5 to 70% for 10 min.

A characteristic chromatogram analyzing the broth of BL21 (DE3) cells expressing PaGT3 and supplemented with resveratrol is shown in FIG. 20. Piceid and resveratroloside are formed upon bioconversion of resveratrol using E. coli cells expressing a UGT polypeptide. FIG. 20 shows a chromatogram analyzing the broth of BL21 (DE3) cells carrying an empty PaGT3 vector and supplemented with resveratrol.

### Example 10

### Pharmacokinetic Profiling of Glycosylated Resveratrol

Resveratrol, resveratroloside, and piceid were prepared as 1 mg/mL dosing solutions in 20% (2-Hydroxypropyl)-3-cyclodextrin/0.9% saline. Each compound was administered once, either orally (PO) or intravenously (IV), to CD1 male mice (10 mg/kg, 250 μL/25 g mouse). Three mice were injected per treatment group per observation time point (15 min, 30 min, 1 h, 2 h, 4 h, 8 h, and 24 h) for a total of 126 mice. At each of the specified time point, blood samples were collected by cardiac puncture using heparin treated syringes (˜40 IU heparin per mL blood) under terminal inhaled anaesthesia (Isoflurane). Anti-coagulated blood samples were separated to obtain plasma and stored at −80° C. prior to pharmacokinetic analysis. As shown in the Table 7, resveratrol was not detectable after oral administration and was only detected at low levels in the first half hour after IV administration. In contrast, piceid was detectable after both IV and oral administration. Piceid also converted to trans-resveratrol systemically after IV and oral administration.

TABLE 7
Resveratrol detected after oral administration

| | | | | Piceid | | Piceid | |
|---|---|---|---|---|---|---|---|
| Route of | | | | (Detection | | (Detection of | |
| Administration | | Resveratrol | | of piceid) | | trans-resveratrol) | |
| IV | | IV | PO | IV | PO | IV | PO |
| (h) | PO (h) | (μg/mL) | (μg/mL) | (μg/mL) | (μg/mL) | (μg/mL) | (μg/mL) |
| 0.25 | | 0.04 | | 2.83 | | 0.8 | |
| 0.5 | 0.5 | 0.004 | — | 0.32 | 0.005 | 0.12 | 0.002 |
| 1 | 1 | — | — | 0.05 | 0.008 | 0.02 | 0.002 |
| 2 | 2 | — | — | — | 0.004 | — | — |

(—) not detected or did not meet quality criteria

In a separate experiment, resveratrol, resveratroloside, piceid, 3,5-resveratrol diglucoside, and 3,4′-resveratrol diglucoside dosing solutions were prepared as shown in Table 8. Each compound was administered once at 10 mL/kg, either orally (PO) or intravenously (IV), to CD1 mice. Three mice were injected per treatment group per observation time point (0.5 h, 1 h, 2 h, 3 h, 4 h, 8 h, and 24 h) for a total of 210 mice. At each of the specified time point, blood samples were collected by cardiac puncture using heparin treated syringes (˜40 IU heparin per mL blood) under terminal inhaled anaesthesia (Isoflurane). Anti-coagulated blood samples were separated to obtain plasma and stored at −80° C. LC-MS analysis was carried out with the following conditions: Atlantis C18 column (150×2.1 mm, 3 μm particles; Waters), 20 μL injection volume, 0.24 mL/min flow rate, gradient outlined in Table 9, multiple reaction monitoring (MRM), and Turbo ion spray. Resveratrol and resveratrol glucoside levels were quantified according to reference compounds injected at known concentrations.

TABLE 8
Preparation of dosing solutions.

| | | | | | Vol. of |
|---|---|---|---|---|---|
| | | Dosing | | Amount | diluent (20% |
| | Dose | solution | Amount in | used | cyclodextrin) |
| Compound | (mg/kg) | (mg/mL) | Vial (mg) | (mg) | (mL) |
| Resveratrol | 10 | 1 | 46.4 | 16 | 16 |
| Resvera- | 17.1 | 1.71 | 32.6 | 32.6 | 19.1 |
| troloside | | | | | |
| Piceid | 17.1 | 1.71 | 30.5 | 30.5 | 17.8 |
| (Polydatin) | | | | | |
| 3,5-Resveratrol | 24.2 | 2.42 | 40.7 | 40.7 | 16.8 |
| Diglucoside | | | | | |
| 3,4′- | 24.2 | 2.42 | 40.4 | 40.4 | 16.7 |
| Resveratrol | | | | | |
| Diglucoside | | | | | |

TABLE 9
Mobile phase gradient for LC-MS analysis.

| | % A | % B |
|---|---|---|
| | (2% propan-2-ol in | (2% propan-2-ol |
| Time (min) | 5 mM ammonium acetate) | in methanol) |
| 0 | 100 | 0 |
| 4 | 80 | 20 |
| 9 | 20 | 80 |
| 12 | 20 | 80 |
| 16 | 45 | 55 |
| 16.5 | 100 | 0 |
| 22 | 100 | 0 |

Plasma levels of resveratrol, resveratroloside, piceid, 3,5-resveratrol diglucoside, 3,4′-resveratrol diglucoside, and the metabolites monosulphate 1, monosulphate 2, and monogluconoride measured 0.5, 1, 2, 3, 4, 8, and 24 h post-oral or post-IV administration are shown in FIGS. 21A-L. Resveratrol and resveratrol glucoside levels are indicated as ng/mL on the left; metabolite levels are presented as peak area on the right. Plasma levels of the compound administered after IV and oral administration generally did not exceed 1000 ng/mL, and highest levels of the administered compound generally occurred within 0.5 h after administration.

When resveratrol was administered orally, it was cleared quickly (FIG. 21A). Levels of monosulphate 1 increased 1 h post-administration, while levels of monosulphate 2 and monogluconoride declined steadily over 24 h (FIG. 21A). After IV administration of resveratrol, an increase in resveratrol plasma levels appeared 4 h post-administration; an increase in Monosulphate 1 occurred 1 h post-administration, and monosulphate 2 and monogluconoride decreased steeply 2 h post-administration, with a slower decrease thereafter (FIG. 21B).

Metabolite levels following resveratroloside administration (FIGS. 21C, D) resemble those following resveratrol administration (FIGS. 21A, B). Plasma levels of resveratroloside were low following oral and IV administration of resveratroloside, but after 4 h of oral and IV resveratroloside administration, a sharp increase in resveratrol was measured (FIGS. 21C, D).

Piceid administered orally was detected at a low level in plasma 0.5 h after administration (FIG. 21E). After IV administration of piceid, approximately 900 ng/mL of piceid were detected (FIGS. 21F, 23). Following oral and IV administration of piceid, the initially high levels of Monosulphate 1, Monosulphate 2 and Monogluconoride declined steadily over the sampling period (FIGS. 21E, F).

Oral administration of 3,5-resveratrol diglucoside resulted in high levels of 3,5-resveratrol diglucoside 0.5 h post-administration, which declined within 3 h of administration (FIG. 21G). Upon administration of 3,5-resveratrol diglucoside intravenously, highest levels of plasma 3,5-resveratrol diglucoside were measured 1 h post-administration and declined within 4 h of administration (FIG. 21H). Plasma levels of resveratrol remained low after both routes of administration (FIGS. 21G, H).

Plasma levels of 3,4′-resveratrol diglucoside following oral administration were approximately 3-fold higher than for 3,5-resveratrol diglucoside and were cleared within 1 h (FIGS. 21G, I). Plasma levels of 3,4′-resveratrol diglucoside and 3,5-resveratrol diglucoside, however, were relatively equivalent (FIGS. 21H, J).

Overall, plasma levels of 3,5-resveratrol diglucoside and 3,4′-resveratrol diglucoside were significantly higher than resveratrol, resveratroloside, and piceid following oral and intravenous administration (FIGS. 21K, L).

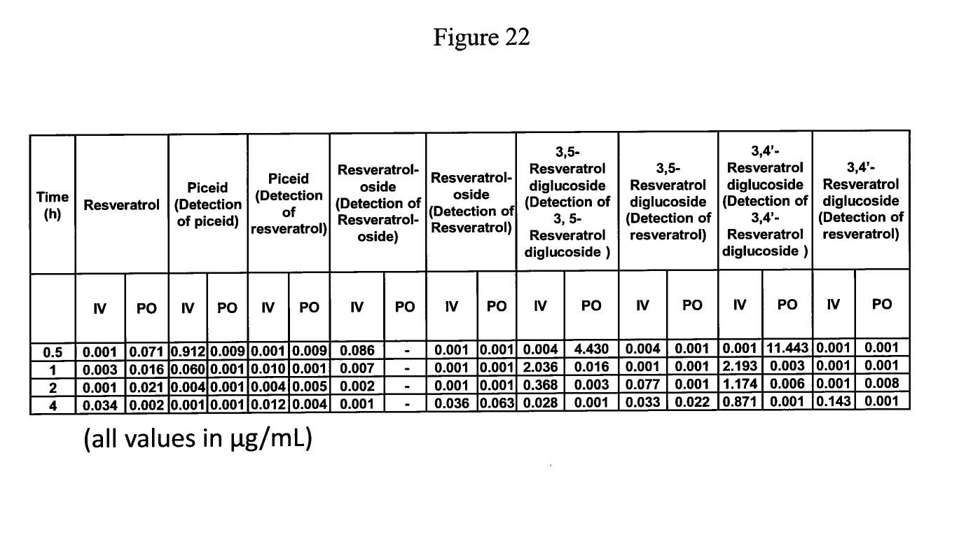

Plasma levels of resveratrol, resveratroloside, piceid, 3,5-resveratrol diglucoside, and 3,4′-resveratrol diglucoside 0.5, 1, 2, and 4 h post-administration, as well as plasma levels of resveratrol following oral or intravenous administration of resveratroloside, piceid, 3,5-resveratrol diglucoside, or 3,4′-resveratrol diglucoside, are summarized in FIG. 22.

### Example 11

### In Vivo Production of Pterostilbene

In addition to resveratrol glycosides, methylated resveratrol was produced in vivo. The structure of resveratrol methylated at the 3 and 5 positions is known as pterostilbene (FIG. 23). A codon-optimized gene encoding a resveratrol O-methyltransferase ROMT polypeptide (SEQ ID NOs: 5, 6) was cloned into a p425GPD vector and used to transform a resveratrol-producing yeast strain. Cultures were grown in Delft media for approximately 72 h at 30° C.

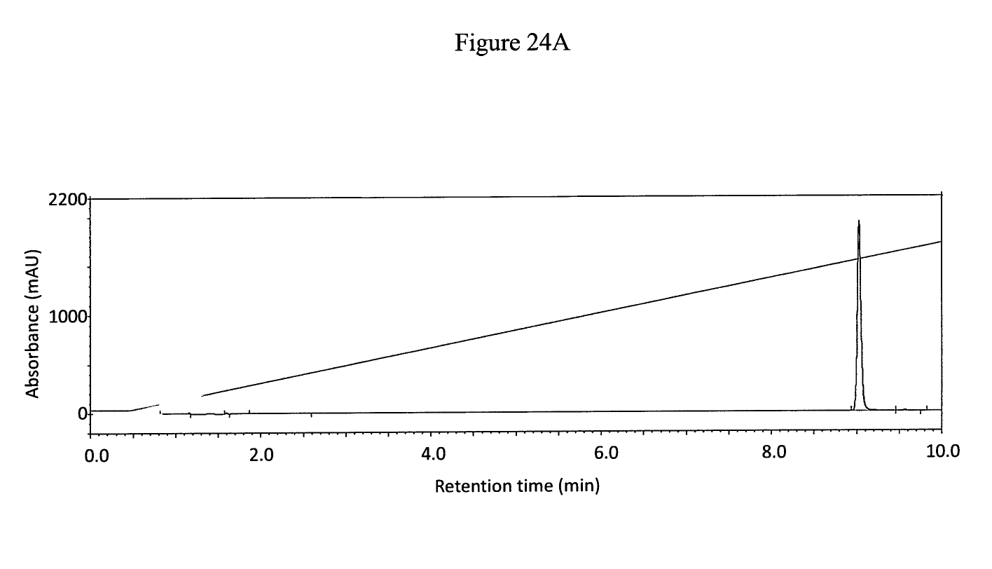

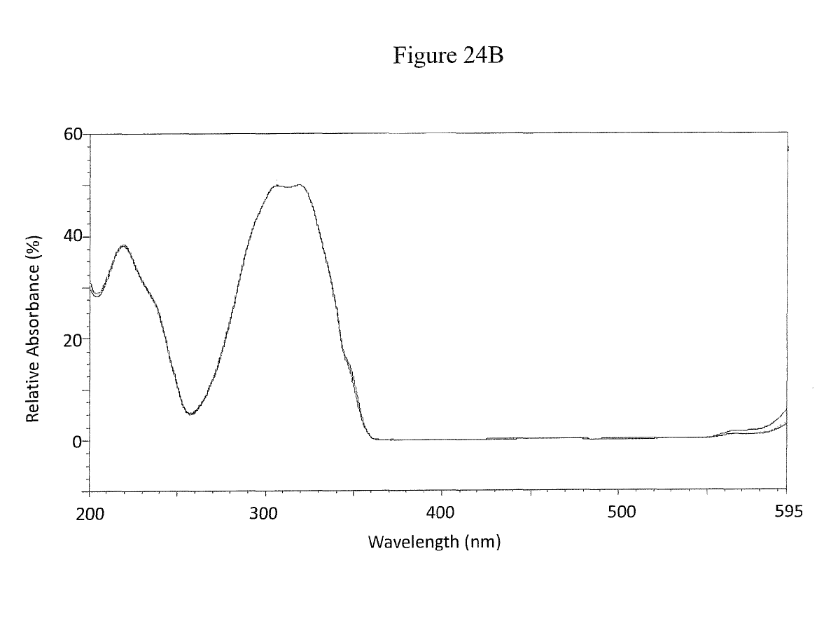

Pterostilbene was detected by HPLC with a mobile phase (flow rate of 1 mL/min) comprising acetonitrile and H₂O and applying a gradient composed of acetonitrile from 5 to 95% for 10 min. Commercial pterostilbene (ALX-385-034-MO25; Enzo Life Sciences) was used as a standard, with a peak eluting with a retention time of 9.03 min (FIGS. 24A, B). As shown in FIG. 25, pterostilbene production was also detected in the broth of a resveratrol-producing strain expressing an ROMT polypeptide (FIG. 25). Additional compounds were identified in the broth of the resveratrol-producing strain, including resveratrol, phloretic acid, coumaric acid, cinnamic acid, and pinosylvin (FIG. 25). Additional unknown peaks were also observed, and it was hypothesized that the ROMT polypeptide methylated compounds other than resveratrol.

### Example 12

### In Vitro Bioconversion of Resveratrol to Pterostilbene

A codon-optimized gene encoding a resveratrol O-methyltransferase ROMT polypeptide (SEQ ID NOs: 5, 6) was cloned into an integrative pROP235 vector vector and used to transform an S. cerevisiae strain that does not produce resveratrol. Delft media (20 mL) comprising 4% glucose was inoculated with ROMT-expressing cells and incubated overnight at 30° C. and 140 rpm shaking. The culture was then supplemented with glucose in the form of two FeedBeads® (Kuhner, 12 mm) and 2.5 g resveratrol in 50% ethanol. The culture was incubated at 30° C. and 140 rpm shaking for 72 h.

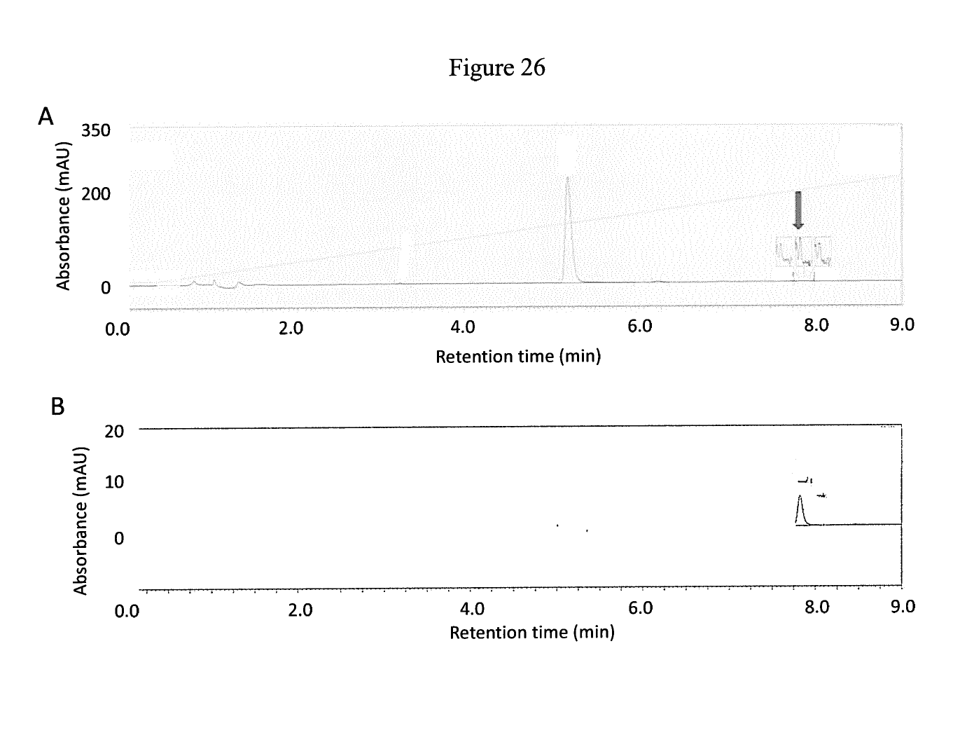

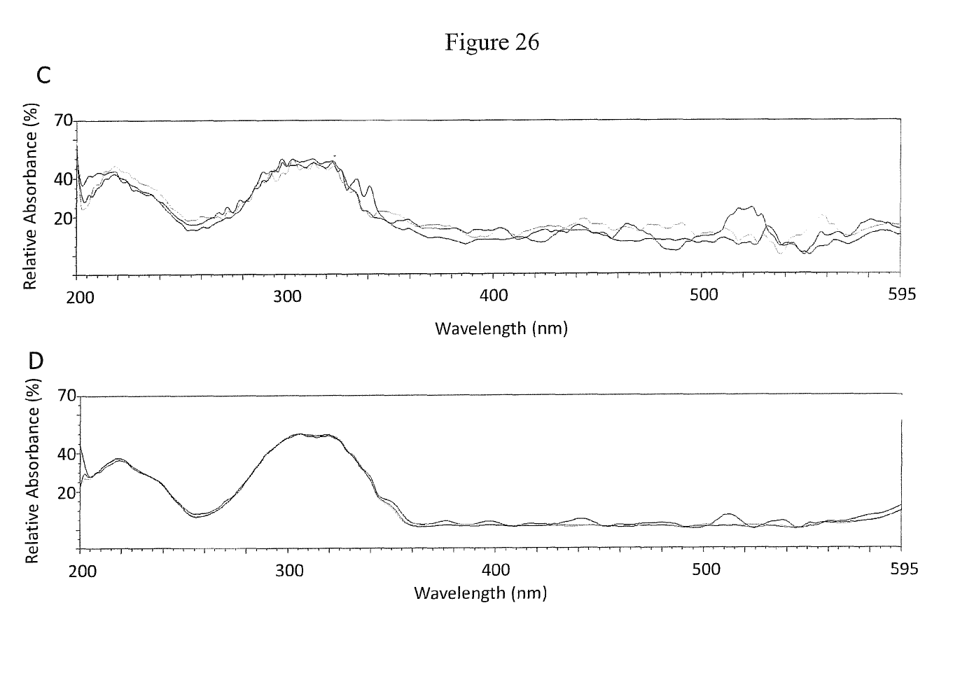

Broth of the resveratrol-treated ROMT-expressing cells was diluted 1:1 with 96% ethanol, and the samples were vortexed, centrifuged for 5 min, and analyzed by HPLC. Commercial pterostilbene (Combi-blocks, Inc., QB-9140-005) was used as a standard. A small peak observed with a retention time of 7.82 min and an absorption wavelength of 306 nm corresponds to pterostilbene (FIGS. 26A, C), consistent with the peaks observed with the pterostilbene standard (FIGS. 26B, D). Thus, resveratrol was converted into pterostilbene upon supplementation of ROMT-expressing yeast with resveratrol.

### Example 13

### In Vitro Glycosylation of Pterostilbene

To glycosylate the 4′ position of pterostilbene (the 3 and 5 positions of pterostilbene are methylated and thus not accessible for glycosylation), pterostilbene (QB-9140-005, Combi-blocks, Inc., QB-9140-005) was dissolved in a buffer comprising 100 mM Tris (pH 8.0), 5 mM MgCl₂, 1 mM KCl, alkaline phosphatase (Fermentas), 100 mM UDP-sugar, and purified UGT72B2_Long enzyme (SEQ ID NO: 18). The final concentration of pterostilbene was 10 mM. UGT72B2_Long was chosen since it has been shown to glycosylate resveratrol in the 4′ position (see, e.g., Example 8). The samples were incubated at 30° C. overnight with agitation. Glycosylated product was detected by HPLC.

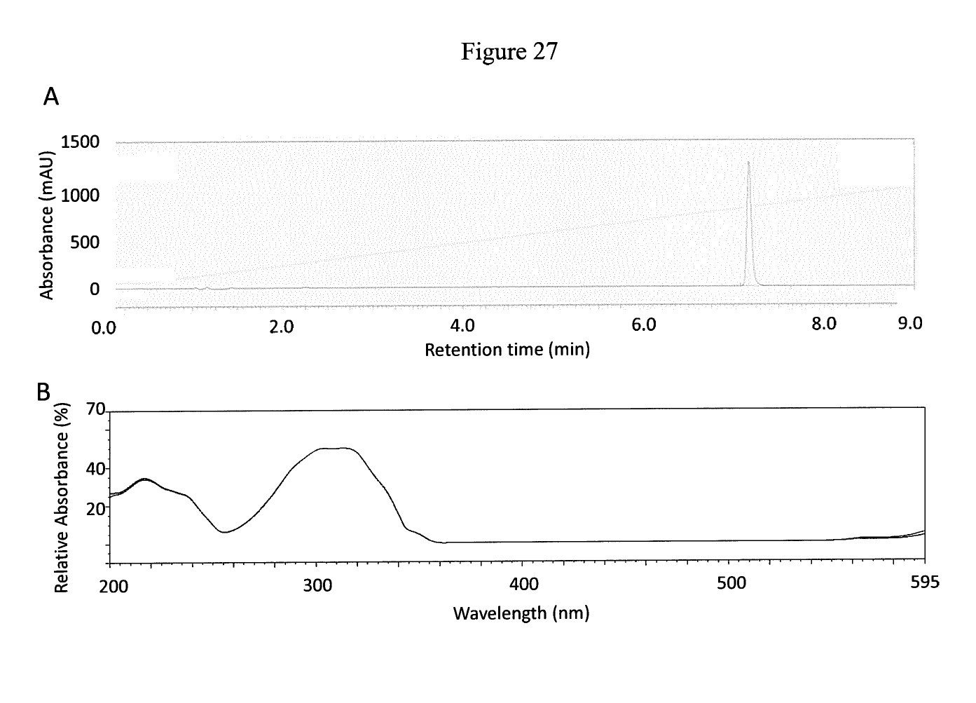

As shown in FIG. 27A, a peak appeared with a retention time of 7.82 min; no pterostilbene peak with a retention time of 7.82 was observed. The UV-Vis spectrum of the glycosylated pterostilbene sample is presented in FIG. 27B.

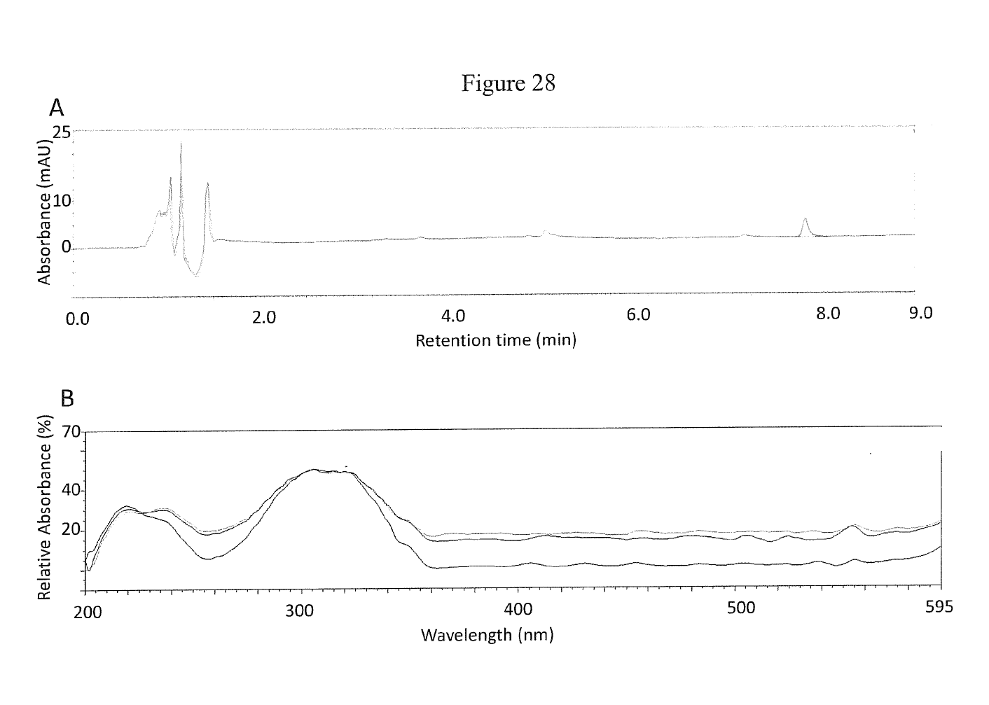

To identify the peak in FIG. 27A, the glycosylated pterostilbene sample was first treated with a β-glucosidase (Depol cellulase, IFF) to determine whether pterostilbene could be recovered. As shown in FIG. 28A, the peak at 7.82 min decreased in size and a peak at 7.82 min corresponding to pterostilbene appeared upon β-glucosidase treatment. The UV-Vis spectrum of pterostilbene is presented in FIG. 28B.

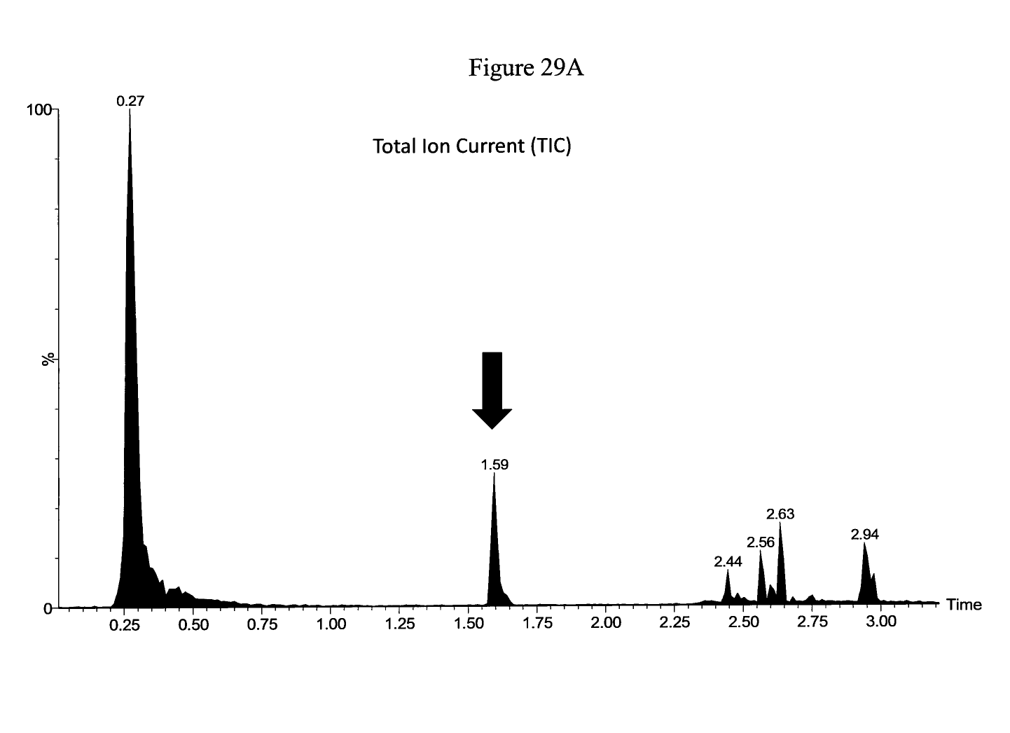

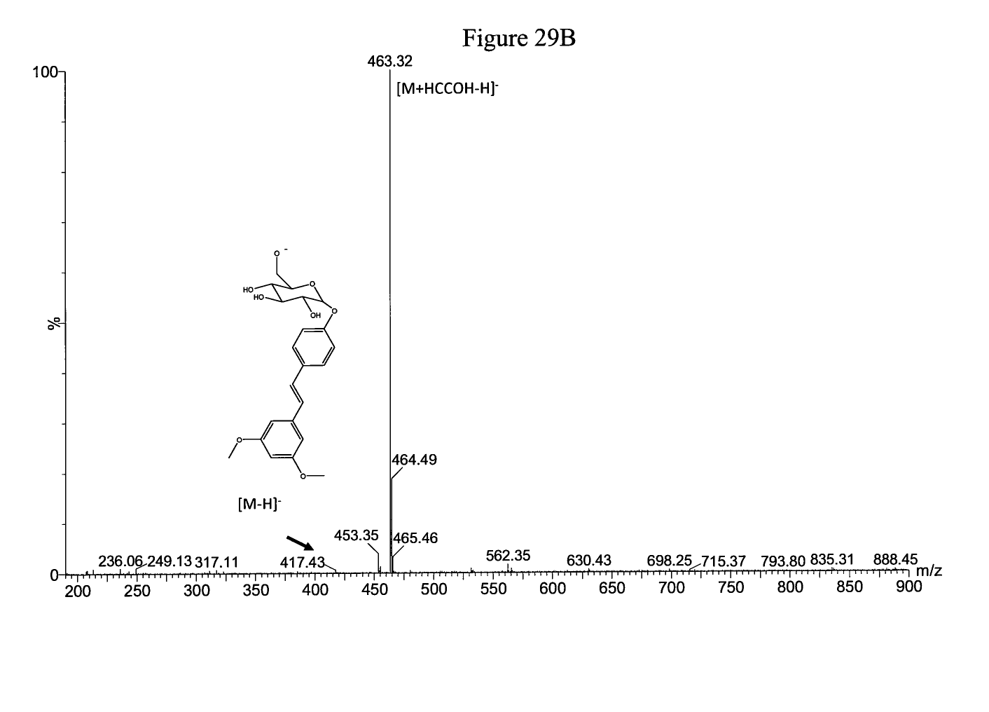

Secondly, to identify the molecular weight of the pterostilbene glucoside, LC-MS was performed. As shown in FIGS. 29A, C, the peak with a retention time of 7.82 min corresponds to a glycosylated pterostilbene plus the formic acid adduct.

Thus, pterostilbene can be glycosylated in vitro by UGT72B2_Long (SEQ ID NOs: 17, 18). Since glycosylated resveratrol can be produced in vivo, as described herein, and UGT72B2_Long has also been shown to function in vivo, it is possible that glycosylated pterostilbene can be produced in vivo as well.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A method for producing a glycosylated stilbene, comprising:
(a) growing a recombinant host in a culture medium, under conditions in which the host produces a stilbene,
wherein the host comprises a gene encoding a glycosyltransferase (UGT) polypeptide capable of in vivo glycosylation of the stilbene comprising a recombinant expression construct;
wherein the gene encoding the UGT polypeptide is expressed in the host, wherein the stilbene is glycosylated in the host thereby; and
(b) recovering the glycosylated stilbene from the culture media.

2. The method of claim 1, wherein the host does not express an exo-1,3-beta-glucanase.

3. The method of claim 1, wherein the UGT polypeptide comprises:
(a) a UGT72B2 polypeptide having at least 55% identity to the amino acid sequence set forth in SEQ ID NO: 18;
(b) a UGT71E1 polypeptide having at least 60% identity to the amino acid sequence set forth in SEQ ID NO: 4;
(c) a UGT73B5 polypeptide having at least 55% identity to the amino acid sequence set forth in SEQ ID NO: 20;
(d) a UGT84B1 polypeptide having at least 40% identity to the amino acid sequence set forth in SEQ ID NO: 32; or
(e) a UGT75B2 polypeptide having at least 50% identity to the amino acid sequence set forth in SEQ ID NO: 22;
(f) a UGT73C5 polypeptide having at least 60% identity to the amino acid sequence set forth in SEQ ID NO: 40; or
(g) a UGT73C3 polypeptide having at least 60% identity to the amino acid sequence set forth in SEQ ID NO: 38.

4. The method of claim 1, wherein the stilbene comprises 3, 4′, and 5 hydroxyl groups, wherein the glycosylated stilbene comprises one or a plurality of sugar moieties covalently linked to the one or more of the 3, 4′, or 5 hydroxyl groups of the stilbene.

5. The method of claim 4, wherein the glycosylated stilbene is monoglycosylated at one of the 3, 4′, or 5 hydroxyl groups, diglycosylated at the 3,4′, 3,5, or 4′,5 hydroxyl groups, or triglyosylated at the 3, 4′, 5 hydroxyl groups.

6. The method of claim 1, further comprising the step of cleavage of sugar moieties of the glycosylated stilbene, wherein the stilbene can be recovered from the culture media.

7. The method of claim 6, wherein cleavage of the sugar moieties of the glycosylated stilbene comprises enzymatic cleavage.

8. The method of claim 7, wherein enzymatic cleavage comprises treating the culture medium with an enzyme capable of cleaving sugar moieties.

9. The method of claim 8, wherein the enzyme comprises β-glucosidase, cellulase, glusulase, cellobiase, β-galactosidase, β-glucuronidase, or EXG1.

10. The method of claim 6, wherein cleavage of the sugar moieties of the glycosylated stilbene comprises chemical cleavage.

11. The method of claim 10, wherein chemical cleavage comprises treating the culture medium with a weak acid or under other conditions capable of cleaving sugar moieties.

12. The method of claim 11, wherein the weak acid comprises an organic acid or an inorganic acid.

13. The method of claim 6, further comprising the step of detecting the recovered stilbene by thin layer chromatography (TLC), high-performance liquid chromatography (HPLC), ultraviolet-visible spectroscopy/spectrophotometry (UV-Vis), liquid chromatography-mass spectrometry (LC-MS), or nuclear magnetic resonance (NMR).

14. The method of any one of claims 1-13, wherein the stilbene comprises resveratrol.

15. The method of any one of claims 1-14, wherein the glycosylated stilbene comprises piceid (3-resveratrol monoglucoside or 5-resverarol monoglucoside), resveratroloside (4′-resveratrol monoglucoside), Mulberroside E (3,4′-resveratrol diglucoside), 3,5-resveratrol diglucoside, and 3,5,4′-resveratrol triglucoside.

16. A method for producing a glycosylated stilbene from a bioconversion reaction, comprising
(a) growing a recombinant host in a culture medium, under conditions in which glycosyltransferase enzymes (UGTs) are produced in said host,
wherein the host comprises a gene encoding a glycosyltransferase (UGT) polypeptide capable of in vivo glycosylation of a stilbene comprising a recombinant expression construct;
wherein the gene encoding the UGT polypeptide is expressed in the host;
(b) contacting the host with a stilbene in a reaction buffer to produce a glycosylated stilbene; and
(c) purifying the glycosylated stilbene.

17. The method of claim 16, wherein the UGT polypeptide comprises:
(a) a UGT71E1 polypeptide having at least 60% identity to the amino acid sequence set forth in SEQ ID NO: 4;
(b) a UGT88A1 polypeptide having at least 50% identity to the amino acid sequence set forth in SEQ ID NO: 8;
(c) a CaUGT2 polypeptide having at least 60% identity to the amino acid sequence set forth in SEQ ID NO: 10;
(d) a UGT73B2 polypeptide having at least 55% identity to the amino acid sequence set forth in SEQ ID NO: 14; or
(e) a PaGT3 polypeptide having at least 60% identity to the amino acid sequence set forth in SEQ ID NO: 120.

18. The method of claim 17, wherein the stilbene comprises a plant-derived or synthetic stilbene.

19. The method of claim 17, the glycosylated stilbene produced comprises mono-, di-, tri- or poly-glycosylated stilbene molecules.

20. The method of any one of claims 16-19, wherein the glycosylated stilbene produced is separated from the culture media through filtration or centrifugation.

21. The method of claim 20, further comprising the step of cleaving sugar moieties of the glycosylated stilbene, wherein cleavage comprises treating the glycosylated stilbene with an enzyme capable of cleaving sugar moieties.

22. The method of claim 21, wherein the enzyme comprises β-glucosidase, cellulase, glusulase, cellobiase, β-galactosidase, β-glucuronidase, or EXG1.

23. The method of any one of claims 16-22, wherein the stilbene comprises resveratrol.

24. The method of any one of claims 16-23, wherein the glycosylated stilbene comprises piceid (3-resveratrol monoglucoside or 5-resverarol monoglucoside), resveratroloside (4′-resveratrol monoglucoside), Mulberroside E (3,4′-resveratrol diglucoside), 3,5-resveratrol diglucoside, and 3,5,4′-resveratrol triglucoside.

25. A method for producing a methylated stilbene, comprising
(a) growing a recombinant host in a culture medium, under conditions in which the host produces a stilbene,
wherein the host comprises a gene encoding a methyltransferase polypeptide capable of in vivo methylation of the stilbene comprising a recombinant expression construct;
wherein the gene encoding the methyltransferase polypeptide is expressed in the host, wherein the stilbene is methylated in the host; and
(b) recovering the methylated stilbene from the culture media.

26. The method of claim 25, wherein the gene encoding the methyltransferase polypeptide comprises a gene encoding a resveratrol O-methyltransferase (ROMT) polypeptide.

27. The method of claim 26, wherein the ROMT polypeptide comprises Vitis vinifera ROMT polypeptide having at least 70% identity to the amino acid sequence set forth in SEQ ID NO: 6.

28. The method of claim 27, wherein the methylated stilbene is methylated at hydroxyl groups of the stilbene, wherein methylation comprises covalently attaching one or a plurality of methyl groups at one or more of the hydroxyl groups of the stilbene.

29. The method of claim 25, wherein the stilbene comprises 3, 4′, and 5 hydroxyl groups, wherein the methylated stilbene is monomethylated at 3, 4′, or 5 hydroxyl groups; dimethylated at 3,4′, 3,5, or 4′,5 hydroxyl groups; or is trimethylated at 3, 4′, 5 hydroxyl groups.

30. The method of claim 25, further comprising the step of detecting recovered the methylated stilbene by thin layer chromatography (TLC), high-performance liquid chromatography (HPLC), ultraviolet-visible spectroscopy/spectrophotometry (UV-Vis), liquid chromatography-mass spectrometry (LC-MS), or nuclear magnetic resonance (NMR).

31. The method of any one of claims 25-30, wherein the stilbene is resveratrol.

32. The method of any one of claims 25-30, wherein the methylated stilbene comprises pterostilbene (3,5-dimethoxy-4′-hydroxy-trans-stilbene), 3,5,4′-trimethoxystilbene, pinostilbene, tetramethoxystilbene, pentamethoxystilbene, and N-Hydroxy-N-(trimethoxphenyl)-trimethoxy-benzamidine.

33. A method for producing a methylated stilbene from a bioconversion reaction, comprising
(a) growing a recombinant host in a culture medium, under conditions in which methyltransferase enzymes are produced in said host,
wherein the host comprises a gene encoding a methyltransferase polypeptide capable of in vivo methylation of a stilbene comprising a recombinant expression construct;
wherein the gene encoding the methyltransferase polypeptide is expressed in the host;
(c) contacting the host with a stilbene in a reaction buffer to produce a methylated stilbene; and
(d) purifying the methylated stilbene.

34. The method of claim 33, wherein the methyltransferase polypeptide comprises a resveratrol O-methyltransferase (ROMT) polypeptide having at least 70% identity to the amino acid sequence set forth in SEQ ID NO: 6.

35. The method of claim 33, wherein the stilbene comprises a plant-derived or synthetic stilbene.

36. The method of claim 33, the methylated stilbene comprises mono-, di-, tri- or poly-methylated stilbene molecules.

37. The method of any one of claims 33-36, wherein the stilbene comprises resveratrol.

38. The method of any one of claims 33-37, wherein the methylated stilbene comprises pterostilbene (3,5-dimethoxy-4′-hydroxy-trans-stilbene), 3,5,4′-trimethoxystilbene, pinostilbene, tetramethoxystilbene, pentamethoxystilbene, and N-Hydroxy-N-(trimethoxphenyl)-trimethoxy-benzamidine.

39. The method of any one of claims 1-38, wherein the host comprises a microorganism that is a yeast cell, a plant cell, a mammalian cell, an insect cell, a fungal cell, or a bacterial cell.

40. The method of claim 39, wherein the bacterial cell comprises Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacterial cells.

41. The method of claim 39, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

42. The method of claim 41, wherein the yeast cell is a Saccharomycete.

43. The method of claim 42, wherein the yeast cell is a cell from the Saccharomyces cerevisiae species.

44. The method of claim 43, wherein the yeast cell comprises an S. cerevisiae yeast cell that does not express EXG1.

45. A recombinant host comprising:
(a) a gene encoding a glycosyltransferase (UGT) polypeptide, wherein the UGT polypeptide is capable of in vivo glycosylation of a stilbene; and/or
(b) a gene encoding a methyltransferase polypeptide, wherein the methyltransferase polypeptide is capable of in vivo methylation of a stilbene;
wherein at least one of said genes is a recombinant gene, wherein the host is capable of producing a stilbene.

46. The host of claim 45, wherein the UGT polypeptide comprises
(a) a UGT72B2 polypeptide having at least 55% identity to the amino acid sequence set forth in SEQ ID NO:18;
(b) a UGT71E1 polypeptide having at least 60% identity to the amino acid sequence set forth in SEQ ID NO:4;
(c) a UGT73B5 polypeptide having at least 55% identity to the amino acid sequence set forth in SEQ ID NO:20;
(d) a UGT84B1 polypeptide having at least 40% identity to the amino acid sequence set forth in SEQ ID NO:32;
(e) a UGT75B2 polypeptide having at least 50% identity to the amino acid sequence set forth in SEQ ID NO: 22;
(f) a UGT73C5 polypeptide having at least 60% identity to the amino acid sequence set forth in SEQ ID NO: 40; or
(g) a UGT73C3 polypeptide having at least 60% identity to the amino acid sequence set forth in SEQ ID NO: 38.

47. The host of claim 45, wherein the gene encoding the methyltransferase polypeptide comprises an ROMT polypeptide having at least 70% identity to the amino acid sequence set forth in SEQ ID NO: 6.

48. The host of claim 45, wherein the stilbene is resveratrol.

49. A recombinant host comprising one or more of:
(a) a gene encoding a L-phenylalanine ammonia lyase (PAL) polypeptide;
(b) a gene encoding a cinnamate-4-hydroxylase (C4H) polypeptide;
(c) a gene encoding a NADPH:cytochrome P450 reductase polypeptide;
(d) a gene encoding a tyrosine ammonia lyase (TAL);
(e) a gene encoding a 4-coumarate-CoA ligase (4CL); or
(f) a gene encoding stilbene synthase (STS);
wherein at least one of said genes is a recombinant gene, wherein the host is capable of producing a stilbene.

50. The host of claim 49, wherein the host produces the stilbene from a carbon source when fed a precursor, wherein the precursor comprises coumaric acid.

51. The host of any one of claims 45-50, wherein the host comprises a microorganism that is a yeast cell, a plant cell, a mammalian cell, an insect cell, a fungal cell, or a bacterial cell.

52. The host of claim 51, wherein the bacterial cell comprises Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacterial cells.

53. The host of claim 51, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

54. The host of claim 53, wherein the yeast cell is a Saccharomycete.

55. The host of claim 54, wherein the yeast cell is a cell from the Saccharomyces cerevisiae species.

56. The host of claim 55, wherein the yeast cell comprises an S. cerevisiae yeast cell that does not express EXG1.

57. A method for producing a glycosylated stilbene from an in vitro reaction comprising contacting a stilbene with one or more UGT polypeptides in the presence of one or more UDP-sugars.

58. The method of claim 57, wherein the one or more UGT polypeptides comprises:
(a) a UGT72B2 polypeptide having at least 55% identity to the amino acid sequence set forth in SEQ ID NO: 18;
(b) a UGT71E1 polypeptide having at least 60% identity to the amino acid sequence set forth in SEQ ID NO: 4;
(c) a UGT73B5 polypeptide having at least 55% identity to the amino acid sequence set forth in SEQ ID NO: 20;
(d) a UGT84B1 polypeptide having at least 40% identity to the amino acid sequence set forth in SEQ ID NO: 32;
(e) a UGT88A1 polypeptide having at least 50% identity to the amino acid sequence set forth in SEQ ID NO: 8;
(f) a UGT75B2 polypeptide having at least 50% identity to the amino acid sequence set forth in SEQ ID NO: 22;
(g) a UGT78D2 polypeptide having at least 55% identity to the amino acid sequence set forth in SEQ ID NO: 30;
(h) a UGT73C5 polypeptide having at least 60% identity to the amino acid sequence set forth in SEQ ID NO: 40;
(i) a UGT73C3 polypeptide having at least 60% identity to the amino acid sequence set forth in SEQ ID NO: 38; or
(j) a UGT73B2 polypeptide having at least 55% identity to the amino acid sequence set forth in SEQ ID NO: 14;
wherein at least one of the UGT polypeptides is a recombinant UGT polypeptide.

59. The method of claim 58, wherein the stilbene comprises a plant-derived or synthetic stilbene.

60. The method of claim 58, wherein the glycosylated stilbene produced comprises mono-, di-, tri- or poly-glycosylated stilbene molecules.

61. The method of claim 58, wherein the one or more UDP-sugars comprise UDP-glucose, UDP-rhamnose, or UDP-xylose.

62. The method of any one of claims 58-61, wherein the stilbene comprises resveratrol.

63. The method of any one of claims 58-62, wherein the glycosylated stilbene comprises piceid (3-resveratrol monoglucoside or 5-resverarol monoglucoside), resveratroloside (4′-resveratrol monoglucoside), Mulberroside E (3,4′-resveratrol diglucoside), 3,5-resveratrol diglucoside, and 3,5,4′-resveratrol triglucoside.

64. A method for producing a methylated stilbene from an in vitro reaction comprising contacting a stilbene with one or more methyltransferase polypeptides.

65. The method of claim 64, wherein the one or more methyltransferase polypeptides comprises an ROMT polypeptide having at least 70% identity to the amino acid sequence set forth in SEQ ID NO: 6.

66. The method of claim 64, wherein the stilbene comprises a plant-derived or synthetic stilbene.

67. The method of claim 64, the methylated stilbene produced comprises mono-, di-, tri- or poly-methylated stilbene molecules.

68. The method of any one of claims 64-67, wherein the stilbene comprises resveratrol.

69. The method of any one of claims 64-68, wherein the methylated stilbene comprises pterostilbene (3,5-dimethoxy-4′-hydroxy-trans-stilbene), 3,5,4′-trimethoxystilbene, pinostilbene, tetramethoxystilbene, pentamethoxystilbene, and N-Hydroxy-N-(trimethoxphenyl)-trimethoxy-benzamidine.

70. A method for producing resveratrol glycosides comprising bioconversion of resveratrol or a plant extract using one or more UGT polypeptides and one or more UDP-sugars, wherein the bioconversion comprises contacting the resveratrol or the plant extract with the one or more UGT polypeptides in the presence of the one or more UDP-sugars, wherein the bioconversion comprises in vitro enzymatic or whole cell bioconversion.

71. The method of claim 70, wherein the one or more UGT polypeptides comprises:
(a) a UGT71E1 polypeptide having at least 60% identity to the amino acid sequence set forth in SEQ ID NO: 4;
(b) a UGT88A1 polypeptide having at least 50% identity to the amino acid sequence set forth in SEQ ID NO: 8;
(c) a CaUGT2 polypeptide having at least 60% identity to the amino acid sequence set forth in SEQ ID NO: 10;
(d) a UGT73B2 polypeptide having at least 55% identity to the amino acid sequence set forth in SEQ ID NO: 14; or
(e) a PaGT3 polypeptide having at least 60% identity to the amino acid sequence set forth in SEQ ID NO: 120;
wherein at least one of the UGT polypeptides is a recombinant UGT polypeptide.

72. The method of claim 70, wherein the one or more UDP-sugars comprise UDP-glucose, UDP-rhamnose, or UDP-xylose.

73. A method for producing methylated resveratrol comprising bioconversion of a resveratrol or a plant extract using one or more methyltransferase polypeptides, wherein the bioconversion comprises contacting the resveratrol or the plant extract with the one or more methyltransferase polypeptides, wherein the bioconversion comprises in vitro enzymatic or whole cell bioconversion.

74. The method of claim 73, wherein the one or more methyltransferase polypeptides comprises an ROMT polypeptide having at least 70% identity to the amino acid sequence set forth in SEQ ID NO: 6.

75. A method for producing glycosylated pterostilbene comprising bioconversion of a pterostilbene using one or more UGT polypeptides and one or more UDP-sugars, wherein the bioconversion comprises contacting the pterostilbene with the one or more UGT polypeptides in the presence of the one or more UDP-sugars, wherein the bioconversion comprises in vitro enzymatic or whole cell bioconversion.

76. The method of claim 75, wherein the one or more UGT polypeptides comprises:
(a) a UGT71E1 polypeptide having at least 60% identity to the amino acid sequence set forth in SEQ ID NO: 4;
(b) a UGT88A1 polypeptide having at least 50% identity to the amino acid sequence set forth in SEQ ID NO: 8;
(c) a CaUGT2 polypeptide having at least 60% identity to the amino acid sequence set forth in SEQ ID NO: 10;
(d) a UGT73B2 polypeptide having at least 55% identity to the amino acid sequence set forth in SEQ ID NO: 14; or
(e) a PaGT3 polypeptide having at least 60% identity to the amino acid sequence set forth in SEQ ID NO: 120;
wherein at least one of the UGT polypeptides is a recombinant UGT polypeptide.

77. The method of claim 75, wherein the one or more UDP-sugars comprise UDP-glucose, UDP-rhamnose, or UDP-xylose.

78. A composition comprising glycosylated or methylated resveratrol, wherein the resveratrol composition does not contain plant-derived contaminant compounds.

79. The composition of claim 78, wherein the resveratrol composition is mono, di, tri or poly-glycosylated and/or mono, di, or tri-methylated.

80. The composition of claim 78, wherein the resveratrol composition is covalently attached to sugar moieties, wherein the sugar moieties are monosaccharides, disaccharides, or polysaccharides.

81. The composition of claim 78, wherein the monosaccharide is glucose, fructose, xylose, rhamnose, arabinose, glucuronic acid, erythrose, ribose, or galactose.

82. The composition of claim 78, wherein the disaccharide is sucrose, maltose, or lactose.


SEQUENCE LISTING (136 sequences)

SEQ ID NO: 1 - DNA, 1317 bp, Artificial Sequence
  Synthetic oligonucleotide
atggattcaaaaatcgattccaagacatttcgagtagtgatgctaccatggttagcatatagtcacatatctcgcttcctagtttttgccaaaagactcacaaaccataactttcatatatacatatgttcatcccaaaccaacatgcaatatctcaaaaacaaccttacttctcaatactctaaatccatccaactcattgaactaaatcttccatcttcaagtgaactccctcttcaatatcacaccactcatggccttccacctcacctcacgaaaaccttatcggacgattatcaaaaatcaggtccggattttgagaccatactcataaaacttaacccgcatttggttatatatgattttaaccaattgtgggccccagaagtggcttcgacgctacacatccctagtattcagttattaagcggatgtgtagcgttatatgctttggatgctcatctctacacaaagccattagatgaaaatttggcaaaatttccttttcctgaaatatatcctaaaaatcgcgatattccaaaaggtggttcgaaatatattgaacgctttgttgattgcatgagaaggtcttgtgagataatattggttagaagtacaatggagttagaagggaagtatattgattatttatctaaaactcttggaaagaaagtattgccggttggtccattagttcaagaagcttctttattacaagatgatcatatttggatcatgaaatggcttgataaaaaagaagaatcttcagttgtgtttgtatgttttggtagtgaatatattttatctgataatgagatagaagatatagcctatggtttggagcttagccaagtgagttttgtatgggctataagggcgaaaactagtgctcttaatgggtttattgacagggttggagataagggtctggttatcgataaatgggtcccacaagccaatattttaagtcattcgagtacgggtggatttattagtcattgtggatggagttcaactatggaaagtattcgttatggtgttccgattatagcaatgcctatgcaatttgatcagccgtataatgcaaggctaatggagacggttggagccggtattgaagttgggagagatggtgaggggaggctgaagagggaggagattgcggcggtggtgaggaaggtggtggtggaggatagtggtgagagtataagggaaaaggctaaggagttgggtgagattatgaagaagaatatggaggcagaggttgatggtatagtgattgagaatttggtcaagctttgtgaaatgaataattga

SEQ ID NO: 2 - protein, 438 aa, Bellis perennis
MDSKIDSKTFRVVMLPWLAYSHISRFLVFAKRLTNHNFHIYICSSQTNMQYLKNNLTSQYSKSIQLIELNLPSSSELPLQYHTTHGLPPHLTKTLSDDYQKSGPDFETILIKLNPHLVIYDFNQLWAPEVASTLHIPSIQLLSGCVALYALDAHLYTKPLDENLAKFPFPEIYPKNRDIPKGGSKYIERFVDCMRRSCEIILVRSTMELEGKYIDYLSKTLGKKVLPVGPLVQEASLLQDDHIWIMKWLDKKEESSVVFVCFGSEYILSDNEIEDIAYGLELSQVSFVWAIRAKTSALNGFIDRVGDKGLVIDKWVPQANILSHSSTGGFISHCGWSSTMESIRYGVPIIAMPMQFDQPYNARLMETVGAGIEVGRDGEGRLKREEIAAVVRKVVVEDSGESIREKAKELGEIMKKNMEAEVDGIVIENLVKLCEMNN

SEQ ID NO: 3 - DNA, 1425 bp, Stevia rebaudiana
atgtccacctcagagcttgttttcatcccatctcccggagctggccatctaccaccaacggtcgagctcgcaaagcttctgttacatcgcgatcaacgactttcggtcacaatcatcgtcatgaatctctggttaggtccaaaacacaacactgaagcacgaccttgtgttcccagtttacggttcgttgacatcccttgcgatgagtccaccatggctctcatctcacccaatacttttatatctgcgttcgttgaacaccacaaaccgcgtgttagagacatagtccgaggtataattgagtctgactcggttcgactcgctgggttcgttcttgatatgttttgtatgccgatgagtgatgttgcaaacgagtttggagttccgagttacaattatttcacatccggtgcagccacgttagggttgatgtttcaccttcaatggaaacgtgatcatgaaggttatgatgcaaccgagttgaaaaactcggatactgagttgtctgttccgagttatgttaacccggttcctgctaaggttttaccggaagtggtgttggataaagaaggtgggtccaaaatgtttcttgaccttgcggaaaggattcgcgagtcgaagggtataatagtaaattcatgtcaggcgattgaaagacacgcgctcgagtacctttcaagcaacaataacggtatcccacctgttttcccggttggtccgattttgaaccttgaaaacaaaaaagacgatgctaaaaccgacgagattatgaggtggttaaatgagcaaccggaaagctcggttgtgtttttatgtttcggaagcatgggtagctttaacgagaaacaagtgaaggagattgcggttgcgattgaaagaagtggacatagatttttatggtcgcttcgtcgtccgacaccgaaagaaaagatagagtttccgaaagaatatgaaaacttggaagaagttcttccagagggattccttaaacgtacatcaagcatcgggaaggtgatcgggtgggccccacaaatggcggtgttgtctcacccgtcagttggtgggtttgtgtcgcattgtggttggaactcgacattggagagtatgtggtgtggggttccgatggcagcttggccattatatgctgaacaaacgttgaatgcttttctacttgtggtggaactgggattggcggcggagattaggatggattatcggacggatacgaaagcggggtatgacggtgggatggaggtgacggtggaggagattgaagatggaattaggaagttgatgagtgatggtgagattagaaataaggtgaaagatgtgaaagagaagagtagagctgcggttgttgaaggtggatcttcttacgcatccattggaaaattcatcgagcatgtatcgaatgttacgatttaa

SEQ ID NO: 4 - protein, 1425 aa, Stevia rebaudiana
ATGTCCACCTCAGAGCTTGTTTTCATCCCATCTCCCGGAGCTGGCCATCTACCACCAACGGTCGAGCTCGCAAAGCTTCTGTTACATCGCGATCAACGACTTTCGGTCACAATCATCGTCATGAATCTCTGGTTAGGTCCAAAACACAACACTGAAGCACGACCTTGTGTTCCCAGTTTACGGTTCGTTGACATCCCTTGCGATGAGTCCACCATGGCTCTCATCTCACCCAATACTTTTATATCTGCGTTCGTTGAACACCACAAACCGCGTGTTAGAGACATAGTCCGAGGTATAATTGAGTCTGACTCGGTTCGACTCGCTGGGTTCGTTCTTGATATGTTTTGTATGCCGATGAGTGATGTTGCAAACGAGTTTGGAGTTCCGAGTTACAATTATTTCACATCCGGTGCAGCCACGTTAGGGTTGATGTTTCACCTTCAATGGAAACGTGATCATGAAGGTTATGATGCAACCGAGTTGAAAAACTCGGATACTGAGTTGTCTGTTCCGAGTTATGTTAACCCGGTTCCTGCTAAGGTTTTACCGGAAGTGGTGTTGGATAAAGAAGGTGGGTCCAAAATGTTTCTTGACCTTGCGGAAAGGATTCGCGAGTCGAAGGGTATAATAGTAAATTCATGTCAGGCGATTGAAAGACACGCGCTCGAGTACCTTTCAAGCAACAATAACGGTATCCCACCTGTTTTCCCGGTTGGTCCGATTTTGAACCTTGAAAACAAAAAAGACGATGCTAAAACCGACGAGATTATGAGGTGGTTAAATGAGCAACCGGAAAGCTCGGTTGTGTTTTTATGTTTCGGAAGCATGGGTAGCTTTAACGAGAAACAAGTGAAGGAGATTGCGGTTGCGATTGAAAGAAGTGGACATAGATTTTTATGGTCGCTTCGTCGTCCGACACCGAAAGAAAAGATAGAGTTTCCGAAAGAATATGAAAACTTGGAAGAAGTTCTTCCAGAGGGATTCCTTAAACGTACATCAAGCATCGGGAAGGTGATCGGGTGGGCCCCACAAATGGCGGTGTTGTCTCACCCGTCAGTTGGTGGGTTTGTGTCGCATTGTGGTTGGAACTCGACATTGGAGAGTATGTGGTGTGGGGTTCCGATGGCAGCTTGGCCATTATATGCTGAACAAACGTTGAATGCTTTTCTACTTGTGGTGGAACTGGGATTGGCGGCGGAGATTAGGATGGATTATCGGACGGATACGAAAGCGGGGTATGACGGTGGGATGGAGGTGACGGTGGAGGAGATTGAAGATGGAATTAGGAAGTTGATGAGTGATGGTGAGATTAGAAATAAGGTGAAAGATGTGAAAGAGAAGAGTAGAGCTGCGGTTGTTGAAGGTGGATCTTCTTACGCATCCATTGGAAAATTCATCGAGCATGTATCGAATGTTACGATTTAA

SEQ ID NO: 5 - DNA, 1071 bp, Artificial Sequence
  Synthetic oligonucleotide
atggatttggccaacggtgttatttccgccgaattattgcatgctcaagctcatgtttggaaccacattttcaacttcatcaagtccatgtctttgaagtgcgctattcaattgggtatcccagatatcattcataaccatggtaagccaatgaccttgccagaattggttgctaaattgccagttcacccaaaaagatctcaatgcgtttacagattgatgagaatcttggtccattctggttttttggctgctcaaagagttcaacaaggtaaagaagaagaaggttacgttttgaccgatgcctcaagattgttgttgatggatgattccttgtccatcagaccattggttttggctatgttagatccaattttgaccaagccatggcattatttgtctgcctggtttcaaaatgatgatccaactccatttcataccgctcacgaaagatcattttgggattatgctggtcatgaaccacaattgaacaactcattcaatgaagctatggcttccgatgctagattattgacttccgtcttgttgaaagaaggtcaaggtgtttttgctggtttgaactcattggttgatgttggtggtggtactggtaaagttgctaaagctattgctaatgccttcccacatttgaactgtaccgttttggatttgccacatgttgttgcaggtttacaaggttctaagaacttgaattacttcgccggtgatatgttcgaagctattccaccagctgatgctattttgttgaaatggatattgcacgactggtccgatgaagaatgtgttaagattttgaagagatgcagagaagccatcccatctaaagaaaatggtggtaaggttatcatcatcgacatgattatgatgaagaatcaaggtgactacaagtccactgaaacccaattattcttcgacatgaccatgatgattttcgctccaggtagagaaagagatgaaaacgaatgggaaaagttgttcttggatgctggtttctcccattacaagattacaccaattttgggtttgagatccttgatcgaagtttaccct

SEQ ID NO: 6 - protein, 357 aa, Vitis vinifera
MDLANGVISAELLHAQAHVWNHIFNFIKSMSLKCAIQLGIPDIIHNHGKPMTLPELVAKLPVHPKRSQCVYRLMRILVHSGFLAAQRVQQGKEEEGYVLTDASRLLLMDDSLSIRPLVLAMLDPILTKPWHYLSAWFQNDDPTPFHTAHERSFWDYAGHEPQLNNSFNEAMASDARLLTSVLLKEGQGVFAGLNSLVDVGGGTGKVAKAIANAFPHLNCTVLDLPHVVAGLQGSKNLNYFAGDMFEAIPPADAILLKWILHDWSDEECVKILKRCREAIPSKENGGKVIIIDMIMMKNQGDYKSTETQLFFDMTMMIFAPGRERDENEWEKLFLDAGFSHYKITPILGLRSLIEVYP

SEQ ID NO: 7 - DNA, 1389 bp, Arabidopsis thaliana
atgggtgaagaagctatagttctgtatcctgcaccaccaataggtcacttagtgtccatggttgagttaggtaaaaccatcctctccaaaaacccatctctctccatccacattatcttagttccaccgccttatcagccggaatcaaccgccacttacatctcctccgtctcctcctccttcccttcaataaccttccaccatcttcccgccgtcacaccgtactcctcctcctccacctctcgccaccaccacgaatctctcctcctagagatcctctgttttagcaacccaagtgtccaccgaactcttttctcactctctcggaatttcaatgtccgagcaatgatcatcgatttcttctgcaccgccgttttagacatcaccgctgacttcacgttcccggtttacttcttctacacctctggagccgcatgtctcgccttttccttctatctcccgaccatcgacgaaacaacccccggaaaaaacctcaaagacattcctacagttcatatccccggcgttcctccgatgaagggctccgatatgcctaaggcggtgctcgaacgagacgatgaggtctacgatgtttttataatgttcggtaaacagctctcgaagtcgtcagggattattatcaatacgtttgatgctttagaaaacagagccatcaaggccataacagaggagctctgttttcgcaatatttatccaattggaccgctcattgtaaacggaagaatcgaagatagaaacgacaacaaggcagtttcttgtctcaattggctggattcgcagccggaaaagagtgttgtgtttctctgttttggaagcttaggtttgttctcaaaagaacaggtgatagagattgctgttggtttagagaaaagtgggcagagattcttgtgggtggtccgtaatccacccgagttagaaaagacagaactggatttgaaatcactcttaccagaaggattcttaagccgaaccgaagacaaagggatggtcgtgaaatcatgggctccgcaagttccggttctgaatcataaagcagtcgggggattcgtcactcattgcggttggaattcaattcttgaagctgtttgtgctggtgtgccgatggtggcttggccgttgtacgctgagcagaggtttaatagagtgatgattgtggatgagatcaagattgcgatttcgatgaatgaatcagagacgggtttcgtgagctctacagaggtggagaaacgagtccaagagataattggggagtgtccggttagggagcgaaccatggctatgaagaacgcagccgaattagccttgacagaaactggttcgtctcataccgcattaactactttactccagtcgtggagcccaaagtga

SEQ ID NO: 8 - protein, 462 aa, Arabidopsis thaliana
MGEEAIVLYPAPPIGHLVSMVELGKTILSKNPSLSIHIILVPPPYQPESTATYISSVSSSFPSITFHHLPAVTPYSSSSTSRHHHESLLLEILCFSNPSVHRTLFSLSRNFNVRAMIIDFFCTAVLDITADFTFPVYFFYTSGAACLAFSFYLPTIDETTPGKNLKDIPTVHIPGVPPMKGSDMPKAVLERDDEVYDVFIMFGKQLSKSSGIIINTFDALENRAIKAITEELCFRNIYPIGPLIVNGRIEDRNDNKAVSCLNWLDSQPEKSVVFLCFGSLGLFSKEQVIEIAVGLEKSGQRFLWVVRNPPELEKTELDLKSLLPEGFLSRTEDKGMVVKSWAPQVPVLNHKAVGGFVTHCGWNSILEAVCAGVPMVAWPLYAEQRFNRVMIVDEIKIAISMNESETGFVSSTEVEKRVQEIIGECPVRERTMAMKNAAELALTETGSSHTALTTLLQSWSPK

SEQ ID NO: 9 - DNA, 1464 bp, Catharanthus roseus
atggttaatcagctccatattttcaacttcccattcatggcacagggccatatgttacccgccttagacatggccaatctattcacttctcgtggagtcaaagtaacattaatcacaacccatcaacatgttcccatgtttacaaaatccatagaaaggagcagaaattctggatttgatatatccattcaatccatcaaattcccagcttcagaagttggtttacctgaaggaatcgaaagtctagatcaagtttcaggggacgacgaaatgcttcctaagttcatgagaggagttaatttactccaacaacctctcgaacaactattgcaagaatctcgtcctcattgtcttctttctgatatgttcttcccttggactactgaatctgctgctaaatttggtattcccagattgctttttcatgggtcctgttcctttgccctctctgcagctgaaagtgtgagaagaaataaacctttcgagaatgtttccacagacacagaggaatttgttgtgcctgatcttccccaccaaattaaattaaccagaacacaaatttcaacatacgaaagggaaaatattgagtcagattttaccaaaatgctgaagaaagttagggattcagaatccacatcttacggagttgtagtcaatagtttctatgaacttgaaccagattatgccgattattacatcaacgttttgggaagaaaagcatggcatatagggccttttttgctttgtaacaaattacaagctgaagataaagcccaaagggggaagaaatcagcaattgatgcagacgaatgtttaaattggcttgattcgaaacaaccaaattccgtaatttatctctgtttcggaagtatggccaatttaaattctgcccaattacacgaaattgcaacagcccttgaatcctccggccaaaatttcatctgggttgttagaaaatgtgtggacgaagaaaacagttcaaaatggtttccagaaggattcgaagaaagaacaaaagaaaaagggctaattataaagggatgggcaccacaaaccctaattcttgaacacgaatcagtaggagcatttgttacccattgtggttggaattcaactcttgaaggaatctgcgcaggggttcctctggtgacttggcctttctttgctgagcaatttttcaatgagaaattgattacagaggtactgaaaacgggatacggagttggggctcggcaatggagtagagtttcaacagagattataaaaggagaagccatagctaatgctattaatcgagtaatggtgggtgatgaagctgttgagatgagaaacagagcaaaagatttgaaggaaaaggcaagaaaagctttggaagaagatggatcttcttatcgtgatcttactgctcttattgaagaattgggggcatatcgttctcaagttgaaagaaagcaacaagactag

SEQ ID NO: 10 - protein, 487 aa, Catharanthus roseus
MVNQLHIFNFPFMAQGHMLPALDMANLFTSRGVKVTLITTHQHVPMFTKSIERSRNSGFDISIQSIKFPASEVGLPEGIESLDQVSGDDEMLPKFMRGVNLLQQPLEQLLQESRPHCLLSDMFFPWTTESAAKFGIPRLLFHGSCSFALSAAESVRRNKPFENVSTDTEEFVVPDLPHQIKLTRTQISTYERENIESDFTKMLKKVRDSESTSYGVVVNSFYELEPDYADYYINVLGRKAWHIGPFLLCNKLQAEDKAQRGKKSAIDADECLNWLDSKQPNSVIYLCFGSMANLNSAQLHEIATALESSGQNFIWVVRKCVDEENSSKWFPEGFEERTKEKGLIIKGWAPQTLILEHESVGAFVTHCGWNSTLEGICAGVPLVTWPFFAEQFFNEKLITEVLKTGYGVGARQWSRVSTEIIKGEAIANAINRVMVGDEAVEMRNRAKDLKEKARKALEEDGSSYRDLTALIEELGAYRSQVERKQQD

SEQ ID NO: 11 - DNA, 4551 bp, Candida albicans
atgtccttttacaaaaaagtaaccaggggtttagccacacctttacaaggatcaataaaccttttcagtggctctcccaatgtcagcggcgacgaaggcacagatgcagataatgagaatcctgaacatagaacaacttatcattccttatcgggacgagtgaatcacgacgatgacgacgaagatgttgccaaattagaagatattgttgggtttttcacagggttattgaataccacgacagtttgtgctggattgggaagtttgaacaacttgaaaaagcattatttggatgagtttatcaaaaaaagtgctttgaatccattgagaccacaaaattatggaccagaaacgagcagtaaacttaataataattcgattgaagaattactcaataatggtgacacaagtttggaaaaagtccgaaaaatgagcctctatgattttgatgaacacacttcagattcagaagaggaagattcagctgacgaagaagaggagctgatagcagaaaacttgaaaccagggaaaagtggtaaagccaggaatcaccctagggattcaagaaccacagcaaccttaatcactacccaaataacaagaacaagaacagcaacagcaacaacagcaacaccgacaccgacaccgaccagttcagtcgatacagatgtcacagatgtcacagaacccattggtaaagtcaccacagagatccctgaagagcaattacaaggtctcaatccattacagaaatccgtggtgaaaaacttggaccctcatcacgtgagagaaggagtattgattaaggttaaaaattcagagacacctttgaaaaatgatcgtcaggaactactcgaaaagattcagctacggttaaaaattgccgataaactacagagagtttttgatttaagcgatgaagatacgttttgtggaaattacagtgcttggttgattaaggatgtattattacaaggacatgtttatctcacaaaagatgcactactatattttgccttcctacccaaacgttttagtctagagaattcttcagaagtattagacgaggataattcttctagtattgtttactcaggaaacttgggacttaaaagtgcaaagtatggtgaagttgtcctcaatacggttcttcaacacagatattgggctgttctaagagctgaaactttgagtatctattcatcatcgaccaatttgtatttccctgtattggtaattgatatcaagaaatgcttgtataccgagattatcgacaaggaaaagttgaatcgagaagctatttcgccagtgaaccgtggcacatacagtcctaatggcggattatcaggtactgcaactccaagagcctctactttagaaaacacagcttcagaacttaatagcatgctctccggtgattcatacagtccaactgaagacaatgtcgaaacaacagcatcaaccgtctggttcaaattagttacaaaaaagaaaacttacaagttcagttgcgatagctcgttttcagccagacaatggtgtaacaatataacaaaattgatttttcaacacaataacgccaattctaatggagaggttttaattaagatcccgatttctaaaatagcagaatacaacaaaagagcgttgtttagcgaagaggaagaagaagacaggactttggatgccacaatgaacgacataccgttaaatgtaacaataaaatacttgggcgataacgataacgaacgaaagagagataaattgaagagaaaatacaaaggtgaggagcctacaatcgaggaagtccactttttatttcccaaaagtggtgttgagtttttcgaaacttttgataaattagtgaatcctgttgttctagacaatgacaaccaatcatcaaagtcttctatcacaagtaccaatttttcggaaaaagcgatatctactttgagcaaatcaccaaaccatttagtgcaaacagtgcttgacttcaacaaacctgttgatgacgatatttcagcatttaaaaagtttgggactacaataacatcgccaacacgaattttcaaagctactatcacgtcaccagaaatgactagcattgatgaaactagtttgagagatagctttgacagtgatcgtcttcaccttccacgagatatgtctgagagagcattgaaaaatttggaagtgtcatttgtgactagtcttaaaaaattggaggacgcatcaaaaaggtacgagaagccccatatggaacacagtcagacaaatcttgcttcaatattgtctgacccttcggaagtgaaaaaagaatcgaaaacagctatactgaagagtatcaaggctctatatagtgttgggacgcattggtcagcaaccccaaatcactactttgaattgggcaaatattatgttaacaaagtgcaggaaagagactcgtctcaaagaaatttccaatctcatttttctacaaattcgaaattgcttgcatcctattatggccatttattgagaacagttccagtatatggtaaaatatacgtgtctgaaacagatgtttgcttcagaagtttattgcctggagtatcaacaaagatggtattaccaatgactgatatcgaagaagtaagagcttcacggggttcaagattgacatatcatgggttgaggcttattgtacgaggaagtgaggaattggatcttgaatttggctcttccaagtcaagagatgattttcaaaaagttgttcttagtgttttagaaagattgcactcgaaggaaggatttagacctgaaccatatcaatggggtagcaattttgaagttgagttatacaagacaagaatggaatacagtgattctgaaaatcgagaaatccaacaatatgataacctgattgatattaaatttgcagaaaagaaaattgaaatggctcgagtgagaatgtttgaagacagactaatggctgcctcgggattggacgtccccattattttggaagattcaccttttttcaaaacagaattgcggccatctacttcttataatatcacgttattgactattggttctcgaggtgatgttcaaccatatattgcgttgggtaagggattggtaaaagagggtcataatgtcaccattgctactcatgcagaatttggagactggataaaaacatttggattagggttcaaagaaattgctggggatccggccgagttgatgtcttttatggtaactcacaattctatgtctgtggggtttttgaagaatgcccaacaaaagttcaggtcgtggatctcaaagttattgactacaagctgggaggcctgtcagggttctgacattttgattgaaagtccgtcggctatgagcgggatacatattggggaagcattggggataccttattttagagcgttcaccatgccctggacaagaaccagagcctatccacatgcattctttgtaccagaacaaaagaaaggaggctcatataattacttaactcatgtattatttgaaaacatattttggaaaggtatttccggacaagtaaataaatggagagttgaggaattagatttgccaaagaccaatttatacaggttgcaacagacaagggtccccttcttgtataatgtttcacccgctatattaccgccatctgttgattttcctgattggattaaagtaactggatactggtttttagatgaaggttctggagattacaagccacctgaagaacttgtacaatttatgaaaaaagcatcccgtgacaaaaagaagattgtttacattggatttggttctattgtagtgaaagatgcaaaatccttaacgaaagctgtggtgtctgctgtgagaagagccgacgttcgttgtattttaaacaagggttggtctgatcgattggataataaagataaaaatgaaattgaaattgagttgccaccggaaatttacaattctggaactatacctcatgattggttgtttccgcgtattgatgctgccgtgcatcacggaggttctgggaccactggtgctactatgagagccgggataccaaccattattaaaccattttttggggatcagtttttctatgcaacaagaattgaagatttgggtgctggtattgccttaaagaaattgacagccaaaactttgggagatgctcttgtaaaggcaacacacgatttgaaaattatagataaagcaaagagagttagtcaacaaataaaacacgaacatggagtgctttcagctatagaatcgatttattcagaattagaatattcgagaaacttgatattgatcaaagatatccataatcaaaattataaacgtcaccatccagtaccatcaggtgttcaaactccagcttatgataccgatagcgatgattatgacgatgacgaggatgatgatgagtctgacaaagacgatgaagaagaagaagaagaagaagaaaattcttatgacggttatgatggaaatggggtcaataatagtcgtagtcaaaattcctccaactag

SEQ ID NO: 12 - protein, 1516 aa, Candida albicans
MSFYKKVTRGLATPLQGSINLFSGSPNVSGDEGTDADNENPEHRTTYHSLSGRVNHDDDDEDVAKLEDIVGFFTGLLNTTTVCAGLGSLNNLKKHYLDEFIKKSALNPLRPQNYGPETSSKLNNNSIEELLNNGDTSLEKVRKMSLYDFDEHTSDSEEEDSADEEEESIAENLKPGKSGKARNHPRDSRTTATLITTQITRTRTATATTATPTPTPTSSVDTDVTDVTEPIGKVTTEIPEEQLQGLNPLQKSVVKNLDPHHVREGVLIKVKNSETPLKNDRQELLEKIQLRLKIADKLQRVFDLSDEDTFCGNYSAWLIKDVLLQGHVYLTKDALLYFAFLPKRFSLENSSEVLDEDNSSSIVYSGNLGLKSAKYGEVVLNTVLQHRYWAVLRAETLSIYSSSTNLYFPVLVIDIKKCLYTEIIDKEKLNREAISPVNRGTYSPNGGLSGTATPRASTLENTASELNSMLSGDSYSPTEDNVETTASTVWFKLVTKKKTYKFSCDSSFSARQWCNNITKLIFQHNNANSNGEVLIKIPISKIAEYNKRALFSEEEEEDRTLDATMNDIPLNVTIKYLGDNDNERKRDKLKRKYKGEEPTIEEVHFLFPKSGVEFFETFDKLVNPVVLDNDNQSSKSSITSTNFSEKAISTLSKSPNHLVQTVLDFNKPVDDDISAFKKFGTTITSPTRIFKATITSPEMTSIDETSLRDSFDSDRLHLPRDMSERALKNLEVSFVTSLKKLEDASKRYEKPHMEHSQTNLASILSDPSEVKKESKTAISKSIKALYSVGTHWSATPNHYFELGKYYVNKVQERDSSQRNFQSHFSTNSKLLASYYGHLLRTVPVYGKIYVSETDVCFRSLLPGVSTKMVLPMTDIEEVRASRGSRLTYHGLRLIVRGSEELDLEFGSSKSRDDFQKVVLSVLERLHSKEGFRPEPYQWGSNFEVELYKTRMEYSDSENREIQQYDNSIDIKFAEKKIEMARVRMFEDRLMAASGLDVPIILEDSPFFKTELRPSTSYNITLLTIGSRGDVQPYIALGKGLVKEGHNVTIATHAEFGDWIKTFGLGFKEIAGDPAELMSFMVTHNSMSVGFLKNAQQKFRSWISKLLTTSWEACQGSDILIESPSAMSGIHIGEALGIPYFRAFTMPWTRTRAYPHAFFVPEQKKGGSYNYLTHVLFENIFWKGISGQVNKWRVEELDLPKTNLYRLQQTRVPFLYNVSPAILPPSVDFPDWIKVTGYWFLDEGSGDYKPPEELVQFMKKASRDKKKIVYIGFGSIVVKDAKSLTKAVVSAVRRADVRCILNKGWSDRLDNKDKNEIEIELPPEIYNSGTIPHDWLFPRIDAAVHHGGSGTTGATMRAGIPTIIKPFFGDQFFYATRIEDLGAGIALKKLTAKTLGDALVKATHDLKIIDKAKRVSQQIKHEHGVLSAIESIYSELEYSRNLILIKDIHNQNYKRHHPVPSGVQTPAYDTDSDDYDDDEDDDESDKDDEEEEEEEENSYDGYDGNGVNNSRSQNSSN

SEQ ID NO: 13 - DNA, 1452 bp, Arabidopsis thaliana
atgggtagtgatcatcatcatcgaaagctccacgttatgttcttccctttcatggcttatggtcacatgataccaactctagacatggctaagcttttctctagcagaggagccaaatccacaatcctcaccacatctctcaactccaagatcctccaaaaacccatcgacacattcaagaatctgaatccgggtctcgaaatcgacatccagatcttcaatttcccttgcgtggagctggggttaccagaaggatgtgaaaacgttgatttcttcacttcaaacaacaatgatgataaaaacgagatgatcgtgaaattctttttctcgacaaggtttttcaaagaccagcttgagaaactcctcgggacaacgagaccagactgtcttatcgccgacatgttcttcccctgggctactgaagctgctgggaagttcaatgtgccaagacttgtgttccacggcactggctacttctctttatgcgctggttattgcatcggagtgcataaaccacagaagagagtggcttcaagctctgagccatttgtgattcccgagctccctgggaacattgtgataactgaagaacagatcatagatggcgatggagaatccgacatgggaaagtttatgactgaagttagggaatcggaagtgaagagctcaggagttgttttgaatagtttctacgagctagaacatgattacgccgatttttacaaaagttgtgtacaaaagagagcgtggcatatcggtccgctatcggtttacaacaggggatttgaggagaaggctgagagaggaaagaaagcgaacattgatgaggctgaatgcctcaaatggcttgactccaagaaaccaaattcagtcatttatgtttcctttgggagcgtggctttcttcaagaatgaacagttattcgagatcgctgcagggttagaagcttccggtacaagtttcatttgggttgttaggaaaaccaaagatgatagagaagaatggttaccagaagggttcgaagagagggtgaaagggaaaggtatgataataagaggatgggcaccacaggtgctgatacttgaccaccaagcaaccggtgggtttgtgacccattgcggctggaactcgcttcttgaaggagtggctgcagggctaccaatggtgacatggcctgtaggagcggagcaattctacaatgagaaattggttacgcaagtgctcagaacaggagtgagcgtgggagcgagcaagcatatgaaagttatgatgggagatttcattagcagagagaaagtggataaagcggtgagggaggttttggctggggaagcagcagaggagaggcggagacgggcaaagaagctagcggcgatggctaaagctgccgtggaagaaggagggtcttccttcaacgatctaaacagcttcatggaagagtttagttcataa

SEQ ID NO: 14 - protein, 483 aa, Arabidopsis thaliana
MGSDHHHRKLHVMFFPFMAYGHMIPTLDMAKLFSSRGAKSTILTTSLNSKILQKPIDTFKNLNPGLEIDIQIFNFPCVELGLPEGCENVDFFTSNNNDDKNEMIVKFFFSTRFFKDQLEKLLGTTRPDCLIADMFFPWATEAAGKFNVPRLVFHGTGYFSLCAGYCIGVHKPQKRVASSSEPFVIPELPGNIVITEEQIIDGDGESDMGKFMTEVRESEVKSSGVVLNSFYELEHDYADFYKSCVQKRAWHIGPLSVYNRGFEEKAERGKKANIDEAECLKWLDSKKPNSVIYVSFGSVAFFKNEQLFEIAAGLEASGTSFIWVVRKTKDDREEWLPEGFEERVKGKGMIIRGWAPQVLILDHQATGGFVTHCGWNSLLEGVAAGLPMVTWPVGAEQFYNEKLVTQVLRTGVSVGASKHMKVMMGDFISREKVDKAVREVLAGEAAEERRRRAKKLAAMAKAAVEEGGSSFNDLNSFMEEFSS

SEQ ID NO: 15 - DNA, 1317 bp, Artificial Sequence
  Synthetic oligonucleotide
atggattcaaaaatcgattccaagacatttcgagtagtgatgctaccatggttagcatatagtcacatatctagcttcctagtttttgccaaaagactcacaaaccataactttcatatatacatatgttcatcccaaaccaacatgcaatatctcaaaaacaaccttacttctcaatactctaaatccatccaactcattgaactaaatcttccatcttcaagtgaactccctcttcaatatcacaccactcatggccttccacctcacctcacgaaaaccttatcggacgattatcaaaaatcaggtccggattttgagaccatactcataaaacttaacccgcatttggttatatatgattttaaccaattgtgggccccagaagtggcttcgacgctacacatccctagtattcagttattaagcggatgtgtagcgttatatgctttggatgctcatctctacacaaagccattagatgaaaatttggcaaaatttccttttcctgaaatatatcctaaaaatcgcgatattccaaaaggtggttcgaaatatattgaacgctttgttgattgcatgagaaggtcttgtgagataatattggttagaagtacaatggagttagaagggaagtatattgattatttatctaaaactcttggaaagaaagtattgccggttggtccattagttcaagaagcttctttattacaagatgatcatatttggatcatgaaatggcttgataaaaaagaagaatcttcagttgtgtttgtatgttttggtagtgaatatattttatctgataatgagatagaagatatagcctatggtttggagcttagccaagtgagttttgtatgggctataagggcgaaaactagtgctcttaatgggtttattgacagggttggagataagggtctggttatcgataaatgggtcccacaagccaatattttaagtcattcgagtacgggtggatttattagtcattgtggatggagttcaactatggaaagtattcgttatggtgttccgattatagcaatgcctatgcaatttgatcagccgtataatgcaaggctaatggagacggttggagccggtattgaagttgggagagatggtgaggggaggctgaagagggaggagattgcggcggtggtgaggaaggtggtggtggaggatagtggtgagagtataagggaaaaggctaaggagttgggtgagattatgaagaagaatatggaggcagaggttgatggtatagtgattgagaatttggtcaagctttgtgaaatgaataattga

SEQ ID NO: 16 - protein, 438 aa, Bellis perennis
MDSKIDSKTFRVVMLPWLAYSHISSFLVFAKRLTNHNFHIYICSSQTNMQYLKNNLTSQYSKSIQLIELNLPSSSELPLQYHTTHGLPPHLTKTLSDDYQKSGPDFETILIKLNPHLVIYDFNQLWAPEVASTLHIPSIQLLSGCVALYALDAHLYTKPLDENLAKFPFPEIYPKNRDIPKGGSKYIERFVDCMRRSCEIILVRSTMELEGKYIDYLSKTLGKKVLPVGPLVQEASLLQDDHIWIMKWLDKKEESSVVFVCFGSEYILSDNEIEDIAYGLELSQVSFVWAIRAKTSALNGFIDRVGDKGLVIDKWVPQANILSHSSTGGFISHCGWSSTMESIRYGVPIIAMPMQFDQPYNARLMETVGAGIEVGRDGEGRLKREEIAAVVRKVVVEDSGESIREKAKELGEIMKKNMEAEVDGIVIENLVKLCEMNN

SEQ ID NO: 17 - DNA, 1443 bp, Arabidopsis thaliana
atggcggaagcaaacactccacacatagcaatcatgccgagtcccggtatgggtcaccttatcccattcgtcgagttagcaaagcgactcgttcagcacgactgtttcaccgtcacaatgatcatctccggtgaaacttcgccgtctaaggcacaaagatccgttctcaactctctcccttcctccatagcctccgtatttctccctcccgccgatctttccgatgttccctccacagcgcgaatcgaaactcgggccatgctcaccatgactcgttccaatccggcgctccgggagctttttggctctttatcaacgaagaaaagtctcccggcggttctcgtcgtcgatatgtttggtgcggatgcgttcgacgtggccgttgacttccacgtgtcaccatacattttctatgcatccaatgcaaacgtcttgtcgttttttcttcacttgccgaaactagacaaaacggtgtcgtgtgagtttaggtacttaaccgaaccgcttaagattcccggctgtgtcccgataaccggtaaggactttcttgatacggttcaagaccgaaacgacgacgcatacaaattgcttctccataacaccaagaggtacaaagaagctaaagggattctagtgaattccttcgttgatttagagtcgaatgcaataaaggccttacaagaaccggctcctgataaaccaacggtatacccgattgggccgctggttaacacaagttcatctaatgttaacttggaagacaagttcggatgtttaagttggctagacaaccaaccattcggctcggttctatacatatcatttggaagcggcggaacacttacatgtgagcagtttaatgagcttgctattggtcttgcggagagcggaaaacggtttatttgggtcatacgaagtccaagcgagatagttagttcgtcgtatttcaatccacacagcgagacagaccccttttcgtttttaccaattgggttcttagaccgaaccaaagagaaaggtttggtggttccatcatgggctccacaggttcaaatcctggctcatccatccacatgcgggtttttaacacactgtggatggaattcgaccttagaaagcattgtaaacggtgtaccactcatagcgtggcctttattcgcggagcaaaagatgaatacattgctactcgtggaggatgttggagcggctctaagaatccatgcgggtgaagatgggattgtacggagggaagaagtggtgagagtggtgaaggcactgatggaaggtgaagagggaaaagccataggaaataaagtgaaggagttgaaagaaggagttgttagagtcttgggtgacgatggattgtccagcaagtcatttggtgaagttttgttaaagtggaaaacgcaccagcgagatatcaaccaagagacgtcccactaa

SEQ ID NO: 18 - protein, 480 aa, Arabidopsis thaliana
MAEANTPHIAIMPSPGMGHLIPFVELAKRLVQHDCFTVTMIISGETSPSKAQRSVLNSLPSSIASVFLPPADLSDVPSTARIETRAMLTMTRSNPALRELFGSLSTKKSLPAVLVVDMFGADAFDVAVDFHVSPYIFYASNANVLSFFLHLPKLDKTVSCEFRYLTEPLKIPGCVPITGKDFLDTVQDRNDDAYKLLLHNTKRYKEAKGILVNSFVDLESNAIKALQEPAPDKPTVYPIGPLVNTSSSNVNLEDKFGCLSWLDNQPFGSVLYISFGSGGTLTCEQFNELAIGLAESGKRFIWVIRSPSEIVSSSYFNPHSETDPFSFLPIGFLDRTKEKGLVVPSWAPQVQILAHPSTCGFLTHCGWNSTLESIVNGVPLIAWPLFAEQKMNTLLLVEDVGAALRIHAGEDGIVRREEVVRVVKALMEGEEGKAIGNKVKELKEGVVRVLGDDGLSSKSFGEVLLKWKTHQRDINQETSH

SEQ ID NO: 19 - DNA, 1455 bp, Arabidopsis thaliana
atgaacagagaagtctctgagagaattcatattttgttcttccccttcatggctcaaggccacatgattccaattttggacatggccaagcttttctcgaggagaggagccaagtcaacccttctcacaaccccaatcaacgctaagatcttcgagaaacctattgaagcattcaaaaatcaaaaccctgatctcgaaatcggaatcaagatcttcaatttcccttgtgtagagcttggattgcctgaaggatgcgagaacgctgactttatcaactcataccaaaaatctgactcaggtgacttgttcttgaagtttcttttctctaccaagtatatgaaacaacagttggagagtttcattgaaacaaccaaaccaagtgctcttgttgccgatatgttcttcccttgggcgacagaatctgctgagaagctcggtgtaccaagacttgtgttccacggtacatctttcttttctttgtgttgttcgtataacatgaggattcataagccacacaagaaagtcgctacgagttctactccttttgtaatccctggtctcccaggagacatagttattacagaagaccaagccaatgttgccaaagaagaaacgccaatgggaaagtttatgaaagaggttagggaatcagagaccaatagctttggtgtattggttaatagcttctacgagctggaatcagcttatgctgatttttatcgtagttttgtggcgaaaagagcttggcatatcggtccgctttcgctatctaacagagagttaggagagaaagccagaagagggaaaaaggctaacattgatgagcaagaatgcctaaaatggctggactctaagacacctggttcagtagtttacttgtcctttgggagcggaactaatttcaccaacgaccagctgttagagatcgcttttggtcttgaaggttctggacaaagtttcatctgggtggttaggaaaaatgaaaaccaaggtgacaatgaagagtggttgcctgaagggtttaaagagaggacaacagggaaagggctaataatacctggatgggcgccgcaagtgctgatacttgaccataaagcaattggaggatttgtgactcattgcggatggaactcggctatagagggcattgccgcggggctgcctatggtaacatggccaatgggggcagaacagttctacaatgagaagctattgacaaaagtgttgagaataggagtgaacgttggagctaccgagttggtgaaaaaaggaaagttgattagtagagcacaagtggagaaggcagtaagggaagtgattggtggtgagaaggcagaggaaaggcggctatgggctaagaagctgggcgagatggctaaagccgctgtggaagaaggagggtcctcttataatgatgtgaacaagtttatggaagagctgaatggtagaaagtag

SEQ ID NO: 20 - protein, 484 aa, Arabidopsis thaliana
MNREVSERIHILFFPFMAQGHMIPILDMAKLFSRRGAKSTLLTTPINAKIFEKPIEAFKNQNPDLEIGIKIFNFPCVELGLPEGCENADFINSYQKSDSGDLFLKFLFSTKYMKQQLESFIETTKPSALVADMFFPWATESAEKLGVPRLVFHGTSFFSLCCSYNMRIHKPHKKVATSSTPFVIPGLPGDIVITEDQANVAKEETPMGKFMKEVRESETNSFGVLVNSFYELESAYADFYRSFVAKRAWHIGPLSLSNRELGEKARRGKKANIDEQECLKWLDSKTPGSVVYLSFGSGTNFTNDQLLEIAFGLEGSGQSFIWVVRKNENQGDNEEWLPEGFKERTTGKGLIIPGWAPQVLILDHKAIGGFVTHCGWNSAIEGIAAGLPMVTWPMGAEQFYNEKLLTKVLRIGVNVGATELVKKGKLISRAQVEKAVREVIGGEKAEERRLWAKKLGEMAKAAVEEGGSSYNDVNKFMEELNGRK

SEQ ID NO: 21 - DNA, 1368 bp, Arabidopsis thaliana
atggcgcaaccgcattttctactggtaacgtttccggcgcaaggtcacgtgaacccatctctccgttttgctcgtcggctcatcaaaacaactggcgcacgtgtaactttcgccacgtgtctctctgtcattcaccgctctatgatcccaaaccacaacaacgtcgaaaatctctctttccttactttctccgacggattcgacgacggagtcatctccaacaccgacgacgtccaaaaccggttggtacacttcgaacgtaatggcgataaagctctatcggatttcatcgaagctaatcagaatggtgactctcccgtaagttgcttgatctacacgattcttcccaactgggttccaaaagtggcgcgtagatttcatcttccctctgttcatctctggatccaaccagccttcgctttcgacatttattacaattactctacaggaaacaactccgttttcgagttcccgaatctaccttctctcgaaatccgcgatctgccttctttcctctcaccttccaacacgaacaaagccgcacaagcagtatatcaagaactgatggattttctcaaagaagaatctaacccgaaaattctcgtcaacacattcgattcgctggagccagagttcttaacagctattccgaatatagaaatggtggcagttggtcctttacttcctgcggagattttcactggaagcgaatcaggtaaagatttatcaagagatcatcaaagtagtagttatacactttggttagactcgaaaacagagtcctctgttatttatgtttcttttggaacaatggttgagttgtcgaagaaacagatagaggaactagcgagagcactcatagaagggggaagaccgttcttgtgggttataactgataaactcaacagagaagcgaaaatagaaggagaagaagagacagagattgagaagatagctggttttagacacgagcttgaagaggttgggatgattgtctcgtggtgttcgcagatagaggttttgagacaccgagccataggttgttttttgactcattgtgggtggagctcatcactggagagtttggttctcggcgttccagtggtggcgtttccgatgtggtcggatcagccagcaaatgcgaagcttttggaagaaatatggaagacaggtgtgagggtgagagagaactcggaaggtttagtagagagaggagagataatgcggtgtttggaagcagtgatggaggcgaaatcggtggagctgagggaaaacgcagagaaatggaagcgtttagcgactgaagcgggtagagaaggaggatcttcggacaagaatgtggaagcttttgtgaagagtctgttttga

SEQ ID NO: 22 - protein, 455 aa, Arabidopsis thaliana
MAQPHFLLVTFPAQGHVNPSLRFARRLIKTTGARVTFATCLSVIHRSMIPNHNNVENLSFLTFSDGFDDGVISNTDDVQNRLVHFERNGDKALSDFIEANQNGDSPVSCLIYTILPNWVPKVARRFHLPSVHLWIQPAFAFDIYYNYSTGNNSVFEFPNLPSLEIRDLPSFLSPSNTNKAAQAVYQELMDFLKEESNPKILVNTFDSLEPEFLTAIPNIEMVAVGPLLPAEIFTGSESGKDLSRDHQSSSYTLWLDSKTESSVIYVSFGTMVELSKKQIEELARALIEGGRPFLWVITDKLNREAKIEGEEETEIEKIAGFRHELEEVGMIVSWCSQIEVLRHRAIGCFLTHCGWSSSLESLVLGVPVVAFPMWSDQPANAKLLEEIWKTGVRVRENSEGLVERGEIMRCLEAVMEAKSVELRENAEKWKRLATEAGREGGSSDKNVEAFVKSLF

SEQ ID NO: 23 - DNA, 1362 bp, Arabidopsis thaliana
atggaagaactaggagtgaagagaaggatagtattggttccagttccagcacaaggtcatgtaactccgattatgcaactcgggaaggctctttactccaagggcttctccatcactgttgttctcacacagtataatcgagttagctcatccaaggacttctctgattttcatttcctcaccatcccaggcagcttgaccgagtctgatctcaaaaaccttggaccattcaagtttctcttcaagctcaatcaaatttgcgaggcaagcttcaagcaatgtattggtcaactattgcaggagcaaggtaatgatatcgcttgtgtcgtctacgatgagtacatgtacttctcccaagctgcagttaaagagtttcaacttcctagcgtcctcttcagcacgacaagtgctactgcctttgtctgtcgctctgttttgtctagagtcaacgcagagtcattcttgcttgacatgaaagatcccaaagtgtcagacaaggaatttccagggttgcatccgctaaggtacaaggacctgccaacttcagcatttgggccattagagagtatactcaaggtttacagtgagactgtcaacattcgaacagcttcggcagttatcatcaactcaacaagctgtctagagagctcatctttggcatggttacaaaaacaactgcaagttccagtgtatcctataggcccacttcacattgcagcttcagcgccttctagtttacttgaagaggacaggagttgccttgagtggttgaacaagcaaaaaataggctcagtgatttacataagtttgggaagcttggctctaatggaaactaaagacatgttggagatggcttggggtttacgtaatagcaaccaacctttcttatgggtgatccgaccgggttctattcccggctcggaatggacagagtctttaccggaggaattcagtaggttggtttcagaaagaggttacattgtgaaatgggcaccacagatagaagttctcagacatcctgcagtgggagggttttggagtcactgcggatggaactcgaccctagagagcatcggggaaggagttccgatgatctgtaggccttttacgggagatcagaaagtcaatgcgaggtacttagagagagtttggagaattggggttcaattggaaggagagctggataaaggaacagtggagagagctgtagagagattgattatggatgaagaaggagcagaaatgaggaagagagttatcaacttgaaagagaagcttcaagcctctgtcaagagtagaggttcctcattcagctcattagacaactttgtcaattccttaaaaatgatgaatttcatgtag

SEQ ID NO: 24 - protein, 453 aa, Arabidopsis thaliana
MEELGVKRRIVLVPVPAQGHVTPIMQLGKALYSKGFSITVVLTQYNRVSSSKDFSDFHFLTIPGSLTESDLKNLGPFKFLFKLNQICEASFKQCIGQLLQEQGNDIACVVYDEYMYFSQAAVKEFQLPSVLFSTTSATAFVCRSVLSRVNAESFLLDMKDPKVSDKEFPGLHPLRYKDLPTSAFGPLESILKVYSETVNIRTASAVIINSTSCLESSSLAWLQKQLQVPVYPIGPLHIAASAPSSLLEEDRSCLEWLNKQKIGSVIYISLGSLALMETKDMLEMAWGLRNSNQPFLWVIRPGSIPGSEWTESLPEEFSRLVSERGYIVKWAPQIEVLRHPAVGGFWSHCGWNSTLESIGEGVPMICRPFTGDQKVNARYLERVWRIGVQLEGELDKGTVERAVERLIMDEEGAEMRKRVINLKEKLQASVKSRGSSFSSLDNFVNSLKMMNFM

SEQ ID NO: 25 - DNA, 1377 bp, Stevia rebaudiana
atggaaaataaaacggagaccaccgttcgccggcgccggagaataatattattcccggtaccatttcaaggccacattaacccaattcttcagctagccaatgtgttgtactctaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcacggatgctctttggtacttcgcgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaaaagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttcaatggttagaccaacaaccgccaagttcggtactgtatgttagttttggtagtactagtgaagtggatgagaaagatttcttggaaatagctcgtgggttggttgatagcaagcagtcgtttttatgggtggttcgacctgggtttgtcaagggttcgacgtgggtcgaaccgttgccagatgggttcttgggtgaaagaggacgtattgtgaaatgggttccacagcaagaagtgctagctcatggagcaataggcgcattctggactcatagcggatggaactctacgttggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaataagaagagttatggtggatgaagaaggagaatacattagacagaatgcaagagttttgaaacaaaaggcagatgtttctttgatgaagggtggttcgtcttacgaatcattagagtctctagtttcttacatttcatcgttgtaa

SEQ ID NO: 26 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 27 - DNA, 1275 bp, Stevia rebaudiana
atgcttcagcttgcaacttacctccattctcaagggatttcaataaccatcgctcagtaccccaacttcaactcgccggattcttccaaccatccagaactaaccttcctcccactatcctccggcaacttatccgtcgccgacatctccggcggctttttcaagttcatccaaactcttaaccataactgcaaaccccatttccgggaataccttgttcagaacatgagttctgatgataaggaatcaatcgttatcatccgtgataatctcatgtttttcgccggagaaatcgccggcgagctgggtctgccttcgatcattttacgtggcagcaatgctgtcatgttgactgctagcgacatcatccctcaacttcatcaagaaggtcgttttccgccaccagattctttgttgcaggaaacaattccagaactggttccattcagatacaaagatctaccatttattggctatccaatacatcaaacccttgaatttagtatcaccatgatgacccccaaatcacctgcttccgccattcttatcaacaccctcgaatttcttgaacaatcggcattaacccagatccgtgatcattacaaagttccagtttttacaatcggaccattgcacaaaatagtcacaactcgttccactagcattcttgaagaagatacaagttgcatcaattggttagataaacaatcacccaaatcagtggtttatgtgagtttaggaagcttagcaaagttggatgaaaaggttgcatctgaaatggcatgtggtttagccatgagtaaccataagttcctatgggtggttcgacccggtatggttcatgggtttgaatgggtcgagtttttgccggatagtttggtgggtgaaatgaaggctagaggtttgattgtgaaatgggcaccccagacgacggttttggcgcataacgcggttggtggattttggagtcattgcggttggaactcgaccatagaatgcttagctgaaggggtcccgatgatgtgtcaaccgttttttgctgatcagttgttgaatgctaggtatgtgagtgatgtttggaagacgggttttgagattgttatcgagaaaggtgagattgcgtgcgcgattaaacgagttttggtggatgaagaaggcgaagaaatgaggcagagagctatggagattaaagaaaaggttaaaattgcaatcaacgatggtggttcttcttatgactcgttcaaggacttggtggcgtttatttcatcactctaa

SEQ ID NO: 28 - protein, 424 aa, Stevia rebaudiana
MLQLATYLHSQGISITIAQYPNFNSPDSSNHPELTFLPLSSGNLSVADISGGFFKFIQTLNHNCKPHFREYLVQNMSSDDKESIVIIRDNLMFFAGEIAGELGLPSIILRGSNAVMLTASDIIPQLHQEGRFPPPDSLLQETIPELVPFRYKDLPFIGYPIHQTLEFSITMMTPKSPASAILINTLEFLEQSALTQIRDHYKVPVFTIGPLHKIVTTRSTSILEEDTSCINWLDKQSPKSVVYVSLGSLAKLDEKVASEMACGLAMSNHKFLWVVRPGMVHGFEWVEFLPDSLVGEMKARGLIVKWAPQTTVLAHNAVGGFWSHCGWNSTIECLAEGVPMMCQPFFADQLLNARYVSDVWKTGFEIVIEKGEIACAIKRVLVDEEGEEMRQRAMEIKEKVKIAINDGGSSYDSFKDLVAFISSL

SEQ ID NO: 29 - DNA, 1383 bp, Arabidopsis thaliana
atgaccaaaccctccgacccaaccagagactcccacgtggcagttctcgcttttcctttcggcactcatgcagctcctctcctcaccgtcacgcgccgcctcgcctccgcctctccttccaccgtcttctctttcttcaacaccgcacaatccaactcttcgttattttcctccggtgacgaagcagatcgtccggcgaacatcagagtatacgatattgccgacggtgttccggagggatacgtgtttagcgggagaccacaggaggcgatcgagctgtttcttcaagctgcgccggagaatttccggagagaaatcgcgaaggcggagacggaggttggtacggaagtgaaatgtttgatgactgatgcgttcttctggttcgcggctgatatggcgacggagataaatgcgtcgtggattgcgttttggaccgccggagcaaactcactctctgctcatctctacacagatctcatcagagaaaccatcggtgtcaaagaagtaggtgagcgtatggaggagacaataggggttatctcaggaatggagaagatcagagtcaaagatacaccagaaggagttgtgtttgggaatttagactctgttttctcaaagatgcttcatcaaatgggtcttgctttgcctcgtgccactgctgttttcatcaattcttttgaagatttggatcctacattgacgaataacctcagatcgagatttaaacgatatctgaacatcggtcctctcgggttattatcttctacattgcaacaactagtgcaagatcctcacggttgtttggcttggatggagaagagatcttctggttctgtggcgtacattagctttggtacggtcatgacaccgcctcctggagagcttgcggcgatagcagaagggttggaatcgagtaaagtgccgtttgtttggtcgcttaaggagaagagcttggttcagttaccaaaagggtttttggataggacaagagagcaagggatagtggttccatgggcaccgcaagtggaactgctgaaacacgaagcaacgggtgtgtttgtgacgcattgtggatggaactcggtgttggagagtgtatcgggtggtgtaccgatgatttgcaggccattttttggggatcagagattgaacggaagagcggtggaggttgtgtgggagattggaatgacgattatcaatggagtcttcacgaaagatgggtttgagaagtgtttggataaagttttagttcaagatgatggtaagaagatgaaatgtaatgctaagaaacttaaagaactagcttacgaagctgtctcttctaaaggaaggtcctctgagaatttcagaggattgttggatgcagttgtaaacattatttga

SEQ ID NO: 30 - protein, 460 aa, Arabidopsis thaliana
MTKPSDPTRDSHVAVLAFPFGTHAAPLLTVTRRLASASPSTVFSFFNTAQSNSSLFSSGDEADRPANIRVYDIADGVPEGYVFSGRPQEAIELFLQAAPENFRREIAKAETEVGTEVKCLMTDAFFWFAADMATEINASWIAFWTAGANSLSAHLYTDLIRETIGVKEVGERMEETIGVISGMEKIRVKDTPEGVVFGNLDSVFSKMLHQMGLALPRATAVFINSFEDLDPTLTNNLRSRFKRYLNIGPLGLLSSTLQQLVQDPHGCLAWMEKRSSGSVAYISFGTVMTPPPGELAAIAEGLESSKVPFVWSLKEKSLVQLPKGFLDRTREQGIVVPWAPQVELLKHEATGVFVTHCGWNSVLESVSGGVPMICRPFFGDQRLNGRAVEVVWEIGMTIINGVFTKDGFEKCLDKVLVQDDGKKMKCNAKKLKELAYEAVSSKGRSSENFRGLLDAVVNII

SEQ ID NO: 31 - DNA, 1371 bp, Arabidopsis thaliana
atgggcagtagtgagggtcaagaaacacatgtcctaatggtaacactaccattccaaggtcacatcaatccaatgctcaaactcgcaaaacatctctcgttatcatcaaagaacctacacatcaatctcgccactattgagtcagcccgtgatctcctctccaccgtagaaaaacctcgttatccggtggacctcgtgttcttctccgatggtctacctaaagaagatccaaaggcccctgaaactcttttgaagtcattgaataaagtcggagccatgaacttgtctaaaatcatcgaagaaaagagatactcttgtatcatctcttcgccttttactccatgggttccagctgttgcagcctctcataacatctcttgtgcaatactttggatccaagcttgtggagcttactcggtttattaccgttactacatgaagacaaactctttccctgatcttgaagatctgaatcaaacggtggagttaccagctttaccattgttggaagttcgagatcttccatcgtttatgttaccttctggtggtgctcacttctataatctaatggcggaatttgcagattgtttgaggtatgtgaaatgggttttggttaattcattctatgaactcgaatcagagataatcgaatcgatggctgatttaaaacctgtaattccaattggtcctctggtttctccatttctgttgggcgatggtgaggaggaaaccctagacggtaaaaacctagatttttgtaaatctgatgattgttgtatggagtggcttgacaagcaagctaggtcttctgttgtgtacatatctttcggaagtatgctcgaaacattggagaatcaggtcgagaccatagcgaaggcgctgaagaacagaggacttccatttctttgggtgataaggccaaaggagaaagcccaaaacgttgctgttttgcaggagatggtgaaagaaggacaaggggttgttctcgagtggagtccacaagagaagattttgagccacgaggcaatctcttgttttgtcacgcattgcggctggaactcgactatggagacggtggtggctggtgttcctgtggtagcgtaccctagctggacggatcagcccattgacgcgcggttgcttgttgatgtgtttggaatcggagtaaggatgaggaatgacagtgtcgatggcgagcttaaggtcgaagaagtagaaagatgcattgaggccgtgacggagggacccgctgccgtggatataagaaggagagcggcggagctaaagcgcgtggcgagattggcgttggcacctggtggatcttcgacacggaatttagacttgttcattagtgatatcacaatcgcctaa

SEQ ID NO: 32 - protein, 456 aa, Arabidopsis thaliana
MGSSEGQETHVLMVTLPFQGHINPMLKLAKHLSLSSKNLHINLATIESARDLLSTVEKPRYPVDLVFFSDGLPKEDPKAPETLLKSLNKVGAMNLSKIIEEKRYSCIISSPFTPWVPAVAASHNISCAILWIQACGAYSVYYRYYMKTNSFPDLEDLNQTVELPALPLLEVRDLPSFMLPSGGAHFYNLMAEFADCLRYVKWVLVNSFYELESEIIESMADLKPVIPIGPLVSPFLLGDGEEETLDGKNLDFCKSDDCCMEWLDKQARSSVVYISFGSMLETLENQVETIAKALKNRGLPFLWVIRPKEKAQNVAVLQEMVKEGQGVVLEWSPQEKILSHEAISCFVTHCGWNSTMETVVAGVPVVAYPSWTDQPIDARLLVDVFGIGVRMRNDSVDGELKVEEVERCIEAVTEGPAAVDIRRRAAELKRVARLALAPGGSSTRNLDLFISDITIA

SEQ ID NO: 33 - DNA, 1473 bp, Arabidopsis thaliana
atgggatccatatcagaaatggtgttcgaaacttgtccatctccaaacccaattcatgtaatgctcgtctcgtttcaaggacaaggccacgtcaaccctcttcttcgtctcggcaagttaattgcttcaaagggtttactcgttaccttcgttacaacggagctttggggcaagaaaatgagacaagccaacaaaatcgttgacggtgaacttaaaccggttggttccggttcaatccggtttgagttctttgatgaagaatgggcagaggatgatgaccggagagctgatttctctttgtacattgctcacctagagagcgttgggatacgagaagtgtctaagcttgtgagaagatacgaggaagcgaacgagcctgtctcgtgtcttatcaataacccgtttatcccatgggtctgccacgtggcggaagagttcaacattccttgtgcggttctctgggttcagtcttgtgcttgtttctctgcttattaccattaccaagatggctctgtttcattccctacggaaacagagcctgagctcgatgtgaagcttccttgtgttcctgtcttgaagaacgacgagattcctagctttctccatccttcttctaggttcacgggttttcgacaagcgattcttgggcaattcaagaatctgagcaagtccttctgtgttctaatcgattcttttgactcattggaacaagaagttatcgattacatgtcaagtctttgtccggttaaaaccgttggaccgcttttcaaagttgctaggacagttacttctgacgtaagcggtgacatttgcaaatcaacagataaatgcctcgagtggttagactcgaggcctaaatcgtcagttgtctacatttcgttcgggacagttgcatatttgaagcaagaacagatcgaagagatcgctcacggagttttgaagtcgggtttatcgttcttgtgggtgattagacctccaccacacgatctgaaggtcgagacacatgtcttgcctcaagaacttaaagagagtagtgctaaaggtaaagggatgattgtggattggtgcccacaagagcaagtcttgtctcatccttcagtggcatgcttcgtgactcattgtggatggaactcgacaatggaatctttgtcttcaggtgttccggtggtttgttgtccgcaatggggagatcaagtgactgatgcagtgtatttgatcgatgttttcaagaccggggttagactaggccgtggagcgaccgaggagagggtagtgccaagggaggaagtggcggagaagcttttggaagcgacagttggggagaaggcagaggagttgagaaagaacgctttgaaatggaaggcggaggcggaagcagcggtggctccaggaggttcgtcggataagaattttagggagtttgtggagaagttaggtgcgggagtaacgaagactaaagataatggatactag

SEQ ID NO: 34 - protein, 490 aa, Arabidopsis thaliana
MGSISEMVFETCPSPNPIHVMLVSFQGQGHVNPLLRLGKLIASKGLLVTFVTTELWGKKMRQANKIVDGELKPVGSGSIRFEFFDEEWAEDDDRRADFSLYIAHLESVGIREVSKLVRRYEEANEPVSCLINNPFIPWVCHVAEEFNIPCAVLWVQSCACFSAYYHYQDGSVSFPTETEPELDVKLPCVPVLKNDEIPSFLHPSSRFTGFRQAILGQFKNLSKSFCVLIDSFDSLEQEVIDYMSSLCPVKTVGPLFKVARTVTSDVSGDICKSTDKCLEWLDSRPKSSVVYISFGTVAYLKQEQIEEIAHGVLKSGLSFLWVIRPPPHDLKVETHVLPQELKESSAKGKGMIVDWCPQEQVLSHPSVACFVTHCGWNSTMESLSSGVPVVCCPQWGDQVTDAVYLIDVFKTGVRLGRGATEERVVPREEVAEKLLEATVGEKAEELRKNALKWKAEAEAAVAPGGSSDKNFREFVEKLGAGVTKTKDNGY

SEQ ID NO: 35 - DNA, 1413 bp, Artificial Sequence
  Synthetic oligonucleotide
atggaaaaaacaccccatatagctattgtaccaagtccaggaatgggacacttgatccctttggttgaatttgccaaaagattgaagaacaaccacaacatcgatgcaactttcatcattccaaatgatggacctctatccaaatctcaacgtgtttatctcgattcactcccaaccggattaaaccatatcattctccctccagttagtttcgatgatctaccacaagatgcaaagatggaaacccgaatcagcctcatggttacacgatctatcgatttccttcgagaagctttcaagtcattagttgcagaaacaaacatggtggcactgtttattgatctttttggtacagatgcatttgatgttgctattgaatttggtgtttcaccatatgtcttctttccatcaactgcaatggctttatctttgtttcttcatttaccaaaacttgatcaaatggtttcatgtgagtatagggacttgcctgaaccggttcagatcccgggttgcataccagttcccggtcgagacctacttgacccggttcaagatagaaagaacgaagcgtataagtgggtgcttcataacgcaaagaggtattcgatggctgagggtatagcggtaaatagcttcaaggagttagaaggtggagccttgaaagctttactagaggaagaaccgggcaaaccaaaggtttatccggttggaccgttgatacagaccggttcaagtactgatgttgatgggtccgagtgtttgaggtggttagacggtcagccatgtggttctgttttgtacgtatcttttggaagtggtggaaccttatcttctaatcagctcaatgagttagcctttggtttggaattaagtgagcaaaggttcatatgggtggttagaagcccgaatgatcaacccaacgcgacttactttaactcacatggtcatatggacccgttgggtttcttaccagaagggtttctagaaagaaccaaaggttttgggcttgtggttccttcttgggccccacaagcccaaatcttgagtcatagttcaaccggtgggtttttaacccactgtggttggaactcgattcttgagactgtagtccatggtgtgccggttatcgcctggccactttacgcagagcagaggatgaacgcggtatctttaaccgagggtataaaagtggcgttaaggcccaacgtggacgaaaatggcatcgtgggccgtgtggagattgcgagggtcgtgaagggtttgttagaaggggaagaaggaaaaccgattaggagtcgaattcgggatcttaaagatgcagctgctaatgttcttagtaaagatgggtgttccacaaaaactttagtgcagttggcttccaagttgaaaacgaagagtaaattaagcatttag

SEQ ID NO: 36 - protein, 470 aa, Stevia rebaudiana
MEKTPHIAIVPSPGMGHLIPLVEFAKRLKNNHNIDATFIIPNDGPLSKSQRVYLDSLPTGLNHIILPPVSFDDLPQDAKMETRISLMVTRSIDFLREAFKSLVAETNMVALFIDLFGTDAFDVAIEFGVSPYVFFPSTAMALSLFLHLPKLDQMVSCEYRDLPEPVQIPGCIPVPGRDLLDPVQDRKNEAYKWVLHNAKRYSMAEGIAVNSFKELEGGALKALLEEEPGKPKVYPVGPLIQTGSSTDVDGSECLRWLDGQPCGSVLYVSFGSGGTLSSNQLNELAFGLELSEQRFIWVVRSPNDQPNATYFNSHGHMDPLGFLPEGFLERTKGFGLVVPSWAPQAQILSHSSTGGFLTHCGWNSILETVVHGVPVIAWPLYAEQRMNAVSLTEGIKVALRPNVDENGIVGRVEIARVVKGLLEGEEGKPIRSRIRDLKDAAANVLSKDGCSTKTLVQLASKLKTKSKLSI

SEQ ID NO: 37 - DNA, 1491 bp, Arabidopsis thaliana
atggctacggaaaaaacccaccaatttcatccttctcttcactttgtcctcttccctttcatggctcaaggccacatgattcccatgattgatattgcaagactcttggctcagcgtggtgtgaccataacaattgtcacgacacctcacaacgcagcaaggtttaagaatgtcctaaaccgagcgatcgagtctggcttggccatcaacatactgcatgtgaagtttccatatcaagagtttggtttgccagaaggaaaagagaatatagattcgttagactcaacggagttgatggtacctttcttcaaagcggtgaacttgcttgaagatccggtcatgaagctcatggaagagatgaaacctagacctagctgtctaatttctgattggtgtttgccttatacaagcataatcgccaagaacttcaatataccaaagatagttttccacggcatgggttgctttaatcttttgtgtatgcatgttctacgcagaaacttagagatcctagagaatgtaaagtcggatgaagagtatttcttggttcctagttttcctgatagagttgaatttacaaagcttcaacttcctgtgaaagcaaatgcaagtggagattggaaagagataatggatgaaatggtaaaagcagaatacacatcctatggtgtgatcgtcaacacatttcaggagttggagccaccttatgtcaaagactacaaagaggcaatggatggaaaagtatggtccattggacccgtttccttgtgtaacaaggcaggtgcagacaaagctgagaggggaagcaaggccgccattgatcaagatgagtgtcttcaatggcttgattctaaagaagaaggttcggtgctctatgtttgccttggaagtatatgtaatcttcctttgtctcagctcaaggagctggggctaggccttgaggaatctcgaagatcttttatttgggtcataagaggttcggaaaagtataaagaactatttgagtggatgttggagagcggttttgaagaaagaatcaaagagagaggacttctcattaaagggtgggcacctcaagtccttatcctttcacatccttccgttggaggattcctgacacactgtggatggaactcgactctcgaaggaatcacctcaggcattccactgatcacttggccgctgtttggagaccaattctgcaaccaaaaactggtcgttcaagtactaaaagccggtgtaagtgccggggttgaagaagtcatgaaatggggagaagaagataaaataggagtgttagtggataaagaaggagtgaaaaaggctgtggaagaattgatgggtgatagtgatgatgcaaaagagaggagaagaagagtcaaagagcttggagaattagctcacaaagctgtggaaaaaggaggctcttctcattctaacatcacactcttgctacaagacataatgcaactagcacaattcaagaattga

SEQ ID NO: 38 - protein, 496 aa, Arabidopsis thaliana
MATEKTHQFHPSLHFVLFPFMAQGHMIPMIDIARLLAQRGVTITIVTTPHNAARFKNVLNRAIESGLAINILHVKFPYQEFGLPEGKENIDSLDSTELMVPFFKAVNLLEDPVMKLMEEMKPRPSCLISDWCLPYTSIIAKNFNIPKIVFHGMGCFNLLCMHVLRRNLEILENVKSDEEYFLVPSFPDRVEFTKLQLPVKANASGDWKEIMDEMVKAEYTSYGVIVNTFQELEPPYVKDYKEAMDGKVWSIGPVSLCNKAGADKAERGSKAAIDQDECLQWLDSKEEGSVLYVCLGSICNLPLSQLKELGLGLEESRRSFIWVIRGSEKYKELFEWMLESGFEERIKERGLLIKGWAPQVLILSHPSVGGFLTHCGWNSTLEGITSGIPLITWPLFGDQFCNQKLVVQVLKAGVSAGVEEVMKWGEEDKIGVLVDKEGVKKAVEELMGDSDDAKERRRRVKELGELAHKAVEKGGSSHSNITLLLQDIMQLAQFKN

SEQ ID NO: 39 - DNA, 1488 bp, Arabidopsis thaliana
atggtttccgaaacaaccaaatcttctccacttcactttgttctcttccctttcatggctcaaggccacatgattcccatggttgatattgcaaggctcttggctcagcgtggtgtgatcataacaattgtcacgacgcctcacaatgcagcgaggttcaagaatgtcctaaaccgtgccattgagtctggcttgcccatcaacttagtgcaagtcaagtttccatatctagaagctggtttgcaagaaggacaagagaatatcgattctcttgacacaatggagcggatgatacctttctttaaagcggttaactttctcgaagaaccagtccagaagctcattgaagagatgaaccctcgaccaagctgtctaatttctgatttttgtttgccttatacaagcaaaatcgccaagaagttcaatatcccaaagatcctcttccatggcatgggttgcttttgtcttctgtgtatgcatgttttacgcaagaaccgtgagatcttggacaatttaaagtcagataaggagcttttcactgttcctgattttcctgatagagttgaattcacaagaacgcaagttccggtagaaacatatgttccagctggagactggaaagatatctttgatggtatggtagaagcgaatgagacatcttatggtgtgatcgtcaactcatttcaagagctcgagcctgcttatgccaaagactacaaggaggtaaggtccggtaaagcatggaccattggacccgtttccttgtgcaacaaggtaggagccgacaaagcagagaggggaaacaaatcagacattgatcaagatgagtgccttaaatggctcgattctaagaaacatggctcggtgctttacgtttgtcttggaagtatctgtaatcttcctttgtctcaactcaaggagctgggactaggcctagaggaatcccaaagacctttcatttgggtcataagaggttgggagaagtacaaagagttagttgagtggttctcggaaagcggctttgaagatagaatccaagatagaggacttctcatcaaaggatggtcccctcaaatgcttatcctttcacatccatcagttggagggttcctaacacactgtggttggaactcgactcttgaggggataactgctggtctaccgctacttacatggccgctattcgcagaccaattctgcaatgagaaattggtcgttgaggtactaaaagccggtgtaagatccggggttgaacagcctatgaaatggggagaagaggagaaaataggagtgttggtggataaagaaggagtgaagaaggcagtggaagaattaatgggtgagagtgatgatgcaaaagagagaagaagaagagccaaagagcttggagattcagctcacaaggctgtggaagaaggaggctcttctcattctaacatctctttcttgctacaagacataatggaactggcagaacccaataattga

SEQ ID NO: 40 - protein, 495 aa, Arabidopsis thaliana
MVSETTKSSPLHFVLFPFMAQGHMIPMVDIARLLAQRGVIITIVTTPHNAARFKNVLNRAIESGLPINLVQVKFPYLEAGLQEGQENIDSLDTMERMIPFFKAVNFLEEPVQKLIEEMNPRPSCLISDFCLPYTSKIAKKFNIPKILFHGMGCFCLLCMHVLRKNREILDNLKSDKELFTVPDFPDRVEFTRTQVPVETYVPAGDWKDIFDGMVEANETSYGVIVNSFQELEPAYAKDYKEVRSGKAWTIGPVSLCNKVGADKAERGNKSDIDQDECLKWLDSKKHGSVLYVCLGSICNLPLSQLKELGLGLEESQRPFIWVIRGWEKYKELVEWFSESGFEDRIQDRGLLIKGWSPQMLILSHPSVGGFLTHCGWNSTLEGITAGLPLLTWPLFADQFCNEKLVVEVLKAGVRSGVEQPMKWGEEEKIGVLVDKEGVKKAVEELMGESDDAKERRRRAKELGDSAHKAVEEGGSSHSNISFLLQDIMELAEPNN

SEQ ID NO: 41 - DNA, 1422 bp, Arabidopsis thaliana
atgaaagtgaacgaggaaaacaacaagccgacaaagacccatgtcttaatcttcccatttccggcgcaaggtcacatgattcccctcctcgacttcacccaccgccttgctctccgcggcggcgccgccttaaaaataaccgtcctagtcactccaaaaaaccttccttttctctctccgcttctctccgccgtagttaacatcgaaccacttatcctcccttttccctcccacccttcaatcccctccggcgtcgaaaacgtccaagacttacctccttcaggcttccctttaatgatccacgcgcttggtaatctccacgcgccgcttatctcttggattacttctcacccttctcctccagtagccatcgtatctgatttcttccttggttggaccaaaaacctcggaatccctcgtttcgatttctctccctccgctgctatcacttgctgcatactcaatactctctggatcgaaatgcccaccaagatcaacgaagatgacgataacgagatcctccactttcccaagatcccgaattgtccaaaataccgttttgatcagatctcctctctttacagaagttacgttcacggagatccagcttgggagttcataagagactcctttagagataacgtggcgagttggggactcgtcgtgaactcgttcaccgccatggaaggtgtttatctcgaacatcttaagcgagagatgggccatgatcgtgtatgggctgtaggcccaattattccgttatctggggataaccgtggtggcccgacttctgtttctgttgatcacgtgatgtcgtggcttgacgcacgtgaggataaccacgtggtgtacgtgtgctttggaagtcaagtagttttgactaaagagcagactcttgcactcgcctctgggcttgagaaaagcggcgtccatttcatatgggccgtaaaggagcccgttgagaaagactcaacacgtggcaacatcctggacggtttcgacgatcgcgtggctgggagaggtctggtgatcagaggatgggctccacaagtagctgtgctacgtcaccgagccgttggcgcgtttttaacgcactgtggttggaactctgtggtggaggcggttgtcgccggcgttttgatgctgacgtggccgatgagagctgaccagtacactgacgcgtctctggtggttgatgagttgaaagtaggtgtgcgtgcttgcgaaggacctgacacggtgcctgacccggacgagttagctcgagttttcgctgattccgtgaccggaaatcaaacggagaggatcaaagccgtggagctgaggaaagcagcgttggatgcgattcaagaacgtgggagctcagtgaatgatttagatggatttatccaacatgtcgttagtttaggactaaacaaatga

SEQ ID NO: 42 - protein, 473 aa, Arabidopsis thaliana
MKVNEENNKPTKTHVLIFPFPAQGHMIPLLDFTHRLALRGGAALKITVLVTPKNLPFLSPLLSAVVNIEPLILPFPSHPSIPSGVENVQDLPPSGFPLMIHALGNLHAPLISWITSHPSPPVAIVSDFFLGWTKNLGIPRFDFSPSAAITCCILNTLWIEMPTKINEDDDNEILHFPKIPNCPKYRFDQISSLYRSYVHGDPAWEFIRDSFRDNVASWGLVVNSFTAMEGVYLEHLKREMGHDRVWAVGPIIPLSGDNRGGPTSVSVDHVMSWLDAREDNHVVYVCFGSQVVLTKEQTLALASGLEKSGVHFIWAVKEPVEKDSTRGNILDGFDDRVAGRGLVIRGWAPQVAVLRHRAVGAFLTHCGWNSVVEAVVAGVLMLTWPMRADQYTDASLVVDELKVGVRACEGPDTVPDPDELARVFADSVTGNQTERIKAVELRKAALDAIQERGSSVNDLDGFIQHVVSLGLNK

SEQ ID NO: 43 - DNA, 1380 bp, Nicotiana tabacum
atgactactcaaaaagctcattgcttgatcttaccatatccagctcagggtcatatcaaccctatgctccaattctccaaacgtttgcaatccaaaggtgtcaaaatcactatagcagccaccaaatcattcttgaaaaccatgcaagaattgtcaacttctgtgtcagtcgaggctatctccgatggctatgatgatggcggacgcgagcaagctggaacctttgtggcctatattacaagattcaaagaagttggctcggatactttgtctcagcttattggaaagttaacaaattgtggttgtcctgtgagttgcatagtttacgatccatttcttccttgggctgttgaagtgggaaataattttggagtagctactgctgcttttttcactcaatcttgtgcagtggataacatttattaccatgtacataaaggggttctaaaacttcctccaactgacgttgataaagaaatctcaattcctggattattaacaattgaggcatcagatgtacctagttttgtttctaatcctgaatcttcaagaatacttgaaatgttggtgaatcagttctcgaatcttgagaacacagattgggtcctaatcaacagtttctatgaattggagaaagaagtaattgattggatggccaagatctatccaatcaagacaattggaccaactataccatcaatgtacctagacaagaggctaccagatgacaaagaatatggccttagtgtcttcaagccaatgacaaatgcatgcctaaactggttaaaccatcaaccagttagctcagtagtatatgtatcatttggaagtttagccaaattagaagcagagcaaatggaagaattagcatggggtttgagtaatagcaacaagaacttcttgtgggtagttagatccactgaagaatccaaacttcccaacaactttttagaggaattagcaagtgaaaaaggattagtcgtgtcatggtgtccacaattacaagtcttggaacataaatcaatagggtgttttctcacgcactgtggctggaattcaactttggaagcaattagtttgggagtaccaatgattgcaatgccacattggtcagaccagccaacaaatgcgaagcttgtggaagatgtttgggagatgggaattagaccaaaacaagatgaaaaaggattagttagaagagaagttattgaagaatgtattaagatagtgatggaggaaaagaaaggaaaaaagattagggaaaatgcaaagaaatggaaggaattggctaggaaagctgtggatgaaggaggaagttcagatagaaatattgaagaatttgtttccaagttggtgactattgcctcagtggaaagctaa

SEQ ID NO: 44 - protein, 459 aa, Nicotiana tabacum
MTTQKAHCLILPYPAQGHINPMLQFSKRLQSKGVKITIAATKSFLKTMQELSTSVSVEAISDGYDDGGREQAGTFVAYITRFKEVGSDTLSQLIGKLTNCGCPVSCIVYDPFLPWAVEVGNNFGVATAAFFTQSCAVDNIYYHVHKGVLKLPPTDVDKEISIPGLLTIEASDVPSFVSNPESSRILEMLVNQFSNLENTDWVLINSFYELEKEVIDWMAKIYPIKTIGPTIPSMYLDKRLPDDKEYGLSVFKPMTNACLNWLNHQPVSSVVYVSFGSLAKLEAEQMEELAWGLSNSNKNFLWVVRSTEESKLPNNFLEELASEKGLVVSWCPQLQVLEHKSIGCFLTHCGWNSTLEAISLGVPMIAMPHWSDQPTNAKLVEDVWEMGIRPKQDEKGLVRREVIEECIKIVMEEKKGKKIRENAKKWKELARKAVDEGGSSDRNIEEFVSKLVTIASVES

SEQ ID NO: 45 - DNA, 1443 bp, Arabidopsis thaliana
atggaggaatccaaaacacctcacgttgcgatcataccaagtccgggaatgggtcatctcataccactcgtcgagtttgctaaacgactcgtccatcttcacggcctcaccgttaccttcgtcatcgccggcgaaggtccaccatcaaaagctcagagaaccgtcctcgactctctcccttcttcaatctcctccgtctttctccctcctgttgatctcaccgatctctcttcgtccactcgcatcgaatctcggatctccctcaccgtgactcgttcaaacccggagctccggaaagtcttcgactcgttcgtggagggaggtcgtttgccaacggcgctcgtcgtcgatctcttcggtacggacgctttcgacgtggccgtagaatttcacgtgccaccgtatattttctacccaacaacggccaacgtcttgtcgttttttctccatttgcctaaactagacgaaacggtgtcgtgtgagttcagggaattaaccgaaccgcttatgcttcctggatgtgtaccggttgccgggaaagatttccttgacccggcccaagaccggaaagacgatgcatacaaatggcttctccataacaccaagaggtacaaagaagccgaaggtattcttgtgaataccttctttgagctagagccaaatgctataaaggccttgcaagaaccgggtcttgataaaccaccggtttatccggttggaccgttggttaacattggtaagcaagaggctaagcaaaccgaagagtctgaatgtttaaagtggttggataaccagccgctcggttcggttttatatgtgtcctttggtagtggcggtaccctcacatgtgagcagctcaatgagcttgctcttggtcttgcagatagtgagcaacggtttctttgggtcatacgaagtcctagtgggatcgctaattcgtcgtattttgattcacatagccaaacagatccattgacatttttaccaccgggatttttagagcggactaaaaaaagaggttttgtgatccctttttgggctccacaagcccaagtcttggcgcatccatccacgggaggatttttaactcattgtggatggaattcgactctagagagtgtagtaagcggtattccacttatagcatggccattatacgcagaacagaagatgaatgcggttttgttgagtgaagatattcgtgcggcacttaggccgcgtgccggggacgatgggttagttagaagagaagaggtggctagagtggtaaaaggattgatggaaggtgaagaaggcaaaggagtgaggaacaagatgaaggagttgaaggaagcagcttgtagggtgttgaaggatgatgggacttcgacaaaagcacttagtcttgtggccttaaagtggaaagcccacaaaaaagagttagagcaaaatggcaaccactaa

SEQ ID NO: 46 - protein, 480 aa, Arabidopsis thaliana
MEESKTPHVAIIPSPGMGHLIPLVEFAKRLVHLHGLTVTFVIAGEGPPSKAQRTVLDSLPSSISSVFLPPVDLTDLSSSTRIESRISLTVTRSNPELRKVFDSFVEGGRLPTALVVDLFGTDAFDVAVEFHVPPYIFYPTTANVLSFFLHLPKLDETVSCEFRELTEPLMLPGCVPVAGKDFLDPAQDRKDDAYKWLLHNTKRYKEAEGILVNTFFELEPNAIKALQEPGLDKPPVYPVGPLVNIGKQEAKQTEESECLKWLDNQPLGSVLYVSFGSGGTLTCEQLNELALGLADSEQRFLWVIRSPSGIANSSYFDSHSQTDPLTFLPPGFLERTKKRGFVIPFWAPQAQVLAHPSTGGFLTHCGWNSTLESVVSGIPLIAWPLYAEQKMNAVLLSEDIRAALRPRAGDDGLVRREEVARVVKGLMEGEEGKGVRNKMKELKEAACRVLKDDGTSTKALSLVALKWKAHKKELEQNGNH

SEQ ID NO: 47 - DNA, 1446 bp, Arabidopsis thaliana
atgagtagtgatcctcatcgtaagctccatgttgtgttcttccctttcatggcttatggtcacatgataccaactctagacatggctaagcttttctctagcagaggagccaaatctacaatcctcaccacacctctcaactccaagatcttccaaaaacccatcgaaagattcaagaacctgaatccgagtttcgaaatcgacatccagatcttcgatttcccttgcgtggatctcgggttaccagaaggatgcgaaaacgtcgatttcttcacctcaaacaacaatgatgatagacagtatctgaccttgaagttctttaagtcgacaaggtttttcaaagatcagcttgagaagctcctcgagacaacgagaccagactgtcttatcgccgacatgttcttcccctgggctacggaagctgctgagaagttcaatgtgccaagacttgtgttccacggtactggctacttttctttatgctctgaatattgcatcagagtgcataacccacaaaacatagtagcttcaaggtacgagccatttgtgattcctgatctcccggggaacatagtgataactcaagaacagatagcagaccgtgacgaagaaagcgagatggggaagtttatgattgaggtcaaagaatctgatgtgaagagctcaggtgttattgtaaacagcttctacgagcttgaacctgattacgccgacttttacaagagtgttgtactgaagagagcgtggcatatcggtccgctttcggtttacaacagaggatttgaggagaaggctgagagaggaaagaaagcaagcattaatgaggttgaatgcctcaaatggcttgactccaagaaaccagattcagtcatttacatttcttttgggagcgtggcttgcttcaagaacgagcagctattcgagatcgctgcaggattagaaacttctggagcaaatttcatctgggttgttaggaaaaacataggtattgaaaaagaagaatggttaccagaagggttcgaagagagggtgaaaggaaaagggatgattataagaggatgggcaccacaggtgctcatacttgatcatcaagcaacttgtgggtttgtgacccattgcggctggaactcgcttctggaaggagtggctgcagggctaccaatggtgacatggcctgtagcagcggagcaattctacaatgagaaattggttacgcaagtgctcagaacaggagtgagcgtgggagcgaaaaagaatgtaagaactacgggagatttcattagcagagagaaagtggttaaagcggtgagggaggtgttggttggggaagaggcggatgagaggcgggagagggcaaagaagttggcagagatggctaaagctgccgtggaaggagggtcttctttcaacgatctaaacagcttcatagaagagtttacctcgtaa

SEQ ID NO: 48 - protein, 481 aa, Arabidopsis thaliana
MSSDPHRKLHVVFFPFMAYGHMIPTLDMAKLFSSRGAKSTILTTPLNSKIFQKPIERFKNLNPSFEIDIQIFDFPCVDLGLPEGCENVDFFTSNNNDDRQYLTLKFFKSTRFFKDQLEKLLETTRPDCLIADMFFPWATEAAEKFNVPRLVFHGTGYFSLCSEYCIRVHNPQNIVASRYEPFVIPDLPGNIVITQEQIADRDEESEMGKFMIEVKESDVKSSGVIVNSFYELEPDYADFYKSVVLKRAWHIGPLSVYNRGFEEKAERGKKASINEVECLKWLDSKKPDSVIYISFGSVACFKNEQLFEIAAGLETSGANFIWVVRKNIGIEKEEWLPEGFEERVKGKGMIIRGWAPQVLILDHQATCGFVTHCGWNSLLEGVAAGLPMVTWPVAAEQFYNEKLVTQVLRTGVSVGAKKNVRTTGDFISREKVVKAVREVLVGEEADERRERAKKLAEMAKAAVEGGSSFNDLNSFIEEFTS

SEQ ID NO: 49 - DNA, 1377 bp, Arabidopsis thaliana
atgcaggttttgggaatggaggaaaagcctgcaaggagaagcgtagtgttggttccatttccagcacaaggacatatatctccaatgatgcaacttgccaaaacccttcacttaaagggtttctcgatcacagttgttcagactaagttcaattactttagcccttcagatgacttcactcatgattttcagttcgtcaccattccagaaagcttaccagagtctgatttcaagaatctcggaccaatacagtttctgtttaagctcaacaaagagtgtaaggtgagcttcaaggactgtttgggtcagttggtgctgcaacaaagtaatgagatctcatgtgtcatctacgatgagttcatgtactttgctgaagctgcagccaaagagtgtaagcttccaaacatcattttcagcacaacaagtgccacggctttcgcttgccgctctgtatttgacaaactatatgcaaacaatgtccaagctcccttgaaagaaactaaaggacaacaagaagagctagttccggagttttatcccttgagatataaagactttccagtttcacggtttgcatcattagagagcataatggaggtgtataggaatacagttgacaaacggacagcttcctcggtgataatcaacactgcgagctgtctagagagctcatctctgtcttttctgcaacaacaacagctacaaattccagtgtatcctataggccctcttcacatggtggcctcagctcctacaagtctgcttgaagagaacaagagctgcatcgaatggttgaacaaacaaaaggtaaactcggtgatatacataagcatgggaagcatagctttaatggaaatcaacgagataatggaagtcgcgtcaggattggctgctagcaaccaacacttcttatgggtgatccgaccagggtcaatacctggttccgagtggatagagtccatgcctgaagagtttagtaagatggttttggaccgaggttacattgtgaaatgggctccacagaaggaagtactttctcatcctgcagtaggagggttttggagccattgtggatggaactcgacactagaaagcatcggccaaggagttccaatgatctgcaggccattttcgggtgatcaaaaggtgaacgctagatacttggagtgtgtatggaaaattgggattcaagtggagggtgagctagacagaggagtggtcgagagagctgtgaagaggttaatggttgacgaagaaggagaggagatgaggaagagagctttcagtttaaaagagcaacttagagcctctgttaaaagtggaggctcttcacacaactcgctagaagagtttgtacacttcataaggactctatga

SEQ ID NO: 50 - protein, 458 aa, Arabidopsis thaliana
MQVLGMEEKPARRSVVLVPFPAQGHISPMMQLAKTLHLKGFSITVVQTKFNYFSPSDDFTHDFQFVTIPESLPESDFKNLGPIQFLFKLNKECKVSFKDCLGQLVLQQSNEISCVIYDEFMYFAEAAAKECKLPNIIFSTTSATAFACRSVFDKLYANNVQAPLKETKGQQEELVPEFYPLRYKDFPVSRFASLESIMEVYRNTVDKRTASSVIINTASCLESSSLSFLQQQQLQIPVYPIGPLHMVASAPTSLLEENKSCIEWLNKQKVNSVIYISMGSIALMEINEIMEVASGLAASNQHFLWVIRPGSIPGSEWIESMPEEFSKMVLDRGYIVKWAPQKEVLSHPAVGGFWSHCGWNSTLESIGQGVPMICRPFSGDQKVNARYLECVWKIGIQVEGELDRGVVERAVKRLMVDEEGEEMRKRAFSLKEQLRASVKSGGSSHNSLEEFVHFIRTL

SEQ ID NO: 51 - DNA, 1446 bp, Arabidopsis thaliana
atggggaagcaagaagatgcagagctcgtcatcatacctttccctttctccggacacattctcgcaacaatcgaactcgccaaacgtctcataagtcaagacaatcctcggatccacaccatcaccatcctctattggggattaccttttattcctcaagctgacacaatcgctttcctccgatccctagtcaaaaatgagcctcgtatccgtctcgttacgttgcccgaagtccaagaccctccaccaatggaactctttgtggaatttgccgaatcttacattcttgaatacgtcaagaaaatggttcccatcatcagagaagctctctccactctcttgtcttcccgcgatgaatcgggttcagttcgtgtggctggattggttcttgacttcttctgcgtccctatgatcgatgtaggaaacgagtttaatctcccttcttacattttcttgacgtgtagcgcagggttcttgggtatgatgaagtatcttccagagagacaccgcgaaatcaaatcggaattcaaccggagcttcaacgaggagttgaatctcattcctggttatgtcaactctgttcctactaaggttttgccgtcaggtctattcatgaaagagacctacgagccttgggtcgaactagcagagaggtttcctgaagctaagggtattttggttaattcatacacagctctcgagccaaacggttttaaatatttcgatcgttgtccggataactacccaaccatttacccaatcgggccgatattatgctccaacgaccgtccgaatttggactcatcggaacgagatcggatcataacttggctagatgaccaacccgagtcatcggtcgtgttcctctgtttcgggagcttgaagaatctcagcgctactcagatcaacgagatagctcaagccttagagatcgttgactgcaaattcatctggtcgtttcgaaccaacccgaaggagtacgcgagcccttacgaggctctaccacacgggttcatggaccgggtcatggatcaaggcattgtttgtggttgggctcctcaagttgaaatcctagcccataaagctgtgggaggattcgtatctcattgtggttggaactcgatattggagagtttgggtttcggcgttccaatcgccacgtggccgatgtacgcggaacaacaactaaacgcgttcacgatggtgaaggagcttggtttagccttggagatgcggttggattacgtgtcggaagatggagatatagtgaaagctgatgagatcgcaggaaccgttagatctttaatggacggtgtggatgtgccgaagagtaaagtgaaggagattgctgaggcgggaaaagaagctgtggacggtggatcttcgtttcttgcggttaaaagattcatcggtgacttgatcgacggcgtttctataagtaagtag

SEQ ID NO: 52 - protein, 481 aa, Arabidopsis thaliana
MGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGYVNSVPTKVLPSGLFMKETYEPWVELAERFPEAKGILVNSYTALEPNGFKYFDRCPDNYPTIYPIGPILCSNDRPNLDSSERDRIITWLDDQPESSVVFLCFGSLKNLSATQINEIAQALEIVDCKFIWSFRTNPKEYASPYEALPHGFMDRVMDQGIVCGWAPQVEILAHKAVGGFVSHCGWNSILESLGFGVPIATWPMYAEQQLNAFTMVKELGLALEMRLDYVSEDGDIVKADEIAGTVRSLMDGVDVPKSKVKEIAEAGKEAVDGGSSFLAVKRFIGDLIDGVSISK

SEQ ID NO: 53 - DNA, 1317 bp, Arabidopsis thaliana
atggtagcattagcattccaaggtcatctcaatccaatgctcaaattcgcaaaacatctcgcacgaaccaatctacacttcactctcgccaccactgagcaagcccgtgacctcctctcttccaccgctgacgaacctcatagaccggtggacctcgctttcttctcagacggtctacctaaagacgatccaagagatcccgacactctcgcaaagtcattgaaaaaagatggagccaagaacttgtcaaaaatcatcgaagaaaagagatttgattgcatcatctctgtgccttttactccctgggttccagctgttgcagctgcacataacattccttgtgcaatcctctggatccaagcttgtggagctttttctgtttattaccgttattacatgaagacaaatcctttccccgaccttgaagatctgaatcaaacagtggagttaccagctttaccattgttggaagtccgagatctcccgtcattgatgttaccttctcaaggagctaatgtcaataccctaatggcggaatttgcagattgtttgaaagatgtgaaatgggttttggttaactcgttttacgaactcgaatcagagatcatcgagtctatgtctgatttaaaacctataatcccaattggtcctcttgtttctccattcctgttgggaaatgatgaagaaaaaaccctagatatgtggaaagttgatgattattgtatggagtggcttgacaagcaagctaggtcttcagttgtttacatatctttcggaagcatactcaaatcattggagaatcaagttgagaccatagcaacggcattaaaaaacagaggagttccatttctttgggtgatacggccgaaggagaaaggcgaaaacgtccaggttttgcaggagatggttaaagaaggtaaaggggttgtaactgaatggggtcaacaagaaaagatattgagccacatggcgatttcttgcttcatcacgcattgtggatggaactcgacgatcgagacggtggtgactggtgttcccgtggtggcgtatccgacttggatagatcagccgcttgatgcgagactgcttgtggatgtgtttggaatcggagtaaggatgaagaacgacgctatcgatggagagcttaaggttgcagaggtggagagatgcattgaggccgtgacagagggacctgccgccgcggatatgaggaggagagcgacggagctgaagcacgccgcaagatcggcgatgtcacctggtggatcttccgctcagaatttagactcgttcattagtgatatcccaatcacttga

SEQ ID NO: 54 - protein, 438 aa, Arabidopsis thaliana
MVALAFQGHLNPMLKFAKHLARTNLHFTLATTEQARDLLSSTADEPHRPVDLAFFSDGLPKDDPRDPDTLAKSLKKDGAKNLSKIIEEKRFDCIISVPFTPWVPAVAAAHNIPCAILWIQACGAFSVYYRYYMKTNPFPDLEDLNQTVELPALPLLEVRDLPSLMLPSQGANVNTLMAEFADCLKDVKWVLVNSFYELESEIIESMSDLKPIIPIGPLVSPFLLGNDEEKTLDMWKVDDYCMEWLDKQARSSVVYISFGSILKSLENQVETIATALKNRGVPFLWVIRPKEKGENVQVLQEMVKEGKGVVTEWGQQEKILSHMAISCFITHCGWNSTIETVVTGVPVVAYPTWIDQPLDARLLVDVFGIGVRMKNDAIDGELKVAEVERCIEAVTEGPAAADMRRRATELKHAARSAMSPGGSSAQNLDSFISDIPIT

SEQ ID NO: 55 - DNA, 1440 bp, Arabidopsis thaliana
atggcgtctcatgctgttacaagcggacaaaaaccacacgtagtttgcatacctttcccggctcaaggccacatcaatccgatgctcaaagtggctaaactcctctatgccagaggcttccatgttaccttcgtcaacactaactacaaccataaccgtctcatccggtcacgtggtcccaactcccttgatgggcttccttcttttcggttcgagtccatccctgacggtctaccggaggaaaacaaggacgtcatgcaggatgtccctaccctttgtgagtccaccatgaaaaactgtctagctcctttcaaggagcttctccggcggatcaacaccacaaaggatgttcctccggtaagctgtattgtatccgacggtgtgatgagctttactcttgatgctgcagaggagcttggagtcccggatgttcttttttggacaccaagtgcttgtggcttcttggcttatctacacttctatcgcttcatcgagaaggggttatcaccaataaaagatgaaagttctttggacacaaaaataaattggataccatcgatgaaaaacctaggacttaaagacatcccaagctttatccgtgcaactaatactgaagacataatgcttaacttttttgtccatgaggctgaccgagccaaacgcgcttccgctatcattctcaacacattcgatagtcttgagcatgatgtcgtccgttctattcaatctatcatacctcaagtgtacactattggaccgcttcatctatttgtgaatcgggatatcgacgaggaaagtgacatcggacagataggaacgaatatgtggagagaggagatggagtgtttggattggcttgatactaagtctccaaacagtgtcgtttatgttaatttcggtagcataacagtgatgagtgcgaaacaactcgtggagtttgcttggggtttagcagcgaccaaaaaagattttttgtgggtgattaggccggatttagtagccggtgatgtgccaatgcttccgccggactttctaatagagacggctaaccgaaggatgctagcgagttggtgtcctcaagaaaaagttctttctcatccggcagttggagggttcttaacgcatagtggatggaattcgactttggagagtctctccggtggagttccaatggtgtgttggccgttctttgcggaacagcaaacaaattgtaaatattgttgtgatgaatgggaagtggggatggagatcggtggagatgtgaggagggaggaggttgaggagttggttagagaactcatggacggagacaaaggaaagaaaatgaggcaaaaggccgaagagtggcagcgcttggctgaggaagcgacgaagcctatttatggttcgtcggaactaaattttcagatggtcgttgacaaggttcttttaggggagtag

SEQ ID NO: 56 - protein, 479 aa, Arabidopsis thaliana
MASHAVTSGQKPHVVCIPFPAQGHINPMLKVAKLLYARGFHVTFVNTNYNHNRLIRSRGPNSLDGLPSFRFESIPDGLPEENKDVMQDVPTLCESTMKNCLAPFKELLRRINTTKDVPPVSCIVSDGVMSFTLDAAEELGVPDVLFWTPSACGFLAYLHFYRFIEKGLSPIKDESSLDTKINWIPSMKNLGLKDIPSFIRATNTEDIMLNFFVHEADRAKRASAIILNTFDSLEHDVVRSIQSIIPQVYTIGPLHLFVNRDIDEESDIGQIGTNMWREEMECLDWLDTKSPNSVVYVNFGSITVMSAKQLVEFAWGLAATKKDFLWVIRPDLVAGDVPMLPPDFLIETANRRMLASWCPQEKVLSHPAVGGFLTHSGWNSTLESLSGGVPMVCWPFFAEQQTNCKYCCDEWEVGMEIGGDVRREEVEELVRELMDGDKGKKMRQKAEEWQRLAEEATKPIYGSSELNFQMVVDKVLLGE

SEQ ID NO: 57 - DNA, 1371 bp, Solanum laciniatum
atgactactcacaaagctcattgcttgatcttgccatatccagtccaaggtcatatcaacccaatgcttcaattctccaaacgtttacgatccaaacgcgttaaaatcactatagcactcacaaaatcctttttgaaaaacatgaaggaattgccaacttctatgtcaattgaggccatatctgatggctatgatgatggtggtcgcgatcaagcaggaactttcgtggcctatattacacgattcaaagaaattggttcggatactctgtctcaacttattcaaaaattggcaattagtggatgtcctgtaaattgcatagtatatgatccattcctcccttgggctgttgaagttgcaaaacaatttggattaattagtgctgcatttttcacacaaaattgtgtagtggataatctttattaccatgtacataaaggggtgataaaacttccacctactcaaaatgacgaagaaatattaattcctggatttccaaattcgatcgatgcatcagatgtaccttcttttgttattagtcctgaagcagaaaggatagttgaaatgttagcaaatcaattctcaaatcttgacaaagttgattgtgttctaatcaatagcttctatgagttggagaaagaggtaattgattggatgtcgaagatatatccaataaagacaattggaccaacaataccatcaatgtacttagacaagagactacatgatgataaagagtatggtcttagtatgttcaagccaatgacaaatgaatgcctaaattggttaaaccatcaaccaattagctcagtgttgtatgtatcatttggaagtttagccaaactaggaagtgagcaaatggaagaattggcatggggtttgaagaatagcaacaagagcttcttgtgggttgttaggtctactgaagagcccaaactccccaacaactttattgaggaattaacaagtgaaaaaggcttagtggtgtcatggtgtccacaattacaagtgttggaacatgagtcgatagggtgttttctgacgcactgtggatggaattcaactctggaagcgattagtttgggagtgccaatggtggcaatgccacaatggtctgatcaaccaacaaatgcaaagcttgtgaaagatgtttgggaaataggtgttagagccaaacaagatgaaaaaggggtagttagaagagaagttatagaagaatgtataaagctagtgatggaagaagataaaggaaaactaattagagaaaatgcaaagaaatggaaggaaatagctagaaatgttgtgaatgaaggaggaagttcagataaaaacattgaagaatttgtttccaagttggttactatttcctaa

SEQ ID NO: 58 - protein, 456 aa, Solanum lycopersicum
MTTHKAHCLILPYPVQGHINPMLQFSKRLRSKRVKITIALTKSFLKNMKELPTSMSIEAISDGYDDGGRDQAGTFVAYITRFKEIGSDTLSQLIQKLAISGCPVNCIVYDPFLPWAVEVAKQFGLISAAFFTQNCVVDNLYYHVHKGVIKLPPTQNDEEILIPGFPNSIDASDVPSFVISPEAERIVEMLANQFSNLDKVDCVLINSFYELEKEVIDWMSKIYPIKTIGPTIPSMYLDKRLHDDKEYGLSMFKPMTNECLNWLNHQPISSVLYVSFGSLAKLGSEQMEELAWGLKNSNKSFLWVVRSTEEPKLPNNFIEELTSEKGLVVSWCPQLQVLEHESIGCFLTHCGWNSTLEAISLGVPMVAMPQWSDQPTNAKLVKDVWEIGVRAKQDEKGVVRREVIEECIKLVMEEDKGKLIRENAKKWKEIARNVVNEGGSSDKNIEEFVSKLVTIS

SEQ ID NO: 59 - DNA, 1341 bp, Arabidopsis thaliana
atgagaggacatgtattagcagtgccatttccaagccaaggacacatcaccccgattcgccaattctgcaaacgacttcactccaaaggtttcaaaaccactcacactctcaccacttttatcttcaacacaatccacctcgacccatctagtcctatctccatagccacaatctccgatggctatgaccagggagggttctcatcagccggttctgtcccggagtacctacaaaacttcaaaaccttcggctccaaaaccgtcgctgatatcatccgcaaacaccagagtactgataaccctattacttgtatcgtctatgattctttcatgccttgggcgcttgaccttgcaatggattttggtctagctgcggctcctttcttcacgcagtcttgcgccgttaactatatcaattatctttcttacataaacaatggtagcttgacacttcccatcaaggatttgcctcttcttgagctccaagatttgcctactttcgtcactcctactggttcacaccttgcttactttgagatggtgcttcaacagttcaccaacttcgacaaagctgatttcgtactcgttaattccttccatgacctcgaccttcatgaagaggagttgttgtcgaaagtatgtcctgtgttgacaattggtccaactgttccatcaatgtacttagaccaacagatcaaatcagacaacgactatgatctgaacctctttgacttaaaagaagctgccttatgcactgactggctagacaagaggccagaaggatcggtagtatatatagcttttgggagcatggctaaactgagtagtgagcagatggaagagattgcttcggcgataagcaacttcagctacctctgggttgtcagagcttcagaggagtcaaagctcccaccagggtttcttgaaacagtggataaagacaagagcttggtcttgaagtggagtcctcagcttcaagttctgtcaaacaaagccatcggttgtttcatgactcactgtggctggaactcaaccatggagggtttgagtttaggggttcccatggtggctatgcctcaatggactgatcaaccaatgaatgcaaagtatatacaagatgtatggaaggttggggttcgtgtgaaagcagagaaagaaagtggcatttgcaaaagagaggagattgagtttagcatcaaggaagtgatggaaggagagaagagcaaagagatgaaagagaatgcgggaaaatggagagacttggctgtgaagtcactcagtgaaggaggttctacagatatcaacattaacgaatttgtatcaaaaattcaaatcaaatag

SEQ ID NO: 60 - protein, 446 aa, Arabidopsis thaliana
MRGHVLAVPFPSQGHITPIRQFCKRLHSKGFKTTHTLTTFIFNTIHLDPSSPISIATISDGYDQGGFSSAGSVPEYLQNFKTFGSKTVADIIRKHQSTDNPITCIVYDSFMPWALDLAMDFGLAAAPFFTQSCAVNYINYLSYINNGSLTLPIKDLPLLELQDLPTFVTPTGSHLAYFEMVLQQFTNFDKADFVLVNSFHDLDLHEEELLSKVCPVLTIGPTVPSMYLDQQIKSDNDYDLNLFDLKEAALCTDWLDKRPEGSVVYIAFGSMAKLSSEQMEEIASAISNFSYLWVVRASEESKLPPGFLETVDKDKSLVLKWSPQLQVLSNKAIGCFMTHCGWNSTMEGLSLGVPMVAMPQWTDQPMNAKYIQDVWKVGVRVKAEKESGICKREEIEFSIKEVMEGEKSKEMKENAGKWRDLAVKSLSEGGSTDININEFVSKIQIK

SEQ ID NO: 61 - DNA, 1440 bp, Arabidopsis thaliana
atggacccgtctcgtcatactcatgtgatgctcgtatctttccccggccaaggtcacgtaaaccctctacttcgtctcggaaagctcatagcctctaaaggcttactcgtcacctttgtcaccacagagaagccatggggcaagaagatgcgtcaagccaacaagattcaagacggtgtgctcaaaccggtcggtctaggtttcatccggtttgagttcttctctgacggcttcgccgacgacgatgaaaaaagattcgacttcgatgccttccgaccacaccttgaagctgtcggaaaacaagagatcaagaatctcgttaagagatataacaaggagccggtgacgtgtctcataaacaacgcttttgtcccatgggtatgtgatgtcgccgaggagcttcacatcccttcggctgttctatgggtccagtcttgtgcttgtctcacggcttattactattaccaccaccggttagttaagttcccgaccaaaaccgagccggacatcagcgttgaaatcccttgcttgccattgttaaagcatgacgagatcccaagctttcttcacccttcgtctccgtatacagcttttggagatatcattttagaccagttaaagagattcgaaaaccacaagtctttctatcttttcatcgacacttttcgcgaactagaaaaagacatcatggaccacatgtcacaactttgtcctcaagccatcatcagtcctgtcggtccgctcttcaagatggctcaaaccttgagttctgacgttaagggagatatatccgagccagcgagtgactgcatggaatggcttgactcaagagaaccatcctcagtcgtttacatctcctttgggactatagccaacttgaagcaagagcagatggaggagatcgctcatggcgttttgagctctggcttgtcggtcttatgggtggttcggcctcccatggaagggacatttgtagaaccacatgttttgcctcgagagctcgaagaaaagggtaaaatcgtggaatggtgtccccaagagagagtcttggctcatcctgcgattgcttgtttcttaagtcactgcggatggaactcgacaatggaggctttaactgccggagtccccgttgtttgttttccgcaatggggagatcaagtgactgatgcggtgtacttggctgatgttttcaagacaggagtgagactaggccgcggagccgctgaggagatgattgtttcgagggaggttgtagcagagaagctgcttgaggccacagttggggaaaaggcggtggagctgagagaaaacgctcggaggtggaaggcggaggccgaggccgccgtggcggacggtggatcatctgatatgaactttaaagagtttgtggacaagttggttacgaaacatgtgacgagagaagacaacggagaacactag

SEQ ID NO: 62 - protein, 479 aa, Arabidopsis thaliana
MDPSRHTHVMLVSFPGQGHVNPLLRLGKLIASKGLLVTFVTTEKPWGKKMRQANKIQDGVLKPVGLGFIRFEFFSDGFADDDEKRFDFDAFRPHLEAVGKQEIKNLVKRYNKEPVTCLINNAFVPWVCDVAEELHIPSAVLWVQSCACLTAYYYYHHRLVKFPTKTEPDISVEIPCLPLLKHDEIPSFLHPSSPYTAFGDIILDQLKRFENHKSFYLFIDTFRELEKDIMDHMSQLCPQAIISPVGPLFKMAQTLSSDVKGDISEPASDCMEWLDSREPSSVVYISFGTIANLKQEQMEEIAHGVLSSGLSVLWVVRPPMEGTFVEPHVLPRELEEKGKIVEWCPQERVLAHPAIACFLSHCGWNSTMEALTAGVPVVCFPQWGDQVTDAVYLADVFKTGVRLGRGAAEEMIVSREVVAEKLLEATVGEKAVELRENARRWKAEAEAAVADGGSSDMNFKEFVDKLVTKHVTREDNGEH

SEQ ID NO: 63 - DNA, 1443 bp, Artificial Sequence
  Synthetic oligonucleotide
atggctgaagctaacaccccacatattgctattatgccatctccaggtatgggtcatttgattccatttgttgaattggccaagagattggttcaacatgattgcttcactgtcaccatgattatttctggtgaaacctctccatctaaggcccaaagatctgttttgaactctttgccatcttccattgcctctgtttttttgccaccagctgatttgtctgatgttccatctactgctagaattgaaaccagagctatgttgactatgaccagatctaatccagccttgagagaattattcggttctttgtctaccaagaagtctttgccagctgttttggttgttgatatgtttggtgctgatgctttcgatgttgctgttgattttcatgtctccccatatatcttctacgcttctaatgctaacgtcttgtccttcttcttgcatttgccaaagttggataagaccgtcagttgcgagtttagatacttgactgaaccattgaagatcccaggttgtgttccaattactggtaaggatttcttggataccgttcaagatagaaacgatgatgcctacaagttgttgttgcataacaccaagagatacaaagaagccaagggtatcttggttaactccttcgttgatttggaatccaatgctattaaggccttgcaagaaccagctccagataagccaactgtttatccaattggtcctttggttaacacctcctcttctaacgttaacttggaagataagttcggttgcttgtcctggttggataatcaaccatttggttccgtcttgtacatctcttttggttctggtggtactttgacctgtgaacaattcaacgaattggctattggtttggctgaatctggtaagagattcatctgggttattagatccccatccgaaatcgtttcttccagttactttaacccacactctgaaactgatccattctcttttttgccaatcggtttcttggatagaacaaaagaaaagggtttggttgttccatcttgggctccacaagttcaaattttggctcatccatctacttgtggtttcttgactcattgtggttggaactctactttggaatctatcgttaatggtgttccattgattgcttggcctttgtttgctgaacaaaagatgaacaccttgttgttggttgaagatgttggtgctgctttgagaatccatgctggtgaagatggtatagtcagaagagaagaagttgttagagttgttaaggccttgatggaaggtgaagaaggtaaagctattggtaacaaggtcaaagaattgaaagaaggtgttgtcagagttttgggtgatgatggtttgtcctctaagtcttttggtgaagttttgttgaagtggaaaacccaccaaagagacatcaatcaagaaacctctcactaa

SEQ ID NO: 64 - DNA, 1425 bp, Artificial Sequence
  Synthetic oligonucleotide
atgtccacctctgaattggtttttattccatctccaggtgctggtcatttgccaccaactgttgaattggctaagttgttgttgcacagagatcaaagattgtccgttaccatcatcgttatgaatttgtggttgggtccaaagcacaatactgaagctagaccatgtgttccatctttgagattcgttgatatcccatgtgatgaatctaccatggctttgatttctccaaacaccttcatttccgctttcgttgaacatcataagccaagagttagagatatcgtcagaggtatcatcgaatccgattctgttagattggctggtttcgttttggatatgttctgtatgccaatgtccgatgttgctaatgaatttggtgttccttcctacaactacttcacttctggtgctgctactttgggtttgatgtttcacttgcaatggaagagagatcacgaaggttacgatgctactgaattgaagaactctgataccgaattgtctgtcccatcttacgttaatccagttccagctaaagttttgccagaagttgttttggacaaagaaggtggttctaagatgttcttggatttggccgaaagaatcagagaatccaagggtattatcgtcaactcttgccaagctattgaaagacatgccttggaatacttgtcctctaacaacaatggtatcccaccagtttttccagttggtccaattttgaacttggaaaacaaaaaggatgatgccaagaccgacgaaattatgagatggttgaatgaacaaccagaatcctccgttgttttcttgtgttttggttctatgggttcctttaacgaaaagcaagtcaaagaaattgccgtcgccattgaaagatctggtcatagatttttgtggtccttgagaagaccaaccccaaaagaaaagatcgaatttccaaaagaatacgaaaacttggaagaagtcttgcctgaaggtttcttgaagagaacttcttctatcggtaaggttattggttgggctccacaaatggctgttttgtctcatccatctgttggtggttttgtttctcattgcggttggaattctaccttggaatctatgtggtgtggtgttccaatggctgcttggccattatatgctgaacaaactttgaacgccttcttgttggttgtagaattgggtttggctgccgaaattagaatggattacagaactgataccaaggccggttatgatggtggtatggaagttactgttgaagaaatcgaagatggtatcagaaagttgatgtccgacggtgaaatcagaaacaaagttaaggacgtaaaagaaaaatccagagccgctgttgttgaaggtggtagttcttatgcttccatcggtaaattcatcgaacacgtttctaacgtcaccatctga

SEQ ID NO: 65 - DNA, 1446 bp, Arabidopsis thaliana
atgcatatcacaaaaccacacgccgccatgttttccagtcccggaatgggccatgtcatcccggtgatcgagcttggaaagcgtctctccgctaacaacggcttccacgtcaccgtcttcgtcctcgaaaccgacgcagcctccgctcaatccaagttcctaaactcaaccggcgtcgacatcgtcaaacttccatcgccggacatttatggtttagtggaccccgacgaccatgtagtgaccaagatcggagtcattatgcgtgcagcagttccagccctccgatccaagatcgctgccatgcatcaaaagccaacggctctgatcgttgacttgtttggcacagatgcgttatgtctcgcaaaggaatttaacatgttgagttatgtgtttatccctaccaacgcacgttttctcggagtttcgatttattatccaaatttggacaaagatatcaaggaagagcacacagtgcaaagaaacccactcgctataccggggtgtgaaccggttaggttcgaagatactctggatgcatatctggttcccgacgaaccggtgtaccgggattttgttcgtcatggtctggcttacccaaaagccgatggaattttggtaaatacatgggaagagatggagcccaaatcattgaagtcccttctaaacccaaagctcttgggccgggttgctcgtgtaccggtctatccaatcggtcccttatgcagaccgatacaatcatccgaaaccgatcacccggttttggattggttaaacgaacaaccgaacgagtcggttctctatatctccttcgggagtggtggttgtctatcggcgaaacagttaactgaattggcgtggggactcgagcagagccagcaacggttcgtatgggtggttcgaccaccggtcgacggttcgtgttgtagcgagtatgtctcggctaacggtggtggaaccgaagacaacacgccagagtatctaccggaagggttcgtgagtcgtactagtgatagaggtttcgtggtcccctcatgggccccacaagctgaaatcctgtcccatcgggccgttggtgggtttttgacccattgcggttggagctcgacgttggaaagcgtcgttggcggcgttccgatgatcgcatggccactttttgccgagcagaatatgaatgcggcgttgctcagcgacgaactgggaatcgcagtcagattggatgatccaaaggaggatatttctaggtggaagattgaggcgttggtgaggaaggttatgactgagaaggaaggtgaagcgatgagaaggaaagtgaagaagttgagagactcggcggagatgtcactgagcattgacggtggtggtttggcgcacgagtcgctttgcagagtcaccaaggagtgtcaacggtttttggaacgtgtcgtggacttgtcacgtggtgcttag

SEQ ID NO: 66 - protein, 481 aa, Arabidopsis thaliana
MHITKPHAAMFSSPGMGHVIPVIELGKRLSANNGFHVTVFVLETDAASAQSKFLNSTGVDIVKLPSPDIYGLVDPDDHVVTKIGVIMRAAVPALRSKIAAMHQKPTALIVDLFGTDALCLAKEFNMLSYVFIPTNARFLGVSIYYPNLDKDIKEEHTVQRNPLAIPGCEPVRFEDTLDAYLVPDEPVYRDFVRHGLAYPKADGILVNTWEEMEPKSLKSLLNPKLLGRVARVPVYPIGPLCRPIQSSETDHPVLDWLNEQPNESVLYISFGSGGCLSAKQLTELAWGLEQSQQRFVWVVRPPVDGSCCSEYVSANGGGTEDNTPEYLPEGFVSRTSDRGFVVPSWAPQAEILSHRAVGGFLTHCGWSSTLESVVGGVPMIAWPLFAEQNMNAALLSDELGIAVRLDDPKEDISRWKIEALVRKVMTEKEGEAMRRKVKKLRDSAEMSLSIDGGGLAHESLCRVTKECQRFLERVVDLSRGA

SEQ ID NO: 67 - DNA, 1419 bp, Artificial Sequence
  Synthetic oligonucleotide
atggggaagcaagaagatgcagagctcgtcatcatacctttccctttctccggacacattctcgcaacaatcgaactcgccaaacgtctcataagtcaagacaatcctcggatccacaccatcaccatcctctattggggattaccttttattcctcaagctgacacaatcgctttcctccgatccctagtcaaaaatgagcctcgtatccgtctcgttacgttgcccgaagtccaagaccctccaccaatggaactctttgtggaatttgccgaatcttacattcttgaatacgtcaagaaaatggttcccatcatcagagaagctctctccactctcttgtcttcccgcgatgaatcgggttcagttcgtgtggctggattggttcttgacttcttctgcgtccctatgatcgatgtaggaaacgagtttaatctcccttcttacattttcttgacgtgtagcgcagggttcttgggtatgatgaagtatcttccagagagacaccgcgaaatcaaatcggaattcaaccggagcttcaacgaggagttgaatctcattcctggttatgtcaactctgttcctactaaggttttgccgtcaggtctattcatgaaagagacctacgagccttgggtcgaactagcagagaggtttcctgaagctaagggtattttggttaattcatacacagctctcgagccaaacggttttaaatatttcgatcgttgtccggataactacccaaccatttacccaatcattctatgctccaacgatcgtccgaatttggatttatcggaacgagaccggatcttgaaatggctcgatgaccaacccgagtcatctgttgtgtttctctgcttcgggagcttgaagagtctcgctgcgtctcagattaaagagatcgctcaagccttagagctcgtcggaatcagattcctctggtcgattcgaacggacccgaaggagtacgcgagcccgaacgagattttaccggacgggtttatgaaccgagtcatgggtttgggccttgtttgtggttgggctcctcaagttgaaattctggcccataaagcaattggagggttcgtgtcacactgcggttggaactcgatattggagagtttgcgtttcggagttccaattgccacgtggccaatgtacgcggaacaacaactaaacgcgttcacgattgtgaaggagcttggtttggcgttggagatgcggttggattacgtgtcggaatatggagaaatcgtgaaagctgatgaaatcgcaggagccgtacgatctttgatggacggtgaggatgtgccgaggaggaaactgaaggagattgcggaggcgggaaaagaggctgtgatggacggtggatcttcgtttgttgcggttaaaagattcatagatgggctttga

SEQ ID NO: 68 - protein, 474 aa, Artificial Sequence
  Synthetic peptide
MGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGYVNSVPTKVLPSGLFMKETYEPWVELAERFPEAKGILVNSYTALEPNGFKYFDRCPDNYPTIYPIGPILCSNDRPNLDLSERDRILKWLDDQPESSVVFLCFGSLKSLAASQIKEIAQALELVGIRFLWSIRTDPKEYASPNEILPDGFMNRVMGLGLVCGWAPQVEILAHKAIGGFVSHCGWNSILESLRFGVPIATWPMYAEQQLNAFTIVKELGLALEMRLDYVSEYGEIVKADEIAGAVRSLMDGEDVPRRKLKEIAEAGKEAVMDGGSSFVAVKRFIDGL

SEQ ID NO: 69 - DNA, 1473 bp, Artificial Sequence
  Synthetic oligonucleotide
atggctatggggaagcaagaagatgcagagctcgtcatcatacctttccctttctccggacacattctcgcaacaatcgaactcgccaaacgtctcataagtcaagacaatcctcggatccacaccatcaccatcctctattggggattaccttttattcctcaagctgacacaatcgctttcctccgatccctagtcaaaaatgagcctcgtatccgtctcgttacgttgcccgaagtccaagaccctccaccaatggaactctttgtggaatttgccgaatcttacattcttgaatacgtcaagaaaatggttcccatcatcagagaagctctctccactctcttgtcttcccgcgatgaatcgggttcagttcgtgtggctggattggttcttgacttcttctgcgtccctatgatcgatgtaggaaacgagtttaatctcccttcttacattttcttgacgtgtagcgcagggttcttgggtatgatgaagtatcttccagagagacaccgcgaaatcaaatcggaattcaaccggagcttcaacgaggagttgaatctcattcctggttatgtcaactctgttcctactaaggttttgccgtcaggtctattcatgaaagagacctacgagccttgggtcgaactagcagagaggtttcctgaagctaagggtattttggttaattcatacacagctctcgagccaaacggttttaaatatttcgatcgttgtccggataactacccaaccatttacccaatcattttgaaccttgaaaacaaaaaagacgatgctaaaaccgacgagattatgaggtggttaaatgagcaaccggaaagctcggttgtgtttttatgtttcggaagcatgggtagctttaacgagaaacaagtgaaggagattgcggttgcgattgaaagaagtggacatagatttttatggtcgcttcgtcgtccgacaccgaaagaaaagatagagtttccgaaagaatatgaaaacttggaagaagttcttccagagggattccttaaacgtacatcaagcatcgggaaggtgatcgggtgggccccacaaatggcggtgttgtctcacccgtcagttggtgggtttgtgtcgcattgtggttggaactcgacattggagagtatgtggtgtggggttccgatggcagcttggccattatatgctgaacaaacgttgaatgcttttctacttgtggtggaactgggattggcggcggagattaggatggattatcggacggatacgaaagcggggtatgacggtgggatggaggtgacggtggaggagattgaagatggaattaggaagttgatgagtgatggtgagattagaaataaggtgaaagatgtgaaagagaagagtagagctgcggttgttgaaggtggatcttcttacgcatccattggaaaattcatcgagcatgtatcgaatgttacgatttaa

SEQ ID NO: 70 - protein, 492 aa, Artificial Sequence
  Synthetic peptide
MAMGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGYVNSVPTKVLPSGLFMKETYEPWVELAERFPEAKGILVNSYTALEPNGFKYFDRCPDNYPTIYPIGPILNLENKKDDAKTDEIMRWLNEQPESSVVFLCFGSMGSFNEKQVKEIAVAIERSGHRFLWSLRRPTPKEKIEFPKEYENLEEVLPEGFLKRTSSIGKVIGWAPQMAVLSHPSVGGFVSHCGWNSTLESMWCGVPMAAWPLYAEQTLNAFLLVVELGLAAEIRMDYRTDTKAGYDGGMEVTVEEIEDGIRKLMSDGEIRNKVKDVKEKSRAAVVEGGSSYASIGKFIEHVSNVTI

SEQ ID NO: 71 - DNA, 1473 bp, Artificial Sequence
  Synthetic oligonucleotide
atggctatggcgaagcagcaagaagcagagctcatcttcatcccatttccaatccccggacacattctcgccacaatcgaactcgcgaaacgtctcatcagtcaccaacctagtcggatccacaccatcaccatcctccattggagcttaccttttcttcctcaatctgacactatcgccttcctcaaatccctaatcgaaacagagtctcgtatccgtctcattaccttacccgatgtccaaaaccctccaccaatggagctatttgtgaaagcttccgaatcttacattcttgaatacgtcaagaaaatggttcctttggtcagaaacgctctctccactctcttgtcttctcgtgatgaatcggattcagttcatgtcgccggattagttcttgatttcttctgtgtccctttgatcgatgtcggaaacgagtttaatctcccttcttacatcttcttgacgtgtagcgcaagtttcttgggtatgatgaagtatcttctggagagaaaccgcgaaaccaaaccggaacttaaccggagctctgacgaggaaacaatatcagttcctggttttgttaactccgttccggttaaagttttgccaccgggtttgttcacgactgagtcttacgaagcttgggtcgaaatggcggaaaggttccctgaagccaagggtattttggtcaattcatttgaatctctagaacgtaacgcttttgattatttcgatcgtcgtccggataattacccacccgtttacccaatcattttgaaccttgaaaacaaaaaagacgatgctaaaaccgacgagattatgaggtggttaaatgagcaaccggaaagctcggttgtgtttttatgtttcggaagcatgggtagctttaacgagaaacaagtgaaggagattgcggttgcgattgaaagaagtggacatagatttttatggtcgcttcgtcgtccgacaccgaaagaaaagatagagtttccgaaagaatatgaaaacttggaagaagttcttccagagggattccttaaacgtacatcaagcatcgggaaggtgatcgggtgggccccacaaatggcggtgttgtctcacccgtcagttggtgggtttgtgtcgcattgtggttggaactcgacattggagagtatgtggtgtggggttccgatggcagcttggccattatatgctgaacaaacgttgaatgcttttctacttgtggtggaactgggattggcggcggagattaggatggattatcggacggatacgaaagcggggtatgacggtgggatggaggtgacggtggaggagattgaagatggaattaggaagttgatgagtgatggtgagattagaaataaggtgaaagatgtgaaagagaagagtagagctgcggttgttgaaggtggatcttcttacgcatccattggaaaattcatcgagcatgtatcgaatgttacgatttaa

SEQ ID NO: 72 - protein, 492 aa, Artificial Sequence
  Synthetic peptide
MAMAKQQEAELIFIPFPIPGHILATIELAKRLISHQPSRIHTITILHWSLPFLPQSDTIAFLKSLIETESRIRLITLPDVQNPPPMELFVKASESYILEYVKKMVPLVRNALSTLLSSRDESDSVHVAGLVLDFFCVPLIDVGNEFNLPSYIFLTCSASFLGMMKYLLERNRETKPELNRSSDEETISVPGFVNSVPVKVLPPGLFTTESYEAWVEMAERFPEAKGILVNSFESLERNAFDYFDRRPDNYPPVYPIGPILNLENKKDDAKTDEIMRWLNEQPESSVVFLCFGSMGSFNEKQVKEIAVAIERSGHRFLWSLRRPTPKEKIEFPKEYENLEEVLPEGFLKRTSSIGKVIGWAPQMAVLSHPSVGGFVSHCGWNSTLESMWCGVPMAAWPLYAEQTLNAFLLVVELGLAAEIRMDYRTDTKAGYDGGMEVTVEEIEDGIRKLMSDGEIRNKVKDVKEKSRAAVVEGGSSYASIGKFIEHVSNVTI

SEQ ID NO: 73 - DNA, 1437 bp, Artificial Sequence
  Synthetic oligonucleotide
atgaagattgagcttgtgttcatacctttgccggggattggtcatctcaggccaaccgtgaagctagcgaagcaactcataggcagcgaaaaccgtctttcgatcaccataatcatcatcccttcaagatttgacgccggtgatgcatccgcctgtatcgcatctctcaccacgttgtctcaagatgatcgcctccattacgaatccatatccgtcgcaaaacaaccaccaacctccgacccggatcctgttccggctcaagtgtacatagagaaacaaaagacgaaagtgagagatgcagtcgcggcgagaatcgtcgatccaacaagaaagctcgcgggattcgtggtggacatgttctgttcctcgatgatcgatgtagctaacgagtttggagttccgtgttatatggtatacacatcgaacgctacgtttttaggaaccatgcttcacgttcaacaaatgtacgatcaaaagaagtatgacgtcagcgagttagaaaactcggtcaccgagttggagtttccgtctctgactcgtccttatccagtgaagtgtcttcctcatatcctcacttcaaaggagtggttacctctctctctagctcaagctaggtgtttccggaagatgaagggtattttggtaaatacagttgctgagcttgaacctcacgctttgaaaatgttcaatattaatggtgacgatcttcctcaagtttatcctgttggaccagtgttgcatctcgaaaacggcaatgacgatgatgagaagcaatcggaaattttgcggtggctcgacgagcaaccgtctaaatctgttgtgtttctctgctttgggagcttgggaggtttcactgaagaacaaacaagagaaaccgctgtggccctagatagaagcggtcagcggtttctttggtgtcttcgtcacgcatcgccaaatataaaaacagatcgtcccagagattacacgaatcttgaggaggttttaccggaggggttcttggaacggactttggatagagggaaagtgattggatgggcaccacaagtggcggtactagagaagccggcgataggagggtttgtcactcactgcggttggaactctattttagagagcttgtggttcggtgttccaatggtgacgtggccgctatacgcggaacagaaggttaacgcgtttgagatggttgaggagctgggtttggcggtggagatacggaagtacttaaaaggagatttgttcgccggagagatggagacggttaccgcggaggatatagagagagccattaggcgtgtgatggagcaagacagtgacgttaggaacaacgtgaaagagatggcggagaagtgccacttcgcgttaatggacggtggatcttcgaaggcggctttggaaaagtttattcaagacgtgatagagaatatggactag

SEQ ID NO: 74 - protein, 478 aa, Arabidopsis thaliana
MKIELVFIPLPGIGHLRPTVKLAKQLIGSENRLSITIIIIPSRFDAGDASACIASLTTLSQDDRLHYESISVAKQPPTSDPDPVPAQVYIEKQKTKVRDAVAARIVDPTRKLAGFVVDMFCSSMIDVANEFGVPCYMVYTSNATFLGTMLHVQQMYDQKKYDVSELENSVTELEFPSLTRPYPVKCLPHILTSKEWLPLSLAQARCFRKMKGILVNTVAELEPHALKMFNINGDDLPQVYPVGPVLHLENGNDDDEKQSEILRWLDEQPSKSVVFLCFGSLGGFTEEQTRETAVALDRSGQRFLWCLRHASPNIKTDRPRDYTNLEEVLPEGFLERTLDRGKVIGWAPQVAVLEKPAIGGFVTHCGWNSILESLWFGVPMVTWPLYAEQKVNAFEMVEELGLAVEIRKYLKGDLFAGEMETVTAEDIERAIRRVMEQDSDVRNNVKEMAEKCHFALMDGGSSKAALEKFIQDVIENMD

SEQ ID NO: 75 - DNA, 1374 bp, Arabidopsis thaliana
atggaacttcacggagctctagtggctagtccgggcatgggacatgccgtacccatcttagaactcggtaaacatctcctgaaccaccacgggttcgaccgtgtcactgtcttcctagtcacagacgatgtctcacgttcgaaatccctaattggaaaaacgttgatggaagaagatccaaaatttgtgatcaggtttattccactcgatgtttcgggtcaagatctgagtggttcactattgactaaactagcagagatgatgaggaaggcattaccagagatcaagtcttcagtcatggagttagaaccgcggcctagggttttcgtagttgacttgttgggcacggaagctttagaggtggctaaggagcttgggatcatgagaaaacatgttctggttactaccagtgcttggtttctagcttttacggtttatatggcgagtcttgacaaacaggagttgtataagcagttgagtagcataggagcattgcttatacccggatgcagcccggttaagtttgagcgggctcaagatccgagaaaatatattcgggaactcgctgagtctcagcgtattggggatgaggtgataaccgcagatggggtgtttgtgaatacgtggcacagtctggagcaagtgaccatcgggtctttcttggatccagagaatctcggtcgggttatgagaggagtgccggtttatcctgttggaccgctggttagaccagcagaaccaggtttgaaacatggcgtgctggactggcttgacttacaacccaaagagtcagtggtttatgtttcttttgggagtggtggggcactaaccttcgagcagacaaacgagctggcttacggtttggagctgactggccacagatttgtttgggtagtcagaccaccggctgaagacgacccatcggcatcaatgttcgacaagaccaagaatgagacagaacctctcgatttcttacccaacgggtttctagaccgaaccaaagacatcggtttggtggtccgtacatgggcaccacaagaagagattctggcacacaagtcaacaggagggtttgtgactcactgcggatggaactcagttttggagagtattgtgaatggtgtgccaatggtagcttggccgttgtactcagagcagaagatgaacgcgaggatggtttctggggagctaaagattgcgttgcagattaatgttgcagatgggattgtaaagaaggaggtgatagctgaaatggtgaagagagtgatggatgaagaagaaggaaaagagatgagaaagaatgttaaggaactgaagaagacagcagaagaagctctcaacatgactcacattccatctgcttacttctgctag

SEQ ID NO: 76 - protein, 457 aa, Arabidopsis thaliana
MELHGALVASPGMGHAVPILELGKHLLNHHGFDRVTVFLVTDDVSRSKSLIGKTLMEEDPKFVIRFIPLDVSGQDLSGSLLTKLAEMMRKALPEIKSSVMELEPRPRVFVVDLLGTEALEVAKELGIMRKHVLVTTSAWFLAFTVYMASLDKQELYKQLSSIGALLIPGCSPVKFERAQDPRKYIRELAESQRIGDEVITADGVFVNTWHSLEQVTIGSFLDPENLGRVMRGVPVYPVGPLVRPAEPGLKHGVLDWLDLQPKESVVYVSFGSGGALTFEQTNELAYGLELTGHRFVWVVRPPAEDDPSASMFDKTKNETEPLDFLPNGFLDRTKDIGLVVRTWAPQEEILAHKSTGGFVTHCGWNSVLESIVNGVPMVAWPLYSEQKMNARMVSGELKIALQINVADGIVKKEVIAEMVKRVMDEEEGKEMRKNVKELKKTAEEALNMTHIPSAYFC

SEQ ID NO: 77 - DNA, 1467 bp, Arabidopsis thaliana
atgggaactcctgtcgaagtctctaagctccatttcttgctcttccctttcatggctcatggccatatgataccaactctagacatggctaagctctttgccaccaaaggagctaaatccactatcctcactacacctctcaatgccaagctcttcttcgagaaacccatcaaatcattcaaccaagacaacccgggactcgaagacatcaccatccagatccttaatttcccttgcacagagcttggtttgcctgatggctgtgagaatactgatttcatcttctccacacctgacctaaacgtaggtgacttgagtcaaaagtttttactcgcaatgaaatatttcgaagagccactagaggagctcctcgtgacaatgagaccagactgtcttgtcggtaacatgttcttcccttggtccactaaagttgctgagaagttcggagtaccgagacttgtgttccacggcacaggctacttctctttatgtgcttctcattgcataaggctccctaagaatgtggcaacaagttctgagccctttgtgattcctgatctcccgggagacattttgattacagaggaacaggtcatggagacagaagaagagtctgtaatggggaggtttatgaaggcaataagagactcagagagagatagctttggcgtgttggtgaacagcttctacgagcttgaacaggcttactcagattatttcaagagctttgtggcgaaaagagcgtggcatatcggtccgctttccttaggaaatagaaagttcgaggagaaagcagaaagaggcaaaaaggcaagcattgatgagcatgaatgtttgaaatggctcgactccaagaaatgtgattcagtgatttacatggcctttggaaccatgtctagctttaaaaacgagcagctgatagagattgcagctggtttagatatgtcaggacatgattttgtctgggtggttaacagaaaaggcagccaagttgagaaggaagattggttaccagaggggtttgaagagaagaccaagggaaaaggattgataatccgagggtgggcgccacaagtgctgatacttgagcacaaagcaattggcggatttttgacgcattgtggatggaactcgttattagaaggggtggcagcgggcctgccaatggtgacatggcccgtgggagccgagcagttctacaacgagaaattggtgacacaagtgttgaaaacaggagtgagtgtgggagtgaagaagatgatgcaagtagttggagacttcattagcagagagaaagtggagggagcggtgagggaagtgatggttggagaagagaggaggaaacgggccaaggagttagcagaaatggcgaaaaatgcggtgaaagaaggaggatcttcagatctagaggtagataggttgatggaagagcttacgttagttaaactgcaaaaagagaaggtataa

SEQ ID NO: 78 - protein, 488 aa, Arabidopsis thaliana
MGTPVEVSKLHFLLFPFMAHGHMIPTLDMAKLFATKGAKSTILTTPLNAKLFFEKPIKSFNQDNPGLEDITIQILNFPCTELGLPDGCENTDFIFSTPDLNVGDLSQKFLLAMKYFEEPLEELLVTMRPDCLVGNMFFPWSTKVAEKFGVPRLVFHGTGYFSLCASHCIRLPKNVATSSEPFVIPDLPGDILITEEQVMETEEESVMGRFMKAIRDSERDSFGVLVNSFYELEQAYSDYFKSFVAKRAWHIGPLSLGNRKFEEKAERGKKASIDEHECLKWLDSKKCDSVIYMAFGTMSSFKNEQLIEIAAGLDMSGHDFVWVVNRKGSQVEKEDWLPEGFEEKTKGKGLIIRGWAPQVLILEHKAIGGFLTHCGWNSLLEGVAAGLPMVTWPVGAEQFYNEKLVTQVLKTGVSVGVKKMMQVVGDFISREKVEGAVREVMVGEERRKRAKELAEMAKNAVKEGGSSDLEVDRLMEELTLVKLQKEKV

SEQ ID NO: 79 - DNA, 1455 bp, Arabidopsis thaliana
atgaacagagagcaaattcatattttgttcttccccttcatggctcatggccacatgattccactcttagacatggccaagcttttcgctagaagaggagccaaatcaactctcctcacaaccccaataaatgctaagatcttggagaaacccattgaagcattcaaagttcaaaatcctgatctcgaaatcggaatcaagatcctcaatttcccttgtgtagagcttggattgccagaaggatgcgagaaccgtgacttcattaactcataccaaaaatctgactcatttgacttgttcttgaagtttcttttctctaccaagtatatgaaacagcagttggagagtttcattgaaacaaccaaaccgagtgctcttgtagccgatatgttcttcccttgggcaacagaatccgcggagaagatcggtgttccaagacttgtgttccacggcacatcatcctttgccttgtgttgttcgtataacatgaggattcataagccacacaagaaagtcgcttcgagttctactccatttgtaatccctggtctccctggagacatagttattacagaagaccaagccaatgtcaccaacgaagaaactccattcggaaagttttggaaagaagtcagggaatcagagaccagtagctttggtgttttggtgaatagcttctacgagctggaatcatcttatgctgatttttaccgtagttttgtggcgaaaaaagcgtggcatataggtccactttcactatccaacagagggattgcagagaaagccggaagagggaaaaaggcaaacattgatgagcaagaatgcctcaaatggcttgactctaagacacctggctcagtagtttacttgtcctttggtagcggaaccggcttacccaacgaacagctgttagagattgctttcggccttgaaggctctggacaaaatttcatttgggtggttagcaaaaatgaaaaccaagttgggacaggtgaaaatgaagattggttgcctaaagggtttgaagagaggaataaaggaaaagggctgataatacgcggatgggccccgcaagtgctgatacttgaccacaaagcaatcggaggatttgtgacgcattgcggatggaactcgactttggagggcattgccgcagggctgcctatggtgacttggccgatgggggcagaacagttctacaacgagaagttattgacaaaagtgttgagaataggagtgaacgttggagctaccgagttggtgaaaaaaggaaagttgattagtagagcacaagtggagaaggcagtaagggaagtgattggtggtgagaaggcagaggaaaggcggctaagggctaaggagctgggcgagatggctaaagccgctgtggaagaaggagggtcttcttataatgatgtgaacaagtttatggaagagctgaatggtagaaagtag

SEQ ID NO: 80 - protein, 484 aa, Arabidopsis thaliana
MNREQIHILFFPFMAHGHMIPLLDMAKLFARRGAKSTLLTTPINAKILEKPIEAFKVQNPDLEIGIKILNFPCVELGLPEGCENRDFINSYQKSDSFDLFLKFLFSTKYMKQQLESFIETTKPSALVADMFFPWATESAEKIGVPRLVFHGTSSFALCCSYNMRIHKPHKKVASSSTPFVIPGLPGDIVITEDQANVTNEETPFGKFWKEVRESETSSFGVLVNSFYELESSYADFYRSFVAKKAWHIGPLSLSNRGIAEKAGRGKKANIDEQECLKWLDSKTPGSVVYLSFGSGTGLPNEQLLEIAFGLEGSGQNFIWVVSKNENQVGTGENEDWLPKGFEERNKGKGLIIRGWAPQVLILDHKAIGGFVTHCGWNSTLEGIAAGLPMVTWPMGAEQFYNEKLLTKVLRIGVNVGATELVKKGKLISRAQVEKAVREVIGGEKAEERRLRAKELGEMAKAAVEEGGSSYNDVNKFMEELNGRK

SEQ ID NO: 81 - DNA, 1476 bp, Arabidopsis thaliana
atggcatcggaatttcgtcctcctcttcattttgttctcttccctttcatggctcaaggccacatgatcccaatggtagatattgcaaggctcctggctcagcgcggggtgactataaccattgtcactacacctcaaaacgcaggccggttcaagaacgttcttagccgggctatccaatccggcttgcccatcaatctcgtgcaagtaaagtttccatctcaagaatcgggttcaccggaaggacaggagaatttggacttgctcgattcattgggggcttcattaaccttcttcaaagcatttagcctgctcgaggaaccagtcgagaagctcttgaaagagattcaacctaggccaaactgcataatcgctgacatgtgtttgccttatacaaacagaattgccaagaatcttggtataccaaaaatcatctttcatggcatgtgttgcttcaatcttctttgtacgcacataatgcaccaaaaccacgagttcttggaaactatagagtctgacaaggaatacttccccattcctaatttccctgacagagttgagttcacaaaatctcagcttccaatggtattagttgctggagattggaaagacttccttgacggaatgacagaaggggataacacttcttatggtgtgattgttaacacgtttgaagagctcgagccagcttatgttagagactacaagaaggttaaagcgggtaagatatggagcatcggaccggtttccttgtgcaacaagttaggagaagaccaagctgagaggggaaacaaggcggacattgatcaagacgagtgtattaaatggcttgattctaaagaagaagggtcggtgctatatgtttgccttggaagtatatgcaatcttcctctgtctcagctcaaagagctcggcttaggcctcgaggaatcccaaagacctttcatttgggtcataagaggttgggagaagtataacgagttacttgaatggatctcagagagcggttataaggaaagaatcaaagaaagaggccttctcataacaggatggtcgcctcaaatgcttatccttacacatcctgccgttggaggattcttgacacattgtggatggaactctactcttgaaggaatcacttcaggcgttccattactcacgtggccactgtttggagaccaattctgcaatgagaaattggcggtgcagatactaaaagccggtgtgagagctggggttgaagagtccatgagatggggagaagaggagaaaataggagtactggtggataaagaaggagtaaagaaggcagtggaggaattgatgggtgatagtaatgatgctaaggagagaagaaaaagagtgaaagagcttggagaattagctcacaaggctgtggaagaaggaggctcttctcattccaacatcacattcttgctacaagacataatgcaattagaacaacccaagcgctag

SEQ ID NO: 82 - protein, 491 aa, Arabidopsis thaliana
MASEFRPPLHFVLFPFMAQGHMIPMVDIARLLAQRGVTITIVTTPQNAGRFKNVLSRAIQSGLPINLVQVKFPSQESGSPEGQENLDLLDSLGASLTFFKAFSLLEEPVEKLLKEIQPRPNCIIADMCLPYTNRIAKNLGIPKIIFHGMCCFNLLCTHIMHQNHEFLETIESDKEYFPIPNFPDRVEFTKSQLPMVLVAGDWKDFLDGMTEGDNTSYGVIVNTFEELEPAYVRDYKKVKAGKIWSIGPVSLCNKLGEDQAERGNKADIDQDECIKWLDSKEEGSVLYVCLGSICNLPLSQLKELGLGLEESQRPFIWVIRGWEKYNELLEWISESGYKERIKERGLLITGWSPQMLILTHPAVGGFLTHCGWNSTLEGITSGVPLLTWPLFGDQFCNEKLAVQILKAGVRAGVEESMRWGEEEKIGVLVDKEGVKKAVEELMGDSNDAKERRKRVKELGELAHKAVEEGGSSHSNITFLLQDIMQLEQPKR

SEQ ID NO: 83 - DNA, 1410 bp, Arabidopsis thaliana
atggcgccaccgcattttctactggtaacgtttccggcgcaaggtcacgtgaacccatctctccgttttgctcgtcggctcatcaaaagaaccggcgcacgtgtcactttcgtcacttgtgtctccgtcttccacaactccatgatcgcaaaccacaacaaagtcgaaaatctctctttccttactttctccgacggtttcgacgatggaggcatttccacctacgaagaccgtcagaaaaggtcggtgaatctcaaggttaacggcgataaggcactatcggatttcatcgaagctactaagaatggtgactctcccgtgacttgcttgatctacacgattcttctcaattgggctccaaaagtagcacgtagatttcaacttccctccgctcttctctggatccaaccggctttggttttcaacatctattacactcatttcatgggaaacaagtccgttttcgagttacctaatctgtcttctctggaaatcagagatcttccatctttcctcacaccttccaacacaaacaaaggcgcatacgatgcgtttcaagaaatgatggagtttctcataaaagaaaccaaaccgaaaattctcatcaacactttcgattcgctggaaccagaggccttaacggctttcccgaatatcgatatggtggcggttggtcctttacttcccacggagattttctcaggaagcaccaacaaatcagttaaagatcaaagtagtagttatacactttggctagactcgaaaacagagtcctctgttatttacgtttcctttggaacaatggttgagttgtccaagaaacagatagaggaactagcgagagcactcatagaagggaaacgaccgtttttgtgggttataactgataaatccaacagagaaacgaaaacagaaggagaagaagagacagagattgagaagatagctggattcagacacgagcttgaagaggttgggatgattgtgtcgtggtgttcgcagatagaggttttaagtcaccgagccgtaggttgttttgtgactcattgtgggtggagctcgacgctggagagtttggttcttggcgttccggttgtggcgtttccgatgtggtcggatcaaccgacgaacgcgaagctactggaagaaagttggaagactggtgtgagggtaagagagaacaaggatggtttggtggagagaggagagatcaggaggtgtttggaagccgtgatggaggagaagtcggtggagttgagggaaaacgcaaagaaatggaagcgtttagcgatggaagcgggtagagaaggaggatcttcggataagaacatggaggcttttgtggaggatatttgtggagaatctcttattcaaaacttgtgtgaagcagaggaggtaaaagtacgctag

SEQ ID NO: 84 - protein, 469 aa, Arabidopsis thaliana
MAPPHFLLVTFPAQGHVNPSLRFARRLIKRTGARVTFVTCVSVFHNSMIANHNKVENLSFLTFSDGFDDGGISTYEDRQKRSVNLKVNGDKALSDFIEATKNGDSPVTCLIYTILLNWAPKVARRFQLPSALLWIQPALVFNIYYTHFMGNKSVFELPNLSSLEIRDLPSFLTPSNTNKGAYDAFQEMMEFLIKETKPKILINTFDSLEPEALTAFPNIDMVAVGPLLPTEIFSGSTNKSVKDQSSSYTLWLDSKTESSVIYVSFGTMVELSKKQIEELARALIEGKRPFLWVITDKSNRETKTEGEEETEIEKIAGFRHELEEVGMIVSWCSQIEVLSHRAVGCFVTHCGWSSTLESLVLGVPVVAFPMWSDQPTNAKLLEESWKTGVRVRENKDGLVERGEIRRCLEAVMEEKSVELRENAKKWKRLAMEAGREGGSSDKNMEAFVEDICGESLIQNLCEAEEVKVK

SEQ ID NO: 85 - DNA, 1425 bp, Arabidopsis thaliana
atggccaacaacaattccaactctcccaccggtccacactttctattcgtaacatttccagcccaaggtcacatcaacccatctctcgagctagccaaacgcctcgccggaacaatctctggtgctcgagtcaccttcgccgcctcaatctctgcctacaaccgccgcatgttctctacagaaaacgtccccgaaaccctaatcttcgctacctactccgatggccacgacgacggtttcaaatcctctgcttactccgacaaatctcgtcaagacgccactggaaacttcatgtctgagatgagacgacgtggcaaagagacactaaccgaactaatcgaagataaccggaaacaaaacaggccttttacttgcgtggtttacacgattctcctcacttgggtcgctgagctagcgcgtgagtttcatcttccttctgctcttctttgggtccaaccagtaacagtcttctccattttttaccattacttcaatggctacgaagatgcaatctcagagatggctaataccccctctagttctattaaattaccttctctgccactgcttactgtccgtgatattccttctttcattgtctcttccaatgtctacgcgtttcttctacccgcgtttcgagaacagattgattcactgaaggaagaaataaaccctaagatcctcatcaacactttccaagagcttgagccagaagccatgagctcggttccagataatttcaagattgtccctgtcggtccgttactaacgttgagaacggatttttcgagtcgcggtgaatacatagagtggttggatactaaagcggattcgtctgtgctttatgtttcgttcgggacgcttgccgtgttgagcaagaaacagcttgtggagctttgtaaagcgttgatacaaagtcggagaccattcttgtgggtgattacggataagtcgtacagaaataaagaagatgagcaagagaaggaagaagattgcataagtagtttcagagaagagctcgatgagataggaatggtggtttcatggtgtgatcagtttagggttttgaatcatagatcgataggttgtttcgtgacgcattgcgggtggaactctacgctggagagcttggtttcaggagttccggtggtggcgtttccgcaatggaatgatcagatgatgaacgcgaagcttttagaagattgttggaaaacaggtgtaagagtgatggagaagaaggaagaagaaggagttgtggtggtggatagtgaggagatacggcggtgcattgaggaagttatggaagacaaggcggaggagtttagaggaaatgccacgaggtggaaggatttagcggcggaggctgtgagagaaggaggctcttcctttaatcatctcaaagcttttgtcgatgagcacatctag

SEQ ID NO: 86 - protein, 474 aa, Arabidopsis thaliana
MANNNSNSPTGPHFLFVTFPAQGHINPSLELAKRLAGTISGARVTFAASISAYNRRMFSTENVPETLIFATYSDGHDDGFKSSAYSDKSRQDATGNFMSEMRRRGKETLTELIEDNRKQNRPFTCVVYTILLTWVAELAREFHLPSALLWVQPVTVFSIFYHYFNGYEDAISEMANTPSSSIKLPSLPLLTVRDIPSFIVSSNVYAFLLPAFREQIDSLKEEINPKILINTFQELEPEAMSSVPDNFKIVPVGPLLTLRTDFSSRGEYIEWLDTKADSSVLYVSFGTLAVLSKKQLVELCKALIQSRRPFLWVITDKSYRNKEDEQEKEEDCISSFREELDEIGMVVSWCDQFRVLNHRSIGCFVTHCGWNSTLESLVSGVPVVAFPQWNDQMMNAKLLEDCWKTGVRVMEKKEEEGVVVVDSEEIRRCIEEVMEDKAEEFRGNATRWKDLAAEAVREGGSSFNHLKAFVDEHI

SEQ ID NO: 87 - DNA, 1344 bp, Arabidopsis thaliana
atggagaaaaatgcagagaagaaaagaatagtgttggttccatttccattacaaggacatatcactccaatgatgcaacttggtcaagcacttaacctgaaaggcttctcgattaccgttgctcttggagattccaatcgagtaagttctacgcaacacttccctggttttcaatttgtcacaatacctgaaaccataccactatctcaacacgaggcactcggagttgtcgagtttgtggttacgctcaacaaaacaagcgagacaagtttcaaggactgtatagctcatttgttgctgcaacatggaaatgatattgcttgtatcatttacgacgagctcatgtacttctctgaagctacagctaaggatttaaggattcctagtgtcatattcaccactggtagtgctacaaatcatgtttgttcttgtattttaagcaaactcaacgccgagaagttcttgatcgacatgaaagatcctgaagtgcaaaacatggtggtggaaaatttacatccactaaaatacaaagacttaccaacttcaggaatggggccgctagagcgatttttggagatttgtgccgaagttgtcaacaaaagaacagcttccgctgttataatcaatacgtcaagttgtctagagagctcgtctctgtcatggctgaaacaagaactcagtattccagtgtatccattaggccctcttcacattacaacttcagcaaattttagtttacttgaagaggacaggagctgcattgaatggctgaacaagcagaaactgaggtcagttatatacataagcgtaggaagcatagctcacatggaaaccaaggaagtattggagatggcttggggattgtataatagcaaccaaccttttctatgggtaatccgacccggtacagagtcaatgccagtggaagtcagtaagattgtctcggaaagaggatgcattgtgaaatgggcgccacagaatgaagtacttgtgcatcctgcagtgggaggtttctggagccactgtggatggaactcaacactcgagagtattgtggaaggagttccaatgatttgcagaccgtttaacggtgagcagaagttaaacgcgatgtatatagaaagtgtttggagagtaggggttctgcttcaaggagaagtggagagaggatgtgtagagagagctgtgaagaggttgattgtggatgatgaaggtgtaggaatgagggagagagcccttgttttaaaagagaagctcaatgcctctgtaagaagtggaggctcttcatacaatgcattggatgagctcgtccattacttggaggcagagtatagaaatacttga

SEQ ID NO: 88 - protein, 447 aa, Arabidopsis thaliana
MEKNAEKKRIVLVPFPLQGHITPMMQLGQALNLKGFSITVALGDSNRVSSTQHFPGFQFVTIPETIPLSQHEALGVVEFVVTLNKTSETSFKDCIAHLLLQHGNDIACIIYDELMYFSEATAKDLRIPSVIFTTGSATNHVCSCILSKLNAEKFLIDMKDPEVQNMVVENLHPLKYKDLPTSGMGPLERFLEICAEVVNKRTASAVIINTSSCLESSSLSWLKQELSIPVYPLGPLHITTSANFSLLEEDRSCIEWLNKQKLRSVIYISVGSIAHMETKEVLEMAWGLYNSNQPFLWVIRPGTESMPVEVSKIVSERGCIVKWAPQNEVLVHPAVGGFWSHCGWNSTLESIVEGVPMICRPFNGEQKLNAMYIESVWRVGVLLQGEVERGCVERAVKRLIVDDEGVGMRERALVLKEKLNASVRSGGSSYNALDELVHYLEAEYRNT

SEQ ID NO: 89 - DNA, 1383 bp, Arabidopsis thaliana
atggaagagagaaaagggaggagaataatcatgttccctcttccatttccagggcacttcaaccccatgatcgagctcgctggaatattccaccaccgtggcttctccgtgacgatcctccacacttcctacaacttccccgatccttctcgccacccacacttcacttttcgaaccatctctcacaacaaagaaggagaagaagatcctctgtctcagtcagaaacttcgagtatggacctaatcgttctcgttcgtcggctgaaacaacgctacgccgaaccgtttcgtaagtctgtggcggcggaagtaggtggaggagagacggtgtgttgtttggtctccgacgctatatgggggaagaacacggaggttgtagcggaagagattggagttcgtagggtggtgttgaggacaggtggtgcgtcgtcgttttgtgcttttgccgctttccctctccttagggataagggttacctccctatacaagattctagattagatgagccagtgacagagcttccacctttgaaagtgaaggatcttccggtaatggaaacgaatgagccggaggaactttaccgggtagttaacgacatggtggaaggagccaagtcttcttcaggagtcatatggaacacatttgaagatcttgaaagactatcacttatgaattgtagcagcaaattacaagttccatttttcccgatcggaccgtttcacaaatatagcgaagatcctacaccgaagacagagaacaaggaagataccgattggctcgacaagcaagacccacagtcggtggtctatgcgagtttcggaagccttgcagctatagaagagaaggagtttctcgagattgcttggggtctaagaaacagtgaacgaccgtttttgtgggtggttaggccggggtctgtcagggggaccgagtggctcgagtcattgcctttagggtttatggaaaacattggagataagggaaaaatcgtgaaatgggcgaatcagttagaggtattggcgcatcctgccattggagcgttttggacacattgtggatggaactcgacactagagagcatatgtgaaggtgttcctatgatatgtacgtcatgtttcacggaccagcatgtgaacgcgagatacatcgttgatgtatggcgagtcgggatgttgttagagagaagtaagatggaaaagaaggagattgaaaaggtgctaagaagtgtaatgatggagaagggagatggattgagggaaaggagtttgaagttgaaagagagagctgatttttgcttaagtaaagatgggtcttcttccaagtatttagacaaacttgtgagtcatgtcctgtcttttgattcttatgcttttgcaagctag

SEQ ID NO: 90 - protein, 460 aa, Arabidopsis thaliana
MEERKGRRIIMFPLPFPGHFNPMIELAGIFHHRGFSVTILHTSYNFPDPSRHPHFTFRTISHNKEGEEDPLSQSETSSMDLIVLVRRLKQRYAEPFRKSVAAEVGGGETVCCLVSDAIWGKNTEVVAEEIGVRRVVLRTGGASSFCAFAAFPLLRDKGYLPIQDSRLDEPVTELPPLKVKDLPVMETNEPEELYRVVNDMVEGAKSSSGVIWNTFEDLERLSLMNCSSKLQVPFFPIGPFHKYSEDPTPKTENKEDTDWLDKQDPQSVVYASFGSLAAIEEKEFLEIAWGLRNSERPFLWVVRPGSVRGTEWLESLPLGFMENIGDKGKIVKWANQLEVLAHPAIGAFWTHCGWNSTLESICEGVPMICTSCFTDQHVNARYIVDVWRVGMLLERSKMEKKEIEKVLRSVMMEKGDGLRERSLKLKERADFCLSKDGSSSKYLDKLVSHVLSFDSYAFAS

SEQ ID NO: 91 - DNA, 1380 bp, Arabidopsis thaliana
atggccaaaccctcgcagccaacgcgagactcccacgtggcagttctcgttttccccttcggcactcatgcagctcctctcctcgccgtcacgtgccgtctcgccaccgctgctccctccaccgtcttctccttcttcagcaccgcacgatccaactcgtcgttactctcctccgatatccccacaaacattcgtgtccacaacgtcgatgacggtgttcctgagggattcgtgttgacggggaatccacagcacgctgttgagctgtttcttgaagcggcgccagagattttccgaagagaaatcaaggcggccgagaccgaagttggtaggaagttcaagtgcatccttacggatgcgttcctctggttagcagcggagacggcggctgcggagatgaaagcgtcgtgggttgcgtactatggaggcggagcaacctcgctcactgctcatctctacacagatgccatcagagaaaacgtcggtgtcaaagaagtaggtgagcgtatggaggagacaatagggtttatctcaggaatggagaagatcagagtcaaagacacacaagaaggcgttgtgtttgggaacttagactctgttttctctaaaacgttgcaccaaatgggtcttgctttacctcgtgccactgctgttttcatcaattcctttgaagaattggatcctacgtttacaaatgatttcagatcggaattcaaacgttacctaaacatcggtcctctcgctttattatcttctccatcgcaaacatcaacgctagtgcacgatcctcacggttgcttggcttggatcgagaagcggtccactgcttctgtagcgtacattgcctttggtagagtcgcgacaccgcctcctgtagagcttgtggcgatagcacaaggattggaatcgagtaaagtgccttttgtttggtcgctacaagagatgaaaatgactcatttaccagaaggctttttggatcggaccagagagcaagggatggtggttccatgggcaccacaagtggagctgctaaaccatgaagcaatgggtgtgtttgtttcgcatggtgggtggaactcagtgttggagagtgtgtcggcaggtgtaccgatgatttgtagaccgattttcggggatcatgcaatcaatgcaagatctgtggaagctgtgtgggagatcggagtgacgattagtagtggagtcttcacgaaggatggatttgaggagagtttggatcgggttttggttcaagatgatggcaagaagatgaaggttaatgctaaaaagcttgaagaactagcacaagaagctgtctctaccaaaggaagctcctttgagaattttggaggattgttggacgaagttgtgaactttggataa

SEQ ID NO: 92 - protein, 459 aa, Arabidopsis thaliana
MAKPSQPTRDSHVAVLVFPFGTHAAPLLAVTCRLATAAPSTVFSFFSTARSNSSLLSSDIPTNIRVHNVDDGVPEGFVLTGNPQHAVELFLEAAPEIFRREIKAAETEVGRKFKCILTDAFLWLAAETAAAEMKASWVAYYGGGATSLTAHLYTDAIRENVGVKEVGERMEETIGFISGMEKIRVKDTQEGVVFGNLDSVFSKTLHQMGLALPRATAVFINSFEELDPTFTNDFRSEFKRYLNIGPLALLSSPSQTSTLVHDPHGCLAWIEKRSTASVAYIAFGRVATPPPVELVAIAQGLESSKVPFVWSLQEMKMTHLPEGFLDRTREQGMVVPWAPQVELLNHEAMGVFVSHGGWNSVLESVSAGVPMICRPIFGDHAINARSVEAVWEIGVTISSGVFTKDGFEESLDRVLVQDDGKKMKVNAKKLEELAQEAVSTKGSSFENFGGLLDEVVNFG

SEQ ID NO: 93 - DNA, 1359 bp, Arabidopsis thaliana
atgggccaaaagattcacgcttttatgttcccctggtttgcttttggtcatatgactccgtacttgcatctaggcaacaagttagccgagaaaggtcatagggttactttcttgctacctaagaaagctcagaaacaattggaacatcagaatctatttccacacggtatcgtctttcatcctcttgttattcctcatgttgatggcctccctgctggtgccgagacagcctcggatatccccatctcgttggtgaagttcttgtctatagccatggatcttacacgcgatcagatcgaagccgcgattggtgccttgagaccggacctaatcttgttcgatttagctcactgggttccggaaatggctaaagcgcttaaagtcaagagtatgttgtataacgtgatgtcagctacctctatagctcacgaccttgtcccaggtggtgaacttggagttgctccacctggttatccttcatcaaaggcgttgtaccgcgaacacgatgctcacgccttgttaaccttctccggcttctacaagaggttttatcaccggttcaccacaggtcttatgaattgcgatttcatttcgattcggacatgtgaagaaatcgaaggtaaattttgtgactatattgagagtcaatacaagaagaaggttcttttaaccggtccaatgcttcccgagcctgacaagagtaaaccacttgaagatcaatggagtcattggctgagtgggtttggacaaggctctgtagtgttctgtgcattgggaagtcaaaccattctagagaaaaaccaattccaagaactctgtttaggaatagagcttacgggtttaccatttcttgtcgcggttaagccaccaaaaggcgcaaacacaattcatgaagcgttaccagaagggttcgaggaaagggtgaagggtcgtggaatagtttggggagaatgggtgcagcaaccatcctggcaaccattgatattggctcatccatcagtaggttgctttgtgagccattgcggattcgggtcaatgtgggaatctttaatgagtgattgtcaaatagtctttattccagttttgaatgatcaagttctcaccacgagagtaatgacggaggaactcgaggtctccgttgaggtacagagagaagaaacaggatggttctcaaaagaaaacttgagtggtgcaatcatgtctttgatggaccaagacagcgagatagggaaccaagtgaggaggaaccattctaaattgaaggagactttggctagtcctggattattaaccggttacaccgataaatttgttgacactttggagaatctagtcaacgaacaaggatacatatcctag

SEQ ID NO: 94 - protein, 496 aa, Arabidopsis thaliana
MELESSPPLPPHVMLVSFPGQGHVNPLLRLGKLLASKGLLITFVTTESWGKKMRISNKIQDRVLKPVGKGYLRYDFFDDGLPEDDEASRTNLTILRPHLELVGKREIKNLVKRYKEVTKQPVTCLINNPFVSWVCDVAEDLQIPCAVLWVQSCACLAAYYYYHHNLVDFPTKTEPEIDVQISGMPLLKHDEIPSFIHPSSPHSALREVIIDQIKRLHKTFSIFIDTFNSLEKDIIDHMSTLSLPGVIRPLGPLYKMAKTVAYDVVKVNISEPTDPCMEWLDSQPVSSVVYISFGTVAYLKQEQIDEIAYGVLNADVTFLWVIRQQELGFNKEKHVLPEEVKGKGKIVEWCSQEKVLSHPSVACFVTHCGWNSTMEAVSSGVPTVCFPQWGDQVTDAVYMIDVWKTGVRLSRGEAEERLVPREEVAERLREVTKGEKAIELKKNALKWKEEAEAAVARGGSSDRNLEKFVEKLGAKPVGKVQNGSHNHVLAGSIKSY

SEQ ID NO: 95 - DNA, 1470 bp, Arabidopsis thaliana
atgggatctcagatcattcataactcacaaaaaccacatgtagtttgtgttccatatccggctcaaggccacatcaaccctatgatgagagtggctaaactcctccacgccagaggcttctacgtcaccttcgtcaacaccgtctacaaccacaatcgtttccttcgttctcgtgggtccaatgccctagatggacttccttcgttccgatttgagtccattgctgacggtctaccagagacagacatggatgccacgcaggacatcacagctctttgcgagtccaccatgaagaactgtctcgctccgttcagagagcttctccagcggatcaacgctggagataatgttcctccggtaagctgtattgtatctgacggttgtatgagctttactcttgatgttgcggaggagcttggagtcccggaggttcttttttggacaaccagtggctgtgcgttcctggcttatctacacttttatctcttcatcgagaagggcttatgtccgctaaaagatgagagttacttgacgaaggagtacttagaagacacggttatagattttataccaaccatgaagaatgtgaaactaaaggatattcctagcttcatacgtaccactaatcctgatgatgttatgattagtttcgccctccgcgagaccgagcgagccaaacgtgcttctgctatcattctaaacacatttgatgaccttgagcatgatgttgttcatgctatgcaatctatcttacctccggtttattcagttggaccgcttcatctcttagcaaaccgggagattgaagaaggtagtgagattggaatgatgagttcgaatttatggaaagaggagatggagtgtttggattggcttgatactaagactcaaaatagtgtcatttatatcaactttgggagcataacggttttgagtgtgaagcagcttgtggagtttgcttggggtttggcgggaagtgggaaagagtttttatgggtgatccggccagatttagtagcgggagaggaggctatggttccgccggactttttaatggagactaaagaccgcagtatgctagcgagttggtgtcctcaagagaaagtactttctcatcctgctattggagggtttttgacgcattgcgggtggaactcgatattggaaagtctttcgtgtggagttccgatggtgtgttggccattttttgctgaccagcaaatgaattgtaagttttgttgtgacgagtgggatgttgggattgagataggtggagatgtgaagagagaggaagttgaggcggtggttagagagctcatggatggagagaagggaaagaaaatgagagaaaaggcggtagagtggcagcgcttagccgagaaagcgacggaacataaacttggttcttccgttatgaattttgagacggttgttagcaagtttcttttgggacaaaaatcacaggactag

SEQ ID NO: 96 - protein, 489 aa, Arabidopsis thaliana
MGSQIIHNSQKPHVVCVPYPAQGHINPMMRVAKLLHARGFYVTFVNTVYNHNRFLRSRGSNALDGLPSFRFESIADGLPETDMDATQDITALCESTMKNCLAPFRELLQRINAGDNVPPVSCIVSDGCMSFTLDVAEELGVPEVLFWTTSGCAFLAYLHFYLFIEKGLCPLKDESYLTKEYLEDTVIDFIPTMKNVKLKDIPSFIRTTNPDDVMISFALRETERAKRASAIILNTFDDLEHDVVHAMQSILPPVYSVGPLHLLANREIEEGSEIGMMSSNLWKEEMECLDWLDTKTQNSVIYINFGSITVLSVKQLVEFAWGLAGSGKEFLWVIRPDLVAGEEAMVPPDFLMETKDRSMLASWCPQEKVLSHPAIGGFLTHCGWNSILESLSCGVPMVCWPFFADQQMNCKFCCDEWDVGIEIGGDVKREEVEAVVRELMDGEKGKKMREKAVEWQRLAEKATEHKLGSSVMNFETVVSKFLLGQKSQD

SEQ ID NO: 97 - DNA, 1368 bp, Arabidopsis thaliana
atggatccaaatgaatctccaccaaaccaatttcgccacgtggtggccatgccttatccaggtcgaggacacatcaaccctatgatgaacctctgcaaacgccttgtccgtcgataccctaaccttcacgtcaccttcgtcgtcacagaagaatggctcgggtttattggacccgacccgaaacccgaccggatccatttctccactctccctaatctcatcccttccgagcttgtcagggccaaagacttcataggcttcattgatgccgtctacacaagattggaagaaccattcgagaagcttcttgacagcctcaattcaccacctccgagtgtaatattcgccgacacttacgtcatttgggctgtgcgagtcggcagaaaaaggaatattccggtggtttctctctggaccatgtcagccacgattctctccttcttcctccactctgatctactcataagtcatggccatgctctgttcgaaccatcagaagaagaggttgttgattacgtccccggtttatctccgacgaaactccgagatttgccgccgatatttgacggttacagcgaccgagtcttcaagacagctaagttgtgtttcgatgaactaccaggagctaggtctttactcttcaccaccgcctatgagcttgaacacaaagctattgacgctttcacctccaagctcgatatcccggtctacgctattggtcctttaataccttttgaagaactttctgttcaaaatgataacaaggaacctaattacatccagtggcttgaggaacaaccggaaggctctgttctttacatatctcagggaagttttctttcggtctcggaagctcagatggaggaaatagtgaaaggactgagagaaagtggagtccggtttctttgggtggctcgtgggggcgagttaaagcttaaggaggctcttgaaggtagcttaggtgtagtggtgagctggtgtgatcagcttcgggtgctgtgtcacaaagctgtaggcgggttttggactcattgcgggtttaactcgacattggaagggatatattcaggagtaccaatgctagcgtttccgttgttttgggatcagattctgaacgctaagatgattgttgaggactggagagtcggaatgaggatcgagaggacgaaaaagaatgagttgttgatagggagagaggagatcaaggaagtagtgaagaggtttatggatagagagagtgaagaagggaaagagatgagaagaagggcttgtgaccttagtgaaatcagtcgaggagctgttgcgaaaagcggttcgtctaatgtaaacatcgatgagttcgttcggcatattaccaatacaaactag

SEQ ID NO: 98 - protein, 455 aa, Arabidopsis thaliana
MDPNESPPNQFRHVVAMPYPGRGHINPMMNLCKRLVRRYPNLHVTFVVTEEWLGFIGPDPKPDRIHFSTLPNLIPSELVRAKDFIGFIDAVYTRLEEPFEKLLDSLNSPPPSVIFADTYVIWAVRVGRKRNIPVVSLWTMSATILSFFLHSDLLISHGHALFEPSEEEVVDYVPGLSPTKLRDLPPIFDGYSDRVFKTAKLCFDELPGARSLLFTTAYELEHKAIDAFTSKLDIPVYAIGPLIPFEELSVQNDNKEPNYIQWLEEQPEGSVLYISQGSFLSVSEAQMEEIVKGLRESGVRFLWVARGGELKLKEALEGSLGVVVSWCDQLRVLCHKAVGGFWTHCGFNSTLEGIYSGVPMLAFPLFWDQILNAKMIVEDWRVGMRIERTKKNELLIGREEIKEVVKRFMDRESEEGKEMRRRACDLSEISRGAVAKSGSSNVNIDEFVRHITNTN

SEQ ID NO: 99 - DNA, 1404 bp, Arabidopsis thaliana
atggagttagaaaaagttcacgtggttttgttcccatacttgtccaaagggcacatgattcctatgctccaattagctcgtctcctcttatcccactccttcgccggagacatctccgtcaccgtcttcaccactcctttgaaccgtcctttcatcgttgactcactctccggcaccaaagcgaccatcgtcgacgtacctttccctgataacgtcccggagatcccacccggcgtcgagtgcactgacaaactccctgctttgtcgtcctccctcttcgttcctttcacaagagccaccaagtcaatgcaggcagactttgagcgagagctcatgtcactgccacgtgtcagtttcatggtctcagacggtttcttgtggtggacgcaagagtcagctcgaaagctagggtttcctcggcttgttttctttggtatgaattgcgcttccaccgttatatgtgacagtgtttttcaaaaccagcttctatctaatgttaagtccgagacggagccagtttctgtaccggagtttccgtggattaaggttaggaaatgtgatttcgttaaagatatgtttgatccaaaaaccaccacagatcctggattcaagcttatcctagatcaagtcacgtctatgaatcaaagccaaggtatcatattcaatacatttgacgaccttgaacccgtgtttattgatttctacaagcgtaaacgcaaactcaagctttgggcagttggaccgctttgttacgtaaataacttcttggatgatgaagtagaagagaaggtcaaacctagttggatgaaatggctagatgaaaagcgagacaagggatgcaatgttctgtatgtggctttcgggtcacaagccgagatctcgagagaacaactagaggagattgcgttagggttggaagaatcgaaggtgaacttcttgtgggtggtcaaaggaaatgaaataggaaaagggtttgaagagagagtgggagaaagaggaatgatggtgagagatgaatgggttgatcagaggaagatattagagcacgagagtgttagagggttcttgagccattgtgggtggaattctctgacggagagcatttgctcggaggttccaatcttggcgtttcctttagcagcggagcaacctctgaatgcgattttggtggtggaagagctgagagtggcggagagagtggtggcggcgagtgaaggggttgtgagaagagaagagattgcagagaaagtgaaggagttgatggagggagagaaagggaaagagctgaggaggaatgtcgaggcatatggtaagatggcgaagaaggctttggaggaaggtattggttcgtctaggaagaatttagacaaccttatcaacgagttttgtaacaatggaacatga

SEQ ID NO: 100 - protein, 467 aa, Arabidopsis thaliana
MELEKVHVVLFPYLSKGHMIPMLQLARLLLSHSFAGDISVTVFTTPLNRPFIVDSLSGTKATIVDVPFPDNVPEIPPGVECTDKLPALSSSLFVPFTRATKSMQADFERELMSLPRVSFMVSDGFLWWTQESARKLGFPRLVFFGMNCASTVICDSVFQNQLLSNVKSETEPVSVPEFPWIKVRKCDFVKDMFDPKTTTDPGFKLILDQVTSMNQSQGIIFNTFDDLEPVFIDFYKRKRKLKLWAVGPLCYVNNFLDDEVEEKVKPSWMKWLDEKRDKGCNVLYVAFGSQAEISREQLEEIALGLEESKVNFLWVVKGNEIGKGFEERVGERGMMVRDEWVDQRKILEHESVRGFLSHCGWNSLTESICSEVPILAFPLAAEQPLNAILVVEELRVAERVVAASEGVVRREEIAEKVKELMEGEKGKELRRNVEAYGKMAKKALEEGIGSSRKNLDNLINEFCNNGT

SEQ ID NO: 101 - DNA, 1401 bp, Arabidopsis thaliana
atggccgagccaaaaccgaagcttcatgttgcagtgttcccatggttagctttaggtcacatgattccttacttgcaactctcaaagctcatagcaaggaaaggccatactgtgtccttcatctccacagctcgtaacatttcacgtcttcccaatatatcctccgacctttccgtgaatttcgtttctttgccgttaagtcaaaccgtcgaccatctcccagagaacgctgaggccaccactgatgtcccggagactcacatagcttatctgaagaaagcatttgatgggctttctgaagctttcacagagtttttagaagcttccaaaccaaactggatagtgtatgatatcttgcaccattgggtcccgcctatcgctgagaagctcggcgtgagacgagccatcttctgcacgttcaacgcagcttccatcatcatcatcggtgggccagcatcagtcatgattcaaggtcatgaccctcgaaagactgctgaagatcttatcgtgcctccaccatgggtcccgtttgagaccaacatagtttaccgtctctttgaagctaagaggatcatggagtatcccacggcaggtgtaactggagttgaattgaacgacaactgtagattgggtttggcttacgttggctctgaggttattgtgattagatcatgtatggaactcgaacctgagtggattcaattgctcagtaaactccaaggaaagcctgtgattccaattggtttactcccggctacaccaatggatgatgcagatgacgagggaacatggttagacatcagagaatggctagacagacatcaagcaaagtctgtggtttatgtagccttaggaactgaagtgacaattagtaacgaagagattcaaggtttagctcatgggttggagctttgcaggttacctttcttttggacgctaaggaagaggactagagcttctatgctactacctgatgggttcaaagagagagtcaaagagcgtggagtcatttggaccgagtgggtacctcagaccaagatactgagccatggttcagttggtgggtttgttactcattgtggttggggatcagctgtggaagggcttagctttggtgtccctttgatcatgtttccatgtaacctagaccagccgctagtggctaggttgctcagtgggatgaatataggcttggagattccaaggaatgagcgagacgggctgttcacgagtgcttctgttgcagagacaatcagacatgttgttgtggaagaagaaggaaagatctacaggaacaatgctgcatctcagcaaaagaaaatattcgggaacaagagattgcaagatcagtatgcggatggttttatcgagtttctggagaatcctatagcaggagtgtag

SEQ ID NO: 102 - protein, 466 aa, Arabidopsis thaliana
MAEPKPKLHVAVFPWLALGHMIPYLQLSKLIARKGHTVSFISTARNISRLPNISSDLSVNFVSLPLSQTVDHLPENAEATTDVPETHIAYLKKAFDGLSEAFTEFLEASKPNWIVYDILHHWVPPIAEKLGVRRAIFCTFNAASIIIIGGPASVMIQGHDPRKTAEDLIVPPPWVPFETNIVYRLFEAKRIMEYPTAGVTGVELNDNCRLGLAYVGSEVIVIRSCMELEPEWIQLLSKLQGKPVIPIGLLPATPMDDADDEGTWLDIREWLDRHQAKSVVYVALGTEVTISNEEIQGLAHGLELCRLPFFWTLRKRTRASMLLPDGFKERVKERGVIWTEWVPQTKILSHGSVGGFVTHCGWGSAVEGLSFGVPLIMFPCNLDQPLVARLLSGMNIGLEIPRNERDGLFTSASVAETIRHVVVEEEGKIYRNNAASQQKKIFGNKRLQDQYADGFIEFLENPIAGV

SEQ ID NO: 103 - protein, 1425 aa, Artificial Sequence
  Synthetic oligonucleotide
ATGGGGAAGCAAGAAGATGCAGAGCTCGTCATCATACCTTTCCCTTTCTCCGGACACATTCTCGCAACAATCGAACTCGCCAAACGTCTCATAAGTCAAGACAATCCTCGGATCCACACCATCACCATCCTCTATTGGGGATTACCTTTTATTCCTCAAGCTGACACAATCGCTTTCCTCCGATCCCTAGTCAAAAATGAGCCTCGTATCCGTCTCGTTACGTTGCCCGAAGTCCAAGACCCTCCACCAATGGAACTCTTTGTGGAATTTGCCGAATCTTACATTCTTGAATACGTCAAGAAAATGGTTCCCATCATCAGAGAAGCTCTCTCCACTCTCTTGTCTTCCCGCGATGAATCGGGTTCAGTTCGTGTGGCTGGATTGGTTCTTGACTTCTTCTGCGTCCCTATGATCGATGTAGGAAACGAGTTTAATCTCCCTTCTTACATTTTCTTGACGTGTAGCGCAGGGTTCTTGGGTATGATGAAGTATCTTCCAGAGAGACACCGCGAAATCAAATCGGAATTCAACCGGAGCTTCAACGAGGAGTTGAATCTCATTCCCGGGTTTGTTAACTCCGTTCCGGTTAAAGTTTTGCCACCGGGTTTGTTCACGACTGAGTCTTACGAAGCTTGGGTCGAAATGGCGGAAAGGTTCCCTGAAGCCAAGGGTATTTTGGTCAATTCATTTGAATCTCTAGAACGTAACGCTTTTGATTATTTCGATCGTCGTCCGGATAATTACCCACCCGTTTACCCAATCGGGCCAATTCTATGCTCCAACGATCGTCCGAATTTGGATTTATCGGAACGAGACCGGATCTTGAAATGGCTCGATGACCAACCCGAGTCATCTGTTGTGTTTCTCTGCTTCGGGAGCTTGAAGAGTCTCGCTGCGTCTCAGATTAAAGAGATCGCTCAAGCCTTAGAGCTCGTCGGAATCAGATTCCTCTGGTCGATTCGAACGGACCCGAAGGAGTACGCGAGCCCGAACGAGATTTTACCGGACGGGTTTATGAACCGAGTCATGGGTTTGGGCCTTGTTTGTGGTTGGGCTCCTCAAGTTGAAATTCTGGCCCATAAAGCAATTGGAGGGTTCGTGTCACACTGCGGTTGGAACTCGATATTGGAGAGTTTGCGTTTCGGAGTTCCAATTGCCACGTGGCCAATGTACGCGGAACAACAACTAAACGCGTTCACGATTGTGAAGGAGCTTGGTTTGGCGTTGGAGATGCGGTTGGATTACGTGTCGGAATATGGAGAAATCGTGAAAGCTGATGAAATCGCAGGAGCCGTACGATCTTTGATGGACGGTGAGGATGTGCCGAGGAGGAAACTGAAGGAGATTGCGGAGGCGGGAAAAGAGGCTGTGATGGACGGTGGATCTTCGTTTGTTGCGGTTAAAAGATTCATAGATGGGCTTTGA

SEQ ID NO: 104 - protein, 474 aa, Artificial Sequence
  Synthetic peptide
MGKQEDAELVIIPFPFSGHILATIELAKRLISQDNPRIHTITILYWGLPFIPQADTIAFLRSLVKNEPRIRLVTLPEVQDPPPMELFVEFAESYILEYVKKMVPIIREALSTLLSSRDESGSVRVAGLVLDFFCVPMIDVGNEFNLPSYIFLTCSAGFLGMMKYLPERHREIKSEFNRSFNEELNLIPGFVNSVPVKVLPPGLFTTESYEAWVEMAERFPEAKGILVNSFESLERNAFDYFDRRPDNYPPVYPIGPILCSNDRPNLDLSERDRILKWLDDQPESSVVFLCFGSLKSLAASQIKEIAQALELVGIRFLWSIRTDPKEYASPNEILPDGFMNRVMGLGLVCGWAPQVEILAHKAIGGFVSHCGWNSILESLRFGVPIATWPMYAEQQLNAFTIVKELGLALEMRLDYVSEYGEIVKADEIAGAVRSLMDGEDVPRRKLKEIAEAGKEAVMDGGSSFVAVKRFIDGL

SEQ ID NO: 105 - DNA, 1374 bp, Arabidopsis thaliana
atgagtgaagcaaagaagggtcacgtactgttttttccatatccattacaaggccacattaacccaatgatccaactcgctaaacgcttatccaaaaagggcatcaccagcacactcatcatcgcctccaaagaccaccgtgaaccttacacctccgacgactactccatcaccgtccacaccatccacgacggtttctttccacatgaacaccctcacgccaagttcgtagatcttgaccgtttccacaactctacttctcgaagcctgaccgatttcatctctagtgcgaagttgtcggacaatcctccaaaagctctgatctatgatccatttatgccctttgcattggacatagccaaggacttggatctatacgtagtggcatatttcactcaaccatggttggctagtcttgtttactaccatatcaacgaaggcacctacgatgttcccgttgatagacacgagaacccaacacttgcatcgtttcctggtttcccattgttaagccaagatgatctgccttcgttcgcctgcgaaaaagggtcgtaccctcttctacacgagtttgtggttaggcaattctctaatttattgcaagctgattgcattctctgcaacacttttgatcaacttgaaccaaaggtagtgaaatggatgaatgatcaatggccggtgaagaacattggaccggtggttccatcgaagttcttggataaccggttgccagaagacaaagattacgaactcgagaactccaagacagagccagacgagtctgttttgaagtggttgggaaacaggccggcgaagtcggtggtttacgtggcgtttgggacattggtggctttgagcgaaaaacagatgaaggaaattgcaatggcgattagccaaaccggatatcacttcttgtggtctgttagagaatccgagagaagcaaactaccctctggttttatcgaagaggcagaggagaaagactctggacttgtggctaagtgggttcctcagctagaggttttagcacatgaatcaatcgggtgtttcgtgtcacactgtggatggaactcgacattggaggcactatgcttaggggttccaatggtgggcgtgcctcagtggactgatcagcccacaaatgctaagtttatagaggatgtgtggaagattggggttagagtgaggaccgatggagaagggctttcgagtaaagaagagattgcgagatgcattgttgaggtcatggaaggagagagagggaaagagataaggaagaatgttgagaagcttaaggtgttggctcgcgaagctatctctgaaggaggtagttccgacaagaagattgatgagtttgttgctcttttgacttaa

SEQ ID NO: 106 - protein, 457 aa, Arabidopsis thaliana
MSEAKKGHVLFFPYPLQGHINPMIQLAKRLSKKGITSTLIIASKDHREPYTSDDYSITVHTIHDGFFPHEHPHAKFVDLDRFHNSTSRSLTDFISSAKLSDNPPKALIYDPFMPFALDIAKDLDLYVVAYFTQPWLASLVYYHINEGTYDVPVDRHENPTLASFPGFPLLSQDDLPSFACEKGSYPLLHEFVVRQFSNLLQADCILCNTFDQLEPKVVKWMNDQWPVKNIGPVVPSKFLDNRLPEDKDYELENSKTEPDESVLKWLGNRPAKSVVYVAFGTLVALSEKQMKEIAMAISQTGYHFLWSVRESERSKLPSGFIEEAEEKDSGLVAKWVPQLEVLAHESIGCFVSHCGWNSTLEALCLGVPMVGVPQWTDQPTNAKFIEDVWKIGVRVRTDGEGLSSKEEIARCIVEVMEGERGKEIRKNVEKLKVLAREAISEGGSSDKKIDEFVALLT

SEQ ID NO: 107 - DNA, 1350 bp, Arabidopsis thaliana
atggagcataagagaggacatgtattagcagtgccgtacccaacgcaaggacacatcacaccattccgccaattctgcaaacgacttcacttcaaaggtctcaaaaccactctcgctctcaccactttcgtcttcaactccatcaatcctgacctatccggtccaatctccatagccaccatctccgatggctatgaccatgggggtttcgagacagctgactccatcgacgactacctcaaagactttaaaacttccggctcgaaaaccattgcagacatcatccaaaaacaccagactagtgataaccccatcacttgtatcgtctatgatgctttcctgccttgggcacttgacgttgctagagagtttggtttagttgcgactcctttctttacgcagccttgtgctgttaactatgtttattatctttcttacataaacaatggaagcttgcaacttcccattgaggaattgccttttcttgagctccaagatttgccttctttcttctctgtttctggctcttatcctgcttactttgagatggtgcttcaacagttcataaatttcgaaaaagctgatttcgttctcgttaatagcttccaagagttggaactgcatgagaatgaattgtggtcgaaagcttgtcctgtgttgacaattggtccaactattccatcaatttacttagaccaacgtatcaaatcagacaccggctatgatcttaatctctttgaatcgaaagatgattccttctgcattaactggctcgacacaaggccacaagggtcggtggtgtacgtagcattcggaagcatggctcagctgactaatgtgcagatggaggagcttgcttcagcagtaagcaacttcagcttcctgtgggtggtcagatcttcagaggaggaaaaactcccatcagggtttcttgagacagtgaataaagaaaagagcttggtcttgaaatggagtcctcagcttcaagttctgtcaaacaaagccatcggttgtttcttgactcactgtggctggaactcaaccatggaggctttgaccttcggggttcccatggtggcaatgccccaatggactgatcaaccgatgaacgcaaagtacatacaagatgtgtggaaggctggagttcgtgtgaagacagagaaggagagtgggattgccaagagagaggagattgagtttagcattaaggaagtgatggaaggagagaggagcaaagagatgaagaagaacgtgaagaaatggagagacttggctgtcaagtcactcaatgaaggaggttctacggatactaacattgatacatttgtatcaagggttcagagcgcctag

SEQ ID NO: 108 - protein, 449 aa, Arabidopsis thaliana
MEHKRGHVLAVPYPTQGHITPFRQFCKRLHFKGLKTTLALTTFVFNSINPDLSGPISIATISDGYDHGGFETADSIDDYLKDFKTSGSKTIADIIQKHQTSDNPITCIVYDAFLPWALDVAREFGLVATPFFTQPCAVNYVYYLSYINNGSLQLPIEELPFLELQDLPSFFSVSGSYPAYFEMVLQQFINFEKADFVLVNSFQELELHENELWSKACPVLTIGPTIPSIYLDQRIKSDTGYDLNLFESKDDSFCINWLDTRPQGSVVYVAFGSMAQLTNVQMEELASAVSNFSFLWVVRSSEEEKLPSGFLETVNKEKSLVLKWSPQLQVLSNKAIGCFLTHCGWNSTMEALTFGVPMVAMPQWTDQPMNAKYIQDVWKAGVRVKTEKESGIAKREEIEFSIKEVMEGERSKEMKKNVKKWRDLAVKSLNEGGSTDTNIDTFVSRVQSA

SEQ ID NO: 109 - DNA, 1383 bp, Stevia rebaudiana
atggcggaacaacaaaagatcaagaaatcaccacacgttctactcatcccattccctttacaaggccatataaaccctttcatccagtttggcaaacgattaatctccaaaggtgtcaaaacaacacttgttaccaccatccacaccttaaactcaaccctaaaccacagtaacaccaccaccacctccatcgaaatccaagcaatttccgatggttgtgatgaaggcggttttatgagtgcaggagaatcatatttggaaacattcaaacaagttgggtctaaatcactagctgacttaatcaagaagcttcaaagtgaaggaaccacaattgatgcaatcatttatgattctatgactgaatgggttttagatgttgcaattgagtttggaatcgatggtggttcgtttttcactcaagcttgtgttgtaaacagcttatattatcatgttcataagggtttgatttctttgccattgggtgaaactgtttcggttcctggatttccagtgcttcaacggtgggagacaccgttaattttgcagaatcatgagcaaatacagagcccttggtctcagatgttgtttggtcagtttgctaatattgatcaagcacgttgggtcttcacaaatagtttttacaagctcgaggaagaggtaatagagtggacgagaaagatatggaacttgaaggtaatcgggccaacacttccatccatgtaccttgacaaacgacttgatgatgataaagataacggatttaatctctacaaagcaaaccatcatgagtgcatgaactggttagacgataagccaaaggaatcagttgtttacgtagcatttggtagcctggtgaaacatggacccgaacaagtggaagaaatcacacgggctttaatagatagtgatgtcaacttcttgtgggttatcaaacataaagaagagggaaagctcccagaaaatctttcggaagtaataaaaaccggaaagggtttgattgtagcatggtgcaaacaattggatgtgttagcacacgaatcagtaggatgctttgttacacattgtgggttcaactcaactcttgaagcaataagtcttggagtccccgttgttgcaatgcctcaattttcggatcaaactacaaatgccaagcttctagatgaaattttgggtgttggagttagagttaaggctgatgagaatgggatagtgagaagaggaaatcttgcgtcatgtattaagatgattatggaggaggaaagaggagtaataatccgaaagaatgcggtaaaatggaaggatttggctaaagtagccgttcatgaaggtggtagctcagacaatgatattgtcgaatttgtaagtgagctaattaaggcttaa

SEQ ID NO: 110 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 111 - DNA, 1371 bp, Arabidopsis thaliana
atggccacttccgtcaatggttcccatcgtcgtccacattacttgcttgtaacattcccagcgcaaggtcacatcaacccggcgcttcaactagccaaccgcctcatccaccacggtgcaaccgtcacatactccaccgcagtctctgctcaccgacgtatgggcgagccaccttccacaaaaggtctatccttcgcttggttcaccgatggattcgacgacggtctcaagtcattcgaagaccagaaaatctacatgtccgaactcaaacgatgtggttcaaacgccctgagagacatcatcaaagccaatcttgacgccaccaccgaaacagagcctatcaccggggtaatctactctgttctcgtcccgtgggtttctacggtagcgcgtgagtttcacctcccaactacacttctctggattgaaccagctactgtactagacatctactactactacttcaacacctcttacaaacatctcttcgacgttgaaccgattaaattaccgaaactgccactgatcaccaccggtgacctcccgtcgtttcttcaaccttcgaaggcattaccgtcagctcttgtgactctaagagaacatatcgaagctctcgaaacggaatcaaaccctaagattcttgttaacacattctctgctttggaacacgatgctttaacctctgttgagaaactcaagatgatcccaatcggaccgttggtttcttcctccgagggtaaaaccgatcttttcaaatcttccgacgaggattacacgaaatggttagactcgaagctcgagagatcagtgatttacatttccttaggcacacacgccgatgatttaccagagaaacacatggaagcgcttactcacggcgtgttagctacaaacagaccgtttttatggatcgtgagggagaaaaatccagaagagaagaagaagaatcggtttcttgaattgatcagaggaagtgatcgaggattggtggtgggatggtgttctcagacagctgttttggcgcattgtgctgtgggatgttttgtgactcattgtggttggaattcgacgttggagagtttagagagtggtgttccggtggttgcgtttccgcagtttgctgatcagtgtacaacggcgaagcttgtggaggatacgtggaggattggagtgaaggtgaaggttggggaggaaggagatgtggatggggaggagattagaaggtgtttggagaaggtgatgagtggtggagaagaggcggaggagatgagagagaatgcagagaagtggaaggcgatggctgttgatgcggcagcggaaggtggaccgtcggatttgaatcttaaaggttttgtggacgaggatgagtag

SEQ ID NO: 112 - protein, 456 aa, Arabidopsis thaliana
MATSVNGSHRRPHYLLVTFPAQGHINPALQLANRLIHHGATVTYSTAVSAHRRMGEPPSTKGLSFAWFTDGFDDGLKSFEDQKIYMSELKRCGSNALRDIIKANLDATTETEPITGVIYSVLVPWVSTVAREFHLPTTLLWIEPATVLDIYYYYFNTSYKHLFDVEPIKLPKLPLITTGDLPSFLQPSKALPSALVTLREHIEALETESNPKILVNTFSALEHDALTSVEKLKMIPIGPLVSSSEGKTDLFKSSDEDYTKWLDSKLERSVIYISLGTHADDLPEKHMEALTHGVLATNRPFLWIVREKNPEEKKKNRFLELIRGSDRGLVVGWCSQTAVLAHCAVGCFVTHCGWNSTLESLESGVPVVAFPQFADQCTTAKLVEDTWRIGVKVKVGEEGDVDGEEIRRCLEKVMSGGEEAEEMRENAEKWKAMAVDAAAEGGPSDLNLKGFVDEDE

SEQ ID NO: 113 - DNA, 1344 bp, Arabidopsis thaliana
atggagactagagaaacaaaaccagtgatctttctcttccctttccctttacaaggtcacttaaacccaatgtttcagctcgccaacatcttcttcaacagaggcttctccatcactgtgatccacactgagttcaactctccaaactcttccaatttccctcatttcactttcgtatccatccccgatagcttgtctgaacctgaatcctatcccgatgtcatcgagattctccatgacctcaattccaagtgtgttgctccttttggtgattgcttaaagaagcttatatctgaagaaccaacagcagcttgtgtgattgttgacgctctttggtacttcactcacgatttaaccgagaaattcaatttcccgaggattgttctccgaaccgttaacctctcagctttcgtcgctttctcaaagtttcatgttttacgagagaaagggtatctttctttacaagagactaaggcagactcaccggttccggagcttccgtatcttagaatgaaggatcttccatggttccagacagaagatccaagatcaggggataagttacagataggtgtgatgaagtcactaaagtcttcctcaggaatcatattcaacgccattgaagatcttgaaacagatcagcttgatgaagcccgcatagaattcccagttccactcttctgtattggaccctttcacaggtacgtttcagcttcatccagtagcttacttgcacacgacatgacttgtctctcctggttagacaagcaagcaacaaattccgtaatctacgcaagtcttggaagcattgcttcgatcgatgaatctgaattcttggagattgcttggggtctaagaaacagcaaccaaccttttctatgggtggttagacccggtttaatccacgggaaagaatggatcgagattctgcctaaagggttcatcgaaaatctcgagggccggggtaaaatagtgaaatgggcacctcagcctgaagttttagctcaccgtgcaacaggcggattcttaacacattgtggatggaactcaacacttgagggcatatgtgaagctataccaatgatatgcagaccatcttttggggaccagagggtgaatgctagatacattaacgatgtttggaagatcggattgcatttggaaaacaaggtagagagactagtgatcgaaaacgcggttagaacactaatgacgagctcggaaggggaagagatccgcaagaggattatgcccatgaaggaaactgttgaacaatgccttaagcttggaggttcatcatttcggaatctcgaaaacttaattgcttatatattgtctttctaa

SEQ ID NO: 114 - protein, 447 aa, Arabidopsis thaliana
METRETKPVIFLFPFPLQGHLNPMFQLANIFFNRGFSITVIHTEFNSPNSSNFPHFTFVSIPDSLSEPESYPDVIEILHDLNSKCVAPFGDCLKKLISEEPTAACVIVDALWYFTHDLTEKFNFPRIVLRTVNLSAFVAFSKFHVLREKGYLSLQETKADSPVPELPYLRMKDLPWFQTEDPRSGDKLQIGVMKSLKSSSGIIFNAIEDLETDQLDEARIEFPVPLFCIGPFHRYVSASSSSLLAHDMTCLSWLDKQATNSVIYASLGSIASIDESEFLEIAWGLRNSNQPFLWVVRPGLIHGKEWIEILPKGFIENLEGRGKIVKWAPQPEVLAHRATGGFLTHCGWNSTLEGICEAIPMICRPSFGDQRVNARYINDVWKIGLHLENKVERLVIENAVRTLMTSSEGEEIRKRIMPMKETVEQCLKLGGSSFRNLENLIAYILSF

SEQ ID NO: 115 - DNA, 1350 bp, Artificial Sequence
  Synthetic oligonucleotide
atggaggaaaagcaagtgaaggagacaaggatagtgttggttccagttccagctcaaggtcatgtaactccgatgatgcaactaggaaaagctcttcactcaaagggtttctccatcactgttgttctgacacagtctaatcgagttagctcttccaaagacttctctgatttccatttcctcaccatcccaggcagcttaactgagtctgatctccaaaacctaggaccacaaaagtttgtgctcaagctcaatcaaatttgtgaggcaagcttcaagcagtgtataggtcaactattgcatgaacaatgtaataatgatattgcttgtgtcgtctacgatgagtacatgtacttctctcatgctgcagtaaaagagtttcaacttcctagtgtcgtctttagcacgacaagtgctactgcttttgtctgtcgctctgttttgtctagagtcaacgcagagtcgttcttgatcgacatgaaagatcctgaaacacaagacaaagtatttccagggttgcatcctctgaggtacaaggatctaccaacttcagtatttgggccaatagagagtacgctcaaggtttacagtgagactgtgaacactcgaacagcttccgctgttatcatcaactcagcaagctgtttagagagctcatctttggcaaggttgcaacaacaactgcaagttccggtgtatcctataggcccacttcatattacagcttcagcgccttctagtttactagaagaagacaggagttgcgttgagtggttgaacaagcaaaaatcaaattcagttatttacataagcttgggaagcttggctctaatggacaccaaagacatgttggagatggcttggggattaagtaatagcaaccaacctttcttatgggtggtcagaccgggctctattccggggtcagaatggacagagtccttaccagaggaattcaataggttggtttcagaaagaggttacattgtgaaatgggctccgcagatggaagttctcagacatcctgcagtaggagggttttggagtcactgtggatggaactcaacagtagagagcatcggggaaggagttccgatgatatgtaggcctttcaccggggatcagaaagtcaatgcgaggtacttagagagagtttggagaattggggttcaattggagggagatctggataaagaaactgtggagagagctgtagagtggttgcttgtggatgaagaaggagcagaaatgaggaagagagccattgacttggaagaaaagattgaaacctctgttagaagtggaggttcctcatgcagctcactagacgactttgttaattccatgtga

SEQ ID NO: 116 - protein, 449 aa, Artificial Sequence
  Synthetic peptide
MEEKQVKETRIVLVPVPAQGHVTPMMQLGKALHSKGFSITVVLTQSNRVSSSKDFSDFHFLTIPGSLTESDLQNLGPQKFVLKLNQICEASFKQCIGQLLHEQCNNDIACVVYDEYMYFSHAAVKEFQLPSVVFSTTSATAFVCRSVLSRVNAESFLIDMKDPETQDKVFPGLHPLRYKDLPTSVFGPIESTLKVYSETVNTRTASAVIINSASCLESSSLARLQQQLQVPVYPIGPLHITASAPSSLLEEDRSCVEWLNKQKSNSVIYISLGSLALMDTKDMLEMAWGLSNSNQPFLWVVRPGSIPGSEWTESLPEEFNRLVSERGYIVKWAPQMEVLRHPAVGGFWSHCGWNSTVESIGEGVPMICRPFTGDQKVNARYLERVWRIGVQLEGDLDKETVERAVEWLLVDEEGAEMRKRAIDLEEKIETSVRSGGSSCSSLDDFVNSM

SEQ ID NO: 117 - DNA, 1422 bp, Artificial Sequence
  Synthetic oligonucleotide
atggctaccagtgactccatagttgacgaccgtaagcagcttcatgttgcgacgttcccatggcttgctttcggtcacatcctcccttaccttcagctttcgaaattgatagctgaaaagggtcacaaagtctcgtttctttctaccaccagaaacattcaacgtctctcttctcatatctcgccactcataaatgttgttcaactcacacttccacgtgtccaagagctgccggaggatgcagaggcgaccactgacgtccaccctgaagatattccatatctcaagaaggcttctgatggtcttcaaccggaggtcacccggtttctagaacaacactctccggactggattatttatgattatactcactactggttgccatccatcgcggctagcctcggtatctcacgagcccacttctccgtcaccactccatgggccattgcttatatgggaccctcagctgacgccatgataaatggttcagatggtcgaaccacggttgaggatctcacgacaccgcccaagtggtttccctttccgaccaaagtatgctggcggaagcatgatcttgcccgactggtgccttacaaagctccggggatatctgatggataccgtatggggatggttcttaagggatctgattgtttgctttccaaatgttaccatgagtttggaactcaatggctacctcttttggagacactacaccaagtaccggtggttccggtgggattactgccaccggaaatacccggagacgagaaagatgaaacatgggtgtcaatcaagaaatggctcgatggtaaacaaaaaggcagtgtggtgtacgttgcattaggaagcgaggctttggtgagccaaaccgaggttgttgagttagcattgggtctcgagctttctgggttgccatttgtttgggcttatagaaaaccaaaaggtcccgcgaagtcagactcggtggagttgccagacgggttcgtggaacgaactcgtgaccgtgggttggtctggacgagttgggcacctcagttacgaatactgagccatgagtcggtttgtggtttcttgactcattgtggttctggatcaattgtggaagggctaatgtttggtcaccctctaatcatgctaccgatttttggggaccaacctctgaatgctcgattactggaggacaaacaggtgggaatcgagataccaagaaatgaggaagatggttgcttgaccaaggagtcggttgctagatcactgaggtccgttgttgtggaaaaagaaggggagatctacaaggcgaacgcgagggagctgagtaaaatctataacgacactaaggttgaaaaagaatatgtaagccaattcgtagactatttggaaaagaatgcgcgtgcggttgccatcgatcatgagagttaa

SEQ ID NO: 118 - protein, 473 aa, Artificial Sequence
  Synthetic peptide
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGMVLKGSDCLLSKCYHEFGTQWLPLLETLHQVPVVPVGLLPPEIPGDEKDETWVSIKKWLDGKQKGSVVYVALGSEALVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 119 - DNA, 1458 bp, Artificial Sequence
  Synthetic oligonucleotide
atgggtgctgaaccacaacaattgcatgttgttttcttcccaattatggcccatggtcatatgattccaactttggatattgccagattattcgctgctagaaacgttagagctaccattattactactccattgaacgctcatactttcaccaaggctattgaaatgggtaagaagaatggttccccaaccattcacttggaattattcaaattcccagcccaagatgttggtttgcctgaaggttgtgaaaatttggaacaagctttgggttcctccttgatcgaaaaattctttaaaggtgtcggtttgttgagagaacaattggaagcttacttggaaaagaccagaccaaactgtttagttgccgatatgttttttccatgggctactgattctgctgccaagtttaacattccaagattggtttttcacggtacatctttcttttctttgtgcgccttggaagtcgtcagattatatgaaccacataagaacgtcagttccgacgaagaattattctctttgcctttgttcccacacgacatcaaaatgatgagattgcaattacctgaagatgtctggaaacacgaaaaagctgaaggtaagactagattgaagttgatcaaagaatccgaattgaagtcctacggtgttatcgttaactccttctatgaattggaacctaactacgccgaattcttcagaaaagaattgggtagaagagcctggaatattggtccagtttcattgtgtaacagatccactgaagataaggctcaaagaggtaagcaaacctccattgatgaacatgaatgtttgaagtggttgaactccaagaagaagaactccgttatctacatttgtttcggttctaccgctcatcaaattgccccacaattatacgaaattgctatggctttagaagcctccggtcaagaattcatttgggttgttagaaacaacaacaacaacgacgatgacgatgatgactcttggttgccaagaggttttgaacaaagagttgaaggtaaaggtttgatcattagaggttgggctccacaagttttgattttggaacatgaagctattggtgccttcgttactcattgtggttggaattctactttggaaggtattactgctggtgttccaatggttacttggccaattttcgctgaacaattctacaacgaaaagttggtcaatcaaatcttgaagatcggtgttccagttggtgctaacaaatggtctagagaaacctctatcgaagatgtcattaagaaggatgccattgaaaaggccttgagagaaattatggttggtgatgaagctgaagaaagaagatccagagccaaaaagttgaaagaaatggcttggaaggctgttgaagaaggtggttcttcttattctgatttgtccgccttgattgaagaattgagaggttatcatgcctaa

SEQ ID NO: 120 - protein, 485 aa, Phytolacca americana
MGAEPQQLHVVFFPIMAHGHMIPTLDIARLFAARNVRATIITTPLNAHTFTKAIEMGKKNGSPTIHLELFKFPAQDVGLPEGCENLEQALGSSLIEKFFKGVGLLREQLEAYLEKTRPNCLVADMFFPWATDSAAKFNIPRLVFHGTSFFSLCALEVVRLYEPHKNVSSDEELFSLPLFPHDIKMMRLQLPEDVWKHEKAEGKTRLKLIKESELKSYGVIVNSFYELEPNYAEFFRKELGRRAWNIGPVSLCNRSTEDKAQRGKQTSIDEHECLKWLNSKKKNSVIYICFGSTAHQIAPQLYEIAMALEASGQEFIWVVRNNNNNDDDDDDSWLPRGFEQRVEGKGLIIRGWAPQVLILEHEAIGAFVTHCGWNSTLEGITAGVPMVTWPIFAEQFYNEKLVNQILKIGVPVGANKWSRETSIEDVIKKDAIEKALREIMVGDEAEERRSRAKKLKEMAWKAVEEGGSSYSDLSALIEELRGYHA

SEQ ID NO: 121 - DNA, 1407 bp, Artificial Sequence
  Synthetic oligonucleotide
atgcaccatcatcatcatcattctggatccatggaaaatgaaacggagaccaccgttcgccggcgccggagaataatattattcccggtaccatttcaaggccacgttaacccaattcttcagctagccaatgtgttgtactctaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcacggatgctctttggtacttcgcgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaaaagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttcaatggttagaccaacaaccgccaagttcggtactgtatgttagttttggtagtactagtgaagtggatgagaaagatttcttggaaatagctcgtgggttggttgatagcaagcagtcgtttttatgggtggttcgacctgggtttgtcaagggttcgacgtgggtcgaaccgttgccagatgggttcttgggtgaaagaggacgtattgtgaaatgggttccacagcaagaagtgctagctcatggagcaataggcgcattctggactcatagcggatggaactctacgttggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaataagaagagttatggtggatgaagaaggagaatacattagacagaatgcaagagttttgaaacaaaaggcagatgtttctttgatgaagggtggttcgtcttacgaatcattagagtctctagtttcttacatttcatcgttgtag

SEQ ID NO: 122 - protein, 468 aa, Artificial Sequence
  Synthetic peptide
MHHHHHHSGSMENETETTVRRRRRIILFPVPFQGHVNPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 123 - DNA, 1347 bp, Saccharomyces cerevisiae
atgctttcgcttaaaacgttactgtgtacgttgttgactgtgtcatcagtactcgctaccccagtccctgcaagagacccttcttccattcaatttgttcatgaggagaacaagaaaagatactacgattatgaccacggttccctcggagaaccaatccgtggtgtcaacattggtggttggttacttcttgaaccatacattactccatctttgttcgaggctttccgtacaaatgatgacaacgacgaaggaattcctgtcgacgaatatcacttctgtcaatatttaggtaaggatttggctaaaagccgtttacagagccattggtctactttctaccaagaacaagatttcgctaatattgcttcccaaggtttcaaccttgtcagaattcctatcggttactgggctttccaaactttggacgatgatccttatgttagcggcctacaggaatcttacctagaccaagccatcggttgggctagaaacaacagcttgaaagtttgggttgatttgcatggtgccgctggttcgcagaacgggtttgataactctggtttgagagattcatacaagtttttggaagacagcaatttggccgttactacaaatgtcttgaactacatattgaaaaaatactctgcggaggaatacttggacactgttattggtatcgaattgattaatgagccattgggtcctgttctagacatggataaaatgaagaatgactacttggcacctgcttacgaatacttgagaaacaacatcaagagtgaccaagttatcatcatccatgacgctttccaaccatacaattattgggatgacttcatgactgaaaacgatggctactggggtgtcactatcgaccatcatcactaccaagtctttgcttctgatcaattggaaagatccattgatgaacatattaaagtagcttgtgaatggggtaccggagttttgaatgaatcccactggactgtttgtggtgagtttgctgccgctttgactgattgtacaaaatggttgaatagtgttggcttcggcgctagatacgacggttcttgggtcaatggtgaccaaacatcttcttacattggctcttgtgctaacaacgatgatatagcttactggtctgacgaaagaaaggaaaacacaagacgttatgtggaggcacaactagatgcctttgaaatgagagggggttggattatctggtgttacaagacagaatctagtttggaatgggatgctcaaagattgatgttcaatggtttattccctcaaccattgactgacagaaagtatccaaaccaatgtggcacaatttctaactaa

SEQ ID NO: 124 - protein, 448 aa, Saccharomyces cerevisiae
MLSLKTLLCTLLTVSSVLATPVPARDPSSIQFVHEENKKRYYDYDHGSLGEPIRGVNIGGWLLLEPYITPSLFEAFRTNDDNDEGIPVDEYHFCQYLGKDLAKSRLQSHWSTFYQEQDFANIASQGFNLVRIPIGYWAFQTLDDDPYVSGLQESYLDQAIGWARNNSLKVWVDLHGAAGSQNGFDNSGLRDSYKFLEDSNLAVTTNVLNYILKKYSAEEYLDTVIGIELINEPLGPVLDMDKMKNDYLAPAYEYLRNNIKSDQVIIIHDAFQPYNYWDDFMTENDGYWGVTIDHHHYQVFASDQLERSIDEHIKVACEWGTGVLNESHWTVCGEFAAALTDCTKWLNSVGFGARYDGSWVNGDQTSSYIGSCANNDDIAYWSDERKENTRRYVEAQLDAFEMRGGWIIWCYKTESSLEWDAQRLMFNGLFPQPLTDRKYPNQCGTISN

SEQ ID NO: 125 - DNA, 942 bp, Saccharomyces cerevisiae
atgcgtttctctactacactcgctactgcagctactgcgctatttttcacagcctcccaagtttcagctattggtgaactagcctttaacttgggtgtcaagaacaacgatggtacttgtaagtccacttccgactatgaaaccgaattacaagctttgaagagctacacttccaccgtcaaagtttacgctgcctcagattgtaacactttgcaaaacttaggtcctgctgctgaagctgagggatttactatctttgtcggtgtttggccaacagacgacagtcattacgctgctgaaaaggctgctttgcaaacctatttgccaaaaattaaagaatccactgttgctggtttcttggttggttctgaagccttataccgtaacgatttgactgcctctcaattatcagacaaaattaatgacgtccgtagtgtcgttgctgacatttccgattctgacggaaagtcatactctggtaagcaagtcggtactgtcgattcctggaatgttttggttgctggttacaattctgccgttatcgaagcttccgattttgttatggctaacgcgttctcctactggcaaggtcaaaccatgcaaaatgcctcttactcattctttgatgatattatgcaagctctacaggttatccaatctactaaaggttctaccgatattaccttctgggttggtgagaccggttggccaactgatggtaccaactttgaaagttcttacccatctgttgacaacgccaaacaattctggaaagaaggtatctgttccatgagagcttggggtgttaacgttattgtttttgaagcctttgatgaagattggaagccaaacacctctggtacctctgatgtcgagaagcactggggtgttttcacttcaagtgacaatttgaaatactccttggactgtgacttttcatga

SEQ ID NO: 126 - protein, 313 aa, Saccharomyces cerevisiae
MRFSTTLATAATALFFTASQVSAIGELAFNLGVKNNDGTCKSTSDYETELQALKSYTSTVKVYAASDCNTLQNLGPAAEAEGFTIFVGVWPTDDSHYAAEKAALQTYLPKIKESTVAGFLVGSEALYRNDLTASQLSDKINDVRSVVADISDSDGKSYSGKQVGTVDSWNVLVAGYNSAVIEASDFVMANAFSYWQGQTMQNASYSFFDDIMQALQVIQSTKGSTDITFWVGETGWPTDGTNFESSYPSVDNAKQFWKEGICSMRAWGVNVIVFEAFDEDWKPNTSGTSDVEKHWGVFTSSDNLKYSLDCDFS

SEQ ID NO: 127 - DNA, 1689 bp, Saccharomyces cerevisiae
atgcctttgaagtcgttttttttttcagcatttctagttttatgcctgtctaaattcacgcaaggcgttggcaccacagagaaggaagaatcgttatcgcctttggaactaaatattttacaaaacaaattcgcctcctactatgcaaacgacactatcaccgtgaaaggtattactattggcggctggctagtaacagaaccttatatcacgccatcattatatcgtaatgctacgtcactggcaaaacagcaaaactcttccagcaatatctccattgtcgacgaatttactctttgtaaaaccttaggatataacacctctctaactttattggataatcacttcaaaacttggattacagaggatgattttgaacaaatcaaaaccaacggtttcaatttagttaggatccccatcggatattgggcgtggaaacaaaatactgataaaaacttgtacatcgataacataactttcaatgatccatacgtaagtgatggattacaactgaaatatttaaataatgctctcgaatgggcgcaaaagtacgaactaaatgtatggttagatctacatggtgctcctggatcccagaatggattcgataattccggtgaaagaatactctatggcgatttaggctggttaaggttgaataatactaaagaactgactctggctatttggagagatatgttccagacatttttaaataaaggtgacaaaagtcctgtggtgggtattcaaatcgtcaacgaaccgcttggtggcaaaatcgatgtttcagacataacggagatgtattacgaagcatttgacttgctcaagaaaaatcagaattcgagtgacaacactacgtttgttattcatgacggttttcaaggaatcggtcactggaacttggagctaaacccaacctaccagaatgtatcgcatcattatttcaatttgactggtgcaaattacagctctcaagatatattggtcgaccatcatcattatgaagtgtttactgatgcgcaattggccgaaactcagtttgcacgtattgaaaacattatcaattatggggactctatccacaaagaactttcttttcacccagcagtagtcggagaatggtcaggcgctattactgattgtgcaacctggctaaatggtgttggggtgggtgcacgttacgatggatcatactacaatacaacgttgtttaccaccaacgacaagccagttggaacatgtatatcccaaaatagcttagctgattggacgcaagattaccgtgaccgtgtgagacaattcattgaggcacagctagccacttattcgtcaaaaacaacgggatggattttttggaattggaagaccgaagacgccgtagaatgggattatttgaagctaaaagaagctaaccttttcccttcccctttcgacaactacacgtacttcaaagcagatggatctatcgaagaaaaattctcatcctctttatcagcacaggcatttccaagaacaacgtcatcggttttgtcctccactacgacttccaggaagagtaagaatgctgcaatttctaataaactaacaacttcgcagctattaccaatcaaaaatatgagtttgacctggaaagcgagcgtatgcgcactcgctatcaccattgccgctctttgcgcttctctttaa

SEQ ID NO: 128 - protein, 562 aa, Saccharomyces cerevisiae
MPLKSFFFSAFLVLCLSKFTQGVGTTEKEESLSPLELNILQNKFASYYANDTITVKGITIGGWLVTEPYITPSLYRNATSLAKQQNSSSNISIVDEFTLCKTLGYNTSLTLLDNHFKTWITEDDFEQIKTNGFNLVRIPIGYWAWKQNTDKNLYIDNITFNDPYVSDGLQLKYLNNALEWAQKYELNVWLDLHGAPGSQNGFDNSGERILYGDLGWLRLNNTKELTLAIWRDMFQTFLNKGDKSPVVGIQIVNEPLGGKIDVSDITEMYYEAFDLLKKNQNSSDNTTFVIHDGFQGIGHWNLELNPTYQNVSHHYFNLTGANYSSQDILVDHHHYEVFTDAQLAETQFARIENIINYGDSIHKELSFHPAVVGEWSGAITDCATWLNGVGVGARYDGSYYNTTLFTTNDKPVGTCISQNSLADWTQDYRDRVRQFIEAQLATYSSKTTGWIFWNWKTEDAVEWDYLKLKEANLFPSPFDNYTYFKADGSIEEKFSSSLSAQAFPRTTSSVLSSTTTSRKSKNAAISNKLTTSQLLPIKNMSLTWKASVCALAITIAALCASL

SEQ ID NO: 129 - DNA, 1338 bp, Saccharomyces cerevisiae
atggtttcgttcagagggctgactacactaacactactttttaccaaattagtaaactgtaatcctgtttccacaaaaaatagggactctatacagtttatttataaagaaaaggatagtatatactctgccatcaacaatcaagccatcaatgaaaaaattcatggagtcaatttgggtgggtggctagtattggagccgtatattacaccttctttattcgagactttccgtactaatccgtacaacgatgacggtattcctgttgatgaataccatttttgtgaaaaattaggctatgaaaaggcaaaggaacgcctttatagtcattggtcgacgttctataaagaggaagacttcgcgaaaattgcttctcaaggcttcaatttggttagaattcctattgggtattgggcctttacaacgttgagtcatgatccctatgttaccgccgagcaggaatattttctagaccgggctatcgattgggctaggaaatatggtttgaaagtatggattgatcttcatggagccgctggttcacaaaacggatttgataactcagggttgagggattcatataagtttctggaagatgaaaatttaagcgccaccatgaaagctttaacgtatattttaagcaaatactcaacagacgtatacctggacaccgttattggaatcgaattgctcaatgaaccgttaggtccagttattgacatggaaagattgaaaaatttgcttttgaagccggcttatgactatttgagaaataaaattaatagcaaccagatcattgtaatacatgatgcttttcaaccttaccattattgggatgggtttttgaatgatgaaaagaacgaatatggcgtcataattgaccatcatcattatcaggtgttctcgcaggtggaattaacaagaaaaatgaatgaacgtatcaaaatcgcctgccaatgggggaaagatgccgtaagcgagaagcattggtccgtagcaggcgaattttcagcggccttaacagattgtacaaagtggttaaatggggttggtctaggtgccagatatgatggaagttggaccaaagacaatgaaaaatctcattacataaatacgtgtgcaaacaacgagaatattgccttgtggcccgaagagagaaaacagaacactagaaagtttatcgaggctcaattagatgcttttgaaatgactggtggatggataatgtggtgttacaagacagagaatagtatcgaatgggatgttgaaaaattgattcaactcaacatttttccgcaacctatcaacgataggaaatatcctaaccaatgtcattga

SEQ ID NO: 130 - protein, 445 aa, Saccharomyces cerevisiae
MVSFRGLTTLTLLFTKLVNCNPVSTKNRDSIQFIYKEKDSIYSAINNQAINEKIHGVNLGGWLVLEPYITPSLFETFRTNPYNDDGIPVDEYHFCEKLGYEKAKERLYSHWSTFYKEEDFAKIASQGFNLVRIPIGYWAFTTLSHDPYVTAEQEYFLDRAIDWARKYGLKVWIDLHGAAGSQNGFDNSGLRDSYKFLEDENLSATMKALTYILSKYSTDVYLDTVIGIELLNEPLGPVIDMERLKNLLLKPAYDYLRNKINSNQIIVIHDAFQPYHYWDGFLNDEKNEYGVIIDHHHYQVFSQVELTRKMNERIKIACQWGKDAVSEKHWSVAGEFSAALTDCTKWLNGVGLGARYDGSWTKDNEKSHYINTCANNENIALWPEERKQNTRKFIEAQLDAFEMTGGWIMWCYKTENSIEWDVEKLIQLNIFPQPINDRKYPNQCH

SEQ ID NO: 131 - DNA, 2340 bp, Saccharomyces cerevisiae
atgtgttacagtaggcaagccattcctccacctgttcctaatagacctggcggtactaccaatagaggacctcctcccttaccacctcgcgcaaatgttcagccaccagtttgttcttctgagaactccagtaaacctcgagaaaatagggtggcaggtgaatcactgcgcacaccaagcagttctaaccctttagctgacagccaggtgaatagtgacaatatttttcaatcacctgtcctttctaacttaaaagctcctccttccgtcttcaacaaagttcaacacccggtaccaaagccgaatattgatgatcaatctgttgatccattggagacaaacaagttctacacaaacatgttattagacgataatacacaacctatatggacacatccttattcaatttggttttctcgtgatccagaattgtttggtttggccgccaatcatactttagcgtctcaaagggtttttgatacaactacaaatcctcccagattctacttcaatcccacaaatataaaatcatttgtattcaaggccagggaatttgtctcttcaaatgatatcaagcttgaattccgggatatgaaacatatgtccatgtgcttactaatgtctttgagtagttcccagtttatagaattcccgctggttcaggggatgggctttgtcacggctatttaccacgatttgggttttgaattaagaagcgccgttggtttcaggagcttagagcgcataagcgtgaatgaaagatatggtaaatataatatacaacttgagaataataggaattggatactttatttgacctcacctgattactcttttccacaagattttcaaatttcattactcgacagcaatacaataatatcatctcataaaatcaatggtcttatctgtcaattatctgcagactccgtccccagtattgacatggcagcgggctgttatccagtatactgtgacctatcagggcaaactgttgatgaacatttcacaaattacagatttaactatacggttgcaggctactcgcagtctggaactactctaatgtatgctttaccacatcataaagcggcattcacaccagaaatgcaggaacgtgaaattgcctcaagcctggattccacagttaaaggcttaatgactggttatctcacaaacagttttgatatgcaggttcaagtacctcaagaattgggctttgaacctgtcgcgttatctttgaataagaaggcagattacagtcaagaaaagctatctaaaattcgggaagctgctgttcaagaagttcaattgagtgaccctcaacaagaatcaaacatagattctatgtatttctctggtaagatacttgcaaagtatgcgtggattctttatgtgacacattacatccttcatgatgaaaatttaacaaaagaattattatctaaactgactattgcaatggagagattcatcagcaatcagcaagtcttacctttaaattacgacgttagttggaaaggcataatttcatcaggctcttctagccaagactttggtaactcttattataatgatcatcattttcattactcttatcatgtcataacggctgccattatctctttagtcgattctgatttaagtggagttactaacaactcctggttagaaaataatagagattgggttgagtgcttaattagagattactctggtgttgacaacgatgatccgtacttccctcaatttaggtcatttgattggtttaatggccattcatgggccaaaggactatttccaagtggtgacggtaaagatgaagaatctacttcagaagatgtcaattcttgctatgcgataaaattgtgggggttggtaactggaaattctaagttaactgacatagcgaacttacaactcggcatcatgagaaatgttttccagagctacttcctatacgaaagcaataatactgtccaaccgaaagaatttattggtaataaagttagtggtattttattcgaaaataaaattgatcacgccacgtattttggcatggagcctcaatacattcatatgattcacgccattcctataacatcagcatcttcatgggtcagaacaccaaattttgtcaaagaggagtgggaagaaaaaatgcagccgataattgatcaagtgaatgacggttggaaaggaataatcatgttaaatatggctctgcttgatccaaaattttcgtacgactttttcagccaacctgatttcaacagaaatttcctagacaatgggcaaagcttaacttggtctttagcttattcaggtgctttttcttag

SEQ ID NO: 132 - protein, 2340 aa, Saccharomyces cerevisiae
ATGTGTTACAGTAGGCAAGCCATTCCTCCACCTGTTCCTAATAGACCTGGCGGTACTACCAATAGAGGACCTCCTCCCTTACCACCTCGCGCAAATGTTCAGCCACCAGTTTGTTCTTCTGAGAACTCCAGTAAACCTCGAGAAAATAGGGTGGCAGGTGAATCACTGCGCACACCAAGCAGTTCTAACCCTTTAGCTGACAGCCAGGTGAATAGTGACAATATTTTTCAATCACCTGTCCTTTCTAACTTAAAAGCTCCTCCTTCCGTCTTCAACAAAGTTCAACACCCGGTACCAAAGCCGAATATTGATGATCAATCTGTTGATCCATTGGAGACAAACAAGTTCTACACAAACATGTTATTAGACGATAATACACAACCTATATGGACACATCCTTATTCAATTTGGTTTTCTCGTGATCCAGAATTGTTTGGTTTGGCCGCCAATCATACTTTAGCGTCTCAAAGGGTTTTTGATACAACTACAAATCCTCCCAGATTCTACTTCAATCCCACAAATATAAAATCATTTGTATTCAAGGCCAGGGAATTTGTCTCTTCAAATGATATCAAGCTTGAATTCCGGGATATGAAACATATGTCCATGTGCTTACTAATGTCTTTGAGTAGTTCCCAGTTTATAGAATTCCCGCTGGTTCAGGGGATGGGCTTTGTCACGGCTATTTACCACGATTTGGGTTTTGAATTAAGAAGCGCCGTTGGTTTCAGGAGCTTAGAGCGCATAAGCGTGAATGAAAGATATGGTAAATATAATATACAACTTGAGAATAATAGGAATTGGATACTTTATTTGACCTCACCTGATTACTCTTTTCCACAAGATTTTCAAATTTCATTACTCGACAGCAATACAATAATATCATCTCATAAAATCAATGGTCTTATCTGTCAATTATCTGCAGACTCCGTCCCCAGTATTGACATGGCAGCGGGCTGTTATCCAGTATACTGTGACCTATCAGGGCAAACTGTTGATGAACATTTCACAAATTACAGATTTAACTATACGGTTGCAGGCTACTCGCAGTCTGGAACTACTCTAATGTATGCTTTACCACATCATAAAGCGGCATTCACACCAGAAATGCAGGAACGTGAAATTGCCTCAAGCCTGGATTCCACAGTTAAAGGCTTAATGACTGGTTATCTCACAAACAGTTTTGATATGCAGGTTCAAGTACCTCAAGAATTGGGCTTTGAACCTGTCGCGTTATCTTTGAATAAGAAGGCAGATTACAGTCAAGAAAAGCTATCTAAAATTCGGGAAGCTGCTGTTCAAGAAGTTCAATTGAGTGACCCTCAACAAGAATCAAACATAGATTCTATGTATTTCTCTGGTAAGATACTTGCAAAGTATGCGTGGATTCTTTATGTGACACATTACATCCTTCATGATGAAAATTTAACAAAAGAATTATTATCTAAACTGACTATTGCAATGGAGAGATTCATCAGCAATCAGCAAGTCTTACCTTTAAATTACGACGTTAGTTGGAAAGGCATAATTTCATCAGGCTCTTCTAGCCAAGACTTTGGTAACTCTTATTATAATGATCATCATTTTCATTACTCTTATCATGTCATAACGGCTGCCATTATCTCTTTAGTCGATTCTGATTTAAGTGGAGTTACTAACAACTCCTGGTTAGAAAATAATAGAGATTGGGTTGAGTGCTTAATTAGAGATTACTCTGGTGTTGACAACGATGATCCGTACTTCCCTCAATTTAGGTCATTTGATTGGTTTAATGGCCATTCATGGGCCAAAGGACTATTTCCAAGTGGTGACGGTAAAGATGAAGAATCTACTTCAGAAGATGTCAATTCTTGCTATGCGATAAAATTGTGGGGGTTGGTAACTGGAAATTCTAAGTTAACTGACATAGCGAACTTACAACTCGGCATCATGAGAAATGTTTTCCAGAGCTACTTCCTATACGAAAGCAATAATACTGTCCAACCGAAAGAATTTATTGGTAATAAAGTTAGTGGTATTTTATTCGAAAATAAAATTGATCACGCCACGTATTTTGGCATGGAGCCTCAATACATTCATATGATTCACGCCATTCCTATAACATCAGCATCTTCATGGGTCAGAACACCAAATTTTGTCAAAGAGGAGTGGGAAGAAAAAATGCAGCCGATAATTGATCAAGTGAATGACGGTTGGAAAGGAATAATCATGTTAAATATGGCTCTGCTTGATCCAAAATTTTCGTACGACTTTTTCAGCCAACCTGATTTCAACAGAAATTTCCTAGACAATGGGCAAAGCTTAACTTGGTCTTTAGCTTATTCAGGTGCTTTTTCTTAG

SEQ ID NO: 133 - DNA, 3354 bp, Saccharomyces cerevisiae
atgcaattatatctgacacttctttttctattaagtttcgtcgaatgttcatatataagtttcatatcgaataatgcagacgaaatattggaaactgatttaattgaaactttatcttacgctactttgacagtaggtgagccttatgttgcacaatcggtggttgtaacaagagtatctgcagcttctcatagccctctctcagtttcgccaaaaaatagagtgagcgcatctccaattaattcacaagacagtgattctaatacaagaacagcagttcaactgtcattgtcgttatcaaactacgcaagccaagtatctcaaaaaataagcgcacaaaccaataatgatcctgtcactgtttcaaatatttacgcaaatgataattcgaaaagcaaaagttcagtacacaatttgagttcggtttccggcgtcgcctcagtcatgccaagtgcatcaacaatgcgtaaagtcaccacattattatctcaaactgcttcgacttcgacttcgactttattttcttcatctttatctatttcggggacacaattaaatggaactttattgacttctgtttcaaaaggtacaatcgatccactggtaactcaaatgccttcctattcttcccaagaaacaaaaattattccttcttcattgacctcgaataaaacgatatatactatttcagtaaggacaaatgcagctacagctaccggcgaagattccttcattgcttcaactcctgcttcctcgactttgttctatccatccaactcaacccaggatttagtccagacgctcgcatctacaacagcaagccctgcttacccatctaataggacacaaatcactctttctccatctgtatcactatattcgacaactagtccaatttacccttcaaacattacagaaaacggttcttcaccatcaccatcactatcatcgactgttagtccagtttatccatcgagttcaacaggaaacattcttttgtcatcactattttcaacagtagattcttcttcctctccggtttcttcgacattagataccatttatgtatcttcttcaatgcaagctaccatctcttcgtcctcatcttcgagacaaacaaaaacttcctcctcttctctgtcaacctcgaccagtagcaccgctaccacaacggagaactcttcaacaacgaccattgtcaatcttttcaatgctgtttcgacagatgaaccaccaactgtttttgacagatcaccaaatcctatgtcattggctgatggggtgtcaaacgatggtcccattcaaacaaacaagttttatacaaatttgattgttggtagtcaggaatcacctgcctttgtgtatccttattcattgtggaagtacacctcgagttcatatggttttgctgttcaacatacaacagtagatcaatatagctacggtggttatgacagttccggtaatgcagaatatttggtcaatcctttgggaattgctcatgttgttttctcagcttcgaattttgattcaagtatgactatgcaagttgatgaaatgaccttgtcttcaacaagggtagtgctatcagaatccaatgattcttcgaactacctggaaatacctttggttcaaggtatgggatttgcaactggtatatatcatggatctttaaatgccaagattggttccagcgtcggattcaataccattgtttctgaatcatcaagcaatttagctcagggtattctcaaatatcgtatcacccttttgaatggagtcacttggttatgttacgtgataggaccagacgatttaacctcaacggacttttcattggaggtgagctcagaatatgaaattaaagcaagcgctagtgttgatggtcttatcattcaattggccgtcgcaccttctgaaactgattacgaggtcttttatgatcaagccgcaggtatgtacgttactaatttcaagctgcagggtgtctctgacggttctacagctacctatgaattttcttataccacccaaggtgagtcggcatcaggtagcacaatgatttttgccttgccccatcatgaatcttcatttagtgatataatgcaagactactataccggcattcaactcgcttctacaactaagggtgtaatgaacggttatctaacaacgagcctacaattttccacatctctaaacagacaaatatcttggttaccatggtcttctcaacttggctcaaaccttttagagtattctaaagaacaattgcaattattagctgaagttgccaattccgaattacaggttagtatttccgaaagtataagcgggttaaacacatattatttgggtaaagttatcgataagtattcttacattctattaacggtctccgaaatcatccaagatgaagccagtactaagagtactttggaaaacatcaaatcggcctttgatattctgctacaaaatgaacagacgtatccactaatttacgacacaaagttcaacggtttggtcagttcaggagattggggttccaccagcacgcaatacgattttggtaacacttattacaatgaccatcatttccattatggttacattattcatgcagctgctgtcatcggctatgttgactctaaactgaatggtacttgggctgccgacaataaagattgggtcaattcattggtaagagatgtcgcaaatccatctgaaaaagatgaatactttgcacaatcgagaatgtttgattggttcaacggtcattcatgggcagctggactttatgaaaacggtaacggtaagaacgaagaaagtagtagtgaagattacaattttgcctatgctatgaagttatggggggccactatcggtgaccagtcaatggaattgaggggtgacttgatgattagtataatgaaggatgcaatgaacgactatttctattatcaaaatgacaacacagttgagcctgaagaaatcataggaaataaagtgagtggtattttattcgataatattatcgattatactacttattttggaacaaacacagaatatatccacggtattcatatgctacctatcacaccagtttcttctaatattcgttctgagactttcgtcgaagaagaatggcagactaaaatcgagccaattattgaatcgatagaaagcggctggacaggcatattgaagctgaatcaagcactcttcgacccagtagattcgtatgcatttttcagtgattcaacttttgattcatccacatatttggataacggaatgagtcgcacatgggcattagcattttcagggggactggccaactcaattgcttag

SEQ ID NO: 134 - protein, 1117 aa, Saccharomyces cerevisiae
MQLYLTLLFLLSFVECSYISFISNNADEILETDLIETLSYATLTVGEPYVAQSVVVTRVSAASHSPLSVSPKNRVSASPINSQDSDSNTRTAVQLSLSLSNYASQVSQKISAQTNNDPVTVSNIYANDNSKSKSSVHNLSSVSGVASVMPSASTMRKVTTLLSQTASTSTSTLFSSSLSISGTQLNGTLLTSVSKGTIDPLVTQMPSYSSQETKIIPSSLTSNKTIYTISVRTNAATATGEDSFIASTPASSTLFYPSNSTQDLVQTLASTTASPAYPSNRTQITLSPSVSLYSTTSPIYPSNITENGSSPSPSLSSTVSPVYPSSSTGNILLSSLFSTVDSSSSPVSSTLDTIYVSSSMQATISSSSSSRQTKTSSSSLSTSTSSTATTTENSSTTTIVNLFNAVSTDEPPTVFDRSPNPMSLADGVSNDGPIQTNKFYTNLIVGSQESPAFVYPYSLWKYTSSSYGFAVQHTTVDQYSYGGYDSSGNAEYLVNPLGIAHVVFSASNFDSSMTMQVDEMTLSSTRVVLSESNDSSNYLEIPLVQGMGFATGIYHGSLNAKIGSSVGFNTIVSESSSNLAQGILKYRITLLNGVTWLCYVIGPDDLTSTDFSLEVSSEYEIKASASVDGLIIQLAVAPSETDYEVFYDQAAGMYVTNFKLQGVSDGSTATYEFSYTTQGESASGSTMIFALPHHESSFSDIMQDYYTGIQLASTTKGVMNGYLTTSLQFSTSLNRQISWLPWSSQLGSNLLEYSKEQLQLLAEVANSELQVSISESISGLNTYYLGKVIDKYSYILLTVSEIIQDEASTKSTLENIKSAFDILLQNEQTYPLIYDTKFNGLVSSGDWGSTSTQYDFGNTYYNDHHFHYGYIIHAAAVIGYVDSKLNGTWAADNKDWVNSLVRDVANPSEKDEYFAQSRMFDWFNGHSWAAGLYENGNGKNEESSSEDYNFAYAMKLWGATIGDQSMELRGDLMISIMKDAMNDYFYYQNDNTVEPEEIIGNKVSGILFDNIIDYTTYFGTNTEYIHGIHMLPITPVSSNIRSETFVEEEWQTKIEPIIESIESGWTGILKLNQALFDPVDSYAFFSDSTFDSSTYLDNGMSRTWALAFSGGLANSIA

SEQ ID NO: 135 - DNA, 1629 bp, Saccharomyces cerevisiae
atgatttcacccataagctttctatcatcgctgctatgcttaacttatctaacctcagctttaccgattcttcctaagagagaagttgtaacgagagttcacacagcaagcacaactaatgttgtgactgatttttactccactactaccgaagttgttattgccccaactgtcgaattcctaataagcgatagtgttacctttacaacaaccttaattccacaaggtgtgaacccaacagctgaaccaactacgactatcactaaagttctcttaaaaaaggctgaaatgagtacatcttcgcagccaacctctactttgcagccaagcactattccacaatcaacctcctcttttcaagcagaatctactttgcaagccgtctctacgcaacaaactgctatgtctgtttctgcaggtactagtgaggatgtgcaacaattagctactacttctacctctatctcctcctctccttcccctaccactacgtctacttccacacaaaatatcgtgagtggtgtcggtgaagccaatgttgaaggcaacactcaaagtcagcatcattcttaccaagcagctgcgacttcaactttgaatcaacaaacgagtacctcaattgcttctcaggagtccaccgaatcaaccaatacacctacgtcgtcctctacgtcgtcctctacttcttcttctacttcttcttctacttcttcctctacttcttcttctacttcttcctctacttcgtcctctacttcgtcctctacttcatccacacaggaaacagccgctaccacctctgaaggttcctctagtagcagcgctgctataacttcttctccaaaggctattgcatattcaccatataatgatgatggctcttgtaagtctgcagacgctgtctcttctgacttaacgttgatcaaatctaaaggtatttccaaaatccgtgtttatggcaccgactgtaattcctttgaaaccgtccaaccagcagccgtaaaactaggtattaagattaaccaaggtttgtatatcaccagtagcggcgttgattccatagatgattctgttaccacattgattcaatatggtcaaactaacggttgggatgtctttgatttcattactgttggaaatgaagctatcaataatggttggtgttcagtttctgatttgattagtaaaatttcctctgtcaaatccaaattaagcgaagctggctattctggccaaattactaccagtgagccccctgtctcatttgaaaataaccctgatttgtgcaaaaaatctgatattgacttcgttggtatcaacccacatgcctactttgatactagcgccagtgctgaaactgccggtacatttgttaagggtcaagttgaattaattcaaggtgtttgtggtacttctaatgttttcgtcacggaaacaggttacccatcgtctggtattcaaaatggaggcaacattccatccactgctaaccaaattactgcagtccaaaacatcttgaatgaaatggacttggatgttacaatcttatcgacttacaatgattactggaaggctcccggtgattacggtatcgaacaatctttcggtgtgattgaatattttccataa

SEQ ID NO: 136 - protein, 542 aa, Saccharomyces cerevisiae
MISPISFLSSLLCLTYLTSALPILPKREVVTRVHTASTTNVVTDFYSTTTEVVIAPTVEFLISDSVTFTTTLIPQGVNPTAEPTTTITKVLLKKAEMSTSSQPTSTLQPSTIPQSTSSFQAESTLQAVSTQQTAMSVSAGTSEDVQQLATTSTSISSSPSPTTTSTSTQNIVSGVGEANVEGNTQSQHHSYQAAATSTLNQQTSTSIASQESTESTNTPTSSSTSSSTSSSTSSSTSSSTSSSTSSSTSSSTSSSTSSTQETAATTSEGSSSSSAAITSSPKAIAYSPYNDDGSCKSADAVSSDLTLIKSKGISKIRVYGTDCNSFETVQPAAVKLGIKINQGLYITSSGVDSIDDSVTTLIQYGQTNGWDVFDFITVGNEAINNGWCSVSDLISKISSVKSKLSEAGYSGQITTSEPPVSFENNPDLCKKSDIDFVGINPHAYFDTSASAETAGTFVKGQVELIQGVCGTSNVFVTETGYPSSGIQNGGNIPSTANQITAVQNILNEMDLDVTILSTYNDYWKAPGDYGIEQSFGVIEYFP